(19) United States
(12) Patent Application Publication
(10) Pub. No.: US 2025/0170266 A1
(43) Pub. Date: May 29, 2025

(54) IMMUNOMODULATOR PURINE-DERIVED COMPOUNDS, CONJUGATES THEREOF, AND METHODS OF USE THEREOF

(71) Applicant: ZYMEWORKS BC INC., Vancouver (CA)

(72) Inventors: Graham Albert Edwin Garnett, Vancouver (CA); Truman Hirkala-Schaefer, Vancouver (CA); Michael G. Brant, Vancouver (CA); Raffaele Colombo, Vancouver (CA); Zhuang Duan, Vancouver (CA); Stuart Daniel Barnscher, Vancouver (CA)

(21) Appl. No.: 19/033,344

(22) Filed: Jan. 21, 2025

Related U.S. Application Data
(63) Continuation of application No. PCT/CA2023/051006, filed on Jul. 26, 2023.
(60) Provisional application No. 63/392,329, filed on Jul. 26, 2022.

(51) Int. Cl. A61K 47/68; A61P 35/00; C07D 487/04; C07D 519/00

(52) U.S. Cl. CPC A61K 47/6879; A61K 47/6803; A61K 47/6889; A61P 35/00; C07D 487/04; C07D 519/00

(57) ABSTRACT

Purine-derived compounds of Formulae (I)-(IV), immunostimulatory antibody conjugates (ISACs) comprising such compounds, as well as methods for producing and using such compounds, e.g., for the treatment of diseases such as cancer, are disclosed.

### CROSS REFERENCE TO RELATED APPLICATIONS

This is a Continuation of PCT/CA2023/051006, filed Jul. 26, 2023, which claims priority to U.S. Provisional Patent Application No. 63/392,329, filed Jul. 26, 2022, both of which are hereby incorporated by reference.

### TECHNICAL FIELD

The present disclosure relates to the field of therapeutics and, in particular, to purine analogues, conjugates comprising such purine-derived compounds, their use for modulating an immune response in a cell or a subject, as well as the use of such compounds and conjugates for treatment of a disease (e.g., cancer, inflammation, etc.).

### BACKGROUND

Immunostimulatory compounds and immuno-conjugates (e.g., antibody-drug conjugates (ADCs) such as immune-stimulating antibody conjugates (ISACs)) comprising such compounds have previously been evaluated for the treatment of various diseases, including cancer. However, such compounds and conjugates remain clinically unvalidated, mostly due to dose-limiting cytotoxicity (e.g., cytokine-release syndrome (CRS)), which can restrict their use to doses that are unable to provide sufficient efficacy.

Furthermore, current immunotherapies lack efficacy specifically in solid tumors due to highly suppressive tumor microenvironments (TMEs) and associated lack of effector immune cells (e.g., T cells). Compounds that agonize Toll-like receptor 7 (TLR7) are immunostimulatory compounds capable of “reprogramming” a TME locally at a tumor site; however, significant tolerability issues remain unsolved which limits systemic administration of these compounds.

Although clinical data support the use of TLR7 agonists in oncology, such treatment requires local administration. For example, the TLR7 agonist imiquimod has been approved for topical use in dermal oncology applications, including basal cell carcinoma and actinic keratosis (see, Geisse et al. J Am Acad Dermatol. 2004; 50(5):722-33, and Korman et al. Arch Dermatol. 2005; 141(4):467-473). Other uses in invasive skin cancers (e.g., squamous cell carcinoma, Bowen's disease, melanoma, and/or lentigo maligna) are similarly efficacious when applied locally on surface lesions (see, Meyer et al. Expert Opin Investig Drugs. 2008; 17(7):1051-65, and Wolf et al. Arch Dermatol. 2003; 139(3):273-6).

Unfortunately, studies have reported that systemic administration of imiquimod and other TLR agonists leads to dose-limiting toxicity below efficacious doses (see, Dudek et al. Clin Cancer Res 2007; 13:7119-7125). Furthermore, overexpression and/or activation of some TLRs (including TLR7) can result in conflicting anti-tumor/pro-tumor activity, in some cases contributing to, rather than diminishing, inflammation, tumor growth, cell survival, metastasis, and the upregulation of pro-inflammatory cytokines (see, Kaczanowska S, et al. J Leukoc Biol. 2013; 93(6):847-863).

### SUMMARY

Disclosed herein are purine-derived compounds, e.g., compounds having a structure according to any one of Formulae (I)-(IV) herein, as well as conjugates comprising such compounds, e.g., conjugates according to Formula (X). Furthermore, the present disclosure discloses methods of producing the herein described compounds and conjugates and their use for, e.g., the treatment of a disease such as cancer.

As further described herein, in vitro agonism of TLR7 using the compounds and conjugates of the present disclosure has been demonstrated by incubating immune cells expressing TLR7 (e.g., PBMCs) with the compounds and conjugates of the present disclosure and measuring one or more downstream effects of TLR7 agonism, such as cytokine induction (see, e.g., EXAMPLE 3). Moreover, in vivo immunostimulatory antibody-drug conjugate (ISAC) therapy utilizing antibodies targeting tumor-associated antigens (e.g., Her2) conjugated to at least one of the disclosed compounds can generate a potent anti-tumor response, as evidenced by inhibition of tumor growth rate and/or reduction in tumor volume (see, e.g., EXAMPLE 6). Surprisingly, a significant reduction in off-target effects—and hence an increased tolerability—was observed during in vivo testing of certain ISACs comprising a TLR7-agonizing compound of the present disclosure, when compared to the use of conventional immunostimulatory compounds (see, e.g., EXAMPLE 7).

More specifically, in various embodiments, the present disclosure relates to a compound according to Formula (I):
(I)

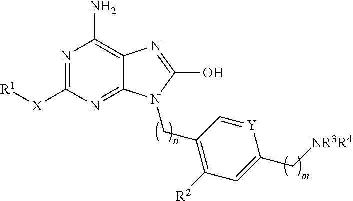

a tautomer, a protected version, and/or a pharmaceutically acceptable salt thereof, wherein:
X is O or NH;
Y is N or CH;
R¹ is optionally substituted C₂-C₆ alkyl or branched and optionally substituted C₃-C₈ hydroxyalkyl;
R² is H, halogen, optionally substituted C₁-C₆ alkyl, or optionally substituted C₁-C₆ alkoxy;
R³ and R⁴ are independently H, Q-R⁵, or R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

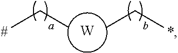

wherein * is the point of attachment to R⁵ and #is the point of attachment to N;
R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R⁶ and R⁷ are independently H, optionally substituted C₁-C₆ alkyl, optionally substituted C₁-C₆ alkoxycarbonyl, or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
n is an integer from 1 to 4;
m is an integer from 0 to 4; and
a and b are independently integers from 0 to 4,
with the proviso that when R² is H, then:
   (i) R¹ is branched and optionally substituted C₃-C₈ hydroxyalkyl;
   (ii) X is NH and R¹ is optionally substituted C₅-C₆ alkyl; and/or
   (iii) m is 0 or 1 and R³ and R⁴ together with the N form an unsubstituted piperazinyl ring or an optionally substituted C₆-C₁₀-heterospirocycloalkyl ring.

In some embodiments, a compound of Formula (I) can have a structure according to Formula (II):

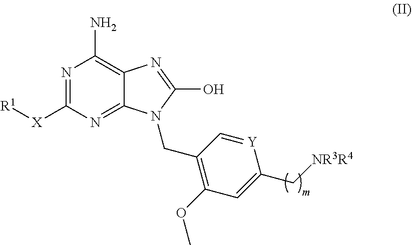

a tautomer, a protected version, and/or a pharmaceutically acceptable salt thereof, wherein:
X is O or NH;
Y is N or CH;
R¹ is optionally substituted C₂-C₆ alkyl or branched and optionally substituted C₃-C₈ hydroxyalkyl;
R³ and R⁴ are independently H, Q-R⁵, or R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

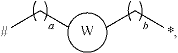

wherein * is the point of attachment to R⁵ and #is the point of attachment to N;
R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R⁶ and R⁷ are independently H, optionally substituted C₁-C₆ alkyl, optionally substituted C₁-C₆ alkoxycarbonyl, or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
m is an integer from 0 to 4; and
a and b are independently integers from 0 to 4.

In various embodiments, the present disclosure relates to compounds of Formula (III):

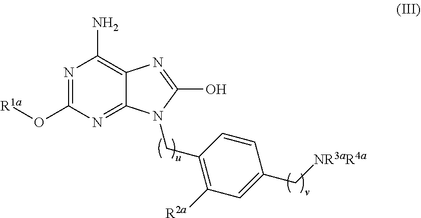

a tautomer, a protected version, and/or a pharmaceutically acceptable salt thereof, wherein:
R^{1a} is optionally substituted C₂-C₆ alkyl or branched and optionally substituted C₃-C₈ hydroxyalkyl;
R^{2a} is halogen, optionally substituted C₁-C₆ alkyl, or optionally substituted C₁-C₆ alkoxy;
R^{3a} and R^{4a} are independently H, Q-R^{5a}, or R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

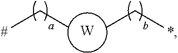

wherein * is the point of attachment to R⁵ and #is the point of attachment to N;
R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R^{6a} and R^{7a} are independently H, optionally substituted C₁-C₆ alkyl, optionally substituted C₁-C₆ alkoxycarbonyl, or R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
u is an integer from 1 to 3;
v is an integer from 0 to 4; and
a and b are independently integers from 0 to 4.

In certain embodiments of the present disclosure, a compound according to Formula (III) can have a structure according to Formula (IV):

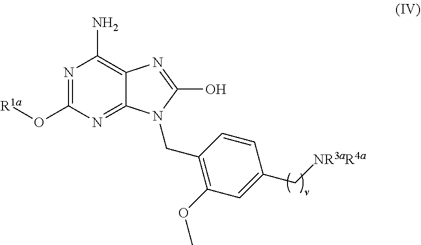

wherein:
R^{1a} is unsubstituted C₂-C₆ alkyl or branched and unsubstituted C₃-C₈ hydroxyalkyl;
R^{3a} and R^{4a} are independently H, Q-R^{5a}, or R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

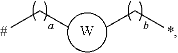

wherein * is the point of attachment to R⁵ and #is the point of attachment to N;
R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R^{6a} and R^{7a} are independently H, optionally substituted C₁-C₆ alkyl, optionally substituted C₁-C₆ alkoxycarbonyl, or R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
v is an integer from 0 to 4; and
a and b are independently integers from 0 to 4.

In some embodiments, the present disclosure relates to methods of agonizing TLR7, the method comprising contacting a cell that expresses TLR7 with a compound of any one of Formulae (I)-(IV), thereby agonizing TLR7.

In some embodiments, the present disclosure relates to a method of inducing release of a cytokine from a cell expressing TLR7, the method comprising contacting the cell with a compound of any one of Formulae (I)-(IV), thereby inducing release of the cytokine from the cell.

In some embodiments, the present disclosure relates to a method of inhibiting the proliferation of cancer cells, the method comprising contacting a cell population comprising the cancer cells and immune cells expressing TLR7 with an effective amount of a compound of any one of Formulae (I)-(IV). Such method can further comprise agonizing the TLR7 with the compound of any one of Formulae (I)-(IV), thereby inhibiting the proliferation of cancer cells.

In some embodiments, this disclosure relates to a method of killing cancer cells, the method comprising contacting a cell population comprising the cancer cells and immune cells expressing TLR7 with an effective amount of a compound of any one of Formulae (I)-(IV). Such method can further comprise agonizing the TLR7 with the compound of any one of Formulae (I)-(IV), thereby killing the cancer cells.

Other embodiments of the present disclosure relate to a method of stimulating an immune response in a subject in need thereof, the method comprising administering to the subject an effective amount of a compound of any one of Formulae (I)-(IV). In some embodiments, the compound agonizes TLR7 in the subject, thereby stimulating the immune response in the subject.

Further embodiments of this disclosure relate to a method of treating a cancer in a subject in need thereof, the method comprising administering to the subject an effective amount of a compound of any one of Formulae (I)-(IV). Such method can further comprise agonizing TLR7 in the subject, thereby treating the cancer in the subject.

In various embodiments, the present disclosure relates to conjugates comprising one or more compounds of any one of Formulae (I)-(IV). In various embodiments, such conjugate has a structure according to Formula (X):

T-[L-(C)ₚ]ᵣ (X)

wherein:
T is a targeting moiety;
L is a linker;
C is a compound of any one of Formulae (I)-(IV);
p is an integer from 1 to 5; and
r has a value from about 1 to about 8.

In various embodiments, the targeting moiety is an antibody or an antigen binding fragment thereof.

The present disclosure also relates to methods of agonizing TLR7, the method comprising contacting a cell that expresses TLR7 with a conjugate of Formula (X), thereby agonizing TLR7.

Further embodiments herein relate to a method of inducing release of a cytokine from a cell expressing TLR7, the method comprising contacting the cell with a conjugate of Formula (X), thereby inducing release of the cytokine from the cell.

Further embodiments herein relate to a method of inhibiting the proliferation of cancer cells, the method comprising contacting a cell population comprising the cancer cells and immune cells expressing TLR7 with an effective amount of a conjugate of Formula (X). Such method can further comprise agonizing the TLR7 with a compound of the conjugate of Formula (X), thereby inhibiting the proliferation of cancer cells.

Other embodiments of this disclosure relate to a method of killing cancer cells, the method comprising contacting a cell population comprising the cancer cells and immune cells expressing TLR7 with an effective amount of a conjugate of Formula (X). Such method can further comprise agonizing the TLR7 with a compound of the conjugate of Formula (X) (e.g., a compound of any one of Formulae (I)-(IV) that is comprised by the conjugate), thereby killing the cancer cells.

Further embodiments herein relate to a method of stimulating an immune response in a subject in need thereof, the method comprising administering to the subject an effective amount of a conjugate of Formula (X). In some embodiments, the conjugate agonizes TLR7, e.g., via a compound of Formulae (I)-(IV), in the subject, thereby stimulating the immune response in the subject.

Further embodiments of this disclosure relate to a method of treating a cancer in a subject in need thereof, the method comprising administering to the subject an effective amount of a conjugate of Formula (X). In some embodiments, the conjugate agonizes TLR7, e.g., via a compound of Formulae (I)-(IV), in the subject, thereby treating the cancer in the subject.

### BRIEF DESCRIPTION OF THE DRAWINGS

The implementations disclosed herein are illustrated by way of example, and not by way of limitation, in the accompanying drawings. The description and drawings are only for the purpose of illustration and as an aid to understanding and are not intended as a definition of the limits of the compounds, conjugates, and methods of the present disclosure.

FIG. 1 shows a general synthetic scheme for producing the purine-derived compounds (e.g., compounds of Formulae (I)-(IV)) according to some embodiments of this disclosure.

FIG. 2 shows the anti-tumor activity of tested ISACs over the course of a murine in vivo study as an inhibition of tumor growth and/or as a reduction in tumor volume, following intravenous (iv) administration of 2.5 mg/kg of the respective ISAC or control article.

FIG. 3 shows the change in body weight of the animals tested in the in vivo study referred to in FIG. 2, as a measurement for tolerability of the tested ISACs.

FIGS. 4A-4G show results of a tolerability study conducted using the following ISACs: Tras-MT-VK-PABC-Compound 101 (FIG. 4A), Tras-MT-VK-PABC-Compound 106 (FIG. 4B), Tras-MT-VC-PABC-Compound 154 (FIG. 4C), Tras-MT-VC-PABC-Compound 155 (FIG. 4D), and Trastuzumab-ME2-Compound 100 (FIG. 4E), at different dose levels and in relation to the trastuzumab-benchmark conjugate (FIG. 4F) as well as unconjugated trastuzumab (abbreviated herein as “Tras”) and vehicle (FIG. 4G).

FIG. 5 shows a correlation between tumor growth rate inhibition shown in FIG. 2 and murine IL-6 (mIL-6) induction for tested ISACs, according to some embodiments of the present disclosure.

### DETAILED DESCRIPTION

In various embodiments, the present disclosure relates to immunostimulatory purine-derived compounds having a structure according to any one of Formulae (I)-(IV) that are capable of agonizing TLR7. Also disclosed herein are compound-linker constructs of Formula (A) that comprise a compound of the present disclosure coupled to a linker moiety. Further disclosed herein are immunostimulatory conjugates of Formula (X) comprising one or more of the purine-derived compounds described herein (e.g., those of Formulae (I)-(IV)) coupled to a targeting moiety via a linker, e.g., by using a compound-linker construct of Formula (A) as described herein. Methods for making and using the compounds, compound-linker constructs, and conjugates, e.g., for the treatment of cancer, are also disclosed.

### Definitions

Unless defined otherwise, all technical and scientific terms used herein have the same meaning as commonly understood by one of ordinary skill in the art.

The term “about,” as used herein in the context of a numerical value or range, generally refers to ±10%, 9%, 8%, 7%, 6%, 5%, 4%, 3%, 2%, or 1% of the numerical value or range recited or claimed, unless otherwise specified. In various embodiments, the term “about” refers to an approximately ±10% variation from a given value. In other embodiments, the term “about” refers to an approximately ±5% variation from a given value. In yet other embodiments, the term “about” refers to an approximately ±1% variation from a given value. It is to be understood that such a variation is always included in any given value described herein, whether it is specifically referred to or not.

The use of the word “a” or “an” when used herein in conjunction with the term “comprising” can mean “one,” but it is also consistent with the meaning of “one or more,” “at least one” and “one or more than one.”

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a compound, composition, use or method, denotes that additional elements and/or method steps can be present, but that these additions do not materially affect the manner in which the recited compound, composition, method or use functions. The term “consisting of,” when used herein in connection with a compound, composition, use or method, excludes the presence of additional elements and/or method steps. A compound, composition, use, or method described herein as comprising certain elements and/or steps can also, in certain embodiments, consist essentially of those elements and/or steps, and, in other embodiments, consist of those elements and/or steps, whether or not these embodiments are specifically referred to.

The terms “alkyloxycarbonyl” and “alkoxycarbonyl” can be used interchangeably herein and refer to the group —C(O)OR, wherein R is alkyl.

The term “alkyl,” as used herein, refers to a straight chain or branched saturated hydrocarbon group containing the specified number of carbon atoms. Examples of alkyl include, but are not limited to, methyl, ethyl, n-propyl, isopropyl, n-butyl, sec-butyl, isobutyl, t-butyl, pentyl, isopentyl, t-pentyl, neo-pentyl, 1-methylbutyl, 2-methylbutyl, n-hexyl, and the like.

The term “alkylcycloalkyl,” as used herein, refers to an alkyl group as defined herein substituted with one cycloalkyl group as defined herein. Examples include (C₁-C₆ alkyl)-cycloalkyl, e.g., (C₁-C₂ alkyl)-cycloalkyl or (C₁-C₄ alkyl)-cycloalkyl. Accordingly, the terms “alkylheterocycloalkyl,” “alkylaryl” and “alkylheteroaryl, as used herein, refer to an alkyl group as defined herein substituted with one heterocycloalkyl group, aryl group, or heteroaryl group, respectively, and as further defined herein. Examples include (C₁-C₆ alkyl)-heterocycloalkyl, (C₁-C₆ alkyl)-aryl and (C₁-C₆ alkyl)-heteroaryl. In various embodiments, any cycloalkyl, heterocycloalkyl, aryl, or heteroaryl group can itself be substituted with one or more of —NH₂, —CO₂H, —OH, carbonyl, halogen, C₁-C₂ alkyl, C₁-C₂ hydroxyalkyl, (C₀-C₂ alkyl)-cycloalkyl, (C₀-C₂ alkyl)-heterocycloalkyl, (C₀-C₂ alkyl)-aryl, and (C₀-C₂ alkyl)-heteroaryl, as described herein.

The term “amido,” as used herein, refers to the group —C(O)NRR′, where R and R′ are independently hydrogen, alkyl, aryl, heteroaryl, cycloalkyl or heterocycloalkyl.

The term “amino,” as used herein, refers to the group —NRR′, wherein R and R′ are independently hydrogen, alkyl, aryl, heteroaryl, cycloalkyl or heterocycloalkyl.

The term “carboxy,” as used herein, refers to the group —C(O)OR, wherein R is H, alkyl, aryl, heteroaryl, cycloalkyl or heterocycloalkyl.

The term “haloalkyl,” as used herein, refers to an alkyl group as defined herein substituted with one or more halogen atoms.

The terms “halogen” and “halo,” as used herein, refer to fluorine (F), bromine (Br), chlorine (Cl) and iodine (I).

The term “aminoalkyl,” as used herein, refers to an alkyl group as defined herein substituted with one or more amino groups, for example, one, two or three amino groups.

The term “aminoaryl,” as used herein, refers to an aryl group as defined herein substituted with one amino group.

The term “aryl,” as used herein, and unless otherwise defined, refers to a 6- to 12-membered mono- or bicyclic hydrocarbon ring system in which at least one ring is aromatic.

Examples of aryl include, but are not limited to, phenyl, naphthalenyl, 1,2,3,4-tetrahydro-naphthalenyl, 5,6,7,8-tetrahydro-naphthalenyl, indanyl, and the like.

The term “cycloalkyl,” as used herein and unless otherwise defined, refers to a mono- or bicyclic saturated hydrocarbon containing the specified number of carbon atoms. Examples of cycloalkyl include, but are not limited to, cyclopropyl, cyclobutyl, cyclopentyl, cyclohexyl, cycloheptane, bicyclo[1.1.1]pentane, bicyclo[2.2.1]heptane, bicyclo[3.1.1]heptane, and the like.

The term “heteroaryl,” as used herein and unless otherwise defined, refers to a 5- to 12-membered mono- or bicyclic ring system containing the specified number of carbon atoms (e.g., C₃-C₇, incl., e.g., imidazole, thiazole, etc.) and in which at least one ring atom is a heteroatom and at least one ring is aromatic. Examples of heteroatoms include, but are not limited to, O, S and N.

Examples of heteroaryl include, but are not limited to: pyridyl, benzofuranyl, pyrazinyl, pyridazinyl, pyrimidinyl, triazinyl, quinolinyl, benzoxazolyl, benzothiazolyl, isoquinolinyl, quinazolinyl, quinoxalinyl, pyrrolyl, indolyl, and the like.

The term “heterocycloalkyl,” as used herein, refers to a mono- or bicyclic non-aromatic ring system containing the specified number of carbon atoms (e.g., C₂-C₆, incl., e.g., aziridine, piperazine, etc.) and in which at least one ring atom is a heteroatom, for example, O, S or N. A heterocyclyl substituent can be attached via any of its available ring atoms, for example, a ring carbon, or a ring nitrogen. Examples of heterocycloalkyl include, but are not limited to, aziridinyl, azetidinyl, piperidinyl, morpholinyl, piperazinyl, pyrrolidinyl, and the like.

As used herein with reference to an alkyl or heteroalkyl ring system, the term “bicyclic” generally includes both fused and spiro ring systems, unless otherwise defined herein.

The terms “hydroxy” and “hydroxyl,” as used herein, refer to the group —OH.

The term “hydroxyalkyl,” as used herein, refers to a linear or branched alkyl group as defined herein substituted with one or more hydroxy groups. In some embodiments, such one or more hydroxy groups can be terminal hydroxy groups.

The term “alkylthio,” as used herein, refers to the group —SR, where R is a C₁-C₆ alkyl group. In some embodiments, the alkyl group can optionally be substituted with one or more of —NH₂, —CO₂H, —OH, carbonyl, halogen, C₁-C₂ alkyl and C₁-C₂ hydroxyalkyl.

The terms “thio” and “thiol,” as used herein, refer to the group —SH.

Unless specifically stated as being “unsubstituted,” any alkyl (e.g., any haloalkyl, hydroxyalkyl, aminoalkyl, etc.), cycloalkyl, heterocycloalkyl, spirocycloalkyl, heterospirocycloalkyl, aryl or heteroaryl group referred to herein is understood to be “optionally substituted,” i.e., each such reference includes both unsubstituted and substituted versions of these groups. For example, reference to a “—C₁-C₆ alkyl” group includes both unsubstituted —C₁-C₆ alkyl and —C₁-C₆ alkyl substituted with one or more substituents further described herein.

In various embodiments, a substituent of any one of Formulae (I)-(IV) in any of the embodiments herein can be optionally substituted with one or more groups selected from the following: —NH₂, —CO₂H, —OH, carbonyl, halogen, C₁-C₄ alkyl, C₁-C₄ hydroxyalkyl, C₁-C₄ aminoalkyl, C₁-C₄ carboxyalkyl, (C₀-C₄ alkyl)-cycloalkyl, (C₀-C₄ alkyl)-heterocycloalkyl, (C₀-C₄ alkyl)-spirocycloalkyl, (C₀-C₄ alkyl)-heterospirocycloalkyl, (C₀-C₄ alkyl)-aryl, and (C₀-C₄ alkyl)-heteroaryl, wherein each of the alkyl, hydroxyalkyl, C₁-C₄ aminoalkyl, C₁-C₄ carboxyalkyl, cycloalkyl, heterocycloalkyl, spirocycloalkyl, heterospirocycloalkyl, aryl or heteroaryl group can itself be substituted with one or more of: —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl, or unsubstituted C₁-C₄ carboxyalkyl.

In other embodiments, a substituent of any one of Formulae (I)-(IV) in any of the embodiments herein can be optionally substituted with one or more groups selected from the following: —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl, unsubstituted C₁-C₄ carboxyalkyl, unsubstituted (C₀-C₄ alkyl)-cycloalkyl, unsubstituted (C₀-C₂ alkyl)-heterocycloalkyl, unsubstituted (C₀-C₂ alkyl)-aryl, and unsubstituted (C₀-C₂ alkyl)-heteroaryl.

In yet other embodiments, a substituent of any one of Formulae (I)-(IV) in any of the embodiments herein can be optionally substituted with one or more groups selected from the following: —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl and unsubstituted C₁-C₄ carboxyalkyl.

Generally, a substituent of any one of Formulae (I)-(IV) described herein as “substituted,” can include one substituent or a plurality of substituents up to the full valence of substitution for that group. For example, a methyl group can include 1, 2, or 3 substituents, and a phenyl group can include 1, 2, 3, 4, or 5 substituents. When a group is substituted with more than one substituent, the substituents can be the same or they can be different.

Throughout the present disclosure, and unless otherwise specified, the following system for numbering ring atoms in the purine moiety of the compounds described herein was used ( [symbol] indicates potential attachment points to substituent(s)):

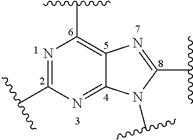 [SMILES: *c1nc(*)c2nc(*)n(*)c2n1]

The terms “subject” and “patient” can be used interchangeably herein and refer to an animal in need of treatment. An animal in need of treatment can be a human or a non-human animal, such as a mammal, bird, or fish. In certain embodiments, the subject or patient is a mammal. In some embodiments, the subject is a human. In other embodiments, the subject is a rodent or a non-human primate.

An “effective amount” of a compound or conjugate described herein in respect of a particular result to be achieved is an amount sufficient to achieve the desired result. For example, an “effective amount” of a compound when referred to in respect of the killing of cancer cells, refers to an amount of that compound sufficient to produce a killing effect.

It is further to be understood that the positive recitation of a feature in one embodiment, serves as a basis for excluding the feature in an alternative embodiment. In particular, where a list of options is presented for a given embodiment or claim, it is to be understood that one or more option can be deleted from the list and the shortened list can form an alternative embodiment, whether or not such an alternative embodiment is specifically referred to.

It is contemplated that any embodiment discussed herein can be implemented with respect to any method, use or composition disclosed herein, and vice versa.

### Compounds of the Disclosure

In various embodiments, the present disclosure discloses purine-derived compounds capable of agonizing TLR7.

In some embodiments, the present disclosure relates to a compound having Formula (I):

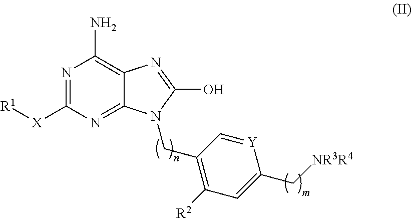

a tautomer, a protected version, and/or a pharmaceutically acceptable salt thereof, wherein:
X is O or NH;
Y is N or CH;
R¹ is optionally substituted C₂-C₆ alkyl or branched and optionally substituted C₃-C₈ hydroxyalkyl;
R² is H, halogen, optionally substituted C₁-C₆ alkyl, or optionally substituted C₁-C₆ alkoxy;
R³ and R⁴ are independently H, Q-R⁵, or R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

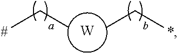

wherein * is the point of attachment to R⁵ and #is the point of attachment to N;
R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R⁶ and R⁷ are independently H, optionally substituted C₁-C₆ alkyl, optionally substituted C₁-C₆ alkoxycarbonyl, or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
n is an integer from 1 to 4;
m is an integer from 0 to 4; and
a and b are independently integers from 0 to 4,
with the proviso that when R² is H, then:
   (i) R¹ is branched and optionally substituted C₃-C₈ hydroxyalkyl;
   (ii) X is NH and R¹ is optionally substituted C₅-C₆ alkyl; and/or
   (iii) m is 0 or 1 and R³ and R⁴ together with the N form an unsubstituted piperazinyl ring or an optionally substituted C₆-C₁₀-heterospirocycloalkyl ring.

In some embodiments, in compounds of Formula (I), R² is halogen, optionally substituted C₁-C₆ alkyl, or optionally substituted C₁-C₆ alkoxy. In some of these embodiments, R² is halogen or optionally substituted C₁-C₆ alkoxy. In certain embodiments, R² is halogen or unsubstituted C₁-C₆ alkoxy. In some embodiments, R² can be fluorine or methoxy. In some embodiments, R² is fluorine. In other embodiments, R² is methoxy.

Disclosed herein are compounds of Formula (I) in which X is O. In other embodiments, in compounds of Formula (I), X is NH.

In some embodiments, in compounds of Formula (I), R¹ is optionally substituted C₂-C₆ alkyl. In some embodiments, R¹ is unsubstituted C₂-C₆ alkyl, such as unsubstituted C₂-C₄ alkyl. In these embodiments, X can be O.

In yet other embodiments, R¹ is branched and optionally substituted C₃-C₈ hydroxyalkyl. In some embodiments, R¹ can be branched and unsubstituted C₃-C₈ hydroxyalkyl. In some embodiments, R¹ is branched and unsubstituted C₄-C₆ hydroxyalkyl.

In such embodiments, the compound of Formula (I) can be:

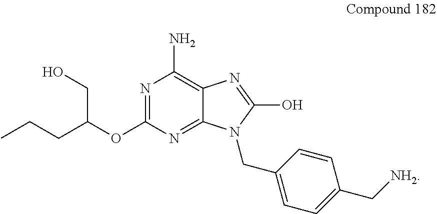 [SMILES: CCCC(CO)Oc1nc(N)c2nc(O)n(Cc3ccc(CN)cc3)c2n1]

In some embodiments, in compounds of Formula (I), R³ and R⁴ are independently H or Q-R⁵. In some embodiments, R³ and R⁴ can both be H. In other embodiments, R³ and R⁴ are both Q-R⁵. In some embodiments in which R³ and R⁴ are both Q-R⁵, Q can be optionally substituted C₁-C₆-alkyl, and R⁵ can be H or OH. In some embodiments in which R³ and R⁴ are both Q-R⁵, Q is unsubstituted C₁-C₆-alkyl and R⁵ is H.

In other embodiments, in compounds of Formula (I), Q is a bond and R⁵ is unsubstituted C₃-C₆-carboxyalkyl.

In various other embodiments, in compounds of Formula (I), Q is a bond, optionally substituted C₁-C₆-alkyl, optionally substituted C₂-C₆-alkenyl or

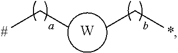

wherein * is the point of attachment to R⁵ and #is the point of attachment to N.

In some embodiments in which both R³ and R⁴ are H, the compound of Formula (I) can be:

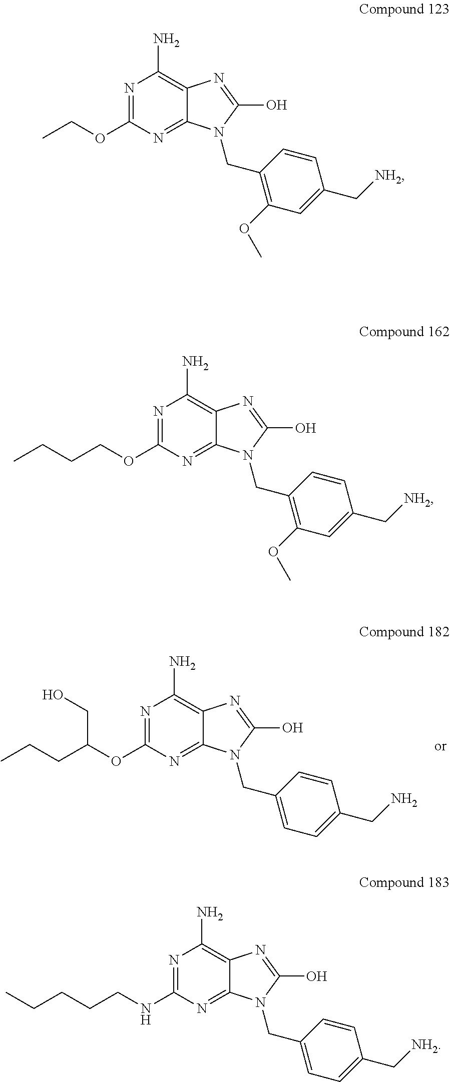 [SMILES: CCOc1nc(N)c2nc(O)n(Cc3ccc(CN)cc3OC)c2n1 ; CCCCOc1nc(N)c2nc(O)n(Cc3ccc(CN)cc3OC)c2n1 ; CCCC(CO)Oc1nc(N)c2nc(O)n(Cc3ccc(CN)cc3)c2n1 ; CCCCCNc1nc(N)c2nc(O)n(Cc3ccc(CN)cc3)c2n1]

In some embodiments in which both R³ and R⁴ are Q-R⁵, the compound of Formula (I) can be:

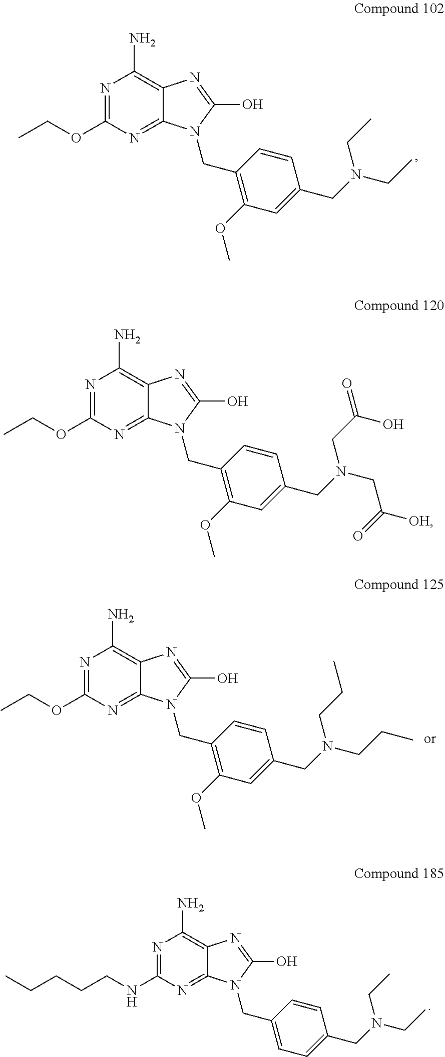

In yet other embodiments, in compounds of Formula (I), R³ is H and R⁴ is Q-R⁵. In such embodiments, Q can be a bond and R⁵ can be optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In such embodiments, the compound of Formula (I) can be selected from the compounds shown in SUBTABLE 1C.

In some embodiments, in compounds of Formula (I), R³ is H and R⁴ is Q-R⁵, wherein Q is optionally substituted C₁-C₆-alkyl and R⁵ is H, OH, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In some embodiments, Q is unsubstituted C₁-C₆-alkyl. In other embodiments, Q is C₁-C₆-alkyl substituted with one or more of —NH₂, —CO₂H, —OH, carbonyl, halogen, C₁-C₂ alkyl, C₁-C₂ hydroxyalkyl, (C₀-C₂ alkyl)-cycloalkyl, (C₀-C₂ alkyl)-heterocycloalkyl, (C₀-C₂ alkyl)-aryl, and (C₀-C₂ alkyl)-heteroaryl. In other embodiments, Q is C₁-C₆-alkyl substituted with one or more of —NH₂, —CO₂H, —OH, carbonyl, halogen, C₁-C₂ alkyl, and C₁-C₂ hydroxyalkyl. In some embodiments, the compound of Formula (I) can be selected from the compounds shown in SUBTABLE 1D.

In some embodiments, in compounds of Formula (I), R³ is H and R⁴ is Q-R⁵, wherein Q is optionally substituted C₂-C₆-alkenyl and R⁵ is H, OH, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In some embodiments, Q is unsubstituted C₂-C₆-alkenyl.

In various embodiments, in compounds of Formula (I), in which one or more of R³ and R⁴ is Q-R⁵, Q is C₁-C₆-alkyl substituted with one or more of C₃-C₆-cycloalkyl, C₂-C₆-heterocycloalkyl, C₃-C₇-heterobicycloalkyl, C₆-C₁₀-heterospirocycloalkyl, aryl, or heteroaryl. In some of these embodiments, such ring substituent (referred to below as “W”) can be a bridging moiety so that Q is:

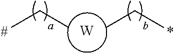

wherein:
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl; and
a and b are independently integers from 0 to 4,
   and wherein * is the point of attachment to R⁵ and #is the point of attachment to N.

In some of these embodiments, W is unsubstituted C₃-C₆-cycloalkyl, unsubstituted C₂-C₆-heterocycloalkyl, unsubstituted C₃-C₇-heterobicycloalkyl, unsubstituted C₆-C₁₀-heterospirocycloalkyl, unsubstituted aryl, or unsubstituted heteroaryl. In such embodiments, Q can be selected from the group consisting of:

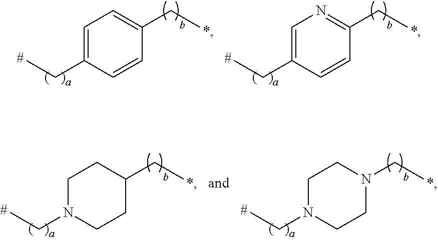

wherein a and b are independently 1, 2, or 3.

In some embodiments, in compounds of Formula (I), R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring, or an optionally substituted heteroaryl ring. In some embodiments, R³ and R⁴ together with the N form a C₃-C₆-heterocycloalkyl ring, a C₃-C₆-heterobicycloalkyl ring, a C₆-C₁₀-heterospirocycloalkyl ring, or a heteroaryl ring, wherein the ring moiety is optionally substituted with one or more of —NH₂, —CO₂H, —OH, carbonyl, halogen, alkylthio, C₁-C₂ alkyl, C₁-C₂ hydroxyalkyl, (C₀-C₂ alkyl)-cycloalkyl, and (C₀-C₂ alkyl)-heterocycloalkyl.

In some embodiments, R³ and R⁴ together with the N form a ring selected from the group consisting of:

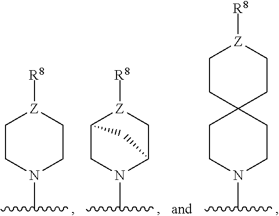

wherein:
Z is N or CH; and
R⁸ is H, NH₂, optionally substituted C₁-C₆-alkyl, optionally substituted C₁-C₆-heteroalkyl, optionally substituted C₁-C₆-aminoalkyl, or optionally substituted C₂-C₆-heterocycloalkyl.

In some embodiments, in compounds of Formula (I), R³ and R⁴ together with the N form a ring selected from the group consisting of:

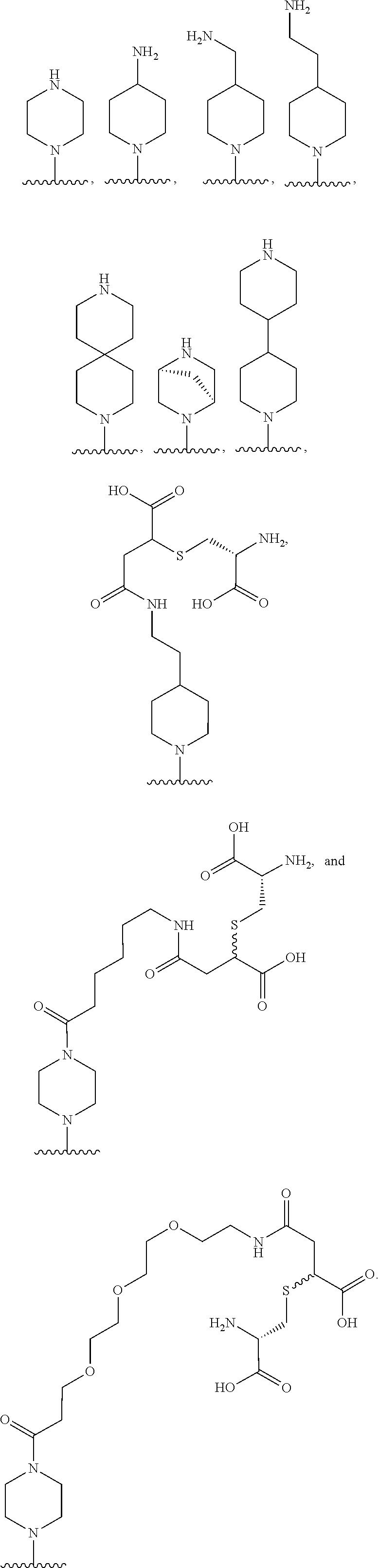

In some embodiments, in compounds of Formula (I), R³ and R⁴ together with the N form a ring moiety as described herein, and wherein the compound is selected from the compounds shown in SUBTABLE 1B.

In some embodiments, in compounds of Formula (I) in which at least one of R³ and R⁴ is Q-R⁵, R⁵ can be NR⁶R⁷. In such embodiments, R⁶ and R⁷ can be independently H, optionally substituted C₁-C₆ alkyl or optionally substituted C₁-C₆ alkoxycarbonyl. In some embodiments, R⁶ and R⁷ are both H. In some embodiments, R⁶ and R⁷ are both optionally substituted C₁-C₆ alkyl, and wherein R⁶ is identical to R⁷. In such embodiments, R⁶ and R⁷ can both be C₁-C₆ alkyl, substituted with one or more of —NH₂, —CO₂H, —OH, carbonyl, halogen, C₁-C₂ alkylthio, C₁-C₂ alkyl, and C₁-C₂ hydroxyalkyl. In some embodiments, R⁶ and R⁷ are C₁-C₂ alkyl substituted with one or more of —NH₂, —CO₂H, —OH, or halogen. In other embodiments, R⁶ is H and R⁷ is optionally substituted C₁-C₆ alkyl. In some embodiments, R⁶ is H and R⁷ can be C₁-C₆ alkyl substituted with one or more of —NH₂, —CO₂H, —OH, carbonyl, halogen, C₁-C₂ alkylthio, C₁-C₂ alkyl, C₁-C₂ hydroxyalkyl, optionally substituted C₃-C₆-cycloalkyl, optionally substituted C₂-C₆-heterocycloalkyl, optionally substituted aryl, and optionally substituted heteroaryl.

In some embodiments, in compounds of Formula (I) in which at least one of R³ and R⁴ is Q-R⁵, R⁵ can be NR⁶R⁷, wherein R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring, or an optionally substituted heteroaryl ring. In some embodiments, such ring moiety can be optionally substituted with one or more of —NH₂, —CO₂H, —OH, carbonyl, halogen, C₁-C₂ alkylthio, C₁-C₂ alkyl, C₁-C₃ aminoalkyl and C₁-C₂ hydroxyalkyl. In some embodiments, R⁶ and R⁷ together with the N form a ring selected from the group consisting of:

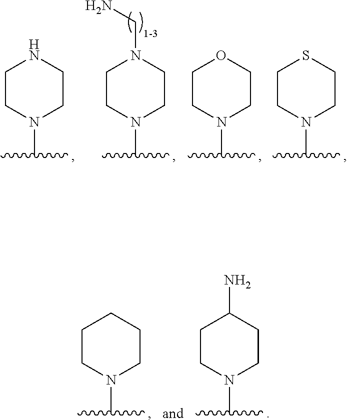

In some embodiments, a compound of Formula (I) is selected from the compounds shown in SUBTABLE 1E.

In some embodiments, in compounds of Formula (I), n is 1.

In some embodiments, in compounds of Formula (I), m is an integer from 0 to 3 or from 1 to 3. In some embodiments, m is 0. In some embodiments, m is 1. In some embodiments, m is 2. In yet other embodiments, m is 3.

In some embodiments, in compounds of Formula (I), R² is H and (a) R¹ is branched and optionally substituted C₃-C₈ hydroxyalkyl, and/or (b) X is NH and R¹ is optionally substituted C₅-C₆ alkyl, and/or (c) m is 0 or 1 and R³ and R⁴ together with the N form an unsubstituted piperazinyl ring or an optionally substituted C₆-C₁₀-heterospirocycloalkyl ring. In some embodiments, R² is H and R¹ is branched and optionally substituted C₃-C₈ hydroxyalkyl. In such embodiments, R¹ can be branched and unsubstituted C₃-C₈ hydroxyalkyl. In other embodiments, R² is H, X is NH, and R¹ is optionally substituted C₅-C₆ alkyl. In some of those embodiments, R¹ is unsubstituted C₅-C₆ alkyl. In yet other embodiments, R² can be H, m can be 0 or 1, and R³ and R⁴ together with the N can form an unsubstituted piperazinyl ring or an optionally substituted C₆-C₁₀-heterospirocycloalkyl ring. In some embodiments, R³ and R⁴ together with the N form an unsubstituted piperazinyl ring. In yet other embodiments, R³ and R⁴ together with the N form an optionally substituted C₆-C₁₀-heterospirocycloalkyl ring.

In some embodiments, a compound of Formula (I) is selected from TABLE 1 herein, which comprises SUBTABLES 1A-1F.

In certain embodiments of the present disclosure, a compound of Formula (I) has the structure of Formula (II):

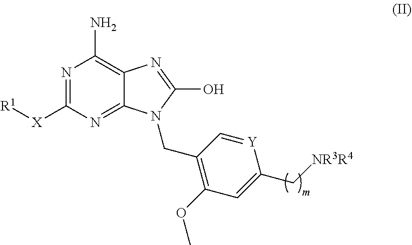

wherein:
X is O or NH;
Y is N or CH;
R¹ is optionally substituted C₂-C₆ alkyl or branched and optionally substituted C₃-C₈ hydroxyalkyl;
R³ and R⁴ are independently H, Q-R⁵, or R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

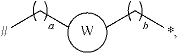

wherein * is the point of attachment to R⁵ and #is the point of attachment to N;
R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R⁶ and R⁷ are independently H, optionally substituted C₁-C₆ alkyl, optionally substituted C₁-C₆ alkoxycarbonyl, or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
m is an integer from 0 to 4; and
a and b are independently integers from 0 to 4.

In some embodiments, in compounds of Formula (II), X is O and R¹ is optionally substituted C₂-C₆ alkyl. In some of these embodiments, R¹ is unsubstituted C₂-C₆ alkyl. In some specific embodiments, R¹ is ethyl or n-butyl.

In some embodiments, in compounds of Formula (II), Y is CH.

In some embodiments, in compounds of Formula (II), m is 0 or 1, 1 or 2, or 1 or 3. In such embodiments, m can be 0. In other embodiments, m is 1, 2, or 3. In some embodiments, m is 1. In some embodiments, m is 2. In yet other embodiments, m is 3.

In some embodiments, in compounds of Formula (II), R³ and R⁴ are both H. In other embodiments, R³ and R⁴ are both Q-R⁵. In such embodiments, Q can be optionally substituted C₁-C₆-alkyl, and R⁵ can be H or OH. In some of these embodiments, Q is unsubstituted C₁-C₆-alkyl and R⁵ is H. In other embodiments, Q is a bond and R⁵ is unsubstituted C₃-C₆-carboxyalkyl.

In yet other embodiments, in compounds of Formula (II), R³ is H and R⁴ is Q-R⁵. In such embodiments, Q can be a bond and R⁵ can be optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

In some embodiments, in compounds of Formula (II), R³ is H and R⁴ is Q-R⁵, wherein Q is optionally substituted C₁-C₆-alkyl and R⁵ is H, OH, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In some embodiments, Q is unsubstituted C₁-C₆-alkyl. In other embodiments, Q is C₁-C₆-alkyl substituted with one or more of —NH₂, —CO₂H, —OH, carbonyl, halogen, C₁-C₂ alkylthio, C₁-C₂ alkyl, C₁-C₂ hydroxyalkyl, (C₀-C₂ alkyl)-cycloalkyl, (C₀-C₂ alkyl)-heterocycloalkyl, (C₀-C₂ alkyl)-aryl, and (C₀-C₂ alkyl)-heteroaryl. In other embodiments, Q is C₁-C₆-alkyl substituted with one or more of —NH₂, —CO₂H, —OH, carbonyl, halogen, C₁-C₂ alkyl, and C₁-C₂ hydroxyalkyl.

In some embodiments, in compounds of Formula (II), R³ is H and R⁴ is Q-R⁵, wherein Q is optionally substituted C₂-C₆-alkenyl and R⁵ is H, OH, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In some embodiments, Q is unsubstituted C₂-C₆-alkenyl.

In some of these embodiments, the compound is:

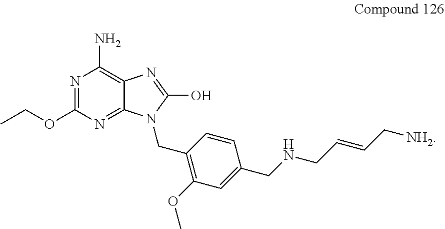 [SMILES: CCOc1nc(N)c2nc(O)n(Cc3ccc(CNC/C=C/CN)cc3OC)c2n1]

In various embodiments, in compounds of Formula (II), in which one or more of R³ and R⁴ is Q-R⁵, Q is C₁-C₆-alkyl substituted with one or more of C₃-C₆-cycloalkyl, C₂-C₆-heterocycloalkyl, C₃-C₇-heterobicycloalkyl, C₆-C₁₀-heterospirocycloalkyl, aryl, or heteroaryl. In some of these embodiments, such ring substituent (referred to below as “W”) can be a bridging moiety so that Q is:

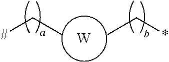

wherein:
W is C optionally substituted ₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl; and
a and b are independently integers from 0 to 4,
   and wherein * is the point of attachment to R⁵ and #is the point of attachment to N.

In such embodiments, W is an unsubstituted ring moiety such that Q can be selected from the group consisting of:

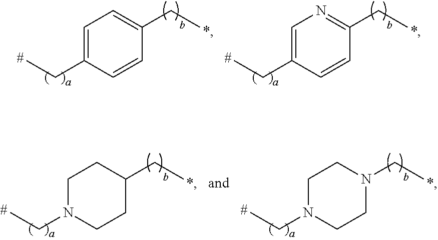

wherein a and b are independently 1, 2, or 3.

In some embodiments, in compounds of Formula (II), R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring. In some embodiments, R³ and R⁴ together with the N form a C₂-C₆-heterocycloalkyl ring, a C₃-C₇-heterobicycloalkyl ring, a C₆-C₁₀-heterospirocycloalkyl ring or a heteroaryl ring, wherein the ring moiety is optionally substituted with one or more of —NH₂, —CO₂H, —OH, carbonyl, halogen, C₁-C₂ alkylthio, C₁-C₂ alkyl, C₁-C₂ hydroxyalkyl, (C₀-C₂ alkyl)-cycloalkyl, or (C₀-C₂ alkyl)-heterocycloalkyl. In some embodiments, R³ and R⁴ together with the N form a ring selected from the group consisting of:

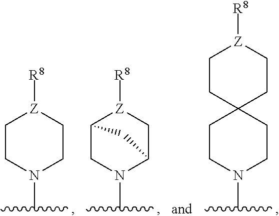

wherein:
Z is N or CH; and
R⁸ is H, NH₂, optionally substituted C₁-C₆-alkyl, optionally substituted C₁-C₆-heteroalkyl, optionally substituted C₁-C₆-aminoalkyl, or optionally substituted C₃-C₆-heterocycloalkyl.

In some embodiments, in compounds of Formula (II), R³ and R⁴ together with the N form a ring selected from the group consisting of:

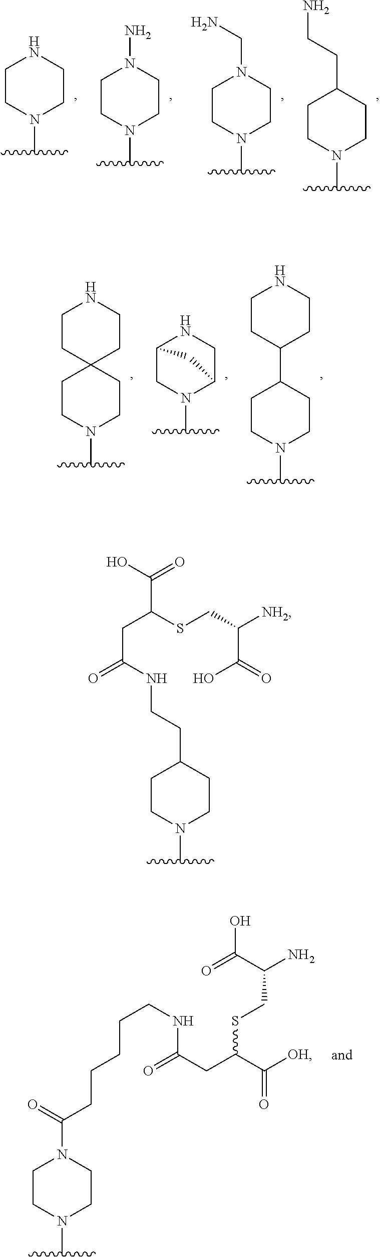

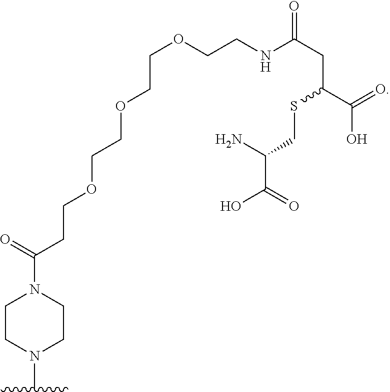 [SMILES: *N1CCN(C(=O)CCOCCOCCOCCNC(=O)CC(SC[C@@H](N)C(=O)O)C(=O)O)CC1]

In some embodiments, in compounds of Formula (II) in which at least one of R³ and R⁴ is Q-R⁵, R⁵ can be NR⁶R⁷. In such embodiments, R⁶ and R⁷ can be independently H, optionally substituted C₁-C₆ alkyl or optionally substituted C₁-C₆ alkoxycarbonyl. In some embodiments, R⁶ and R⁷ are both H. In some embodiments, R⁶ and R⁷ are both optionally substituted C₁-C₆ alkyl such that R⁶ is identical to R⁷. In such embodiments, R⁶ and R⁷ can both be C₁-C₆ alkyl substituted with one or more of —NH₂, —CO₂H, —OH, carbonyl, halogen, C₁-C₂ alkylthio, C₁-C₂ alkyl, and C₁-C₂ hydroxyalkyl. In some embodiments, R⁶ and R⁷ are C₁-C₂ alkyl substituted with one or more of —NH₂, —CO₂H, —OH, and halogen. In other embodiments, R⁶ is H and R⁷ is optionally substituted C₁-C₆ alkyl. In some embodiments, R⁶ is H and R⁷ can be C₁-C₆ alkyl substituted with one or more of —NH₂, —CO₂H, —OH, carbonyl, halogen, C₁-C₂ alkylthio, C₁-C₂ alkyl, C₁-C₂ hydroxyalkyl, optionally substituted C₃-C₆-cycloalkyl, optionally substituted C₂-C₆-heterocycloalkyl, optionally substituted aryl, and optionally substituted heteroaryl.

In some embodiments, in compounds of Formula (II), R⁵ is NR⁶R⁷, wherein R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring. In some embodiments, such ring moiety can be optionally substituted with one or more of —NH₂, —CO₂H, —OH, carbonyl, halogen, C₁-C₂ alkylthio, C₁-C₂ alkyl, C₁-C₃ aminoalkyl and C₁-C₂ hydroxyalkyl. In some embodiments, R⁶ and R⁷ together with the N form a ring selected from the group consisting of:

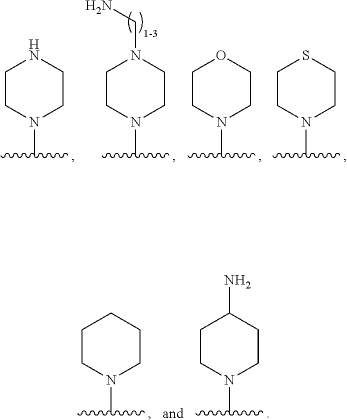

In some embodiments, a compound of Formula (II) is selected from SUBTABLE 1A.

In some embodiments, the compound of Formula (I) or Formula (II) is:

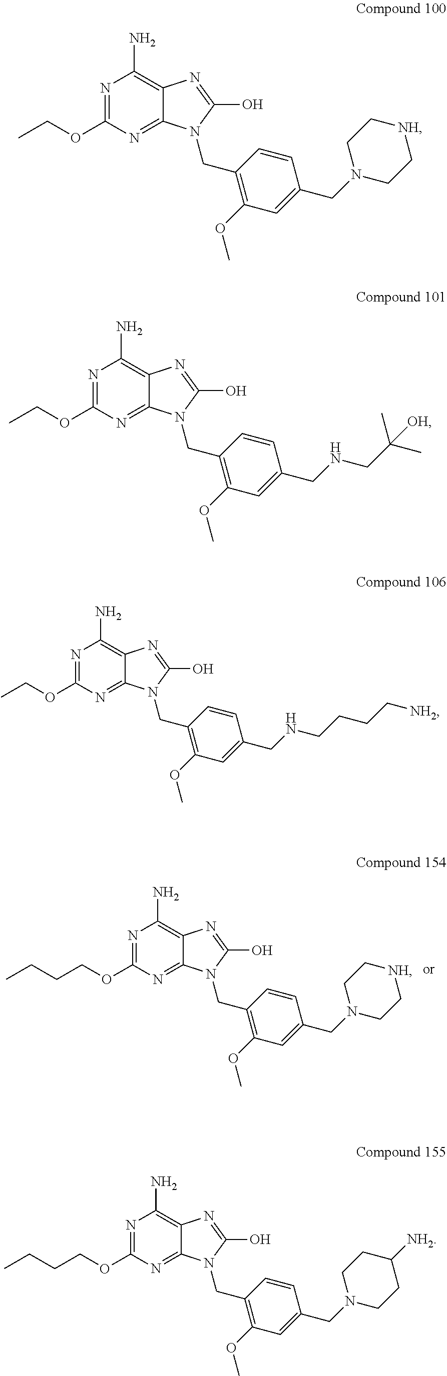

In various embodiments, the present disclosure relates to compounds of Formula (III):

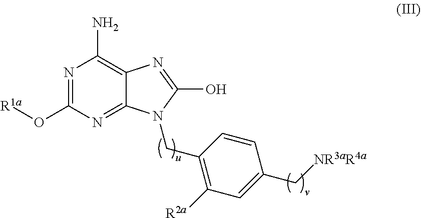

a tautomer, a protected version, and/or a pharmaceutically acceptable salt thereof, wherein:
R^{1a} is optionally substituted C₂-C₆ alkyl or branched and optionally substituted C₃-C₈ hydroxyalkyl;
R^{2a} is halogen, optionally substituted C₁-C₆ alkyl, or optionally substituted C₁-C₆ alkoxy;
R^{3a} and R^{4a} are independently H, Q-R^{5a}, or R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

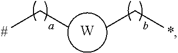

wherein * is the point of attachment to R⁵ and #is the point of attachment to N;
R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R^{6a} and R^{7a} are independently H, optionally substituted C₁-C₆ alkyl, optionally substituted C₁-C₆ alkoxycarbonyl, or R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
u is an integer from 1 to 3;
v is an integer from 0 to 4; and
a and b are independently integers from 0 to 4.

In some embodiments, in compounds of Formula (III), R^{1a} is optionally substituted C₂-C₆ alkyl. In such embodiments, Ria can be unsubstituted C₂-C₆ alkyl. In some of these embodiments, R^{1a} can be ethyl or n-butyl.

In some embodiments, in compounds of Formula (III), R^{2a} is halogen or optionally substituted C₁-C₆ alkoxy. In such embodiments, R^{2a} can be halogen or unsubstituted C₁-C₆ alkoxy. In certain embodiments, R^{2a} is fluorine or methoxy.

In some embodiments, in compounds of Formula (III), R^{3a} and R^{4a} are both H. In other embodiments, R^{3a} and R^{4a} are both Q-R^{5a}. In such embodiments, Q can be optionally substituted C₁-C₆-alkyl, and R^{5a} can be H or OH. In some of these embodiments, Q is unsubstituted C₁-C₆-alkyl and R^{5a} is H. In other embodiments, Q is a bond and R^{5a} is unsubstituted C₃-C₆-carboxyalkyl.

In yet other embodiments, in compounds of Formula (III), R^{3a} is H and R^{4a} is Q-R^{5a}. In such embodiments, Q can be a bond and R^{a} can be optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

In some embodiments, in compounds of Formula (III), R^{3a} is H and R^{4a} is Q-R^{5a}, wherein Q is optionally substituted C₁-C₆-alkyl and R^{5a} is H, OH, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In some embodiments, Q is unsubstituted C₁-C₆-alkyl. In other embodiments, Q is C₁-C₆-alkyl substituted with one or more of —NH₂, —CO₂H, —OH, carbonyl, halogen, C₁-C₂ alkylthio, C₁-C₂ alkyl, C₁-C₂ hydroxyalkyl, (C₀-C₂ alkyl)-cycloalkyl, (C₀-C₂ alkyl)-heterocycloalkyl, (C₀-C₂ alkyl)-aryl, and (C₀-C₂ alkyl)-heteroaryl. In other embodiments, Q is C₁-C₆-alkyl substituted with one or more of —NH₂, —CO₂H, —OH, carbonyl, halogen, C₁-C₂ alkyl, and C₁-C₂ hydroxyalkyl.

In some embodiments, in compounds of Formula (III), R^{3a} is H and R^{4a} is Q-R^{5a}, wherein Q is optionally substituted C₂-C₆-alkenyl and R^{5a} is H, OH, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In some embodiments, Q is unsubstituted C₂-C₆-alkenyl.

In various embodiments, in compounds of Formula (III), in which one or more of R^{3a} and R^{4a} is Q-R^{5a}, Q is C₁-C₆-alkyl substituted with one or more of C₃-C₆-cycloalkyl, C₂-C₆-heterocycloalkyl, C₃-C₇-heterobicycloalkyl, C₆-C₁₀-heterospirocycloalkyl, aryl, or heteroaryl. In some of these embodiments, such ring substituent (referred to below as “W”) can be a bridging moiety so that Q is:

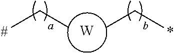

wherein:
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl; and
a and b are independently integers from 0 to 4, and wherein * is the point of attachment to R⁵ and #is the point of attachment to N.

In some of these embodiments, W is an unsubstituted ring moiety such that Q can be selected from the group consisting of:

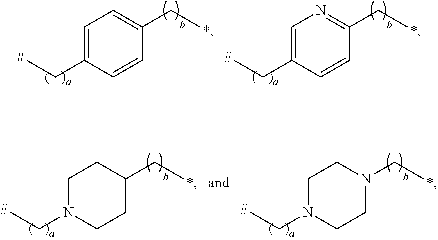

wherein a and b are independently 1, 2, or 3.

In some embodiments, in compounds of Formula (III), R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring. In some embodiments, R^{3a} and R^{4a} together with the N form a C₂-C₆-heterocycloalkyl ring, a C₃-C₇-heterobicycloalkyl ring, a C₆-C₁₀-heterospirocycloalkyl ring or a heteroaryl ring, wherein the ring moiety can be optionally substituted with one or more of —NH₂, —CO₂H, —OH, carbonyl, halogen, C₁-C₂ alkylthio, C₁-C₂ alkyl, C₁-C₂ hydroxyalkyl, (C₀-C₂ alkyl)-cycloalkyl, and (C₀-C₂ alkyl)-heterocycloalkyl.

In some embodiments, R^{3a} and R^{4a} together with the N form a ring selected from the group consisting of:

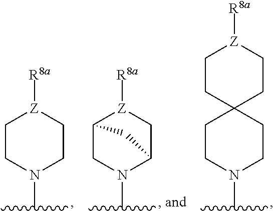

wherein:
Z is N or CH; and
R^{5a} is H, NH₂, optionally substituted C₁-C₆-alkyl, optionally substituted C₁-C₆-heteroalkyl, optionally substituted C₁-C₆-aminoalkyl, or optionally substituted C₂-C₆-heterocycloalkyl.

In some embodiments, in compounds of Formula (III), R^{3a} and R^{4a} together with the N form a ring selected from the group consisting of:

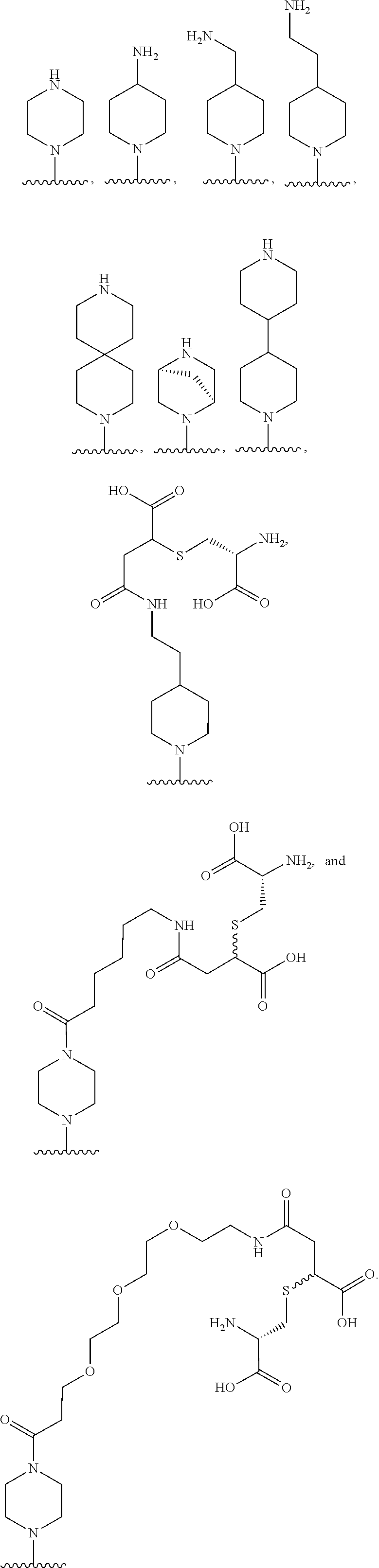

In some embodiments, in compounds of Formula (III), R^{5a} is NR^{6a}R^{7a}. In such embodiments, R^{6a} and R^{7a} can be independently H, optionally substituted C₁-C₆ alkyl or optionally substituted C₁-C₆ alkoxycarbonyl. In some embodiments, R^{6a} and R^{7a} are both H. In other embodiments, R^{6a} and R^{7a} are both optionally substituted C₁-C₆ alkyl, wherein R^{6a} is identical to R^{7a}. In such embodiments, R^{6a} and R^{7a} can both be C₁-C₆ alkyl substituted with one or more of —NH₂, —CO₂H, —OH, carbonyl, halogen, C₁-C₂ alkylthio, C₁-C₂ alkyl, and C₁-C₂ hydroxyalkyl. In some embodiments, R^{6a} and R^{7a} are C₁-C₂ alkyl substituted with one or more of —NH₂, —CO₂H, —OH, or halogen. In other embodiments, R^{6a} is H and R^{7a} is optionally substituted C₁-C₆ alkyl. In some embodiments, R^{6a} is H and R^{7a} can be C₁-C₆ alkyl substituted with one or more of —NH₂, —CO₂H, —OH, carbonyl, halogen, C₁-C₂ alkylthio, C₁-C₂ alkyl, C₁-C₂ hydroxyalkyl, optionally substituted C₃-C₆-cycloalkyl, optionally substituted C₂-C₆-heterocycloalkyl, optionally substituted aryl, and optionally substituted heteroaryl.

In some embodiments, in compounds of Formula (III) in which at least one of R^{3a} and R^{4a} is Q-R^{5a}, R^{5a} can be NR^{6a}R^{7a}, wherein R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring. In some embodiments, such ring moiety can be optionally substituted with one or more of —NH₂, —CO₂H, —OH, carbonyl, halogen, C₁-C₂ alkylthio, C₁-C₂ alkyl, C₁-C₃ aminoalkyl and C₁-C₂ hydroxyalkyl. In some embodiments, R⁶ and R⁷ together with the N form a ring selected from the group consisting of:

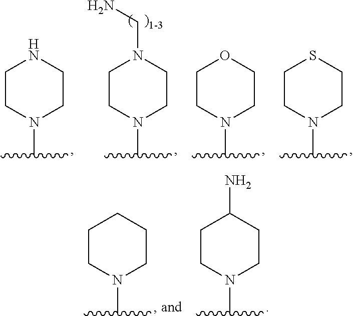

In various embodiments, in compounds of Formula (III), u is 1.

In various embodiments, in compounds of Formula (III), v is an integer from 0 to 4, from 1 to 4, 1 to 3, or from 1 to 2. In some embodiments, v is 0. In some embodiments, v is 1. In other embodiments, v is 2. In yet other embodiments, v is 3.

In some embodiments, a compound of Formula (III) is selected from SUBTABLE 1A.

In various embodiments of this disclosure, a compound herein of Formula (III) is a compound according to Formula (IV):
(IV)

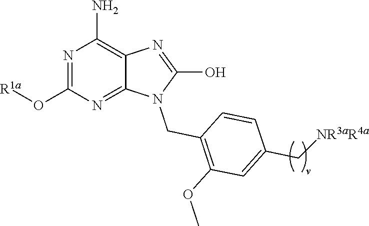

wherein:
R^{1a} is unsubstituted C₂-C₆ alkyl or branched and unsubstituted C₃-C₈ hydroxyalkyl;
R^{3a} and R^{4a} are independently H, Q-R^{5a}, or R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

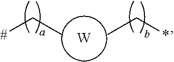

wherein * is the point of attachment to R⁵ and #is the point of attachment to N;
R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R^{6a} and R^{7a} are independently H, optionally substituted C₁-C₆ alkyl, optionally substituted C₁-C₆ alkoxycarbonyl, or R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
v is an integer from 0 to 4; and
a and b are independently integers from 0 to 4.

In some embodiments, in compounds of Formula (IV), R^{1a} is unsubstituted C₂-C₆ alkyl, such as unsubstituted C₂-C₄ alkyl. In other embodiments, R^{1a} is branched and unsubstituted C₃-C₈ hydroxyalkyl.

In some embodiments, in compounds of Formula (IV), R^{3a} and R^{4a} are both H. In other embodiments, R^{3a} and R^{4a} are both Q-R^{5a}. In such embodiments, Q can be optionally substituted C₁-C₆-alkyl, and R^{5a} can be H or OH. In some of these embodiments, Q is unsubstituted C₁-C₆-alkyl and R^{5a} is H. In other embodiments, Q is a bond and R^{5a} is unsubstituted C₃-C₆-carboxyalkyl.

In yet other embodiments, in compounds of Formula (IV), R^{3a} is H and R^{4a} is Q-R^{5a}. In such embodiments, Q can be a bond and R^{5a} can be H, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

In some embodiments, in compounds of Formula (IV), R^{3a} is H and R^{4a} is Q-R^{5a}, wherein Q is optionally substituted C₁-C₆-alkyl and R^{5a} is H, OH, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In some embodiments, Q is unsubstituted C₁-C₆-alkyl. In other embodiments, Q is C₁-C₆-alkyl substituted with one or more of —NH₂, —CO₂H, —OH, carbonyl, halogen, C₁-C₂ alkylthio, C₁-C₂ alkyl, C₁-C₂ hydroxyalkyl, (C₀-C₂ alkyl)-cycloalkyl, (C₀-C₂ alkyl)-heterocycloalkyl, (C₀-C₂ alkyl)-aryl, and (C₀-C₂ alkyl)-heteroaryl. In yet other embodiments, Q is C₁-C₆-alkyl substituted with one or more of —NH₂, —CO₂H, —OH, carbonyl, halogen, C₁-C₂ alkyl, and C₁-C₂ hydroxyalkyl.

In some embodiments, in compounds of Formula (IV), R^{3a} is H and R^{4a} is Q-R^{5a}, wherein Q is optionally substituted C₂-C₆-alkenyl and R^{5a} is H, OH, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl. In some embodiments, Q is unsubstituted C₂-C₆-alkenyl.

In various embodiments, in compounds of Formula (IV), in which one or more of R^{3a} and R^{4a} is Q-R^{5a}, Q is C₁-C₆-alkyl substituted with one or more of C₃-C₆-cycloalkyl, C₂-C₆-heterocycloalkyl, C₃-C₇-heterobicycloalkyl, C₆-C₁₀-heterospirocycloalkyl, aryl, or heteroaryl. In some of these embodiments, such ring substituent (referred to below as “W”) can be a bridging moiety so that Q is:

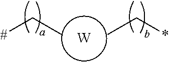

wherein:
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl; and
a and b are independently integers from 0 to 4, and wherein * is the point of attachment to R⁵ and #is the point of attachment to N.

In such embodiments, W is an unsubstituted ring moiety such that Q can be selected from the group consisting of:

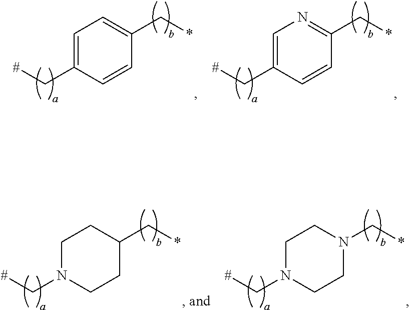

wherein a and b are independently 1, 2, or 3.

In some embodiments, in compounds of Formula (IV), R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring. In some embodiments, R^{3a} and R^{4a} together with the N form a C₂-C₆-heterocycloalkyl ring, a C₃-C₇-heterobicycloalkyl ring, a C₆-C₁₀-heterospirocycloalkyl ring or a heteroaryl ring, wherein the ring moiety can be optionally substituted with one or more of —NH₂, —CO₂H, —OH, carbonyl, halogen, C₁-C₂ alkylthio, C₁-C₂ alkyl, C₁-C₂ hydroxyalkyl, (C₀-C₂ alkyl)-cycloalkyl, or (C₀-C₂ alkyl)-heterocycloalkyl. In some embodiments, R^{3a} and R^{4a} together with the N form a ring selected from the group consisting of:

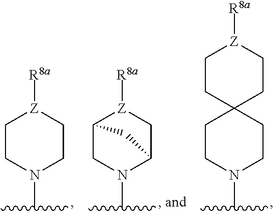

wherein:
Z is N or CH; and
R^{8a} is H, NH₂, optionally substituted C₁-C₆-alkyl, optionally substituted C₁-C₆-heteroalkyl, optionally substituted C₁-C₆-aminoalkyl, or optionally substituted C₃-C₆-heterocycloalkyl.

In some embodiments, in compounds of Formula (IV), R^{3a} and R^{4a} together with the N form a ring selected from the group consisting of:

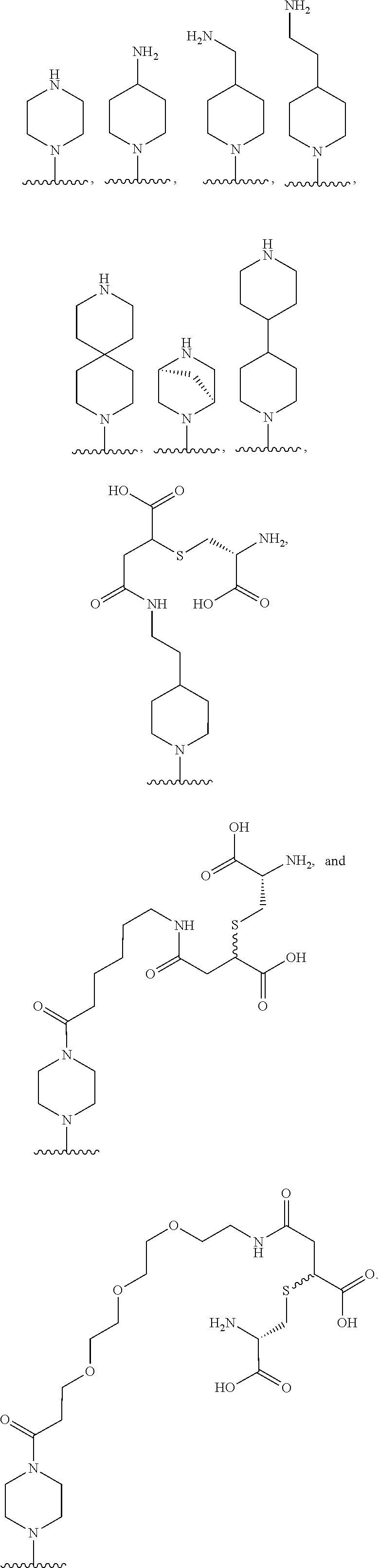

In some embodiments, in compounds of Formula (IV) in which one or more of R^{3a} and R^{4a} is Q-R^{5a}, R^{5a} can be NR^{6a}R^{7a}. In such embodiments, R^{6a} and R^{7a} can be independently H, optionally substituted C₁-C₆ alkyl or optionally substituted C₁-C₆ alkoxycarbonyl. In some embodiments, R^{6a} and R^{7a} are both H. In some embodiments, R^{6a} and R^{7a} are both optionally substituted C₁-C₆ alkyl, wherein R^{6a} is identical to R^{7a}. In such embodiments, R^{6a} and R^{7a} can be C₁-C₆ alkyl substituted with one or more of —NH₂, —CO₂H, —OH, carbonyl, halogen, C₁-C₂ alkylthio, C₁-C₂ alkyl, and C₁-C₂ hydroxyalkyl. In some embodiments, R^{6a} and R^{7a} are C₁-C₂ alkyl substituted with one or more of —NH₂, —CO₂H, —OH, or halogen. In other embodiments, R^{6a} is H and R^{7a} is optionally substituted C₁-C₆ alkyl. In some embodiments, R^{6a} is H and R^{7a} can be C₁-C₆ alkyl substituted with one or more of —NH₂, —CO₂H, —OH, carbonyl, halogen, C₁-C₂ alkylthio, C₁-C₂ alkyl, C₁-C₂ hydroxyalkyl, optionally substituted C₃-C₆-cycloalkyl, optionally substituted C₂-C₆-heterocycloalkyl, optionally substituted aryl, and optionally substituted heteroaryl.

In some embodiments, in compounds of Formula (IV) in which one or more of R^{3a} and R^{4a} is Q-R^{5a}, R^{5a} can be NR^{6a}R^{7a}, wherein R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring. In some embodiments, such ring moiety can be optionally substituted with one or more of —NH₂, —CO₂H, —OH, carbonyl, halogen, C₁-C₂ alkylthio, C₁-C₂ alkyl, C₁-C₃ aminoalkyl and C₁-C₂ hydroxyalkyl. In some embodiments, R⁶ and R⁷ together with the N form a ring selected from the group consisting of:

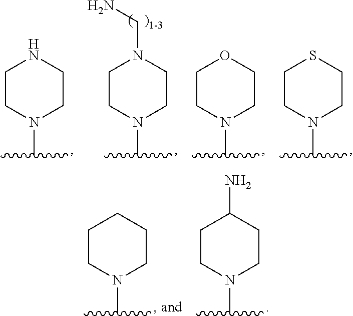

In some embodiments, in compounds of Formula (IV), v is 0. In other embodiments, v is 1. In some embodiments, v is 2. In yet other embodiments, v is 3.

In some embodiments, a compound of Formula (IV) is selected from the compounds shown in SUBTABLE 1A.

In some embodiments, a compound of the present disclosure, e.g., a compound of any one of Formulae (I)-(IV) herein, is selected from TABLE 1, which comprises SUBTABLES 1A-1F.

TABLE 1
Exemplary Purine-derived Compounds of the Present Disclosure

| | | |
|---|---|---|
| Structure | Name | Number |

SUBTABLE 1A:

| | | |
|---|---|---|
| 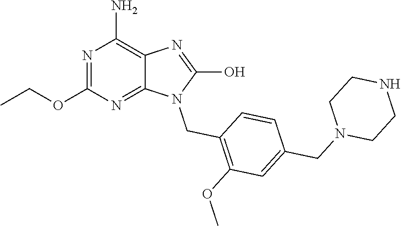 [SMILES: CCOc1nc(N)c2nc(O)n(Cc3ccc(CN4CCNCC4)cc3OC)c2n1] | 6-amino-2-ethoxy-9-(2- methoxy-4-(piperazin-1- ylmethyl)benzyl)-9H- purin-8-ol | Compound 100 |
| 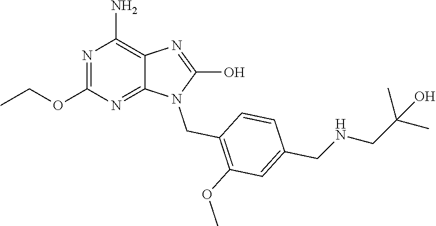 [SMILES: CCOc1nc(N)c2nc(O)n(Cc3ccc(CNCC(C)(C)O)cc3OC)c2n1] | 6-amino-2-ethoxy-9-(4- (((2-hydroxy-2- methylpropyl)amino) methyl)-2- methoxybenzyl)- 9H-purin-8-ol | Compound 101 |
| 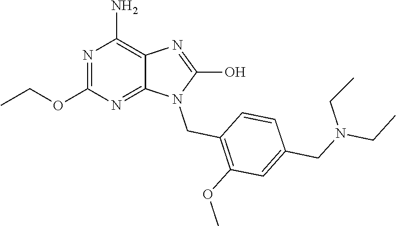 [SMILES: CCOc1nc(N)c2nc(O)n(Cc3ccc(CN(CC)CC)cc3OC)c2n1] | 6-amino-9-(4- ((diethylamino)methyl)- 2-methoxybenzyl)-2- ethoxy-9H-purin-8-ol | Compound 102 |
| 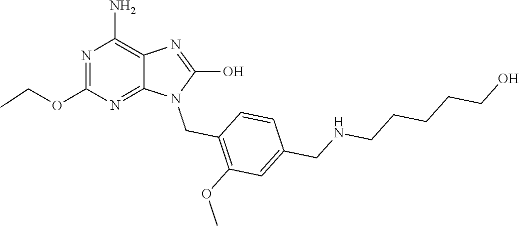 [SMILES: CCOc1nc(N)c2nc(O)n(Cc3ccc(CNCCCCCO)cc3OC)c2n1] | 6-amino-2-ethoxy-9-(4- (((5-hydroxypentyl)- amino)methyl)-2- methoxy-benzyl)-9H- purin-8-ol | Compound 103 |
| 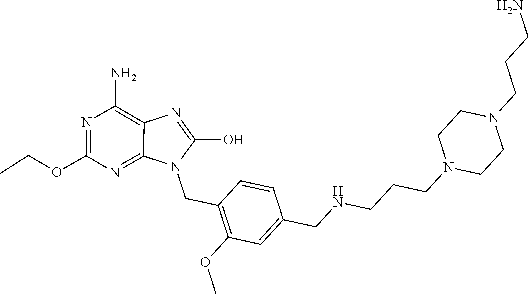 [SMILES: CCOc1nc(N)c2nc(O)n(Cc3ccc(CNCCCN4CCN(CCCN)CC4)cc3OC)c2n1] | 6-amino-9-(4-(((3-(4-(3- aminopropyl)piperazin- 1-yl)propyl)amino)- methyl)-2- methoxybenzyl)-2- ethoxy-9H-purin-8-ol | Compound 104 |
| 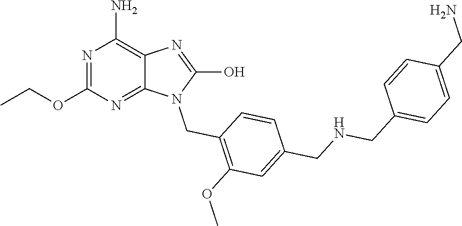 [SMILES: CCOc1nc(N)c2nc(O)n(Cc3ccc(CNCc4ccc(CN)cc4)cc3OC)c2n1] | 6-amino-9-(4-(((4- (aminomethyl)benzyl) amino)methyl)-2- methoxybenzyl)-2- ethoxy-9H-purin-8-ol | Compound 105 |
| 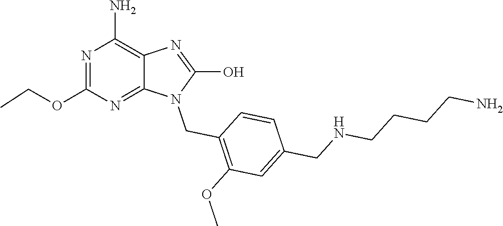 [SMILES: CCOc1nc(N)c2nc(O)n(Cc3ccc(CNCCCCN)cc3OC)c2n1] | 6-amino-9-(4-(((4- aminobutyl)amino) methyl)- 2-methoxybenzyl)-2- ethoxy-9H-purin-8-ol | Compound 106 |
| 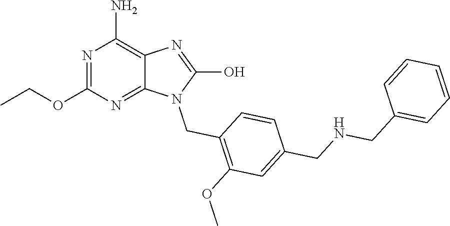 [SMILES: CCOc1nc(N)c2nc(O)n(Cc3ccc(CNCc4ccccc4)cc3OC)c2n1] | 6-amino-9-(4- ((benzylamino)methyl)- 2-methoxybenzyl)-2- ethoxy-9H-purin-8-ol | Compound 107 |
| 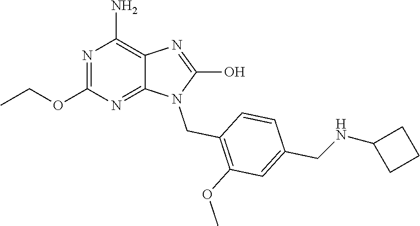 [SMILES: CCOc1nc(N)c2nc(O)n(Cc3ccc(CNC4CCC4)cc3OC)c2n1] | 6-amino-9-(4- ((cyclobutylamino) methyl)- 2-methoxybenzyl)-2- ethoxy-9H-purin-8-ol | Compound 108 |
| 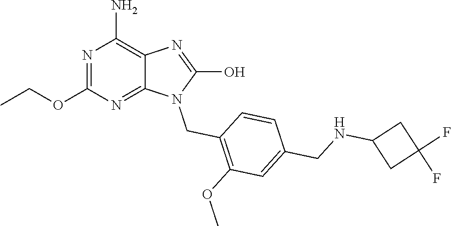 [SMILES: CCOc1nc(N)c2nc(O)n(Cc3ccc(CNC4CC(F)(F)C4)cc3OC)c2n1] | 6-amino-9-(4-(((3,3- difluorocyclobutyl) amino) methyl)-2- methoxybenzyl)-2- ethoxy-9H-purin-8-ol | Compound 109 |
| 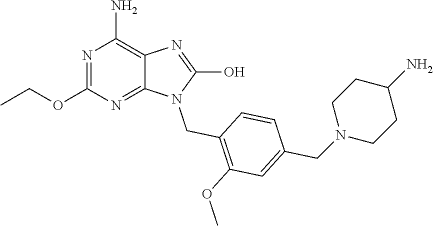 [SMILES: CCOc1nc(N)c2nc(O)n(Cc3ccc(CN4CCC(N)CC4)cc3OC)c2n1] | 6-amino-9-(4-((4- aminopiperidin-1- yl)methyl)-2- methoxybenzyl)-2- ethoxy-9H-purin-8-ol | Compound 110 |
| 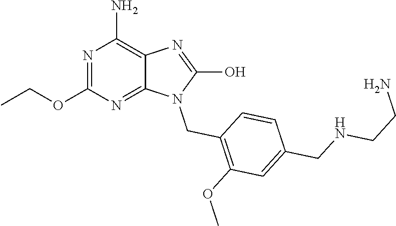 [SMILES: CCOc1nc(N)c2nc(O)n(Cc3ccc(CNCCN)cc3OC)c2n1] | 6-amino-9-(4-(((2- aminoethyl)amino) methyl)- 2-methoxybenzyl)-2- ethoxy-9H-purin-8-ol | Compound 111 |
| 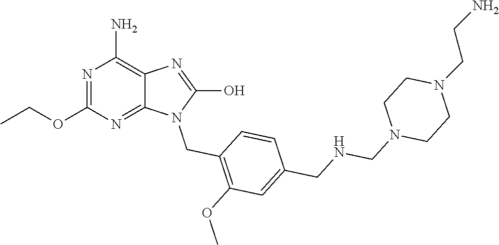 [SMILES: CCOc1nc(N)c2nc(O)n(Cc3ccc(CNCN4CCN(CCN)CC4)cc3OC)c2n1] | 6-amino-9-(4-(((2- aminoethyl)amino) methyl)- 2-methoxybenzyl)-2- ethoxy-9H-purin-8-ol | Compound 112 |
| 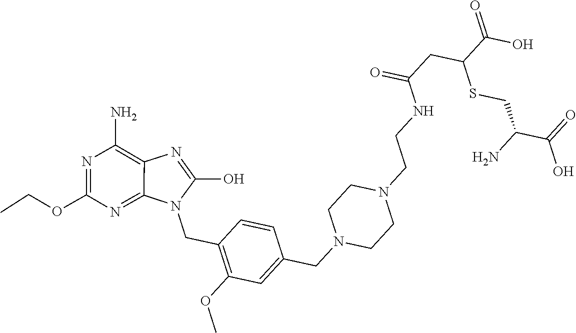 [SMILES: CCOc1nc(N)c2nc(O)n(Cc3ccc(CN4CCN(CCNC(=O)CC(SC[C@@H](N)C(=O)O)C(=O)O)CC4)cc3OC)c2n1] | 2-(((S)-2-amino-2- carboxyethyl)thio)-4-((2- (4-(4-((6-amino-2- ethoxy-8-hydroxy-9H- purin-9-yl)methyl)-3- methoxybenzyl) piperazin- 1-yl)ethyl)amino)-4- oxobutanoic acid | Compound 113 |
| 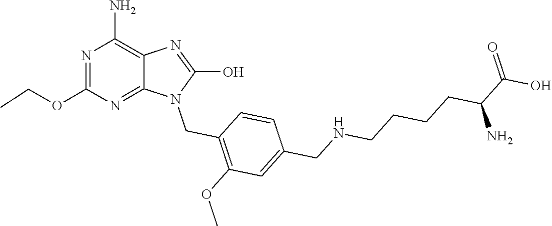 [SMILES: CCOc1nc(N)c2nc(O)n(Cc3ccc(CNCCCC[C@H](N)C(=O)O)cc3OC)c2n1] | N⁶-(4-((6-amino-2- ethoxy-8-hydroxy-9H- purin-9-yl)methyl)-3- methoxybenzyl)-L- lysine | Compound 114 |
| 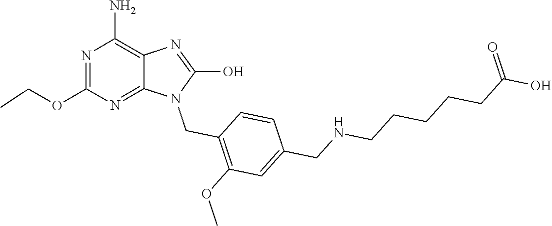 [SMILES: CCOc1nc(N)c2nc(O)n(Cc3ccc(CNCCCCCC(=O)O)cc3OC)c2n1] | 6-((4-((6-amino-2- ethoxy-8-hydroxy-9H- purin-9-yl)methyl)-3- methoxybenzyl)- amino)hexanoic acid | Compound 115 |
| 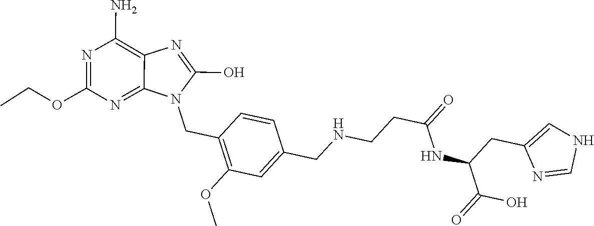 [SMILES: CCOc1nc(N)c2nc(O)n(Cc3ccc(CNCCC(=O)N[C@@H](Cc4c[nH]cn4)C(=O)O)cc3OC)c2n1] | (3-((4-((6-amino-2- ethoxy-8-hydroxy-9H- purin-9-yl)methyl)-3- methoxybenzyl)- amino)- propanoyl)-L- histidine | Compound 116 |
| 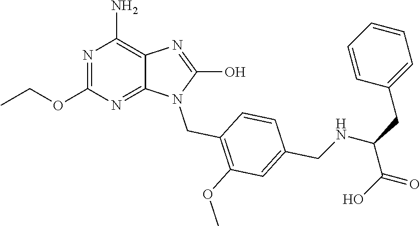 [SMILES: CCOc1nc(N)c2nc(O)n(Cc3ccc(CN[C@@H](Cc4ccccc4)C(=O)O)cc3OC)c2n1] | (4-((6-amino-2-ethoxy- 8-hydroxy-9H-purin-9- yl)methyl)-3- methoxybenzyl)-L- phenylalanine | Compound 117 |
| 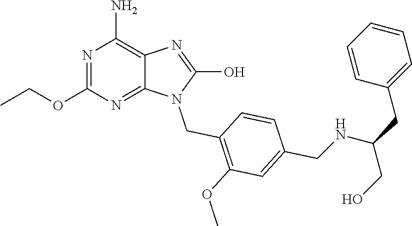 [SMILES: CCOc1nc(N)c2nc(O)n(Cc3ccc(CN[C@H](CO)Cc4ccccc4)cc3OC)c2n1] | (S)-6-amino-2-ethoxy- 9-(4-(((1-hydroxy-3- phenylpropan-2- yl)amino)methyl)-2- methoxybenzyl)-9H- purin-8-ol | Compound 118 |
| 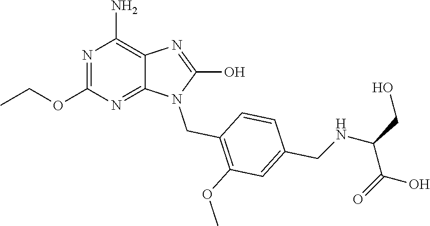 [SMILES: CCOc1nc(N)c2nc(O)n(Cc3ccc(CN[C@@H](CO)C(=O)O)cc3OC)c2n1] | (4-((6-amino-2-ethoxy- 8-hydroxy-9H-purin-9- yl)methyl)-3- methoxybenzyl)-L- serine | Compound 119 |
| 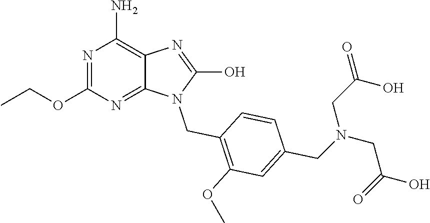 [SMILES: CCOc1nc(N)c2nc(O)n(Cc3ccc(CN(CC(=O)O)CC(=O)O)cc3OC)c2n1] | 2,2′-((4-((6-amino-2- ethoxy-8-hydroxy-9H- purin-9-yl)methyl)-3- methoxy- benzyl)azanediyl) diacetic acid | Compound 120 |
| 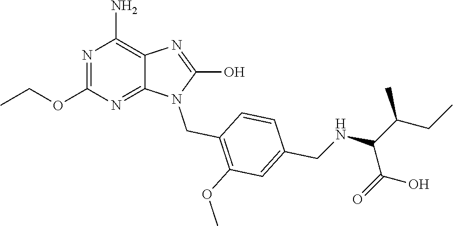 | (4-((6-amino-2-ethoxy- 8-hydroxy-9H-purin-9- yl)methyl)-3- methoxybenzyl)-L- isoleucine | Compound 121 |
| 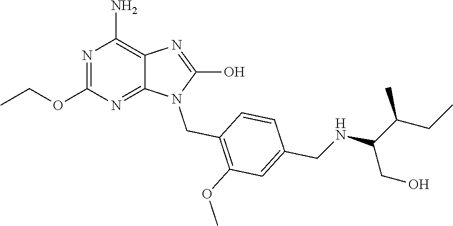 | 6-amino-2-ethoxy-9-(4- ((((2S,3S)-1-hydroxy-3- methylpentan-2- yl)amino)-methyl)-2- methoxybenzyl)-9H- purin-8-ol | Compound 122 |
| 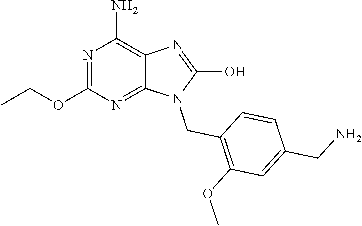 [SMILES: CCOc1nc(N)c2nc(O)n(Cc3ccc(CN)cc3OC)c2n1] | 6-amino-9-(4- (aminomethyl)-2- methoxybenzyl)-2- ethoxy-9H-purin-8-ol | Compound 123 |
| 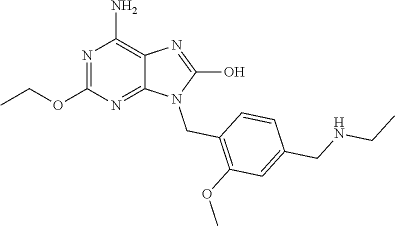 [SMILES: CCNCc1ccc(Cn2c(O)nc3c(N)nc(OCC)nc32)c(OC)c1] | 6-amino-2-ethoxy-9-(4- ((ethylamino)methyl)-2- methoxybenzyl)-9H- purin-8-ol | Compound 124 |
| 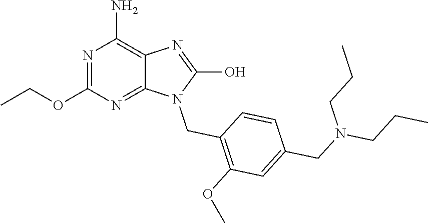 [SMILES: CCCN(CCC)Cc1ccc(Cn2c(O)nc3c(N)nc(OCC)nc32)c(OC)c1] | 6-amino-9-(4- ((dipropylamino) methyl)- 2-methoxybenzyl)-2- ethoxy-9H-purin-8-ol | Compound 125 |
| 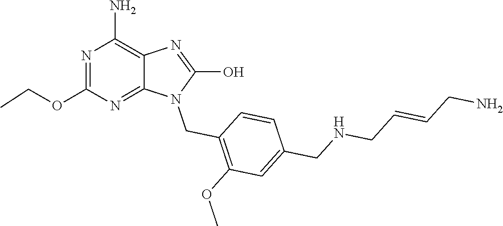 [SMILES: CCOc1nc(N)c2nc(O)n(Cc3ccc(CNC/C=C/CN)cc3OC)c2n1] | (E)-6-amino-9-(4-(((4- aminobut-2-en-1- yl)amino)methyl)-2- methoxybenzyl)-2- ethoxy-9H-purin-8-ol | Compound 126 |
| 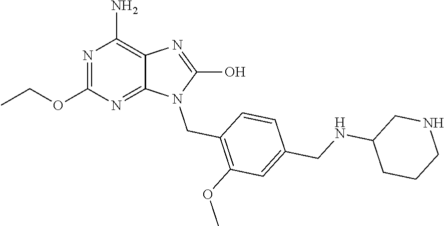 [SMILES: CCOc1nc(N)c2nc(O)n(Cc3ccc(CNC4CCCNC4)cc3OC)c2n1] | 6-amino-2-ethoxy-9-(2- methoxy-4-((piperidin- 3- ylamino)methyl)benzyl)- 9H-purin-8-ol | Compound 127 |
| 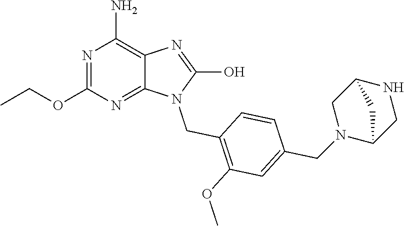 [SMILES: CCOc1nc(N)c2nc(O)n(Cc3ccc(CN4C[C@@H]5C[C@H]4CN5)cc3OC)c2n1] | 9-(4-(((1S,4S)-2,5- diazabicyclo[2.2.1] heptan- 2-yl)methyl)-2- methoxybenzyl)-6- amino-2-ethoxy-9H- purin-8-ol | Compound 128 |
| 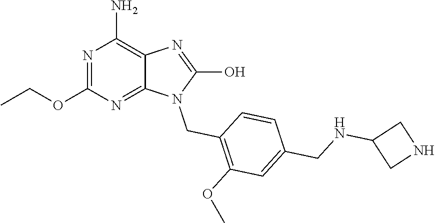 [SMILES: CCOc1nc(N)c2nc(O)n(Cc3ccc(CNC4CNC4)cc3OC)c2n1] | 6-amino-9-(4-((azetidin- 3-ylamino)methyl)-2- methoxybenzyl)-2- ethoxy-9H-purin-8-ol | Compound 129 |
| 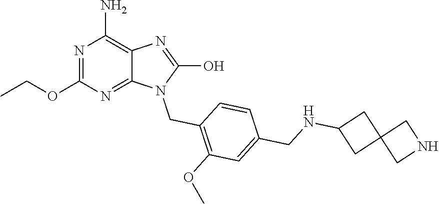 [SMILES: CCOc1nc(N)c2nc(O)n(Cc3ccc(CNC4CC5(CNC5)C4)cc3OC)c2n1] | 9-(4-(((2- azaspiro[3.3]heptan-6- yl)amino)methyl)-2- methoxybenzyl)-6- amino-2-ethoxy-9H- purin-8-ol | Compound 130 |
| 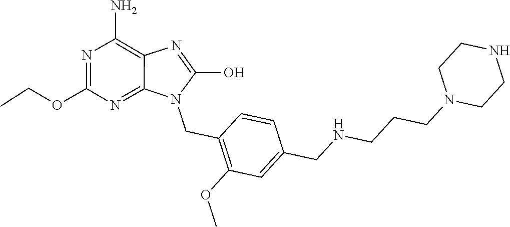 [SMILES: CCOc1nc(N)c2nc(O)n(Cc3ccc(CNCCCN4CCNCC4)cc3OC)c2n1] | 6-amino-2-ethoxy-9-(2- methoxy-4-(((3- (piperazin-1- yl)propyl)amino)methyl)- benzyl)-9H-purin-8-ol | Compound 131 |
| 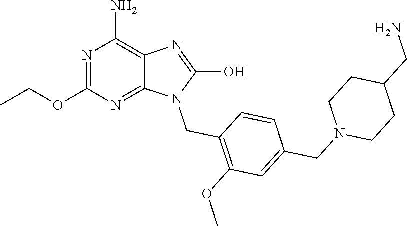 [SMILES: CCOc1nc(N)c2nc(O)n(Cc3ccc(CN4CCC(CN)CC4)cc3OC)c2n1] | 6-amino-9-(4-((4- (aminomethyl)piperidin- 1-yl)methyl)-2- methoxybenzyl)-2- ethoxy-9H-purin-8-ol | Compound 132 |
| 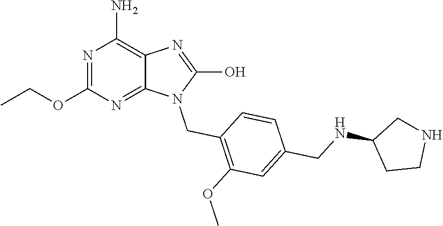 [SMILES: CCOc1nc(N)c2nc(O)n(Cc3ccc(CN[C@@H]4CCNC4)cc3OC)c2n1] | (S)-6-amino-2-ethoxy-9- (2-methoxy-4- ((pyrrolidin-3- ylamino)methyl)benzyl)- 9H-purin-8-ol | Compound 133 |
| 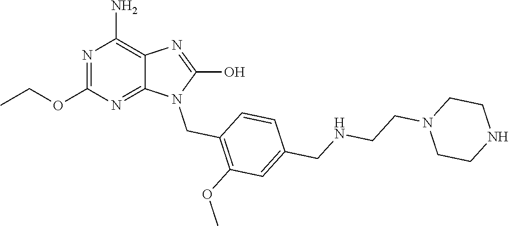 [SMILES: CCOc1nc(N)c2nc(O)n(Cc3ccc(CNCCN4CCNCC4)cc3OC)c2n1] | 6-amino-2-ethoxy-9-(2- methoxy-4-(((2- (piperazin-1- yl)ethyl)amino)methyl) benzyl)-9H-purin-8-ol | Compound 134 |
| 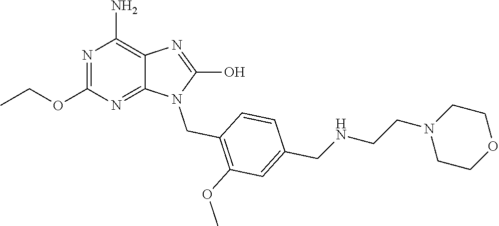 [SMILES: CCOc1nc(N)c2nc(O)n(Cc3ccc(CNCCN4CCOCC4)cc3OC)c2n1] | 6-amino-2-ethoxy-9-(2- methoxy-4-(((2- morpholinoethyl)amino) methyl)benzyl)-9H- purin-8-ol | Compound 135 |
| 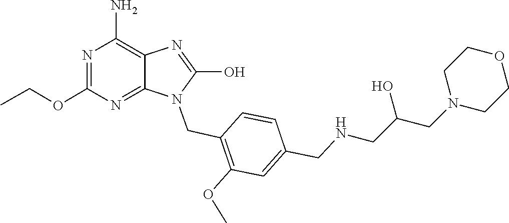 [SMILES: CCOc1nc(N)c2nc(O)n(Cc3ccc(CNCC(O)CN4CCOCC4)cc3OC)c2n1] | 6-amino-2-ethoxy-9-(4- (((2-hydroxy-3- morpholinopropyl)amino) methyl)-2- methoxybenzyl)-9H- purin-8-ol | Compound 136 |
| 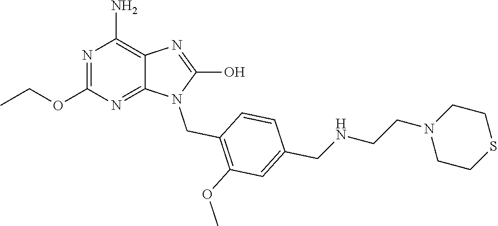 [SMILES: CCOc1nc(N)c2nc(O)n(Cc3ccc(CNCCN4CCSCC4)cc3OC)c2n1] | 6-amino-2-ethoxy-9-(2- methoxy-4-(((2- thiomorpholinoethyl) amino)methyl)-benzyl)- 9H-purin-8-ol | Compound 137 |
| 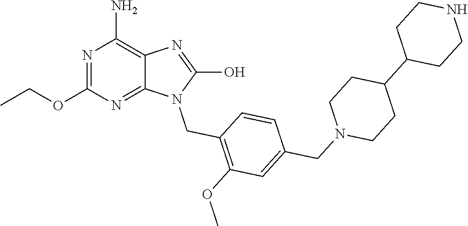 [SMILES: CCOc1nc(N)c2nc(O)n(Cc3ccc(CN4CCC(C5CCNCC5)CC4)cc3OC)c2n1] | 9-(4-([4,4′-bipiperidin]- 1-ylmethyl)-2- methoxybenzyl)-6- amino-2-ethoxy-9H- purin-8-ol | Compound 138 |
| 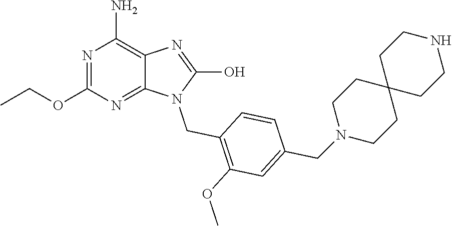 [SMILES: CCOc1nc(N)c2nc(O)n(Cc3ccc(CN4CCC5(CCNCC5)CC4)cc3OC)c2n1] | 9-(4-((3,9- diazaspiro[5.5]undecan- 3-yl)methyl)-2- methoxybenzyl)-6- amino-2-ethoxy-9H- purin-8-ol | Compound 139 |
| 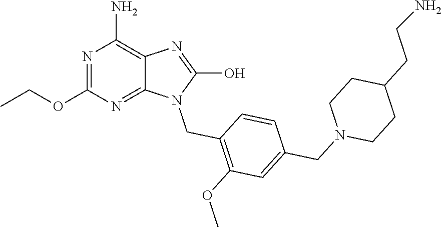 [SMILES: CCOc1nc(N)c2nc(O)n(Cc3ccc(CN4CCC(CCN)CC4)cc3OC)c2n1] | 6-amino-9-(4-((4-(2- aminoethyl)piperidin-1- yl)methyl)-2- methoxybenzyl)-2- ethoxy-9H-purin-8-ol | Compound 140 |
| 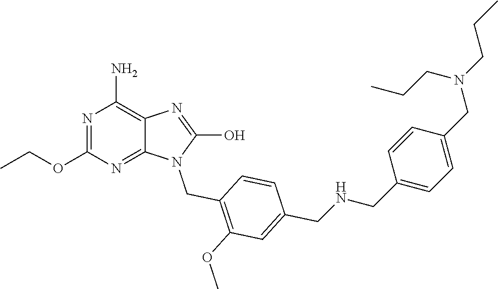 [SMILES: CCCN(CCC)Cc1ccc(CNCc2ccc(Cn3c(O)nc4c(N)nc(OCC)nc43)c(OC)c2)cc1] | 6-amino-9-(4-(((4- ((dipropylamino)methyl) benzyl)amino)methyl)-2- methoxybenzyl)-2- ethoxy-9H-purin-8-ol | Compound 141 |
| 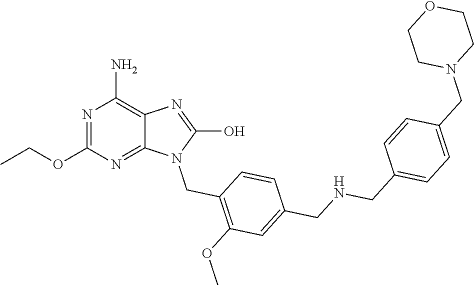 [SMILES: CCOc1nc(N)c2nc(O)n(Cc3ccc(CNCc4ccc(CN5CCOCC5)cc4)cc3OC)c2n1] | 6-amino-2-ethoxy-9-(2- methoxy-4-(((4- (morpholinomethyl) benzyl)amino)- methyl)benzyl)-9H- purin-8-ol | Compound 142 |
| 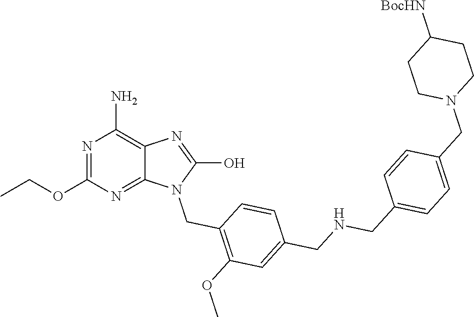 [SMILES: CCOc1nc(N)c2nc(O)n(Cc3ccc(CNCc4ccc(CN5CCC(NC(=O)OC(C)(C)C)CC5)cc4)cc3OC)c2n1] | tert-butyl-(1-(4-(((4-((6- amino-2-ethoxy-8- hydroxy-9H-purin-9- yl)methyl)-3- methoxybenzyl) amino)methyl)benzyl) piperidin-4-yl)carbamate | Compound 143 |
| 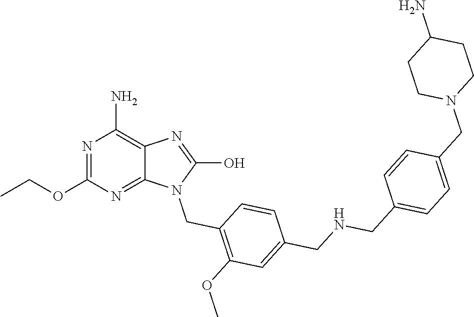 [SMILES: CCOc1nc(N)c2nc(O)n(Cc3ccc(CNCc4ccc(CN5CCC(N)CC5)cc4)cc3OC)c2n1] | 6-amino-9-(4-(((4-((4- aminopiperidin-1- yl)methyl)benzyl)amino) methyl)-2- methoxybenzyl)-2- ethoxy-9H-purin-8-ol | Compound 144 |
| 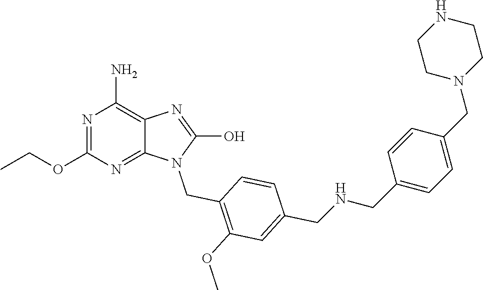 [SMILES: CCOc1nc(N)c2nc(O)n(Cc3ccc(CNCc4ccc(CN5CCNCC5)cc4)cc3OC)c2n1] | 6-amino-2-ethoxy-9-(2- methoxy-4-(((4- (piperazin-1- ylmethyl)benzyl)amino)- methyl)benzyl)-9H- purin-8-ol | Compound 145 |
| 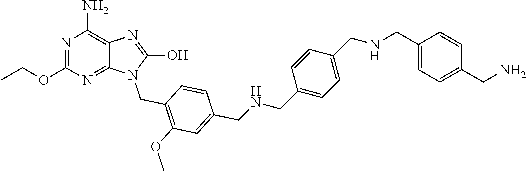 [SMILES: CCOc1nc(N)c2nc(O)n(Cc3ccc(CNCc4ccc(CNCc5ccc(CN)cc5)cc4)cc3OC)c2n1] | 6-amino-9-(4-(((4-(((4- (aminomethyl)benzyl) amino)methyl)benzyl) amino)-methyl)-2- methoxybenzyl)-2- ethoxy-9H-purin-8-ol | Compound 146 |
| 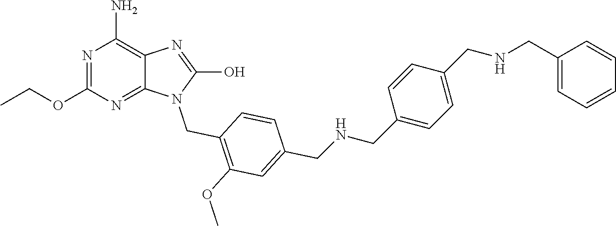 [SMILES: CCOc1nc(N)c2nc(O)n(Cc3ccc(CNCc4ccc(CNCc5ccccc5)cc4)cc3OC)c2n1] | 6-amino-9-(4-(((4- ((benzylamino)methyl) benzyl)amino)methyl)- 2- methoxy-benzyl)-2- ethoxy-9H-purin-8-ol | Compound 147 |
| 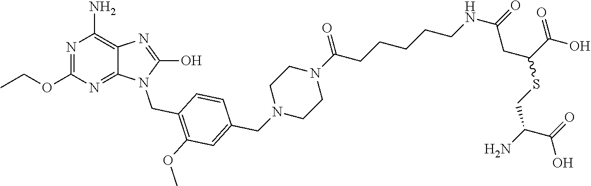 [SMILES: CCOc1nc(N)c2nc(O)n(Cc3ccc(CN4CCN(C(=O)CCCCCNC(=O)CC(SC[C@@H](N)C(=O)O)C(=O)O)CC4)cc3OC)c2n1] | 2-(((S)-2-amino-2- carboxyethyl)thio)-4- ((6-(4-(4-((6- amino-2- ethoxy-8-hydroxy-9H- purin-9-yl)methyl)-3- methoxybenzyl) piperazin-1-yl)-6- oxohexyl)amino)-4- oxobutanoic acid | Compound 148 |
| 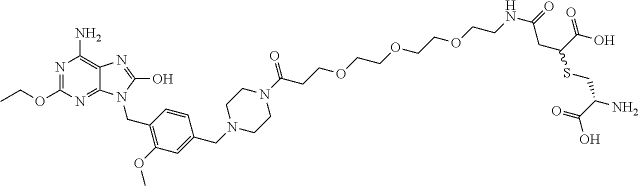 [SMILES: CCOc1nc(N)c2nc(O)n(Cc3ccc(CN4CCN(C(=O)CCOCCOCCOCCNC(=O)CC(SC[C@H](N)C(=O)O)C(=O)O)CC4)cc3OC)c2n1] | (19S)-19-amino-1- (4-(4- ((6-amino-2-ethoxy-8- hydroxy-9H-purin-9- yl)methyl)-3- methoxybenzyl) piperazin-1-yl)- 16-carboxy-1,14- dioxo-4,7,10-trioxa- 17-thia-13-azaicosan- 20-oic acid | Compound 149 |
| 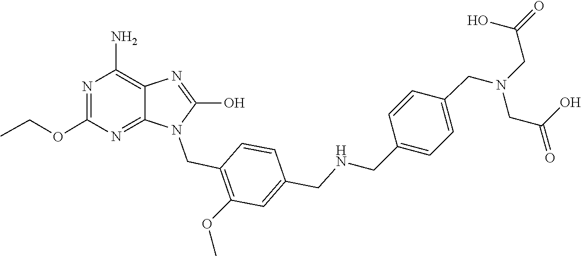 [SMILES: CCOc1nc(N)c2nc(O)n(Cc3ccc(CNCc4ccc(CN(CC(=O)O)CC(=O)O)cc4)cc3OC)c2n1] | 2,2′-((4-(((4-((6-amino- 2-ethoxy-8-hydroxy- 9H-purin-9-yl)methyl)- 3-methoxy-benzyl) amino)methyl)benzyl) azanediyl) diacetic acid | Compound 151 |
| 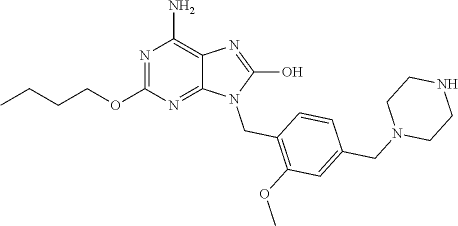 [SMILES: CCCCOc1nc(N)c2nc(O)n(Cc3ccc(CN4CCNCC4)cc3OC)c2n1] | 6-amino-2-butoxy-9- (2-methoxy-4- (piperazin- 1-ylmethyl)benzyl)- 9H-purin-8-ol | Compound 154 |
| 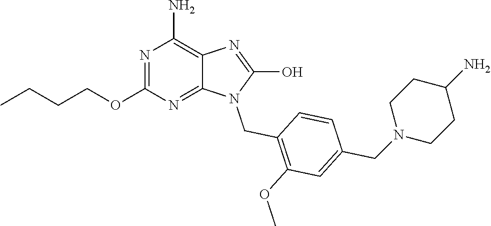 [SMILES: CCCCOc1nc(N)c2nc(O)n(Cc3ccc(CN4CCC(N)CC4)cc3OC)c2n1] | 6-amino-9-(4-((4- aminopiperidin-1- yl)methyl)-2- methoxybenzyl)-2- butoxy-9H-purin-8-ol | Compound 155 |
| 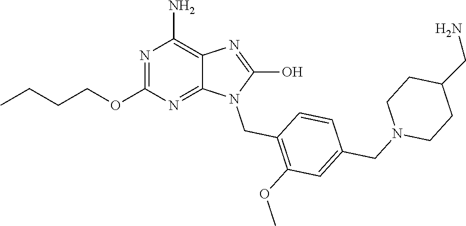 [SMILES: CCCCOc1nc(N)c2nc(O)n(Cc3ccc(CN4CCC(CN)CC4)cc3OC)c2n1] | 6-amino-9-(4-((4- (aminomethyl) piperidin- 1-yl)methyl)-2- methoxybenzyl)-2- butoxy-9H-purin- 8-ol | Compound 156 |
| 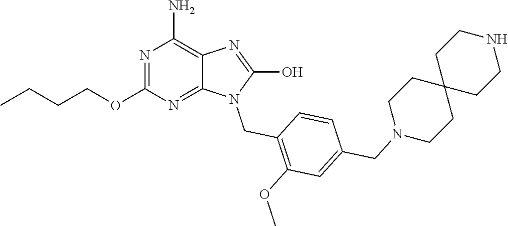 [SMILES: CCCCOc1nc(N)c2nc(O)n(Cc3ccc(CN4CCC5(CCNCC5)CC4)cc3OC)c2n1] | 9-(4-((3,9- diazaspiro[5.5] undecan- 3-yl)methyl)-2- methoxybenzyl)-6- amino-2-butoxy-9H- purin-8-ol | Compound 157 |
| 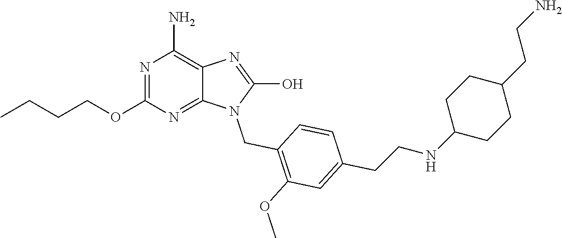 [SMILES: CCCCOc1nc(N)c2nc(O)n(Cc3ccc(CCNC4CCC(CCN)CC4)cc3OC)c2n1] | 6-amino-9-(4-((4-(2- aminoethyl)piperidin- 1-yl)methyl)-2- methoxybenzyl)-2- butoxy-9H- purin-8-ol | Compound 158 |
| 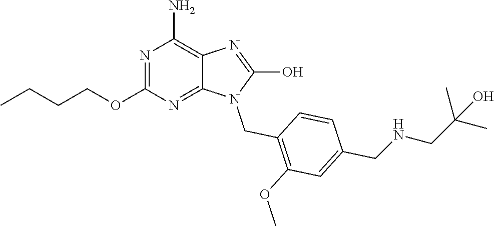 [SMILES: CCCCOc1nc(N)c2nc(O)n(Cc3ccc(CNCC(C)(C)O)cc3OC)c2n1] | 6-amino-2-butoxy-9- (4-(((2-hydroxy-2- methylpropyl)amino) methyl)-2- methoxybenzyl)- 9H-purin-8-ol | Compound 159 |
| 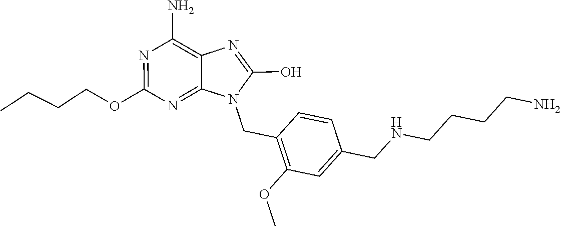 [SMILES: CCCCOc1nc(N)c2nc(O)n(Cc3ccc(CNCCCCN)cc3OC)c2n1] | 6-amino-9-(4-(((4- aminobutyl)amino) methyl)-2- methoxybenzyl)-2- butoxy-9H-purin-8-ol | Compound 160 |
| 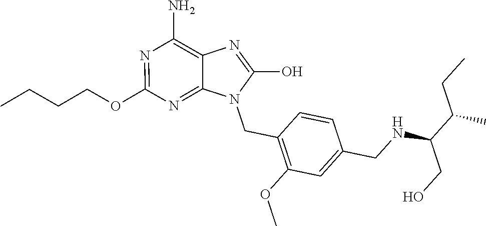 [SMILES: CCCCOc1nc(N)c2nc(O)n(Cc3ccc(CN[C@H](CO)[C@@H](C)CC)cc3OC)c2n1] | 6-amino-2-butoxy-9- (4-((((2R,3S)-1- hydroxy-3- methylpentan-2- yl)amino)-methyl)-2- methoxybenzyl)-9H- purin-8-ol | Compound 161 |
| 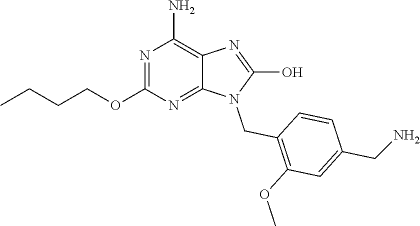 [SMILES: CCCCOc1nc(N)c2nc(O)n(Cc3ccc(CN)cc3OC)c2n1] | 6-amino-9-(4- (aminomethyl)-2- methoxybenzyl)-2- butoxy-9H-purin-8-ol | Compound 162 |
| 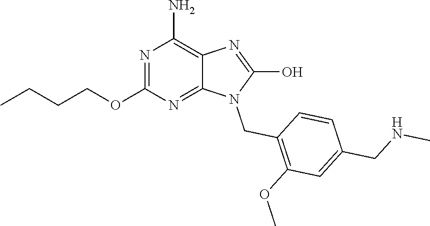 [SMILES: CCCCOc1nc(N)c2nc(O)n(Cc3ccc(CNC)cc3OC)c2n1] | 6-amino-2-butoxy-9- (2-methoxy-4- ((methylamino) methyl)benzyl)- 9H-purin-8-ol | Compound 163 |
| 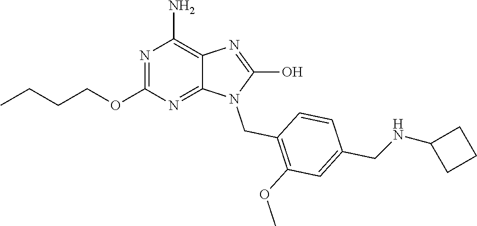 [SMILES: CCCCOc1nc(N)c2nc(O)n(Cc3ccc(CNC4CCC4)cc3OC)c2n1] | 6-amino-2-butoxy-9- (4-((cyclobutylamino) methyl)-2- methoxybenzyl)- 9H-purin-8-ol | Compound 164 |
| 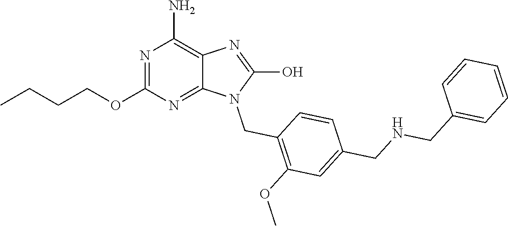 [SMILES: CCCCOc1nc(N)c2nc(O)n(Cc3ccc(CNCc4ccccc4)cc3OC)c2n1] | 6-amino-9-(4- ((benzylamino) methyl)- 2-methoxybenzyl)- 2-butoxy-9H- purin-8-ol | Compound 165 |
| 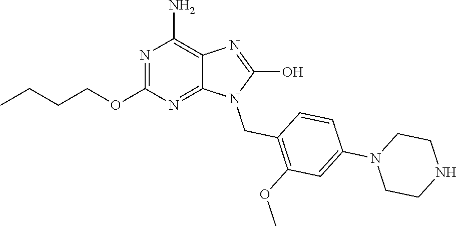 [SMILES: CCCCOc1nc(N)c2nc(O)n(Cc3ccc(N4CCNCC4)cc3OC)c2n1] | 6-amino-2-butoxy-9- (2-methoxy-4- (piperazin-1- yl)benzyl)-9H- purin-8-ol | Compound 169 |
| 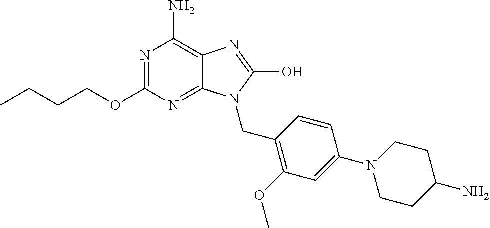 [SMILES: CCCCOc1nc(N)c2nc(O)n(Cc3ccc(N4CCC(N)CC4)cc3OC)c2n1] | 6-amino-9-(4-(4- aminopiperidin-1- yl)-2- methoxybenzyl)-2- butoxy-9H-purin- 8-ol | Compound 170 |
| 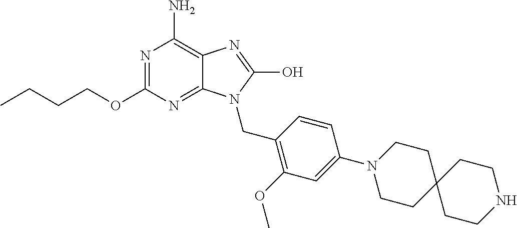 [SMILES: CCCCOc1nc(N)c2nc(O)n(Cc3ccc(N4CCC5(CCNCC5)CC4)cc3OC)c2n1] | 6-amino-2-butoxy-9- (2-methoxy-4-(3,9- diazaspiro[5.5] undecan-3-yl) benzyl)-9H- purin-8-ol | Compound 171 |
| 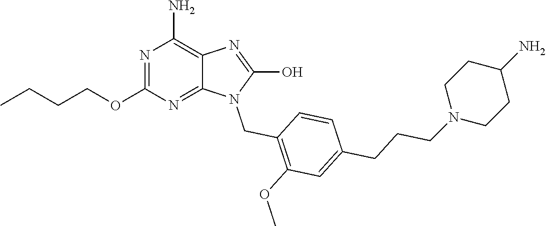 [SMILES: CCCCOc1nc(N)c2nc(O)n(Cc3ccc(CCCN4CCC(N)CC4)cc3OC)c2n1] | 6-amino-9-(4-(3-(4- aminopiperidin-1- yl)propyl)-2- methoxybenzyl)-2- butoxy-9H- purin-8-ol | Compound 172 |
| 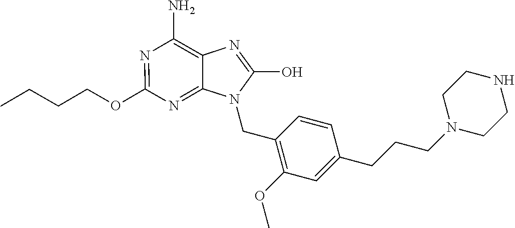 [SMILES: CCCCOc1nc(N)c2nc(O)n(Cc3ccc(CCCN4CCNCC4)cc3OC)c2n1] | 6-amino-2-butoxy-9- (2-methoxy-4-(3- (piperazin-1- yl)propyl)benzyl)- 9H-purin-8-ol | Compound 173 |
| 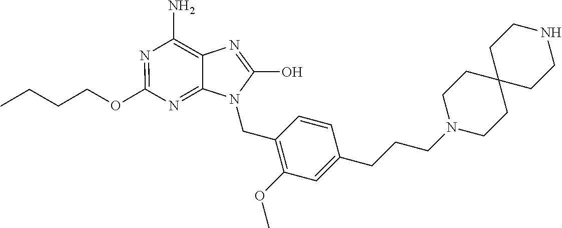 [SMILES: CCCCOc1nc(N)c2nc(O)n(Cc3ccc(CCCN4CCC5(CCNCC5)CC4)cc3OC)c2n1] | 6-amino-9-(4-(3-(4- aminopiperidin-1- yl)propyl)-2- methoxybenzyl)-2- butoxy-9H- purin-8-ol | Compound 174 |
| 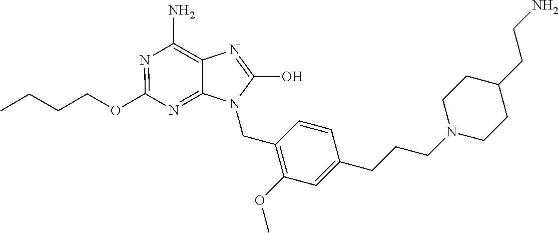 [SMILES: CCCCOc1nc(N)c2nc(O)n(Cc3ccc(CCCN4CCC(CCN)CC4)cc3OC)c2n1] | 6-amino-9-(4-(3- (4-(2-aminoethyl) piperidin-1- yl)propyl)-2- methoxybenzyl)- 2-butoxy-9H- purin-8-ol | Compound 175 |
| 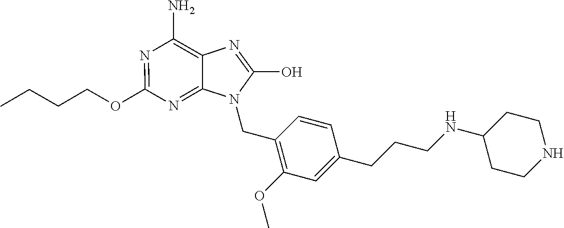 [SMILES: CCCCOc1nc(N)c2nc(O)n(Cc3ccc(CCCNC4CCNCC4)cc3OC)c2n1] | 6-amino-2-butoxy-9- (2-methoxy-4-(3- (piperidin- 4-ylamino)propyl)- benzyl)-9H- purin-8-ol | Compound 176 |
| 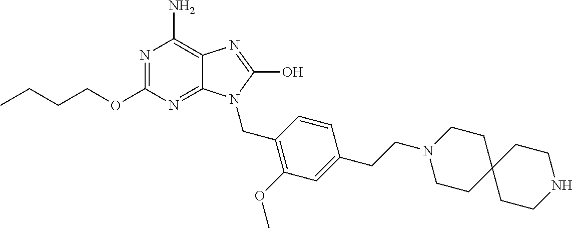 [SMILES: CCCCOc1nc(N)c2nc(O)n(Cc3ccc(CCN4CCC5(CCNCC5)CC4)cc3OC)c2n1] | 9-(4-(2-(3,9- diazaspiro[5.5] undecan- 3-yl)ethyl)-2- methoxybenzyl)-6- amino-2-butoxy- 9H-purin-8-ol | Compound 177 |
| 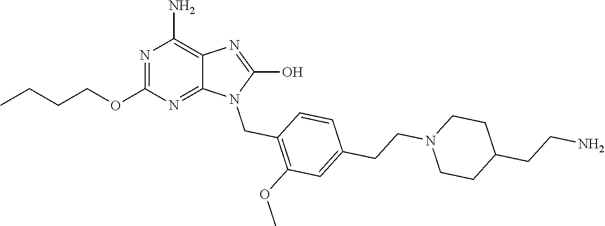 [SMILES: CCCCOc1nc(N)c2nc(O)n(Cc3ccc(CCN4CCC(CCN)CC4)cc3OC)c2n1] | 6-amino-9-(4-(2-(4- (2-aminoethyl) piperidin- 1-yl)ethyl)-2- methoxybenzyl)- 2-butoxy-9H- purin-8-ol | Compound 178 |
| 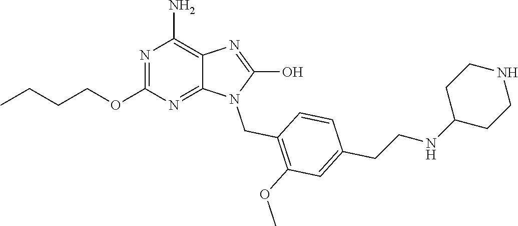 [SMILES: CCCCOc1nc(N)c2nc(O)n(Cc3ccc(CCNC4CCNCC4)cc3OC)c2n1] | 6-amino-2-butoxy-9- (2-methoxy-4-(2- (piperidin- 4-ylamino)ethyl) benzyl)- 9H-purin-8-ol | Compound 179 |
| 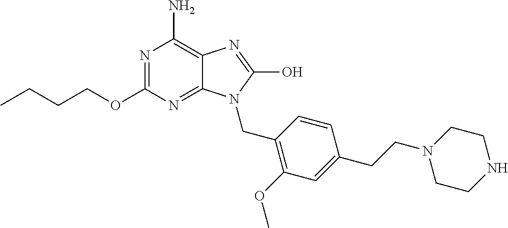 [SMILES: CCCCOc1nc(N)c2nc(O)n(Cc3ccc(CCN4CCNCC4)cc3OC)c2n1] | 6-amino-2-butoxy- 9-(2-methoxy-4-(2- (piperazin-1- yl)ethyl)benzyl)-9H- purin-8-ol | Compound 180 |
| 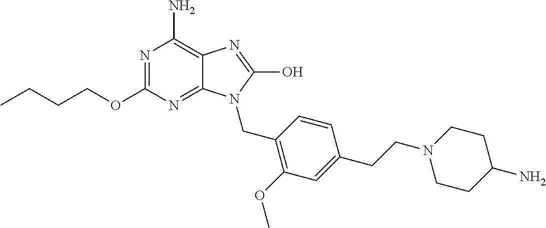 [SMILES: CCCCOc1nc(N)c2nc(O)n(Cc3ccc(CCN4CCC(N)CC4)cc3OC)c2n1] | 6-amino-9-(4-(2-(4- aminopiperidin-1- yl)ethyl)-2- methoxybenzyl)-2- butoxy-9H-purin-8-ol | Compound 181 |

SUBTABLE 1B:

| | | |
|---|---|---|
| 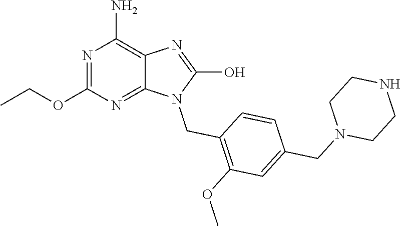 [SMILES: CCOc1nc(N)c2nc(O)n(Cc3ccc(CN4CCNCC4)cc3OC)c2n1] | 6-amino-2-ethoxy- 9-(2- methoxy-4-(piperazin- 1-ylmethyl)benzyl)- 9H-purin-8-ol | Compound 100 |
| 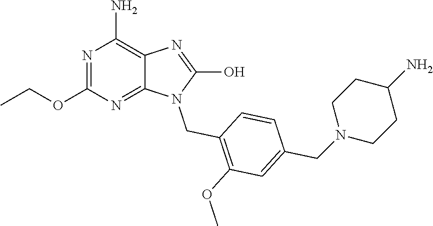 [SMILES: CCOc1nc(N)c2nc(O)n(Cc3ccc(CN4CCC(N)CC4)cc3OC)c2n1] | 6-amino-9-(4-((4- aminopiperidin-1- yl)methyl)-2- methoxybenzyl)-2- ethoxy-9H-purin- 8-ol | Compound 110 |
| 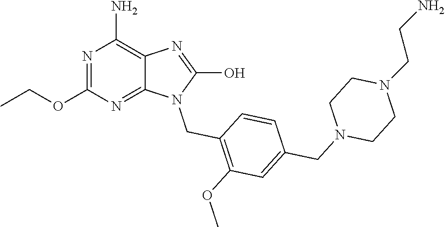 [SMILES: CCOc1nc(N)c2nc(O)n(Cc3ccc(CN4CCN(CCN)CC4)cc3OC)c2n1] | 6-amino-9-(4-(((2- aminoethyl)amino)meth yl)-2-methoxybenzyl)-2- ethoxy-9H-purin-8-ol | Compound 112 |
| 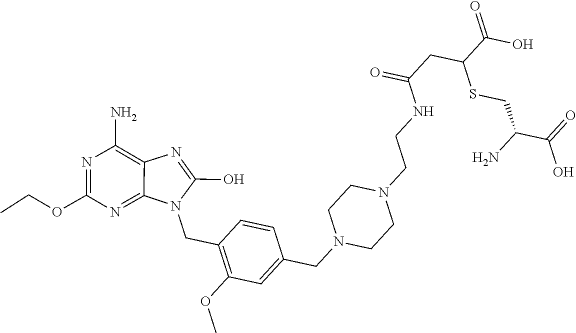 [SMILES: CCOc1nc(N)c2nc(O)n(Cc3ccc(CN4CCN(CCNC(=O)CC(SC[C@@H](N)C(=O)O)C(=O)O)CC4)cc3OC)c2n1] | 2-(((S)-2-amino-2- carboxyethyl)thio)- 4-((2-(4-(4-((6- amino-2- ethoxy-8-hydroxy- 9H-purin-9- yl)methyl)-3- methoxybenzyl) piperazin-1- yl)ethyl)amino)- 4-oxobutanoic acid | Compound 113 |
| 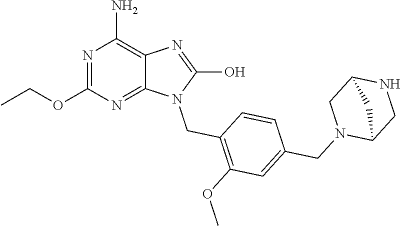 [SMILES: CCOc1nc(N)c2nc(O)n(Cc3ccc(CN4C[C@@H]5C[C@H]4CN5)cc3OC)c2n1] | 9-(4-(((1S,4S)-2,5- diazabicyclo[2.2.1] heptan-2-yl)methyl)- 2-methoxybenzyl)- 6-amino-2-ethoxy- 9H-purin-8-ol | Compound 128 |
| 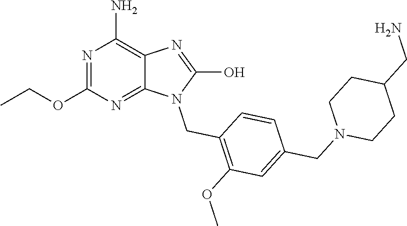 [SMILES: CCOc1nc(N)c2nc(O)n(Cc3ccc(CN4CCC(CN)CC4)cc3OC)c2n1] | 6-amino-9-(4-((4- (aminomethyl) piperidin- 1-yl)methyl)-2- methoxybenzyl)-2- ethoxy-9H- purin-8-ol | Compound 132 |
| 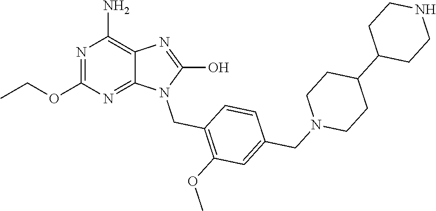 [SMILES: CCOc1nc(N)c2nc(O)n(Cc3ccc(CN4CCC(C5CCNCC5)CC4)cc3OC)c2n1] | 9-(4-([4,4′- bipiperidin]- 1-ylmethyl)-2- methoxybenzyl)-6- amino-2-ethoxy-9H- purin-8-ol | Compound 138 |
| 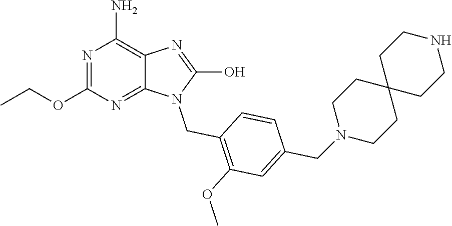 [SMILES: CCOc1nc(N)c2nc(O)n(Cc3ccc(CN4CCC5(CCNCC5)CC4)cc3OC)c2n1] | 9-(4-((3,9- diazaspiro[5.5] undecan- 3-yl)methyl)-2- methoxybenzyl)- 6-amino-2-ethoxy- 9H-purin-8-ol | Compound 139 |
| 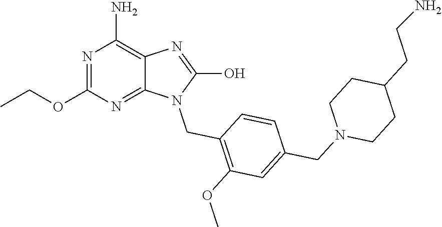 [SMILES: CCOc1nc(N)c2nc(O)n(Cc3ccc(CN4CCC(CCN)CC4)cc3OC)c2n1] | 6-amino-9-(4-((4-(2- aminoethyl)piperidin- 1-yl)methyl)-2- methoxybenzyl)-2- ethoxy-9H-purin-8-ol | Compound 140 |
| 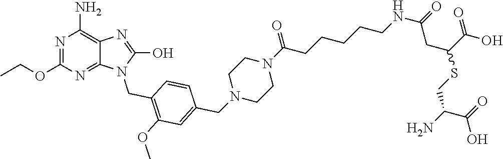 [SMILES: CCOc1nc(N)c2nc(O)n(Cc3ccc(CN4CCN(C(=O)CCCCCNC(=O)CC(SC[C@@H](N)C(=O)O)C(=O)O)CC4)cc3OC)c2n1] | 2-(((S)-2-amino-2- carboxyethyl)thio)- 4-((6-(4-(4- ((6-amino-2- ethoxy-8-hydroxy- 9H-purin-9-yl) methyl)-3- methoxybenzyl) piperazin-1-yl)-6- oxohexyl)amino)- 4-oxobutanoic acid | Compound 148 |
| 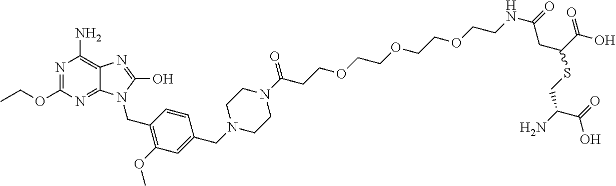 [SMILES: CCOc1nc(N)c2nc(O)n(Cc3ccc(CN4CCN(C(=O)CCOCCOCCOCCNC(=O)CC(SC[C@@H](N)C(=O)O)C(=O)O)CC4)cc3OC)c2n1] | (19S)-19-amino-1- (4-(4-((6-amino-2- ethoxy-8-hydroxy- 9H-purin-9- yl)methyl)-3- methoxybenzyl) piperazin-1-yl)- 16-carboxy-1,14- dioxo-4,7, 10- trioxa-17- thia-13-azaicosan- 20-oic acid | Compound 149 |
| 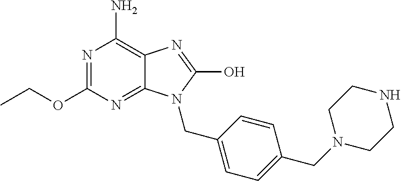 [SMILES: CCOc1nc(N)c2nc(O)n(Cc3ccc(CN4CCNCC4)cc3)c2n1] | 6-amino-2-ethoxy-9- (4-(piperazin-1- ylmethyl)benzyl)-9H- purin-8-ol | Compound 150 |
| 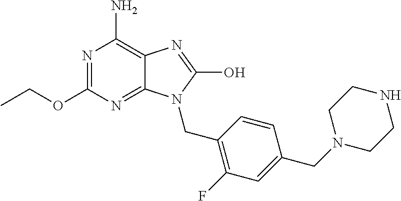 [SMILES: CCOc1nc(N)c2nc(O)n(Cc3ccc(CN4CCNCC4)cc3F)c2n1] | 6-amino-2-ethoxy-9- (2-fluoro-4- (piperazin-1- ylmethyl)benzyl)- 9H-purin-8-ol | Compound 152 |
| 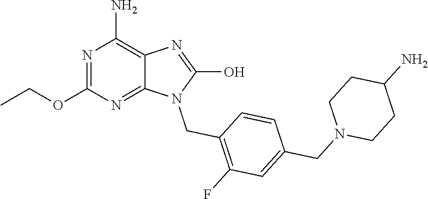 [SMILES: CCOc1nc(N)c2nc(O)n(Cc3ccc(CN4CCC(N)CC4)cc3F)c2n1] | 6-amino-9-(4-((4- aminopiperidin-1- yl)methyl)-2- fluorobenzyl)-2- ethoxy- 9H-purin-8-ol | Compound 153 |
| 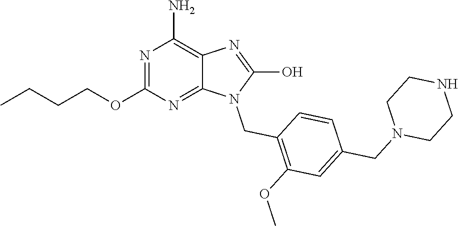 [SMILES: CCCCOc1nc(N)c2nc(O)n(Cc3ccc(CN4CCNCC4)cc3OC)c2n1] | 6-amino-2-butoxy-9- (2-methoxy-4- (piperazin-1- ylmethyl)benzyl)- 9H-purin-8-ol | Compound 154 |
| 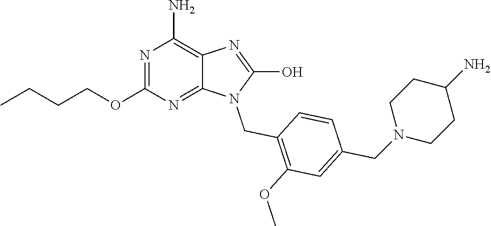 [SMILES: CCCCOc1nc(N)c2nc(O)n(Cc3ccc(CN4CCC(N)CC4)cc3OC)c2n1] | 6-amino-9-(4-((4- aminopiperidin-1- yl)methyl)-2- methoxybenzyl)-2- butoxy-9H-purin-8-ol | Compound 155 |
| 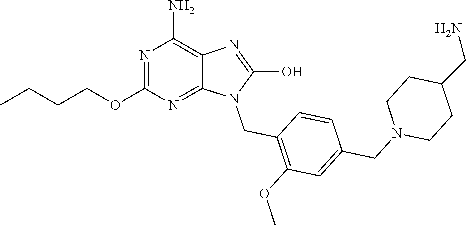 [SMILES: CCCCOc1nc(N)c2nc(O)n(Cc3ccc(CN4CCC(CN)CC4)cc3OC)c2n1] | 6-amino-9-(4-((4- (aminomethyl) piperidin- 1-yl)methyl)-2- methoxybenzyl)-2- butoxy-9H-purin-8-ol | Compound 156 |
| 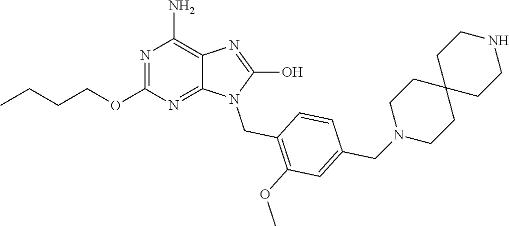 [SMILES: CCCCOc1nc(N)c2nc(O)n(Cc3ccc(CN4CCC5(CCNCC5)CC4)cc3OC)c2n1] | 9-(4-((3,9- diazaspiro[5.5] undecan- 3-yl)methyl)-2- methoxybenzyl)-6- amino-2-butoxy- 9H-purin-8-ol | Compound 157 |
| 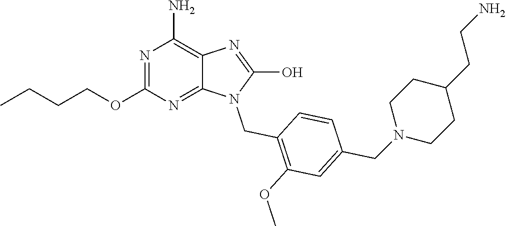 [SMILES: CCCCOc1nc(N)c2nc(O)n(Cc3ccc(CN4CCC(CCN)CC4)cc3OC)c2n1] | 6-amino-9-(4-((4-(2- aminoethyl) piperidin-1- yl)methyl)-2- methoxybenzyl)-2- butoxy-9H- purin-8-ol | Compound 158 |
| 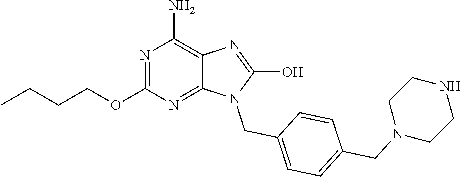 [SMILES: CCCCOc1nc(N)c2nc(O)n(Cc3ccc(CN4CCNCC4)cc3)c2n1] | 6-amino-2-butoxy-9- (4-(piperazin-1- ylmethyl)benzyl)-9H- purin-8-ol | Compound 166 |
| 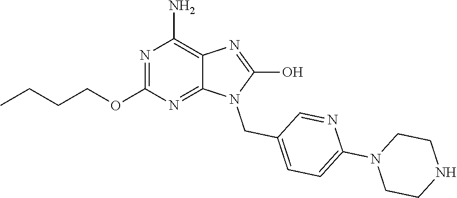 [SMILES: CCCCOc1nc(N)c2nc(O)n(Cc3ccc(N4CCNCC4)nc3)c2n1] | 6-amino-2-butoxy- 9-((6- (piperazin-1-yl)pyridin- 3-yl)methyl)-9H-purin- 8-ol | Compound 167 |
| 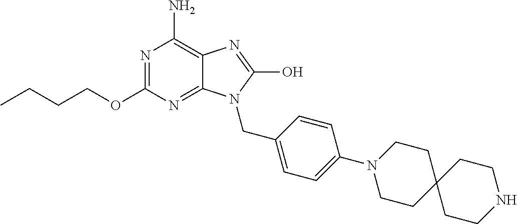 [SMILES: CCCCOc1nc(N)c2nc(O)n(Cc3ccc(N4CCC5(CCNCC5)CC4)cc3)c2n1] | 9-((6-(3,9- diazaspiro[5.5] undecan- 3-yl)pyridin-3- yl)methyl)-6- amino-2- butoxy-9H- purin-8-ol | Compound 168 |
| 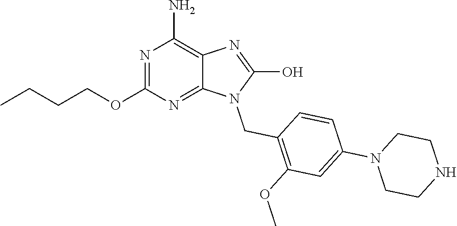 [SMILES: CCCCOc1nc(N)c2nc(O)n(Cc3ccc(N4CCNCC4)cc3OC)c2n1] | 6-amino-2-butoxy- 9-(2- methoxy-4- (piperazin-1- yl)benzyl)-9H- purin-8-ol | Compound 169 |
| 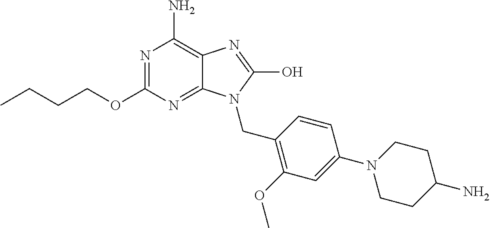 [SMILES: CCCCOc1nc(N)c2nc(O)n(Cc3ccc(N4CCC(N)CC4)cc3OC)c2n1] | 6-amino-9-(4-(4- aminopiperidin-1-yl)- 2-methoxybenzyl)- 2-butoxy-9H- purin-8-ol | Compound 170 |
| 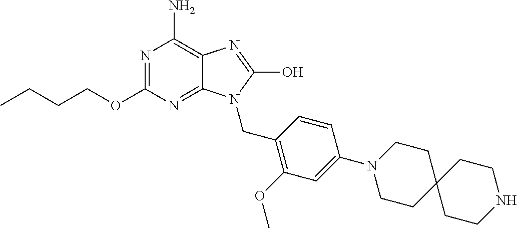 [SMILES: CCCCOc1nc(N)c2nc(O)n(Cc3ccc(N4CCC5(CCNCC5)CC4)cc3OC)c2n1] | 6-amino-2-butoxy-9- (2-methoxy-4-(3,9- diazaspiro[5.5] undecan-3- yl)benzyl)-9H- purin-8-ol | Compound 171 |
| 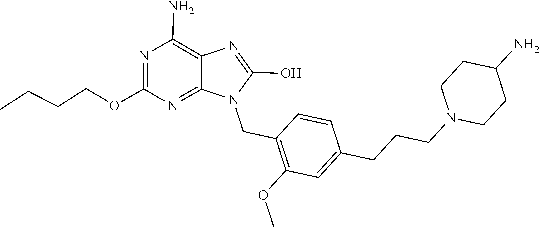 [SMILES: CCCCOc1nc(N)c2nc(O)n(Cc3ccc(CCCN4CCC(N)CC4)cc3OC)c2n1] | 6-amino-9-(4-(3-(4- aminopiperidin-1- yl)propyl)-2- methoxybenzyl)-2- butoxy-9H-purin-8-ol | Compound 172 |
| 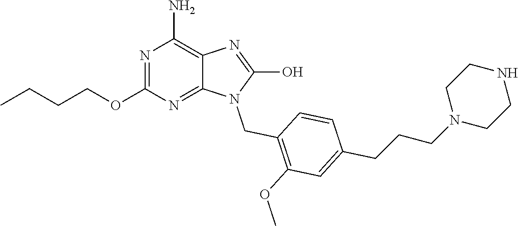 [SMILES: CCCCOc1nc(N)c2nc(O)n(Cc3ccc(CCCN4CCNCC4)cc3OC)c2n1] | 6-amino-2-butoxy-9- (2-methoxy-4-(3- (piperazin-1- yl)propyl)benzyl)-9H- purin-8-ol | Compound 173 |
| 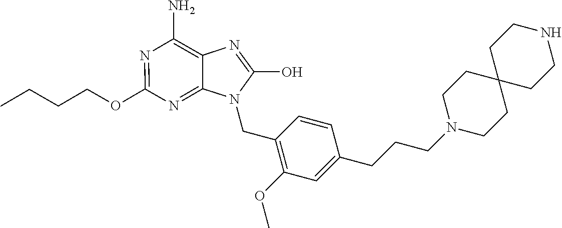 [SMILES: CCCCOc1nc(N)c2nc(O)n(Cc3ccc(CCCN4CCC5(CCNCC5)CC4)cc3OC)c2n1] | 6-amino-9-(4-(3-(4- aminopiperidin-1- yl)propyl)-2- methoxybenzyl)-2- butoxy-9H-purin-8-ol | Compound 174 |
| 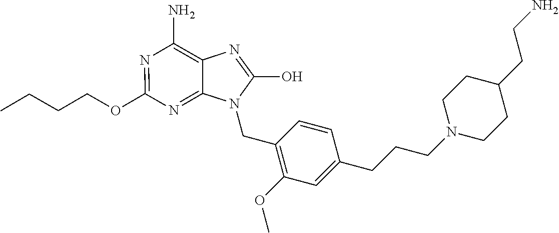 [SMILES: CCCCOc1nc(N)c2nc(O)n(Cc3ccc(CCCN4CCC(CCN)CC4)cc3OC)c2n1] | 6-amino-9-(4-(3- (4-(2- aminoethyl)piperidin- 1-yl)propyl)-2- methoxybenzyl)-2- butoxy-9H-purin-8-ol | Compound 175 |
| 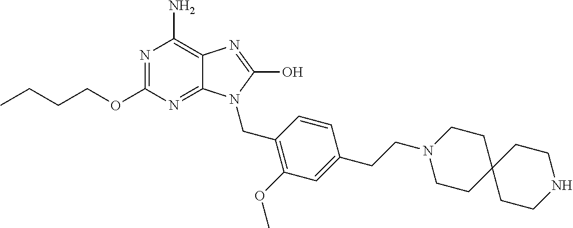 [SMILES: CCCCOc1nc(N)c2nc(O)n(Cc3ccc(CCN4CCC5(CCNCC5)CC4)cc3OC)c2n1] | 9-(4-(2-(3,9- diazaspiro[5.5] undecan- 3-yl)ethyl)-2- methoxybenzyl)- 6-amino-2-butoxy- 9H-purin-8-ol | Compound 177 |
| 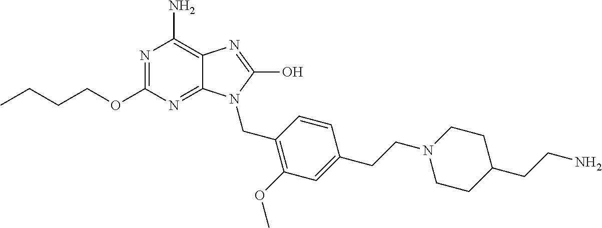 [SMILES: CCCCOc1nc(N)c2nc(O)n(Cc3ccc(CCN4CCC(CCN)CC4)cc3OC)c2n1] | 6-amino-9-(4-(2- (4-(2-aminoethyl) piperidin-1- yl)ethyl)-2- methoxybenzyl)-2- butoxy-9H-purin-8-ol | Compound 178 |
| 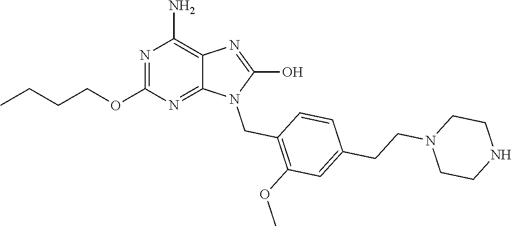 [SMILES: CCCCOc1nc(N)c2nc(O)n(Cc3ccc(CCN4CCNCC4)cc3OC)c2n1] | 6-amino-2-butoxy-9- (2-methoxy-4-(2- (piperazin-1- yl)ethyl)benzyl)- 9H-purin-8-ol | Compound 180 |
| 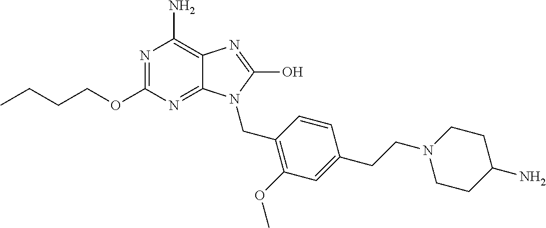 [SMILES: CCCCOc1nc(N)c2nc(O)n(Cc3ccc(CCN4CCC(N)CC4)cc3OC)c2n1] | 6-amino-9-(4-(2-(4- aminopiperidin-1- yl)ethyl)-2- methoxybenzyl)-2- butoxy-9H-purin-8-ol | Compound 181 |
| 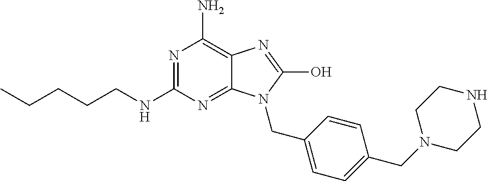 [SMILES: CCCCCNc1nc(N)c2nc(O)n(Cc3ccc(CN4CCNCC4)cc3)c2n1] | 6-amino-2- (pentylamino)-9-(4- (piperazin-1- ylmethyl)benzyl)- 9H-purin-8-ol | Compound 187 |
| 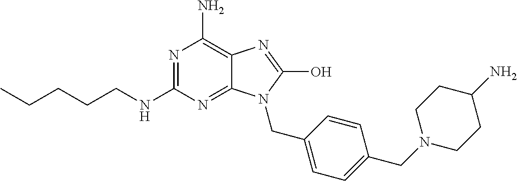 [SMILES: CCCCCNc1nc(N)c2nc(O)n(Cc3ccc(CN4CCC(N)CC4)cc3)c2n1] | 6-amino-9-(4-((4- aminopiperidin-1- yl)methyl)benzyl)-2- (pentylamino)- 9H-purin-8-ol | Compound 188 |
| 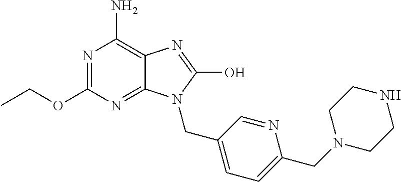 [SMILES: CCOc1nc(N)c2nc(O)n(Cc3ccc(CN4CCNCC4)nc3)c2n1] | 6-amino-2-ethoxy-9- ((6-(piperazin-1- ylmethyl)pyridin-3- yl)methyl)-9H- purin-8-ol | Compound 192 |

SUBTABLE 1C:

| | | |
|---|---|---|
| 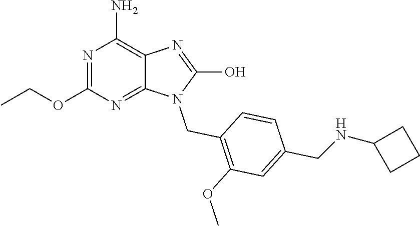 [SMILES: CCOc1nc(N)c2nc(O)n(Cc3ccc(CNC4CCC4)cc3OC)c2n1] | 6-amino-9-(4- ((cyclobutylamino) methyl)-2- methoxybenzyl)-2- ethoxy-9H-purin-8-ol | Compound 108 |
| 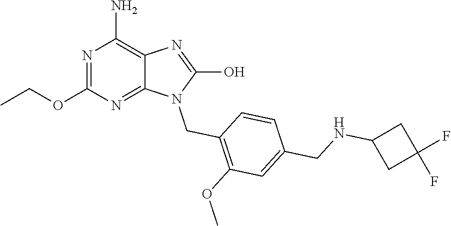 [SMILES: CCOc1nc(N)c2nc(O)n(Cc3ccc(CNC4CC(F)(F)C4)cc3OC)c2n1] | 6-amino-9-(4-(((3,3- difluorocyclobutyl)- amino)methyl)-2- methoxybenzyl)-2- ethoxy-9H-purin-8-ol | Compound 109 |
| 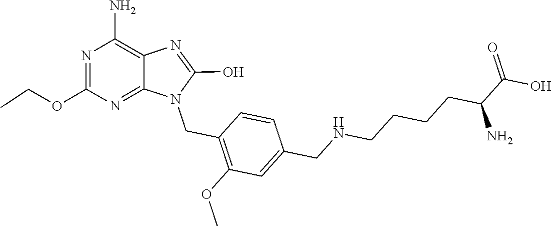 [SMILES: CCOc1nc(N)c2nc(O)n(Cc3ccc(CNCCCC[C@H](N)C(=O)O)cc3OC)c2n1] | N⁶-(4-((6-amino-2- ethoxy-8-hydroxy-9H- purin-9-yl)methyl)-3- methoxybenzyl)-L- lysine | Compound 114 |
| 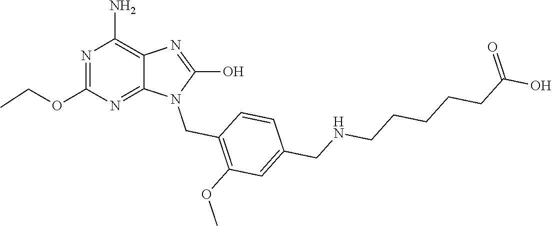 [SMILES: CCOc1nc(N)c2nc(O)n(Cc3ccc(CNCCCCCC(=O)O)cc3OC)c2n1] | 6-((4-((6-amino-2- ethoxy-8-hydroxy-9H- purin-9-yl)methyl)-3- methoxybenzyl)- amino)hexanoic acid | Compound 115 |
| 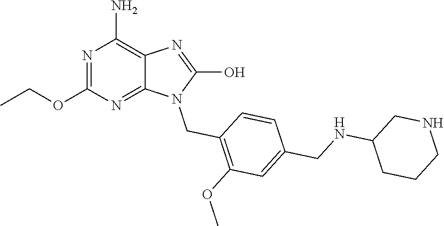 [SMILES: CCOc1nc(N)c2nc(O)n(Cc3ccc(CNC4CCCNC4)cc3OC)c2n1] | 6-amino-2-ethoxy-9-(2- methoxy-4-((piperidin-3 ylamino)methyl)benzyl)- 9H-purin-8-ol | Compounds 127 |
| 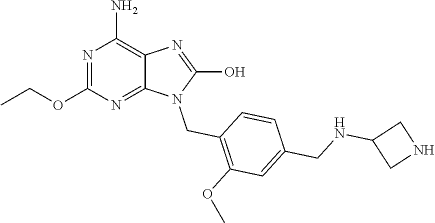 [SMILES: CCOc1nc(N)c2nc(O)n(Cc3ccc(CNC4CNC4)cc3OC)c2n1] | 6-amino-9-(4-((azetidin- 3-ylamino)methyl)-2- methoxybenzyl)-2- ethoxy-9H-purin-8-ol | Compound 129 |
| 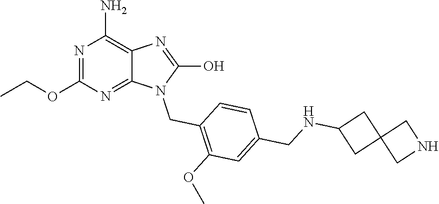 [SMILES: CCOc1nc(N)c2nc(O)n(Cc3ccc(CNC4CC5(CNC5)C4)cc3OC)c2n1] | 9-(4-(((2- azaspiro[3.3]heptan-6- yl)amino)methyl)-2- methoxybenzyl)-6- amino-2-ethoxy-9H- purin-8-ol | Compound 130 |
| 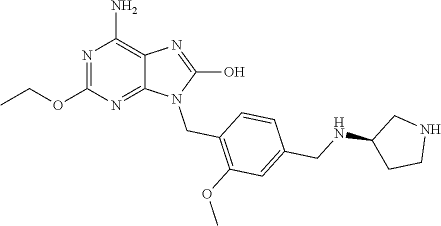 [SMILES: CCOc1nc(N)c2nc(O)n(Cc3ccc(CN[C@@H]4CCNC4)cc3OC)c2n1] | (S)-6-amino-2-ethoxy- 9-(2-methoxy-4- ((pyrrolidin-3- ylamino)methyl) benzyl)- 9H-purin-8-ol | Compound 133 |
| 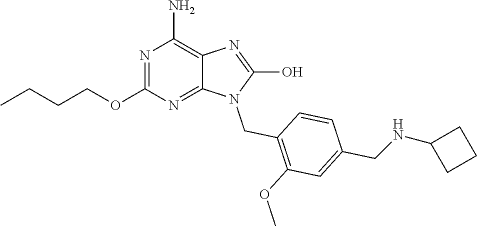 [SMILES: CCCCOc1nc(N)c2nc(O)n(Cc3ccc(CNC4CCC4)cc3OC)c2n1] | 6-amino-2-butoxy- 9-(4- ((cyclobutylamino) methyl)-2- methoxybenzyl)- 9H-purin-8-ol | Compound 164 |
| 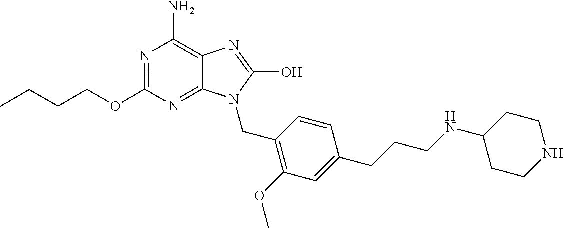 [SMILES: CCCCOc1nc(N)c2nc(O)n(Cc3ccc(CCCNC4CCNCC4)cc3OC)c2n1] | 6-amino-2-butoxy-9- (2-methoxy-4-(3- (piperidin- 4-ylamino)propyl)- benzyl)-9H-purin-8-ol | Compound 176 |
| 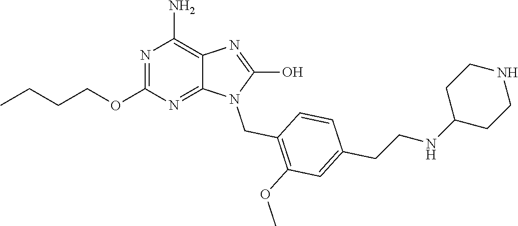 [SMILES: CCCCOc1nc(N)c2nc(O)n(Cc3ccc(CCNC4CCNCC4)cc3OC)c2n1] | 6-amino-2-butoxy-9- (2-methoxy-4-(2- (piperidin- 4-ylamino)ethyl) benzyl)- 9H-purin-8-ol | Compound 179 |
| 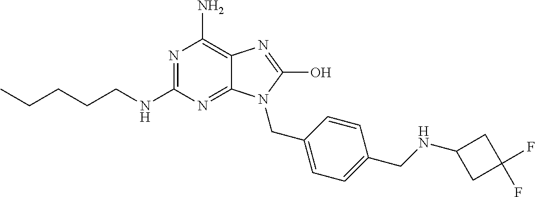 [SMILES: CCCCCNc1nc(N)c2nc(O)n(Cc3ccc(CNC4CC(F)(F)C4)cc3)c2n1] | 6-amino-9-(4-(((3,3- difluorocyclobutyl) amino)methyl) benzyl)-2- (pentyl-amino)-9H- purin-8-ol | Compound 186 |
| 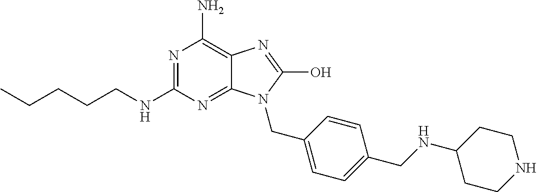 [SMILES: CCCCCNc1nc(N)c2nc(O)n(Cc3ccc(CNC4CCNCC4)cc3)c2n1] | 6-amino-2- (pentylamino)-9-(4- ((piperidin-4- ylamino)methyl) benzyl)- 9H-purin-8-ol | Compound 194 |

SUBTABLE 1D:

| | | |
|---|---|---|
| 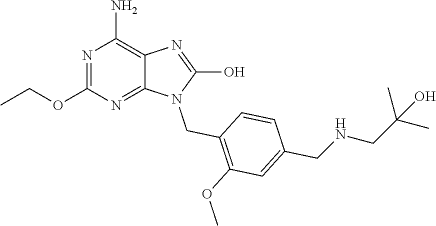 [SMILES: CCOc1nc(N)c2nc(O)n(Cc3ccc(CNCC(C)(C)O)cc3OC)c2n1] | 6-amino-2-ethoxy-9- (4-(((2-hydroxy-2- methylpropyl)amino) methyl)-2- methoxybenzyl)- 9H-purin-8-ol | Compound 101 |
| 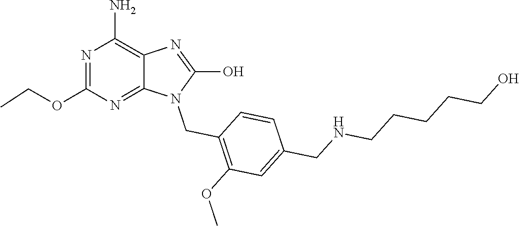 [SMILES: CCOc1nc(N)c2nc(O)n(Cc3ccc(CNCCCCCO)cc3OC)c2n1] | 6-amino-2-ethoxy-9-(4- (((5-hydroxypentyl)- amino)methyl)-2- methoxy-benzyl)-9H- purin-8-ol | Compound 103 |
| 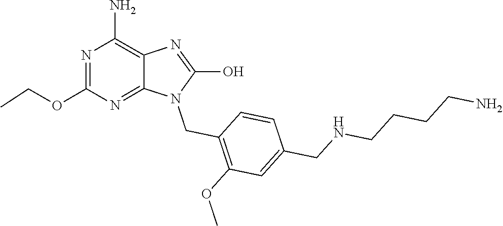 [SMILES: CCOc1nc(N)c2nc(O)n(Cc3ccc(CNCCCCN)cc3OC)c2n1] | 6-amino-9-(4-(((4- aminobutyl)amino) methyl)-2- methoxybenzyl)-2- ethoxy-9H-purin-8-ol | Compound 106 |
| 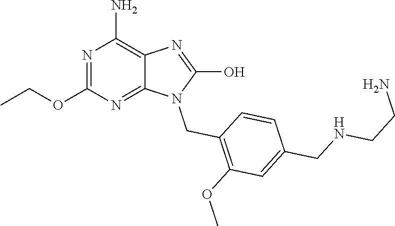 [SMILES: CCOc1nc(N)c2nc(O)n(Cc3ccc(CNCCN)cc3OC)c2n1] | 6-amino-9-(4-(((2- aminoethyl)amino) methyl)-2- methoxybenzyl)- 2-ethoxy-9H- purin-8-ol | Compound 111 |
| 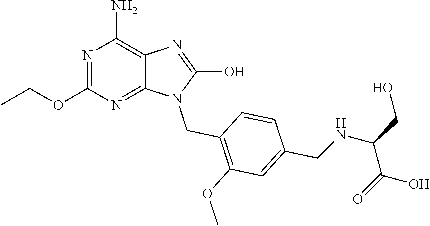 [SMILES: CCOc1nc(N)c2nc(O)n(Cc3ccc(CN[C@@H](CO)C(=O)O)cc3OC)c2n1] | (4-((6-amino-2-ethoxy- 8-hydroxy-9H-purin-9- yl)methyl)-3- methoxybenzyl)-L- serine | Compound 119 |
| 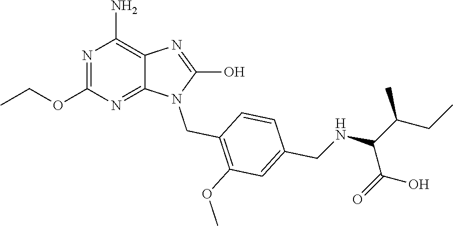 | (4-((6-amino-2-ethoxy- 8-hydroxy-9H-purin-9- yl)methyl)-3- methoxybenzyl)-L- isoleucine | Compound 121 |
| 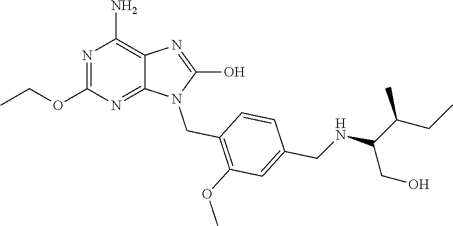 | 6-amino-2-ethoxy-9- (4-((((2S,3S)-1- hydroxy-3- methylpentan-2- yl)amino)-methyl)-2- methoxybenzyl)-9H- purin-8-ol | Compound 122 |
| 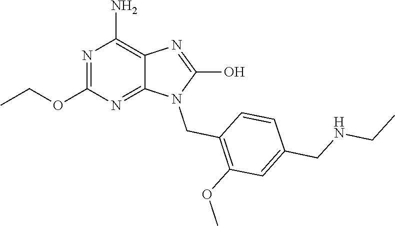 [SMILES: CCNCc1ccc(Cn2c(O)nc3c(N)nc(OCC)nc32)c(OC)c1] | 6-amino-2-ethoxy-9- (4-((ethylamino) methyl)-2- methoxybenzyl)- 9H-purin-8-ol | Compound 124 |
| 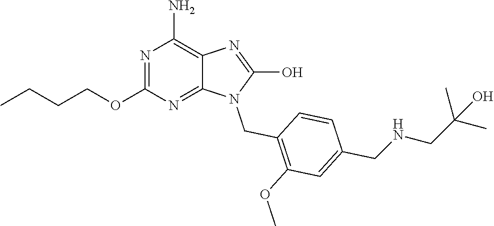 [SMILES: CCCCOc1nc(N)c2nc(O)n(Cc3ccc(CNCC(C)(C)O)cc3OC)c2n1] | 6-amino-2-butoxy-9- (4-(((2-hydroxy-2- methylpropyl)amino) methyl)-2- methoxybenzyl)- 9H-purin-8-ol | Compound 159 |
| 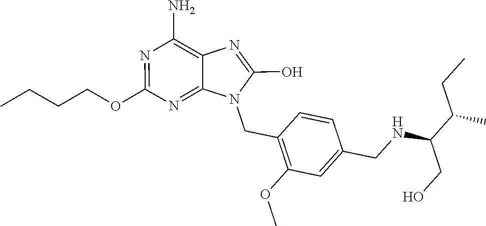 [SMILES: CCCCOc1nc(N)c2nc(O)n(Cc3ccc(CN[C@H](CO)[C@@H](C)CC)cc3OC)c2n1] | 6-amino-2-butoxy-9- (4-((((2R,3S)-1- hydroxy-3- methylpentan-2- yl)amino)-methyl)-2- methoxybenzyl)-9H- purin-8-ol | Compound 161 |
| 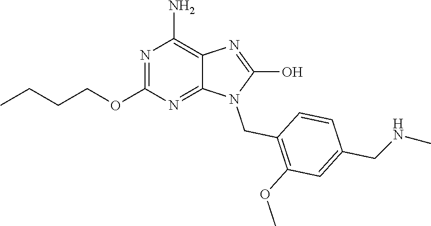 [SMILES: CCCCOc1nc(N)c2nc(O)n(Cc3ccc(CNC)cc3OC)c2n1] | 6-amino-2-butoxy-9- (2-methoxy-4- ((methylamino) methyl)benzyl)- 9H-purin-8-ol | Compound 163 |
| 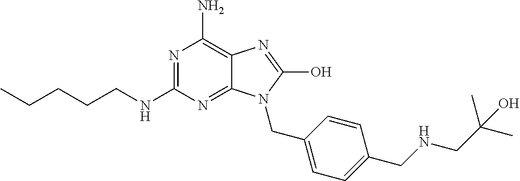 [SMILES: CCCCCNc1nc(N)c2nc(O)n(Cc3ccc(CNCC(C)(C)O)cc3)c2n1] | 6-amino-9-(4-(((2- hydroxy-2- methylpropyl)amino) methyl)benzyl)-2- (pentyl- amino)-9H-purin-8-ol | Compound 184 |
| 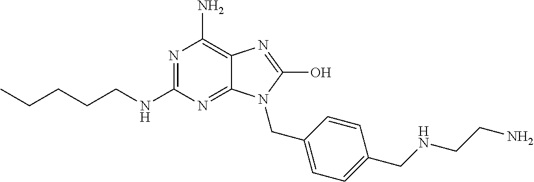 [SMILES: CCCCCNc1nc(N)c2nc(O)n(Cc3ccc(CNCCN)cc3)c2n1] | 6-amino-9-(4-(((2- aminoethyl)amino) methyl)benzyl)-2- (pentylamino)- 9H-purin-8-ol | Compound 189 |
| 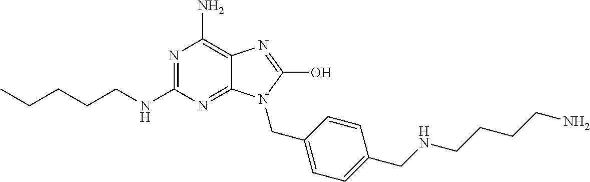 [SMILES: CCCCCNc1nc(N)c2nc(O)n(Cc3ccc(CNCCCCN)cc3)c2n1] | 6-amino-9-(4-(((4- aminobutyl)amino) methyl)benzyl)-2- (pentylamino)-9H- purin-8-ol | Compound 190 |
| 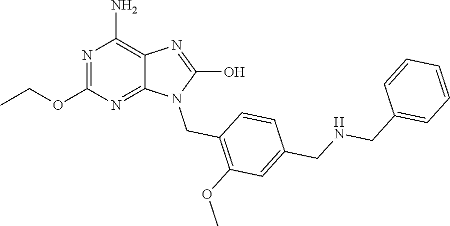 [SMILES: CCOc1nc(N)c2nc(O)n(Cc3ccc(CNCc4ccccc4)cc3OC)c2n1] | 6-amino-9-(4- ((benzylamino) methyl)-2- methoxybenzyl)-2- ethoxy-9H- purin-8-ol | Compound 107 |
| 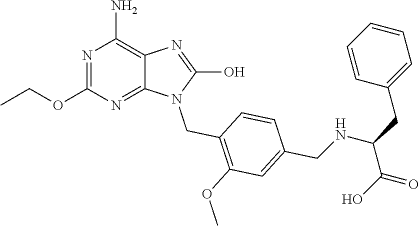 [SMILES: CCOc1nc(N)c2nc(O)n(Cc3ccc(CN[C@@H](Cc4ccccc4)C(=O)O)cc3OC)c2n1] | (4-((6-amino-2- ethoxy-8-hydroxy- 9H-purin-9- yl)methyl)-3- methoxybenzyl)-L- phenylalanine | Compound 117 |
| 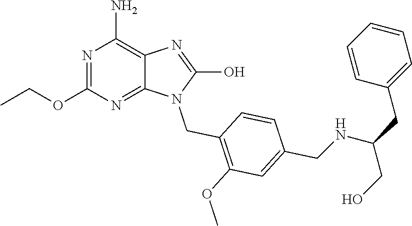 [SMILES: CCOc1nc(N)c2nc(O)n(Cc3ccc(CN[C@H](CO)Cc4ccccc4)cc3OC)c2n1] | (S)-6-amino-2- ethoxy-9- (4-(((1-hydroxy-3- phenylpropan-2- yl)amino)methyl)-2- methoxybenzyl)-9H- purin-8-ol | Compound 118 |
| 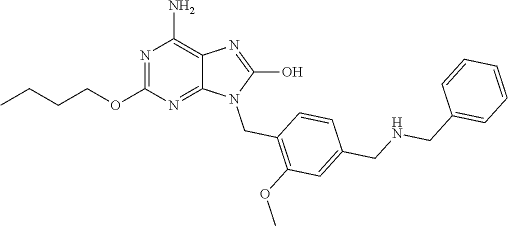 [SMILES: CCCCOc1nc(N)c2nc(O)n(Cc3ccc(CNCc4ccccc4)cc3OC)c2n1] | 6-amino-9-(4- ((benzylamino)methyl)- 2-methoxybenzyl)-2- butoxy-9H-purin-8-ol | Compound 165 |
| 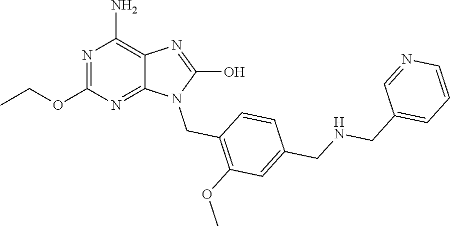 [SMILES: CCOc1nc(N)c2nc(O)n(Cc3ccc(CNCc4cccnc4)cc3OC)c2n1] | 6-amino-9-(4- ((cyclobutylamino) methyl)-2- methoxybenzyl)- 2-ethoxy-9H- purin-8-ol | Compound 193 |

SUBTABLE 1E:

| | | |
|---|---|---|
| 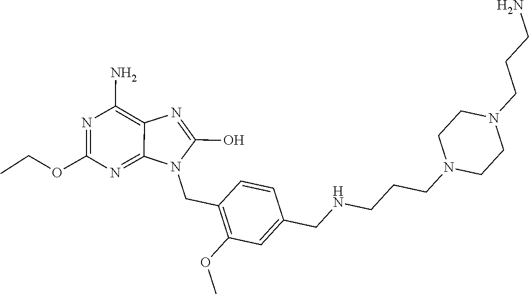 [SMILES: CCOc1nc(N)c2nc(O)n(Cc3ccc(CNCCCN4CCN(CCCN)CC4)cc3OC)c2n1] | 6-amino-9-(4-(((3- (4-(3-aminopropyl) piperazin-1- yl)propyl)amino)- methyl)-2- methoxybenzyl)- 2-ethoxy-9H- purin-8-ol | Compound 104 |
| 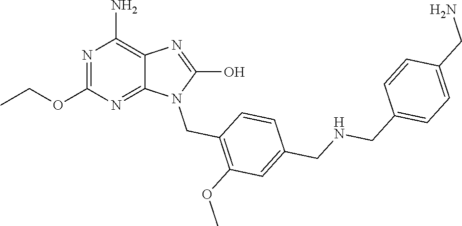 [SMILES: CCOc1nc(N)c2nc(O)n(Cc3ccc(CNCc4ccc(CN)cc4)cc3OC)c2n1] | 6-amino-9-(4-(((4- (aminomethyl)benzyl) amino)methyl)-2- methoxybenzyl)-2- ethoxy-9H- purin-8-ol | Compound 105 |
| 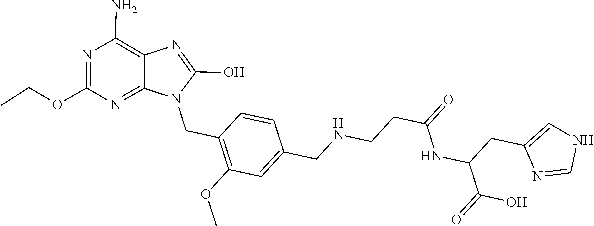 [SMILES: CCOc1nc(N)c2nc(O)n(Cc3ccc(CNCCC(=O)NC(Cc4c[nH]cn4)C(=O)O)cc3OC)c2n1] | (3-((4-((6-amino-2- ethoxy-8-hydroxy- 9H-purin-9- yl)methyl)-3- methoxybenzyl)- amino)- propanoyl)-L- histidine | Compound 116 |
| 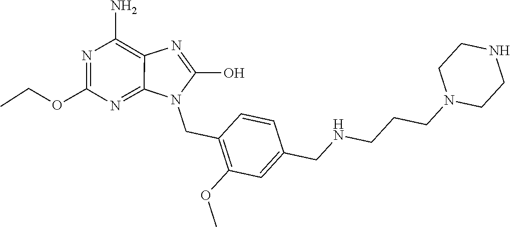 [SMILES: CCOc1nc(N)c2nc(O)n(Cc3ccc(CNCCCN4CCNCC4)cc3OC)c2n1] | 6-amino-2-ethoxy- 9-(2-methoxy-4- (((3-(piperazin-1- yl)propyl)amino) methyl)-benzyl)- 9H-purin-8-ol | Compound 131 |
| 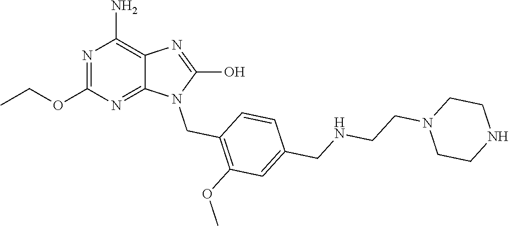 [SMILES: CCOc1nc(N)c2nc(O)n(Cc3ccc(CNCCN4CCNCC4)cc3OC)c2n1] | 6-amino-2-ethoxy-9-(2- methoxy-4-(((2- (piperazin-1- yl)ethyl)amino) methyl)benzyl)- 9H-purin-8-ol | Compound 134 |
| 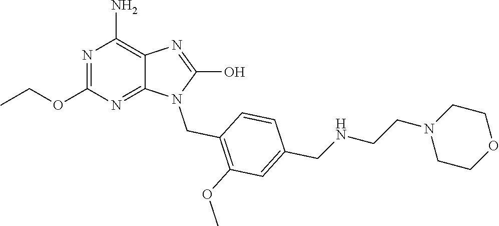 [SMILES: CCOc1nc(N)c2nc(O)n(Cc3ccc(CNCCN4CCOCC4)cc3OC)c2n1] | 6-amino-2-ethoxy- 9-(2-methoxy-4- (((2- morpholinoethyl) amino) methyl)benzyl)- 9H-purin-8-ol | Compound 135 |
| 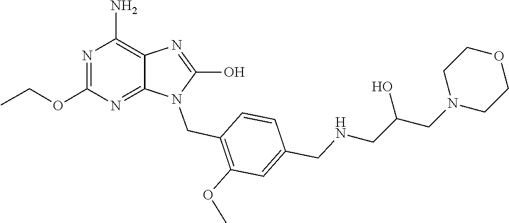 [SMILES: CCOc1nc(N)c2nc(O)n(Cc3ccc(CNCC(O)CN4CCOCC4)cc3OC)c2n1] | 6-amino-2-ethoxy- 9-(4- (((2-hydroxy-3- morpholinopropyl)- amino)methyl)-2- methoxybenzyl)- 9H-purin-8-ol | Compound 136 |
| 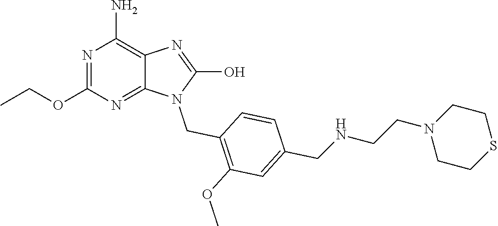 [SMILES: CCOc1nc(N)c2nc(O)n(Cc3ccc(CNCCN4CCSCC4)cc3OC)c2n1] | 6-amino-2-ethoxy- 9-(2- methoxy-4-(((2- thiomorpholinoethyl)- amino)methyl)- benzyl)- 9H-purin-8-ol | Compound 137 |
| 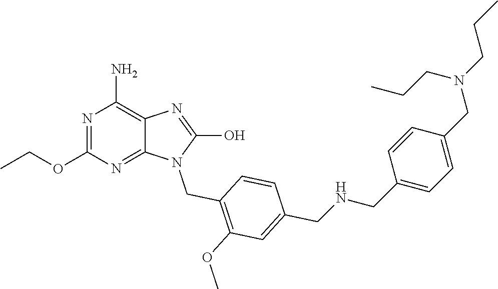 [SMILES: CCCN(CCC)Cc1ccc(CNCc2ccc(Cn3c(O)nc4c(N)nc(OCC)nc43)c(OC)c2)cc1] | 6-amino-9-(4-(((4- ((dipropylamino) methyl) benzyl)amino) methyl)-2- methoxybenzyl)-2- ethoxy-9H- purin-8-ol | Compound 141 |
| 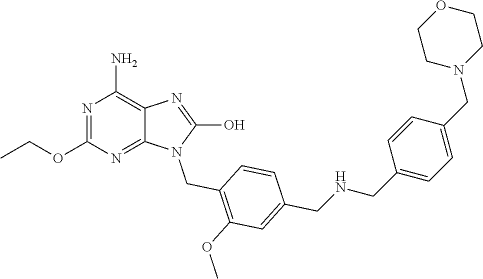 [SMILES: CCOc1nc(N)c2nc(O)n(Cc3ccc(CNCc4ccc(CN5CCOCC5)cc4)cc3OC)c2n1] | 6-amino-2-ethoxy- 9-(2-methoxy-4-(((4- (morpholinomethyl)- benzyl)amino)- methyl)benzyl)-9H- purin-8-ol | Compound 142 |
| 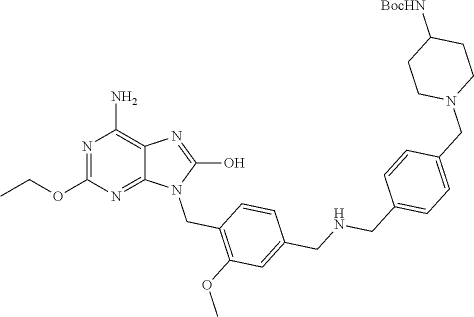 [SMILES: CCOc1nc(N)c2nc(O)n(Cc3ccc(CNCc4ccc(CN5CCC(NC(=O)OC(C)(C)C)CC5)cc4)cc3OC)c2n1] | tert-butyl-(1-(4- (((4-((6- amino-2-ethoxy-8- hydroxy-9H-purin-9- yl)methyl)-3- methoxybenzyl) amino)methyl)benzyl) piperidin-4- yl)carbamate | Compound 143 |
| 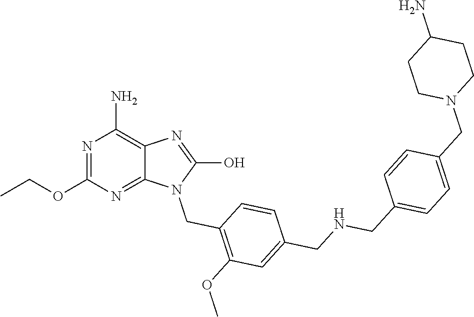 [SMILES: CCOc1nc(N)c2nc(O)n(Cc3ccc(CNCc4ccc(CN5CCC(N)CC5)cc4)cc3OC)c2n1] | 6-amino-9-(4- (((4-((4- aminopiperidin-1- yl)methyl)benzyl) amino)methyl)-2- methoxybenzyl)-2- ethoxy-9H- purin-8-ol | Compound 144 |
| 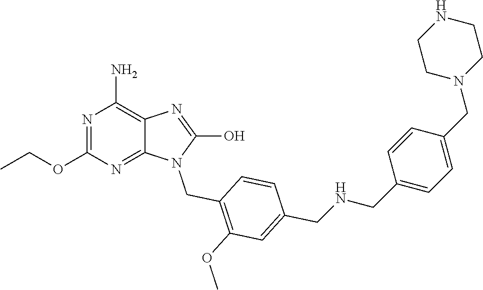 [SMILES: CCOc1nc(N)c2nc(O)n(Cc3ccc(CNCc4ccc(CN5CCNCC5)cc4)cc3OC)c2n1] | 6-amino-2-ethoxy- 9-(2- methoxy-4-(((4- (piperazin-1- ylmethyl)benzyl) amino)- methyl)benzyl)-9H- purin-8-ol | Compound 145 |
| 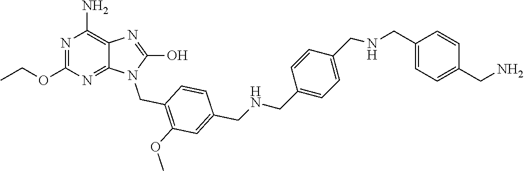 [SMILES: CCOc1nc(N)c2nc(O)n(Cc3ccc(CNCc4ccc(CNCc5ccc(CN)cc5)cc4)cc3OC)c2n1] | 6-amino-9-(4- (((4-(((4- (aminomethyl)benzyl) amino)methyl)benzyl) amino)-methyl)-2- methoxybenzyl)-2- ethoxy-9H- purin-8-ol | Compound 146 |
| 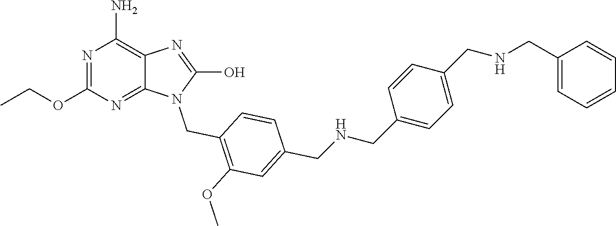 [SMILES: CCOc1nc(N)c2nc(O)n(Cc3ccc(CNCc4ccc(CNCc5ccccc5)cc4)cc3OC)c2n1] | 6-amino-9-(4-(((4- ((benzylamino)methyl) benzyl)amino)methyl)- 2-methoxy- benzyl)-2- ethoxy-9H- purin-8-ol | Compound 147 |
| 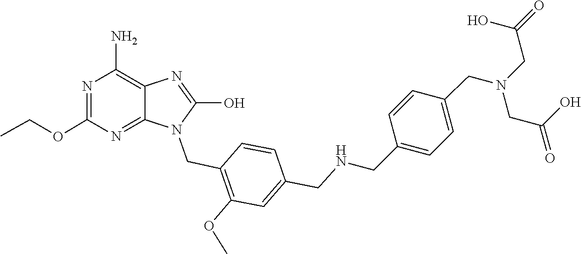 [SMILES: CCOc1nc(N)c2nc(O)n(Cc3ccc(CNCc4ccc(CN(CC(=O)O)CC(=O)O)cc4)cc3OC)c2n1] | 2,2′-((4-(((4- ((6-amino- 2-ethoxy-8- hydroxy-9H- purin-9-yl)methyl)- 3-methoxy- benzyl)amino) methyl)benzyl) azanediyl) diacetic acid | Compound 151 |
| 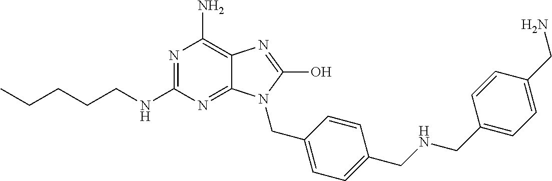 [SMILES: CCCCCNc1nc(N)c2nc(O)n(Cc3ccc(CNCc4ccc(CN)cc4)cc3)c2n1] | 6-amino-9-(4-(((4- (aminomethyl)benzyl) amino)methyl)benzyl)- 2-(pentyl-amino)- 9H-purin-8-ol | Compound 191 |
| 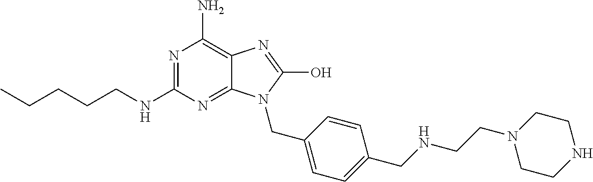 [SMILES: CCCCCNc1nc(N)c2nc(O)n(Cc3ccc(CNCCN4CCNCC4)cc3)c2n1] | 6-amino-2- (pentylamino)-9- (4-(((2- (piperazin-1- yl)ethyl)amino) methyl)benzyl)-9H- purin-8-ol | Compound 195 |

SUBTABLE IF:

| | | |
|---|---|---|
| 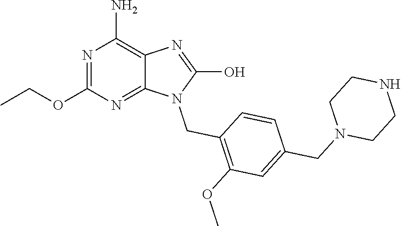 [SMILES: CCOc1nc(N)c2nc(O)n(Cc3ccc(CN4CCNCC4)cc3OC)c2n1] | 6-amino-2-ethoxy- 9-(2-methoxy-4- (piperazin-1- ylmethyl)benzyl)- 9H-purin-8-ol | Compound 100 |
| 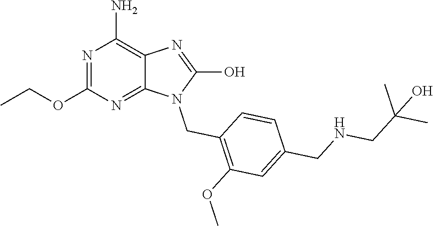 [SMILES: CCOc1nc(N)c2nc(O)n(Cc3ccc(CNCC(C)(C)O)cc3OC)c2n1] | 6-amino-2-ethoxy- 9-(4-(((2-hydroxy-2- methylpropyl)amino) methyl)-2- methoxybenzyl)- 9H-purin-8-ol | Compound 101 |
| 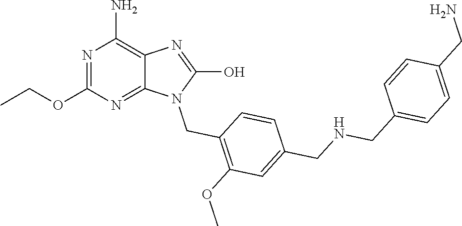 [SMILES: CCOc1nc(N)c2nc(O)n(Cc3ccc(CNCc4ccc(CN)cc4)cc3OC)c2n1] | 6-amino-9-(4-(((4- (aminomethyl)benzyl) amino)methyl)-2- methoxybenzyl)-2- ethoxy-9H-purin-8-ol | Compound 105 |
| 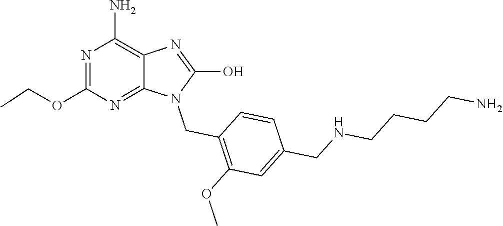 [SMILES: CCOc1nc(N)c2nc(O)n(Cc3ccc(CNCCCCN)cc3OC)c2n1] | 6-amino-9-(4-(((4- aminobutyl)amino) methy1)-2- methoxybenzyl)-2- ethoxy-9H- purin-8-ol | Compound 106 |
| 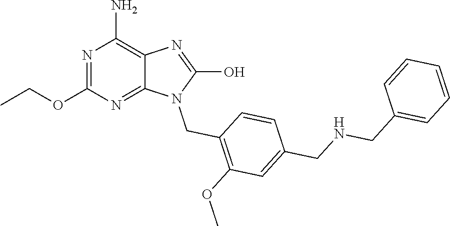 [SMILES: CCOc1nc(N)c2nc(O)n(Cc3ccc(CNCc4ccccc4)cc3OC)c2n1] | 6-amino-9-(4- ((benzylamino) methyl)- 2-methoxybenzyl)- 2-ethoxy-9H- purin-8-ol | Compound 107 |
| 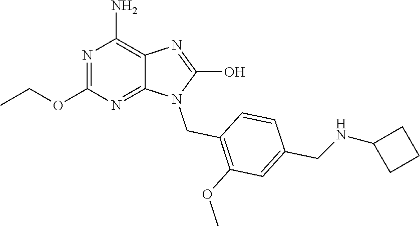 [SMILES: CCOc1nc(N)c2nc(O)n(Cc3ccc(CNC4CCC4)cc3OC)c2n1] | 6-amino-9-(4- ((cyclobutylamino) methyl)-2- methoxybenzyl)-2- ethoxy-9H- purin-8-ol | Compound 108 |
| 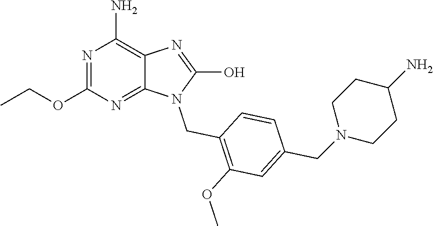 [SMILES: CCOc1nc(N)c2nc(O)n(Cc3ccc(CN4CCC(N)CC4)cc3OC)c2n1] | 6-amino-9-(4-((4- aminopiperidin-1- yl)methyl)-2- methoxybenzyl)-2- ethoxy-9H- purin-8-ol | Compound 110 |
| 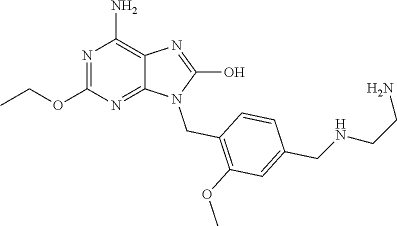 [SMILES: CCOc1nc(N)c2nc(O)n(Cc3ccc(CNCCN)cc3OC)c2n1] | 6-amino-9-(4-(((2- aminoethyl)amino) methyl)-2- methoxybenzyl)-2- ethoxy-9H- purin-8-ol | Compound 111 |
| 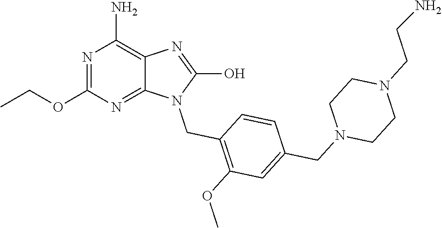 [SMILES: CCOc1nc(N)c2nc(O)n(Cc3ccc(CN4CCN(CCN)CC4)cc3OC)c2n1] | 6-amino-9-(4-(((2- aminoethyl)amino) methyl)-2- methoxybenzyl)- 2-ethoxy-9H- purin-8-ol | Compound 112 |
| 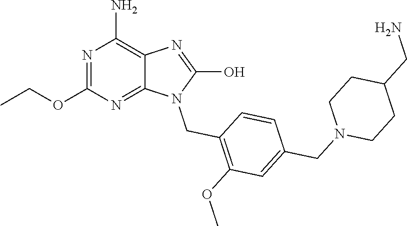 [SMILES: CCOc1nc(N)c2nc(O)n(Cc3ccc(CN4CCC(CN)CC4)cc3OC)c2n1] | 6-amino-9-(4-((4- (aminomethyl) piperidin- 1-yl)methyl)-2- methoxybenzyl)- 2-ethoxy-9H- purin-8-ol | Compound 132 |
| 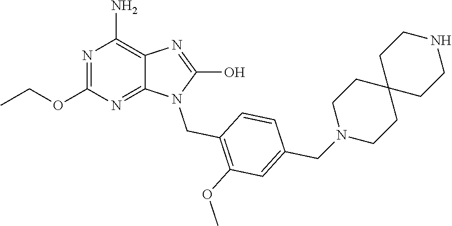 [SMILES: CCOc1nc(N)c2nc(O)n(Cc3ccc(CN4CCC5(CCNCC5)CC4)cc3OC)c2n1] | 9-(4-((3,9- diazaspiro[5.5] undecan- 3-yl)methyl)-2- methoxybenzyl)- 6-amino-2- ethoxy-9H- purin-8-ol | Compound 139 |
| 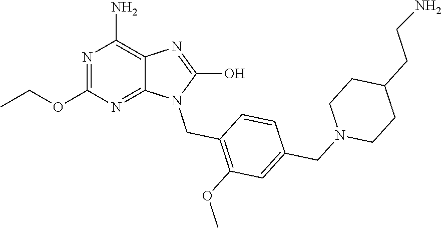 [SMILES: CCOc1nc(N)c2nc(O)n(Cc3ccc(CN4CCC(CCN)CC4)cc3OC)c2n1] | 6-amino-9- (4-((4-(2- aminoethyl)piperidin- 1-yl)methyl)-2- methoxybenzyl)-2- ethoxy-9H- purin-8-ol | Compound 140 |
| 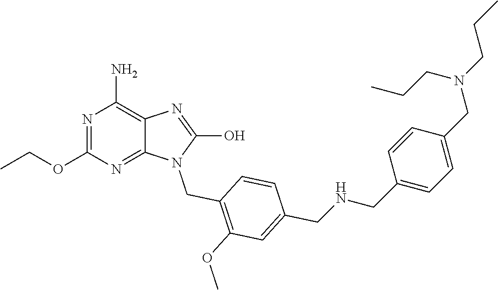 [SMILES: CCCN(CCC)Cc1ccc(CNCc2ccc(Cn3c(O)nc4c(N)nc(OCC)nc43)c(OC)c2)cc1] | 6-amino-9-(4-(((4- ((dipropylamino) methyl) benzyl)amino) methyl)-2- methoxybenzyl)-2- ethoxy-9H- purin-8-ol | Compound 141 |
| 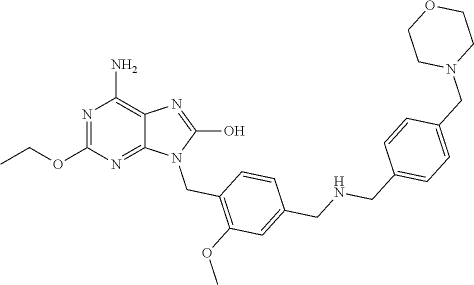 [SMILES: CCOc1nc(N)c2nc(O)n(Cc3ccc(CNCc4ccc(CN5CCOCC5)cc4)cc3OC)c2n1] | 6-amino-2-ethoxy- 9-(2-methoxy-4-(((4- (morpholinomethyl) benzyl)amino)- methyl)benzyl)-9H- purin-8-ol | Compound 142 |
| 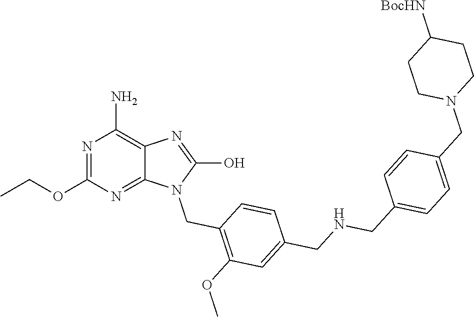 [SMILES: CCOc1nc(N)c2nc(O)n(Cc3ccc(CNCc4ccc(CN5CCC(NC(=O)OC(C)(C)C)CC5)cc4)cc3OC)c2n1] | tert-butyl-(1-(4- (((4-((6- amino-2-ethoxy-8- hydroxy-9H-purin- 9-yl)methyl)-3- methoxybenzyl) amino)methyl)benzyl) piperidin-4- yl)carbamate | Compound 143 |
| 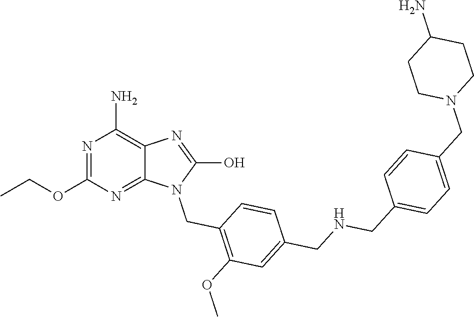 [SMILES: CCOc1nc(N)c2nc(O)n(Cc3ccc(CNCc4ccc(CN5CCC(N)CC5)cc4)cc3OC)c2n1] | 6-amino-9-(4- (((4-((4- aminopiperidin-1- yl)methyl)benzyl) amino) methyl)-2- methoxybenzyl)- 2-ethoxy-9H- purin-8-ol | Compound 144 |
| 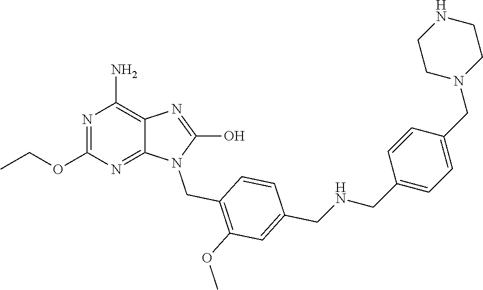 [SMILES: CCOc1nc(N)c2nc(O)n(Cc3ccc(CNCc4ccc(CN5CCNCC5)cc4)cc3OC)c2n1] | 6-amino-2-ethoxy- 9-(2-methoxy-4-(((4- (piperazin-1- ylmethyl)benzyl) amino)- methyl)benzyl)-9H- purin-8-ol | Compound 145 |
| 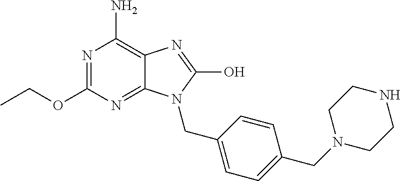 [SMILES: CCOc1nc(N)c2nc(O)n(Cc3ccc(CN4CCNCC4)cc3)c2n1] | 6-amino-2-ethoxy- 9-(4-(piperazin-1- ylmethyl)benzyl)- 9H-purin-8-ol | Compound 150 |
| 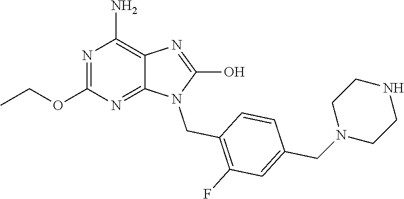 [SMILES: CCOc1nc(N)c2nc(O)n(Cc3ccc(CN4CCNCC4)cc3F)c2n1] | 6-amino-2-ethoxy- 9-(2- fluoro-4-(piperazin-1- ylmethyl)benzyl)-9H- purin-8-ol | Compound 152 |
| 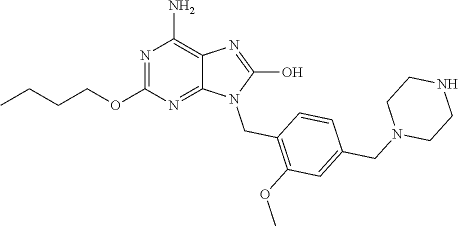 [SMILES: CCCCOc1nc(N)c2nc(O)n(Cc3ccc(CN4CCNCC4)cc3OC)c2n1] | 6-amino-2-butoxy- 9-(2-methoxy-4- (piperazin-1- ylmethyl)benzyl)- 9H-purin-8-ol | Compound 154 |
| 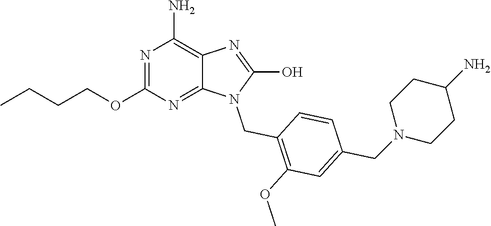 [SMILES: CCCCOc1nc(N)c2nc(O)n(Cc3ccc(CN4CCC(N)CC4)cc3OC)c2n1] | 6-amino-9-(4-((4- aminopiperidin-1- yl)methyl)-2- methoxybenzyl)-2- butoxy-9H- purin-8-ol | Compound 155 |
| 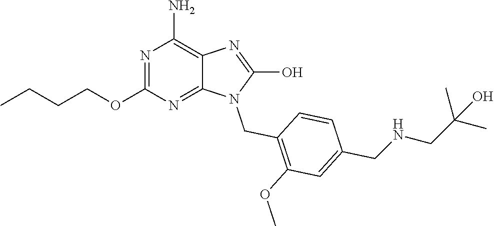 [SMILES: CCCCOc1nc(N)c2nc(O)n(Cc3ccc(CNCC(C)(C)O)cc3OC)c2n1] | 6-amino-2-butoxy- 9-(4-(((2-hydroxy-2- methylpropyl)amino) methyl)-2- methoxybenzyl)- 9H-purin-8-ol | Compound 159 |
| 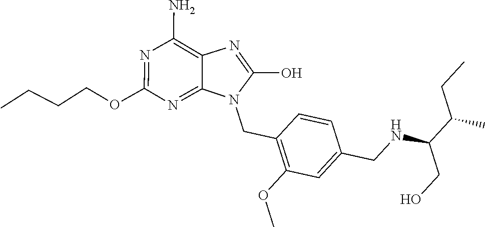 [SMILES: CCCCOc1nc(N)c2nc(O)n(Cc3ccc(CN[C@H](CO)[C@@H](C)CC)cc3OC)c2n1] | 6-amino-2-butoxy- 9-(4-((((2R,3S)- 1-hydroxy-3- methylpentan-2- yl)amino)-methyl)-2- methoxybenzyl)-9H- purin-8-ol | Compound 161 |
| 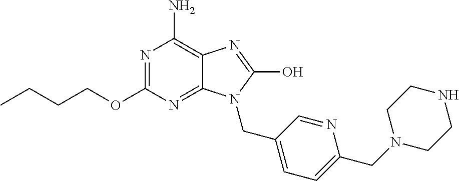 [SMILES: CCCCOc1nc(N)c2nc(O)n(Cc3ccc(CN4CCNCC4)nc3)c2n1] | 6-amino-2-butoxy- 9-(4-(piperazin-1- ylmethyl)benzyl)-9H- purin-8-ol | Compound 166 |
| 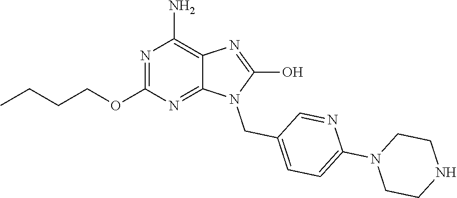 [SMILES: CCCCOc1nc(N)c2nc(O)n(Cc3ccc(N4CCNCC4)nc3)c2n1] | 6-amino-2-butoxy- 9-((6- (piperazin-1-yl) pyridin-3-yl)methyl)- 9H-purin-8-ol | Compound 167 |
| 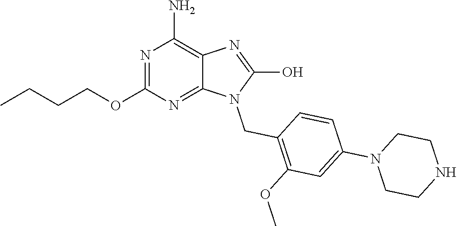 [SMILES: CCCCOc1nc(N)c2nc(O)n(Cc3ccc(N4CCNCC4)cc3OC)c2n1] | 6-amino-2-butoxy- 9-(2- methoxy-4- (piperazin-1- yl)benzyl)- 9H-purin-8-ol | Compound 169 |
| 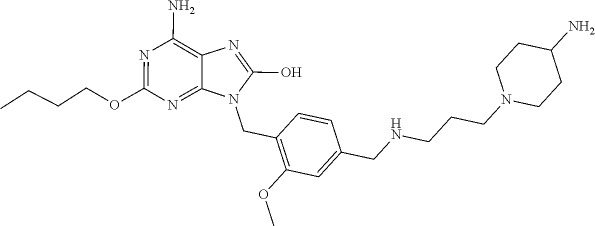 [SMILES: CCCCOc1nc(N)c2nc(O)n(Cc3ccc(CNCCCN4CCC(N)CC4)cc3OC)c2n1] | 6-amino-9-(4-(3-(4- aminopiperidin-1- yl)propyl)-2- methoxybenzyl)-2- butoxy-9H-purin-8-ol | Compound 172 |
| 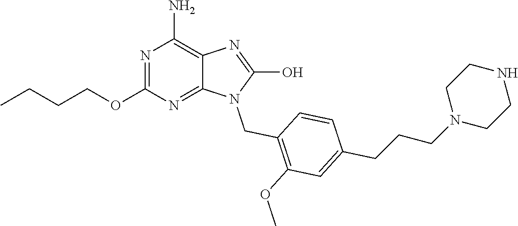 [SMILES: CCCCOc1nc(N)c2nc(O)n(Cc3ccc(CCCN4CCNCC4)cc3OC)c2n1] | 6-amino-2-butoxy- 9-(2-methoxy-4-(3- (piperazin-1- yl)propyl)benzyl)- 9H-purin-8-ol | Compound 173 |
| 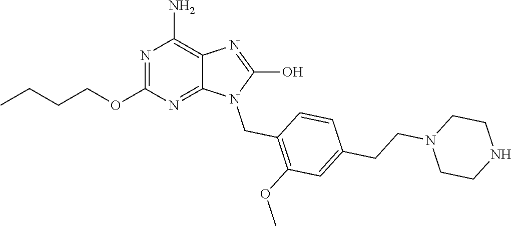 [SMILES: CCCCOc1nc(N)c2nc(O)n(Cc3ccc(CCN4CCNCC4)cc3OC)c2n1] | 6-amino-2-butoxy- 9-(2-methoxy-4-(2- (piperazin-1- yl)ethyl)benzyl)-9H- purin-8-ol | Compound 180 |
| 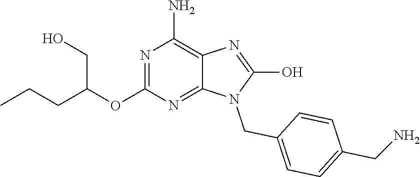 [SMILES: CCCC(CO)Oc1nc(N)c2nc(O)n(Cc3ccc(CN)cc3)c2n1] | 6-amino-9-(4- (aminomethyl)benzyl)- 2-((1-hydroxypentan- 2-yl)oxy)- 9H-purin-8-ol | Compound 182 |
| 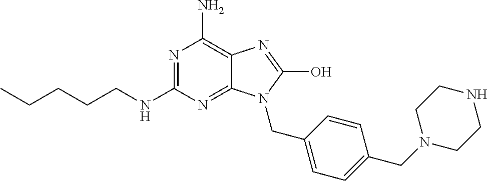 [SMILES: CCCCCNc1nc(N)c2nc(O)n(Cc3ccc(CN4CCNCC4)cc3)c2n1] | 6-amino-2- (pentylamino)-9-(4- (piperazin-1- ylmethyl)benzyl)- 9H-purin-8-ol | Compound 187 |
| 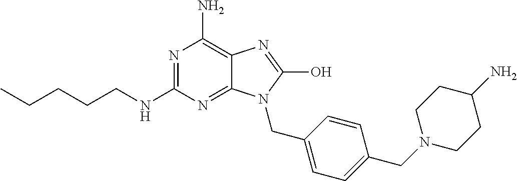 [SMILES: CCCCCNc1nc(N)c2nc(O)n(Cc3ccc(CN4CCC(N)CC4)cc3)c2n1] | 6-amino-9-(4-((4- aminopiperidin-1- yl)methyl)benzyl)-2- (pentylamino)- 9H-purin-8-ol | Compound 188 |
| 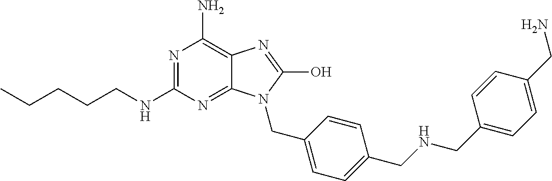 [SMILES: CCCCCNc1nc(N)c2nc(O)n(Cc3ccc(CNCc4ccc(CN)cc4)cc3)c2n1] | 6-amino-9-(4-(((4- (aminomethyl)benzyl) amino)methyl)benzyl)- 2-(pentyl-amino)- 9H-purin-8-ol | Compound 191 |

ADDITIONAL COMPOUNDS

| | | |
|---|---|---|
| 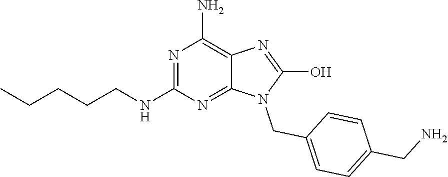 [SMILES: CCCCCNc1nc(N)c2nc(O)n(Cc3ccc(CN)cc3)c2n1] | 6-amino-9-(4- (aminomethyl) benzyl)-2- (pentylamino)- 9H-purin-8-ol | Compound 183 |
| 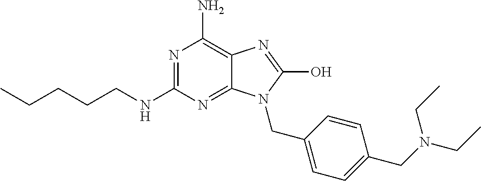 [SMILES: CCCCCNc1nc(N)c2nc(O)n(Cc3ccc(CN(CC)CC)cc3)c2n1] | 6-amino-9-(4- ((diethylamino) methyl) benzyl)-2- (pentylamino)- 9H-purin-8-ol | Compound 185 |

It is to be understood that reference to compounds of Formula (I) throughout this disclosure, includes in various embodiments, compounds of Formulae (I)-(IV), to the same extent as if embodiments reciting each of these Formulae individually were specifically recited.

In certain embodiments, a compound having Formula (I), as described herein, can possess a sufficiently acidic group, a sufficiently basic group, or both functional groups, and accordingly react with a number of organic and inorganic bases, or organic and inorganic acids, to form pharmaceutically acceptable salts. The term “pharmaceutically acceptable salt,” as used herein, refers to a salt of a compound having Formula (I), which is substantially non-toxic to living organisms. Typical pharmaceutically acceptable salts include those salts prepared by reaction of a compound having Formula (I), with a pharmaceutically acceptable mineral or organic acid or an organic or inorganic base. Such salts are known as acid addition and base addition salts.

Acids commonly employed to form acid addition salts are inorganic acids including, but are not limited to, hydrochloric acid, hydrobromic acid, hydroiodic acid, sulphuric acid, phosphoric acid, and organic acids including, but not limited to, p-toluenesulphonic acid, methanesulphonic acid, oxalic acid, p-bromophenylsulphonic acid, carbonic acid, succinic acid, citric acid, benzoic acid, and acetic acid. Examples of pharmaceutically acceptable salts include, but are not limited to, sulphates, pyrosulphates, bisulphates, sulphites, phosphates, monohydrogenphosphates, dihydrogenphosphates, metaphosphates, pyrophosphates, bromides, iodides, acetates, propionates, decanoates, caprylates, acrylates, formates, hydrochlorides, dihydrochlorides, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, hydroxybenzoates, methoxybenzoates, phthalates, xylenesulphonates, phenylacetates, phenylpropionates, phenylbutyrates, citrates, lactates, gamma-hydroxybutyrates, glycolates, tartrates, methanesulphonates, propanesulphonates, naphthalene-1-sulfonates, napththalene-2-sulfonates and mandelates. Pharmaceutically acceptable acid addition salts of particular interest are those formed with mineral acids such as hydrochloric acid and hydrobromic acid, and those formed with organic acids such as maleic acid and methanesulphonic acid.

Salts of amine groups can also comprise quaternary ammonium salts in which the amino nitrogen carries a suitable organic group such as an alkyl, lower alkenyl, lower alkynyl or aralkyl moiety.

Base addition salts include those derived from inorganic bases, such as ammonium or alkali or alkaline earth metal hydroxides, carbonates, bicarbonates, and the like. Bases useful in preparing pharmaceutically acceptable salts include, but are not limited to, sodium hydroxide, potassium hydroxide, ammonium hydroxide, potassium carbonate, sodium carbonate, sodium bicarbonate, potassium bicarbonate, calcium hydroxide and calcium carbonate.

One skilled in the art will understand that the particular counterion forming a part of a pharmaceutically acceptable salt is usually not of a critical nature, so long as the salt as a whole is pharmacologically acceptable and as long as the counterion does not contribute undesired qualities to the salt as a whole.

As described herein, a compound of Formulae (I)-(IV) can comprise one or more free amino, hydroxy, carbonyl (for example, keto or aldehyde) and/or carboxylic acid groups. Also encompassed by the present disclosure are protected versions of the compounds of Formulae (I)-(IV) in which an otherwise free amino, hydroxy, carbonyl (for example, keto or aldehyde) and/or carboxylic acid group is protected with an appropriate protecting group. The term “protecting group” refers to a chemical group that, when attached to a potentially reactive functional group, masks, reduces or prevents the reactivity of the functional group. Typically, a protecting group can be selectively removed as desired during the course of a synthesis.

Protecting groups are well-known in the art and various examples are described, for example, in “Protective Groups in Organic Chemistry” (Greene, W. & Wuts, P. G. M., 2006, John Wiley & Sons). Examples of amino protecting groups include, but are not limited to, formyl, acetyl, trifluoroacetyl, benzyl (Bn), benzoyl (Bz), benzyloxycarbonyl (CBZ), tert-butoxycarbonyl (Boc), trimethylsilyl (TMS), 2-trimethylsilyl-ethanesulfonyl (TES), trityl, substituted trityl, tosyl, phthalimide, alloxycarbonyl (Alloc) and 9-fluorenylmethyloxycarbonyl (FMOC). Examples of hydroxy protecting groups include, but are not limited to, acetyl, benzyl (Bn), t-butyl, benzoyl (Bz), β-methoxyethoxymethyl ether (MEM), dimethoxytrityl (DMT), methoxymethyl ether (MOM), methoxytrityl [(4-methoxyphenyl)diphenylmethyl](MMT), p-methoxybenzyl ether (PMB), p-methoxyphenyl ether (PMP), methylthiomethyl ether, pivaloyl (Piv), tetrahydropyranyl (THP), tetrahydrofuran (THF), trityl, trimethylsilyl (TMS), tert-butyldimethylsilyl (TBDMS or TBS), tri-iso-propylsilyloxymethyl (TOM), and triisopropylsilyl (TIPS). Examples of carbonyl protecting groups include, but are not limited to, acetals, hemi-acetals, and ketals. Examples of carboxylic acid protecting groups include, but are not limited to, methyl esters, benzyl esters, tert-butyl esters, silyl esters, orthoesters and oxazoline.

Certain embodiments relate to pharmaceutically acceptable solvates of a compound having Formula (I)-(IV). One skilled in the art will appreciate that certain compounds having Formula (I)-(IV), can combine with solvents such as water, methanol, ethanol, or acetonitrile to form pharmaceutically acceptable solvates such as the corresponding hydrate, methanolate, ethanolate or acetonitrilate. Other examples of solvents that can be used to prepare solvates include isopropanol, dimethyl sulfoxide, ethyl acetate, acetic acid, ethanolamine, and acetone, as well as miscible formulations of solvate mixtures as would be known by the skilled artisan.

In various embodiments, a compound of the present disclosure, e.g., a compound having a structure according to any one of Formulae (I)-(IV), is an agonist of TLR7. In some embodiments, a compound of the present disclosure (e.g., one according to any one of Formulae (I)-(IV)) is capable of inducing production of one or more cytokine(s) in immune cells (e.g., PBMCs). In such embodiments, the compound can induce production of, e.g., IL6, IFN-α and/or TNF-α in immune cells (e.g., PBMCs) when such cells are contacted with the compound.

In some embodiments, a compound of the present disclosure, e.g., one having a structure according to any one of Formulae (I)-(IV), can have an EC₅₀ of about 1 μM or less, 750 nM or less, 650 nM or less, 500 nM or less, 300 nM or less, 275 nM or less, 250 nM or less, 225 nM or less, 200 nM or less, 175 nM or less, 150 nM or less, 125 nM or less, 100 nM or less, 75 nM or less, 50 nM or less, 25 nM or less, 20 nM or less, 15 nM or less, or about 10 nM or less, for agonism of TLR7, as determined, e.g., in a reporter gene assay. The TLR7 can be a human TLR7 or a murine TLR7, or both. In some embodiments, a compound of any one of Formulae (I)-(IV) has an EC₅₀ value for agonizing human and/or murine TLR7 of about 1 μM or less. In some embodiments, a compound of any one of Formulae (I)-(IV) has an EC₅₀ value for agonizing human and/or murine TLR7 of about 500 nM or less. In some embodiments, a compound of any one of Formulae (I)-(IV) has an EC₅₀ value for agonizing human and/or murine TLR7 of about 300 nM or less. In some embodiments, a compound of any one of Formulae (I)-(IV) has an EC₅₀ value for agonizing human and/or murine TLR7 of about 100 nM or less. In other embodiments, a compound of any one of Formulae (I)-(IV) has an EC₅₀ value for agonizing human and/or murine TLR7 of about 50 nM or less. In yet other embodiments, a compound of any one of Formulae (I)-(IV) has an EC₅₀ value for agonizing human and/or murine TLR7 of about 25 nM or less. As used herein, an EC₅₀ value generally refers to the half maximal effective concentration of the respective compound, wherein the value of the EC₅₀ indicates the concentration of the compound that induces a biological response (e.g., TLR7 agonism, stimulation of the immune system, production of cytokines by immune cells, and/or killing of tumor cells) halfway between the baseline and the maximum after a defined duration of exposure.

In some embodiments, the present disclosure relates to purine-derived compounds, e.g., those having a structure according to any one of Formulae (I)-(IV), that have an EC₅₀ for agonizing TLR7 from about 1 μM to about 750 nM, from about 1 μM to about 500 nM, from about 1 μM to about 300 nM, from about 1 μM to about 200 nM, from about 1 μM to about 100 nM, from about 1 μM to about 50 nM, from about 500 nM to about 25 nM, from about 500 nM to about 15 nM, from about 500 nM to about 5 nM, from about 500 nM to about 1 nM, from about 500 nM to about 0.1 nM, from about 500 nM to about 0.01 nM, from about 300 nM to about 25 nM, from about 300 nM to about 15 nM, from about 300 nM to about 5 nM, from about 300 nM to about 1 nM, from about 300 nM to about 0.1 nM, from about 300 nM to about 0.01 nM, from about 200 nM to about 25 nM, from about 200 nM to about 15 nM, from about 200 nM to about 5 nM, from about 200 nM to about 1 nM, from about 200 nM to about 0.1 nM, from about 200 nM to about 0.01 nM, from about 100 nM to about 25 nM, from about 100 nM to about 15 nM, from about 100 nM to about 5 nM, from about 100 nM to about 1 nM, from about 100 nM to about 0.1 nM, from about 100 nM to about 0.01 nM, from about 50 nM to about 25 nM, from about 50 nM to about 15 nM, from about 50 nM to about 5 nM, from about 50 nM to about 1 nM, from about 50 nM to about 0.1 nM, or from about 50 nM to about 0.01 nM. In various embodiments, the TLR7 is a human TLR7.

An EC₅₀ value for TLR7 agonism of a compound of the present disclosure, e.g., one having a structure according to any one of Formulae (I)-(IV), can be determined using various methods known in the art, e.g., in vitro using a reporter gene assay employing TLR7 reporter cells. In such embodiments, the EC₅₀ value can be determined in vitro using a reporter gene assay employing HEK-Blue™ TLR7 reporter cells (e.g., available from Invivogen, San Diego, CA), and as described in, e.g., EXAMPLE 3 herein.

In some embodiments, and as further described herein, it was surprisingly found that particularly those purine-derived compounds of Formulae (I)-(IV) that contain a substituent at R² (i.e., R²≠H) can provide potent immunomodulatory activity in vitro as well as in vivo, when used both as a free molecule and in form of an immunostimulatory antibody drug conjugate (ISAC). Such compounds can elicit their immunomodulatory activities in both murine and human settings targeting murine as well as human TLR7. In various embodiments, such substituent at the R² position can be an alkoxy moiety or a halogen atom. In specific embodiments, the R² substituent can be methoxy or fluorine. Such compounds herein can have an EC₅₀ for agonizing TLR7 of equal to or less than about 500 nM, 100 nM, 50 nM, 30 nM, 20 nM, equal to or less than about 10 nM, or lower, as determined, e.g., using reporter gene assays (RGAs), as described herein.

In other embodiments, a compound of Formula (I) in which R² is H and either (i) R¹ is branched and optionally substituted C₃-C₈ hydroxyalkyl, (ii) X is NH and R¹ is optionally substituted C₅-C₆ alkyl, or (iii) m is 0 or 1 and R³ and R⁴ together with the N form an unsubstituted piperazinyl, can possess strong immunomodulatory properties that can be useful for the treatment of diseases such as cancer.

In some embodiments, a compound of the present disclosure, e.g., one having a structure according to any one of Formulae (I)-(IV), can have an EC₅₀ for inducing production of one or more human and/or murine cytokines from human or murine immune cells, respectively, of about 5 μM or less, 3 μM or less, 1 μM or less, 750 nM or less, about 500 nM or less, about 300 nM or less, about 275 nM or less, about 250 nM or less, about 225 nM or less, about 200 nM or less, about 175 nM or less, about 150 nM or less, about 125 nM, about 100 nM or less, about 50 nM or less, about 25 nM or less, or about 10 nM or less. In some embodiments, a compound of the present disclosure, e.g., one having a structure according to any one of Formulae (I)-(IV), can have an EC₅₀ for inducing production of one or more human and/or murine cytokines from human or murine immune cells, respectively, from about 5 μM to about 5 nM, from about 1 μM to about 1 nM, from about 750 nM to about 1 nM, from about 500 nM to about 1 nM, from about 300 nM to about 1 nM or from about 200 nM to about 1 nM. In various embodiments, the one or more human and/or murine cytokines comprise IL6. In some embodiments, the human immune cells comprise PBMCs, and the murine immune cells comprise murine splenocytes. In various embodiments, the EC₅₀ values for inducing production of human or murine IL6 from PBMCs or mouse splenocytes are determined using a human PBMC assay or a mouse splenocyte assay, respectively, e.g., as described in EXAMPLE 3 herein.

In some embodiments, a compound of the present disclosure, e.g., one having a structure according to any one of Formulae (I)-(IV), can have an EC₅₀ for inducing production of human interferon-α (hIFN-α) of about 10 nM or less, 5 nM or less, 1 nM or less, or 0.1 nM or less. In some embodiments, a compound of the present disclosure, e.g., one having a structure according to any one of Formulae (I)-(IV), can have an EC₅₀ for inducing production of hIFN-α of from about 10 nM to about 1 nM, from about 1 nM to about 0.1 nM, or lower than 0.1 nM. In various embodiments, the EC₅₀ values for inducing production of hIFN-α from PBMCs are determined using a human PBMC assay, e.g., as described in EXAMPLE 3 herein.

In certain embodiments, the EC₅₀ for inducing production of a cytokine from PBMCs by a compound of the present disclosure, e.g., one having a structure according to any one of Formulae (I)-(IV), is determined in vitro by treating PBMCs isolated from peripheral blood with titrating concentrations of the tested compound followed by assaying for cytokines, e.g., by homogeneous time resolved fluorescence (HTRF). An exemplary method for determining EC₅₀ values for inducing production of a cytokine from PBMCs is provided in EXAMPLE 3 herein.

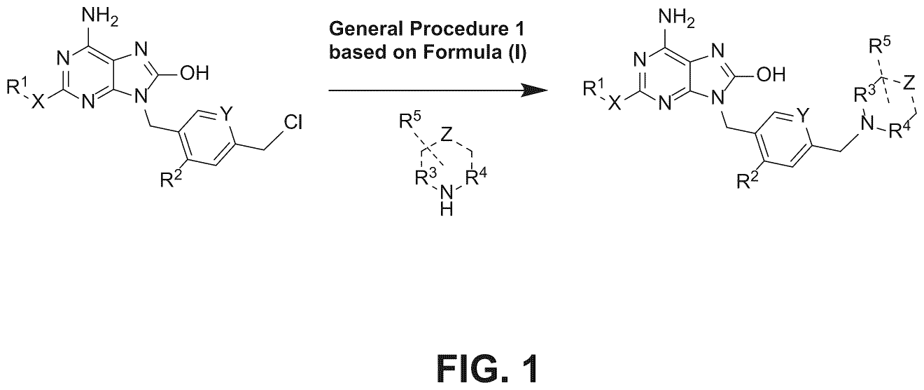

The purine-derived compounds of the present disclosure, e.g., those of Formulae (I)-(IV), can be prepared by standard synthetic organic chemistry methods from commercially available starting materials and reagents. Representative examples of suitable synthetic routes are described in detail in EXAMPLE 1 provided herein (see also, FIG. 1). One skilled in the art will recognize that alternative methods can be employed to synthesize a purine-derived compounds of the present disclosure, and that the approaches described herein are therefore not intended to be exhaustive.

### Compound-Linker Constructs

A purine-derived compound of the present disclosure can be coupled to a linker moiety. In certain embodiments, the present disclosure relates to compound-linker constructs comprising one or more compounds (C) of the present disclosure, e.g., those having a structure according to any one of Formulae (I)-(IV), e.g., any of the compounds listed in TABLE 1, coupled to a linker moiety (L).

In various embodiments, the present disclosure relates to a compound-linker construct having Formula (A):

L-(C)ₚ (A)

wherein:
L is a linker;
C is a compound of any one of Formulae (I)-(IV); and
p is an integer from 1 to 5.

In various embodiments of a compound-linker construct, p can be an integer from 1 to 4. In other embodiments, p is an integer from 1 to 3. In yet other embodiments, p is an integer from 1 to 2. In various embodiments, p is 1. In other embodiments, p is 2. In yet other embodiments, p is 3.

A linker (L) herein can be a bifunctional or multifunctional moiety capable of linking one or more immunomodulatory purine-derived compounds, C, of the present disclosure to another molecule, such as a targeting moiety, T, as further described elsewhere herein.

A bifunctional (or monovalent) linker, L, links a single compound C to a single site (e.g., functional group) on a targeting moiety, T. In such embodiments, p is 1. A multifunctional (or polyvalent) linker, L, links more than one compound, C, to a single site (e.g., functional group) on a targeting moiety, T. A linker that links one compound, C, to more than one site on targeting moiety, T, can also be considered to be multifunctional. However, in embodiments where the linker (L) is polyvalent and couples more than one compound (C) to another moiety, such as a targeting moiety (T), p can be >1, such as an integer from 2 to 5.

A linker, L, of a compound-linker construct herein can comprise a functional group capable of reacting with a target group or groups on a targeting moiety, T, and at least one functional group capable of reacting with a target group on a compound of the present disclosure (e.g., a compound according to any one of Formulae (I)-(IV)), C. Suitable functional groups are known in the art and include those described herein, for example, in Bioconjugate Techniques (G. T. Hermanson, 2013, Academic Press). Functional groups on targeting moiety, T, and the purine-derived compound of the present disclosure, C, that can serve as target groups for linker attachment include, but are not limited to, thiol, hydroxyl, carboxyl, amine, aldehyde, and ketone groups.

In some embodiments, a linker herein comprises or consists of a substituted or unsubstituted alkyl or a substituted or unsubstituted heteroalkyl chain comprising a first and a second terminal functional group. In some embodiments, the first terminal functional group of the linker forms a first linkage or bond with a first reactive functional group on a first conjugation partner (e.g., a targeting moiety, T), and the second terminal functional group of the linker forms a second linkage or bond with a second reactive functional group of a second conjugation partner (e.g., a compound C of Formulae (I)-(IV)). In some embodiments, the linker comprises a substituted or unsubstituted hydrocarbon backbone. In some embodiments, the substituted or unsubstituted hydrocarbon backbone is interrupted by one or more heteroatoms (e.g., O, N, S, P), thereby forming, e.g., a heteroalkyl linker.

In some embodiments, a linker (L) is a cleavable linker. In other embodiments, the linker (L) is a non-cleavable linker. A cleavable linker herein is a linker that is susceptible to cleavage under specific conditions, for example, intracellular conditions (such as in an endosome or lysosome) or within the vicinity of a target cell (such as in the tumor microenvironment). Examples include linkers that are protease-sensitive, acid-sensitive, or reduction-sensitive. Non-cleavable linkers by contrast, can rely on the degradation of a targeting moiety (e.g., antibody)—to which it is attached—in the cell, which typically results in the release of an amino acid-linker-compound moiety. In some embodiments, the linker includes an alkylene oxide, e.g., a poly (alkylene oxide), or a poly (ethylene glycol) (PEG) moiety.

In some embodiments, the linker (L) of a compound-linker construct herein comprises a polyethylene glycol (PEG) moiety. Such PEG moiety can have a molecular weight from about 500 Da to about 5 kDa, from about 500 Da to about 3 kDa, from about 500 Da to about 1 kDa, from about 500 Da to about 1 kDa, or from about 100 Da to 500 Da.

Examples of cleavable linkers include, for example, linkers comprising an amino acid sequence that is a cleavage recognition sequence for a protease. Many such cleavage recognition sequences are known in the art. In embodiments in which a compound-linker construct herein is used in conjugates that are not intended to be internalized by a cell, for example, an amino acid sequence that is recognized and cleaved by a protease present in the extracellular matrix in the vicinity of a target cell, such as a cancer cell, can be employed. Examples of extracellular tumor-associated proteases include, for example, plasmin, matrix metalloproteases (MMPs), elastase and kallikrein-related peptidases.

In embodiments in which a compound-linker construct herein is used in conjugates intended to be internalized by a cell, a linker, L, can comprise an amino acid sequence that is recognized and cleaved by an endosomal or lysosomal protease. Examples of such proteases include, for example, cathepsins B, C, D, H, L and S, and legumain.

Cleavage recognition sequences can be, for example, dipeptides, tripeptides or tetrapeptides. Non-limiting examples of dipeptide recognition sequences that can be included in cleavable linkers described herein include, but are not limited to, Ala-(D)Asp, Ala-Lys, Ala-Phe, Asn-Lys, Asn-(D)Lys, Asp-Val, His-Val, Ile-Cit, Ile-Pro, Ile-Val, Leu-Cit, Me₃Lys-Pro, Met-Lys, Met-(D)Lys, NorVal-(D)Asp, Phe-Arg, Phe-Cit, Phe-Lys, PhenylGly-(D)Lys, Pro-(D)Lys, Trp-Cit, Val-Ala, Val-(D)Asp, Val-Cit, Val-Gly, Val-Gln and Val-Lys. Examples of tri- and tetrapeptide cleavage sequences include, but are not limited to, Ala-Ala-Asn, Ala-Val-Cit, (D)Ala-Phe-Lys, Asp-Val-Ala, Asp-Val-Cit, Gly-Cit-Val, Lys-Val-Ala, Lys-Val-Cit, Met-Cit-Val, (D)Phe-Phe-Lys, Asn-Pro-Val, Ala-Leu-Ala-Leu, Gly-Phe-Leu-Gly, Gly-Gly-Phe-Gly and Gly-Phe-Gly-Gly. In some embodiments, a linker (L) comprises a dipeptide, tripeptide, tetrapeptide, or a combination thereof. In some of these embodiments, the linker (L) comprises a dipeptide. In other embodiments, the linker (L) comprises a tripeptide. In yet other embodiments, the linker (L) comprises a tetrapeptide.

Additional examples of cleavable linkers include disulfide-containing linkers such as N-succinimydyl-4-(2-pyridyldithio) butanoate (SPDB) and N-succinimydyl-4-(2-pyridyldithio)-2-sulfo butanoate (sulfo-SPDB). Disulfide-containing linkers can optionally include additional groups to provide steric hindrance adjacent to the disulfide bond in order to improve the extracellular stability of the linker, for example, inclusion of a geminal dimethyl group. Other cleavable linkers include linkers hydrolyzable at a specific pH or within a pH range, such as hydrazone linkers. Linkers comprising combinations of these functionalities can also be useful, for example, linkers comprising both a hydrazone and a disulfide are known in the art.

A further example of a cleavable linker is a linker comprising a β-glucuronide, which is cleavable by β-glucuronidase, an enzyme present in lysosomes and tumor interstitium (see, for example, De Graaf et al., 2002, Curr. Pharm. Des. 8:1391-1403, and International Patent Publication No. WO 2007/011968). β-glucuronide can also function to improve the hydrophilicity of linker, L.

Another example of a linker that is cleaved internally within a cell and improves hydrophilicity is a linker comprising a pyrophosphate diester moiety (see, for example, Kern et al., 2016, J Am Chem Soc., 138:2430-1445).

In certain embodiments, the linker, L, comprised by a compound-linker construct of Formula (A) is a cleavable linker. In some embodiments, such linker, L, comprises a cleavage recognition sequence. In some embodiments, such linker, L, can comprise an amino acid sequence that, e.g., one that is 2 or 3 residues in length, and is recognized and cleaved by a protease.

Cleavable linkers can optionally further comprise one or more additional functionalities such as self-immolative and self-elimination groups, stretchers, hydrophilic moieties, or a combination thereof.

Self-immolative and self-elimination groups that can find use in linkers herein include, for example, p-aminobenzyl (PAB) and p-aminobenzyloxycarbonyl (PABC) groups, and methylated ethylene diamine (MED). Other examples of self-immolative groups include, but are not limited to, aromatic compounds that are electronically similar to the PAB or PABC group such as heterocyclic derivatives, for example 2-aminoimidazol-5-methanol derivatives as described in U.S. Pat. No. 7,375,078. Other examples include groups that undergo cyclization upon amide bond hydrolysis, such as substituted and unsubstituted 4-aminobutyric acid amides (Rodrigues et al., 1995, Chemistry Biology 2:223-227) and 2-aminophenylpropionic acid amides (Amsberry, et al., 1990, J. Org. Chem. 55:5867-5877). Self-immolative/self-elimination groups are typically attached to an amino or hydroxyl group on the compound, C. Self-immolative/self-elimination groups, alone or in combination are often included in peptide-based linkers but can also be included in other types of linkers.

Stretchers that can find use in linkers for conjugates as further described herein include, for example, alkylene groups and stretchers based on aliphatic acids, diacids, amines or diamines, such as diglycolate, malonate, caproate and caproamide. Other stretchers include, for example, glycine-based stretchers and polyethylene glycol (PEG) or monomethoxy polyethylene glycol (mPEG) stretchers.

PEG and mPEG stretchers can also function as hydrophilic moieties within a linker of the present disclosure. For example, PEG or mPEG can be included in a linker either “in-line” or as pendant groups to increase the hydrophilicity of the linker (see, for example, U.S. Patent Application Publication No. US 2016/0310612). Various PEG-containing linkers are commercially available from companies such as Quanta BioDesign, Ltd (Plain City, OH). Other hydrophilic groups that can optionally be incorporated into linker, L, include, for example, β-glucuronide, sulfonate groups, carboxylate groups and pyrophosphate diesters.

In some embodiments, a compound-linker construct of this disclosure can comprise or consist of a compound of Formulae (I)-(IV) herein coupled to any of the linker moieties L1-L18 shown in TABLE 2A ( [symbol] indicates the attachment to a compound of Formulae (I)-(IV)).

TABLE 2A
Exemplary Linker Moieties for Compound-Linker Constructs

| Structure | Name |
|---|---|
| 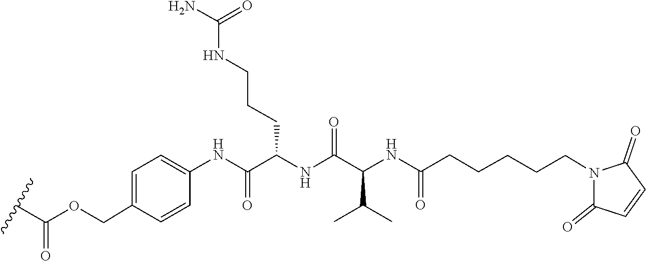 [SMILES: *C(=O)OCc1ccc(NC(=O)[C@H](CCCNC(N)=O)NC(=O)[C@@H](NC(=O)CCCCCN2C(=O)C=CC2=O)C(C)C)cc1] | L1 |
| 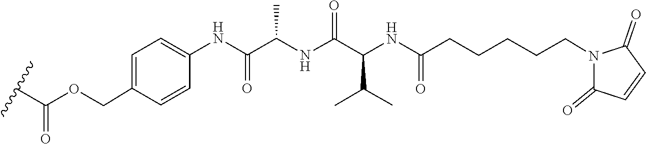 [SMILES: *C(=O)OCc1ccc(NC(=O)[C@H](C)NC(=O)[C@@H](NC(=O)CCCCCN2C(=O)C=CC2=O)C(C)C)cc1] | L2 |
| 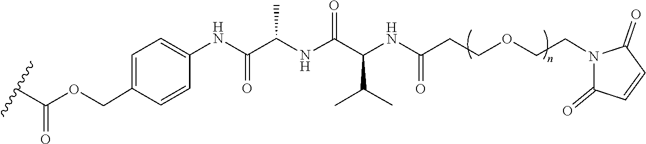 | L3 |
| 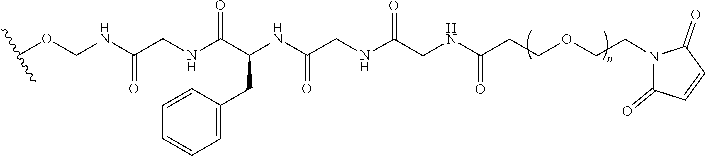 | L4 |
| 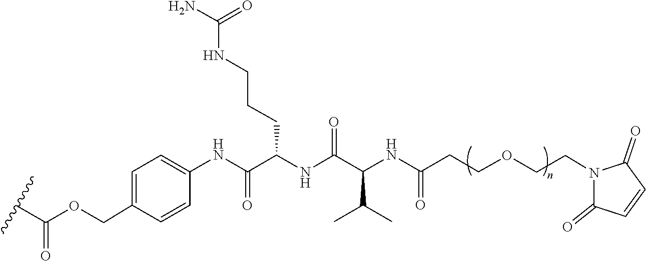 | L5 |
| 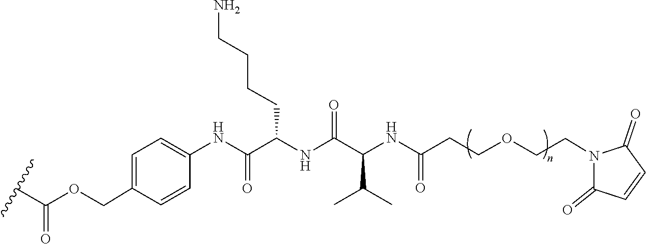 | L6 |
| 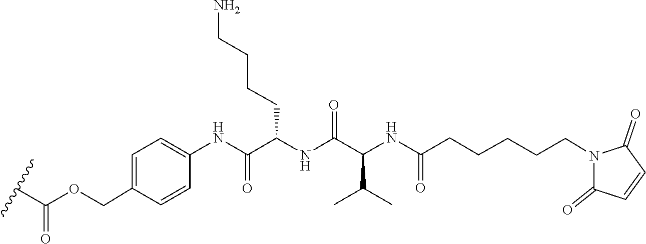 [SMILES: *C(=O)OCc1ccc(NC(=O)[C@H](CCCCN)NC(=O)[C@@H](NC(=O)CCCCCN2C(=O)C=CC2=O)C(C)C)cc1] | L7 |
| 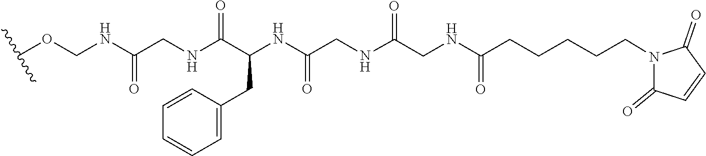 [SMILES: *OCNC(=O)CNC(=O)[C@H](Cc1ccccc1)NC(=O)CNC(=O)CNC(=O)CCCCCN1C(=O)C=CC1=O] | L8 |
| 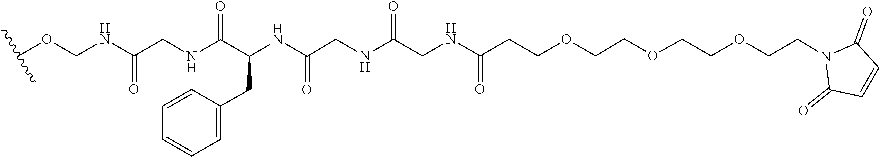 [SMILES: *OCNC(=O)CNC(=O)[C@H](Cc1ccccc1)NC(=O)CNC(=O)CNC(=O)CCOCCOCCOCCN1C(=O)C=CC1=O] | L9 |
| 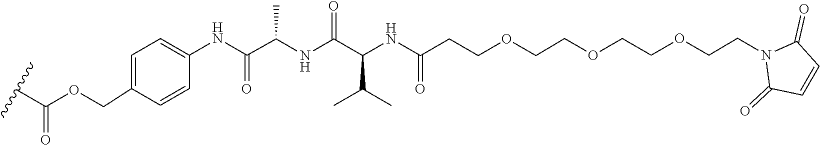 [SMILES: *C(=O)OCc1ccc(NC(=O)[C@H](C)NC(=O)[C@@H](NC(=O)CCOCCOCCOCCN2C(=O)C=CC2=O)C(C)C)cc1] | L10 |
| 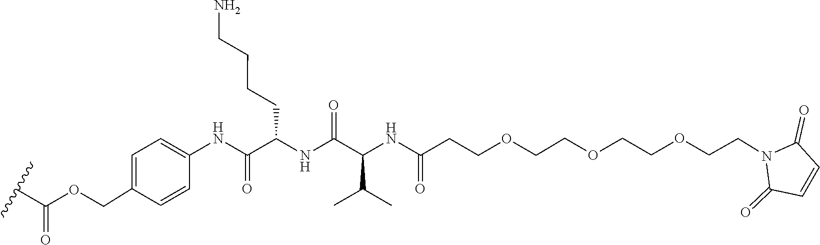 [SMILES: *C(=O)OCc1ccc(NC(=O)[C@H](CCCCN)NC(=O)[C@@H](NC(=O)CCOCCOCCOCCN2C(=O)C=CC2=O)C(C)C)cc1] | L11 |
| 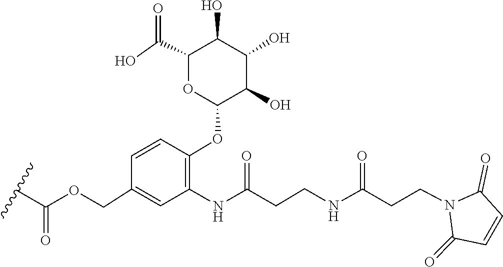 [SMILES: *C(=O)OCc1ccc(O[C@@H]2O[C@H](C(=O)O)[C@@H](O)[C@H](O)[C@H]2O)c(NC(=O)CCNC(=O)CCN2C(=O)C=CC2=O)c1] | L12 |
| 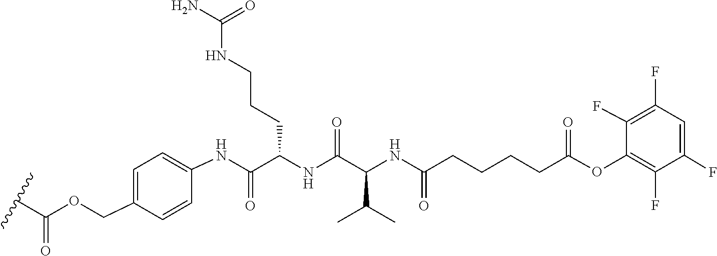 [SMILES: *C(=O)OCc1ccc(NC(=O)[C@H](CCCNC(N)=O)NC(=O)[C@@H](NC(=O)CCCCC(=O)Oc2c(F)c(F)cc(F)c2F)C(C)C)cc1] | L13 |
| 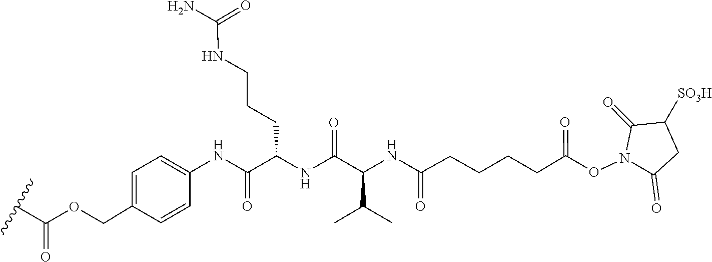 [SMILES: *C(=O)OCc1ccc(NC(=O)[C@H](CCCNC(N)=O)NC(=O)[C@@H](NC(=O)CCCCC(=O)ON2C(=O)CC(S(=O)(=O)O)C2=O)C(C)C)cc1] | L14 |
| 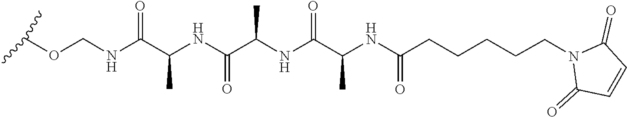 [SMILES: *OCNC(=O)[C@H](C)NC(=O)[C@@H](C)NC(=O)[C@H](C)NC(=O)CCCCCN1C(=O)C=CC1=O] | L15 |
| 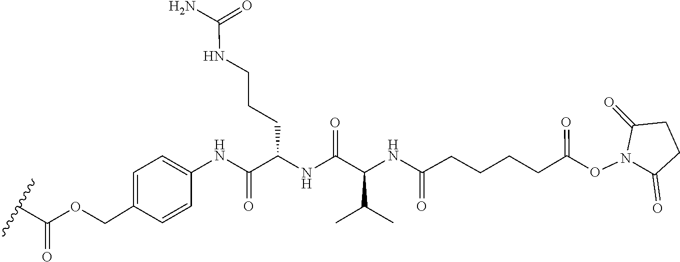 [SMILES: *C(=O)OCc1ccc(NC(=O)[C@H](CCCNC(N)=O)NC(=O)[C@@H](NC(=O)CCCCC(=O)ON2C(=O)CCC2=O)C(C)C)cc1] | L16 |
| 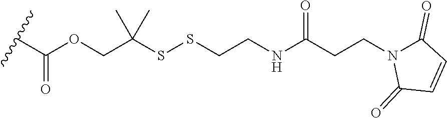 [SMILES: *C(=O)OCC(C)(C)SSCCNC(=O)CCN1C(=O)C=CC1=O] | L17 |
| 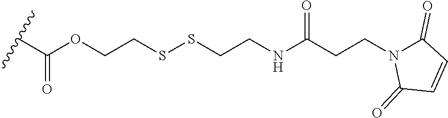 [SMILES: *C(=O)OCCSSCCNC(=O)CCN1C(=O)C=CC1=O] | L18 |

In certain embodiments, compound-linker constructs of Formula (A) can comprise a cleavable linker. In some embodiments, compound-linker constructs of Formula (A) can comprise a peptide-containing linker. In some embodiments, compound-linker constructs of Formula (A) can comprise a protease-cleavable linker.

In various embodiments, and with reference to Formula (I), a linker moiety (L) can be attached to a compound having a structure of Formula (I) at any suitable atom. Suitable groups on compounds of Formulae (I)-(IV), C, for attachment of linker, L, in either of the above approaches include, but are not limited to, thiol groups, amine groups, carboxylic acid groups and hydroxyl groups. In some embodiments of the present disclosure, linker, L, is attached to a compound of Formula (I), C, via a hydroxyl or amine group on the compound.

In some embodiments, the attachment site for the linker moiety is one of the sites shown below in Formula (I-L), based on Formula (I), with “L” indicating potential linker attachment sites:

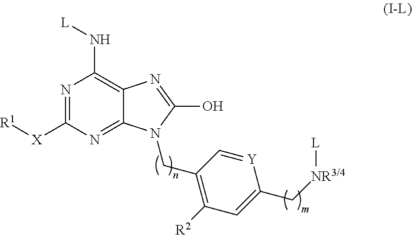

“R^{3/4}” indicates that when either R³ or R⁴ in Formula I is H, such hydrogen atom can be substituted with a linker moiety, e.g., as shown herein for MT-VC-PABC-Compound 105 and MT-VC-PABC-Compound 107. Furthermore, and in embodiments where either R³ and/or R⁴ in Formula I are substituents containing one or more additional nitrogen atoms suitable for attachment of a linker L, such nitrogen atom(s) may be the attachment site for the linker moiety L. Examples thereof include MT-VC-PABC-Compound 100, MT-VC-PABC-Compound 106 and MT-VC-PABC-Compound 111.

In some embodiments, a compound-linker construct herein comprising a purine-derived compound of any one of Formulae (I)-(IV) can be selected from TABLE 2B.

TABLE 2B
Exemplary Compound-Linker Constructs of the Present Disclosure

| Structure | Name |
|---|---|
| 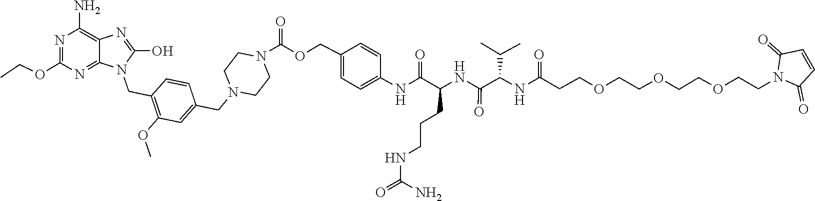 [SMILES: CCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 100 |
| 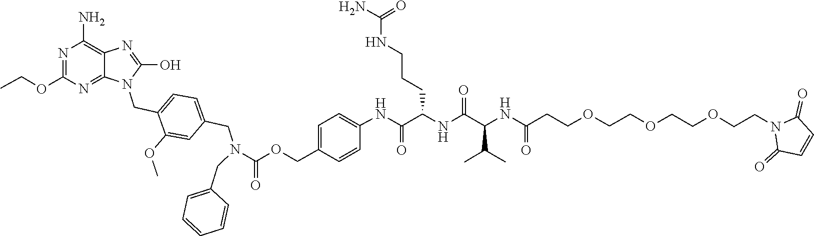 [SMILES: CCOc1nc(N)c2nc(O)n(Cc3ccc(CN(Cc4ccccc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1] | MT- VC- PABC- Com- pound 107 |
| 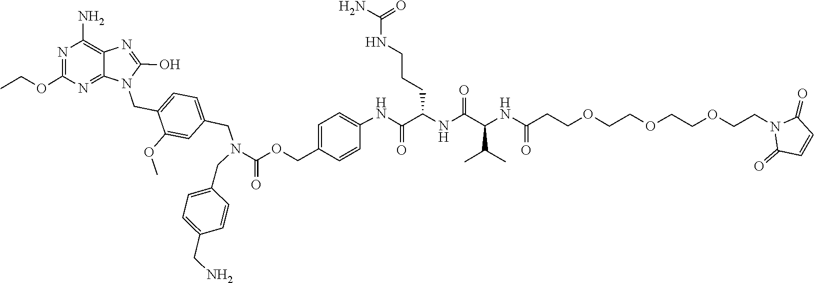 [SMILES: CCOc1nc(N)c2nc(O)n(Cc3ccc(CN(Cc4ccc(CN)cc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1] | MT- VC- PABC- Com- pound 105 |
| 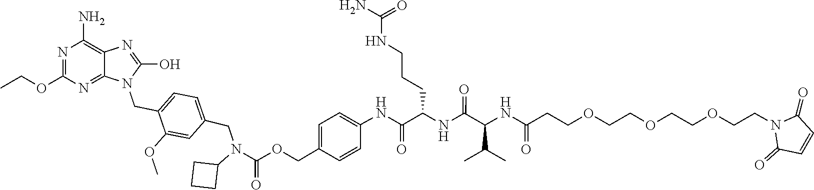 [SMILES: CCOc1nc(N)c2nc(O)n(Cc3ccc(CN(C(=O)OCc4ccc(NC(=O)[C@H](CCCNC(N)=O)NC(=O)[C@@H](NC(=O)CCOCCOCCOCCN5C(=O)C=CC5=O)C(C)C)cc4)C4CCC4)cc3OC)c2n1] | MT- VC- PABC- Com- pound 108 |
| 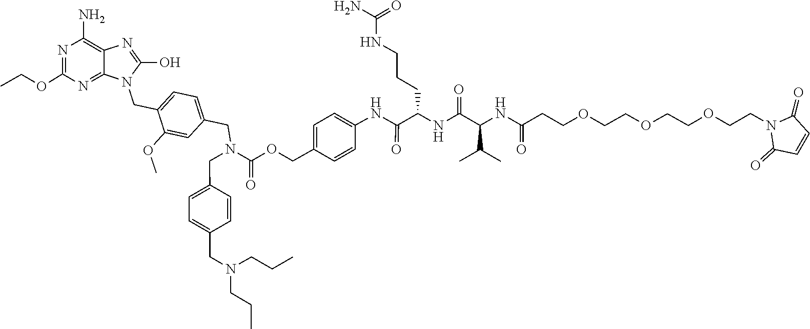 [SMILES: CCCN(CCC)Cc1ccc(CN(Cc2ccc(Cn3c(O)nc4c(N)nc(OCC)nc43)c(OC)c2)C(=O)OCc2ccc(NC(=O)[C@H](CCCNC(N)=O)NC(=O)[C@@H](NC(=O)CCOCCOCCOCCN3C(=O)C=CC3=O)C(C)C)cc2)cc1] | MT- VC- PABC- Com- pound 141 |
| 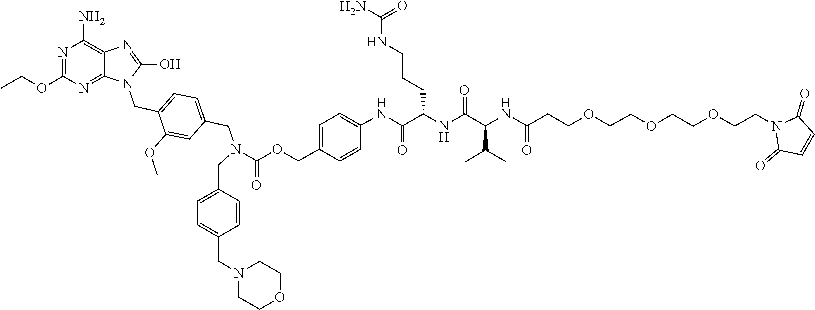 [SMILES: CCOc1nc(N)c2nc(O)n(Cc3ccc(CN(Cc4ccc(CN5CCOCC5)cc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1] | MT- VC- PABC- Com- pound 142 |
| 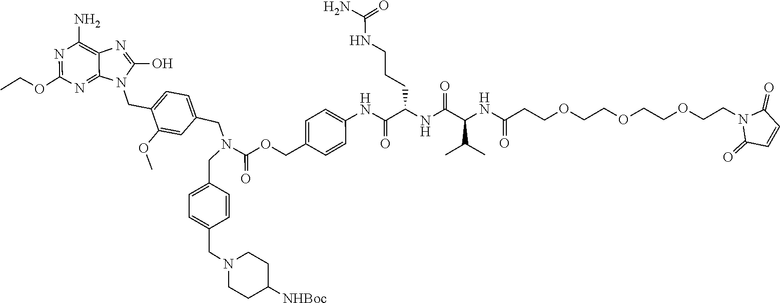 [SMILES: CCOc1nc(N)c2nc(O)n(Cc3ccc(CN(Cc4ccc(CN5CCC(NC(=O)OC(C)(C)C)CC5)cc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1] | MT- VC- PABC- Com- pound 143 |
| 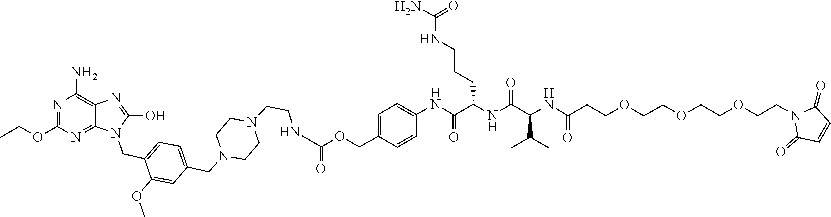 [SMILES: CCOc1nc(N)c2nc(O)n(Cc3ccc(CN4CCN(CCNC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 112 |
| 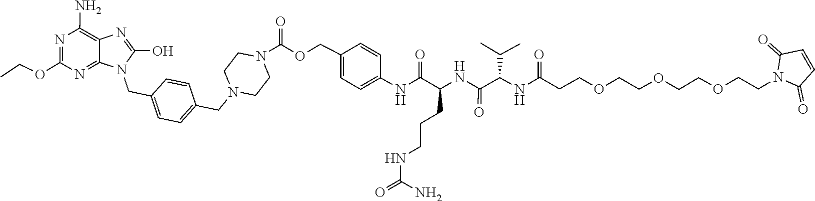 [SMILES: CCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3)c2n1] | MT- VC- PABC- Com- pound 150 |
| 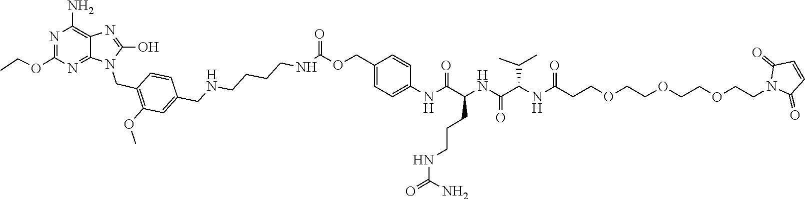 [SMILES: CCOc1nc(N)c2nc(O)n(Cc3ccc(CNCCCCNC(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1] | MT- VC- PABC- Com- pound 106 |
| 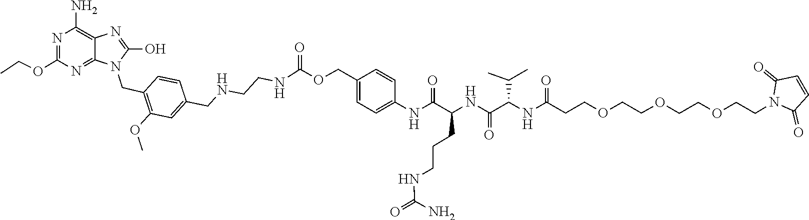 [SMILES: CCOc1nc(N)c2nc(O)n(Cc3ccc(CNCCNC(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1] | MT- VC- PABC- Com- pound 111 |
| 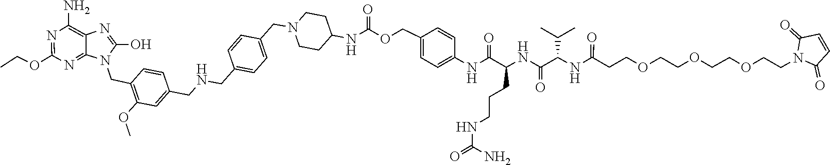 [SMILES: CCOc1nc(N)c2nc(O)n(Cc3ccc(CNCc4ccc(CN5CCC(NC(=O)OCc6ccc(NC(=O)[C@H](CCCNC(N)=O)NC(=O)[C@@H](NC(=O)CCOCCOCCOCCN7C(=O)C=CC7=O)C(C)C)cc6)CC5)cc4)cc3OC)c2n1] | MT- VC- PABC- Com- pound 144 |
| 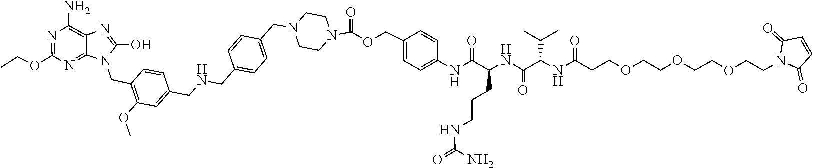 [SMILES: CCOc1nc(N)c2nc(O)n(Cc3ccc(CNCc4ccc(CN5CCN(C(=O)OCc6ccc(NC(=O)[C@H](CCCNC(N)=O)NC(=O)[C@@H](NC(=O)CCOCCOCCOCCN7C(=O)C=CC7=O)C(C)C)cc6)CC5)cc4)cc3OC)c2n1] | MT- VC- PABC- Com- pound 145 |
| 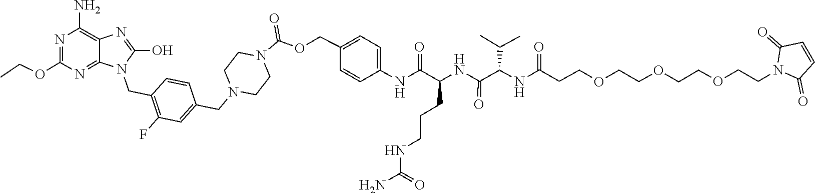 [SMILES: CCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3F)c2n1] | MT- VC- PABC- Com- pound 152 |
| 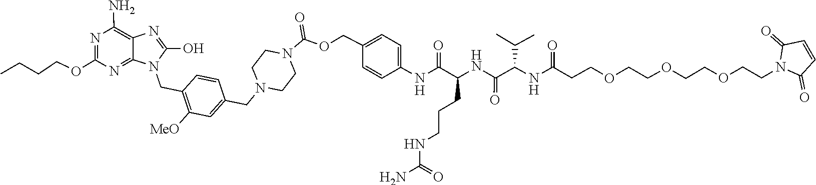 [SMILES: CCCCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 154 |
| 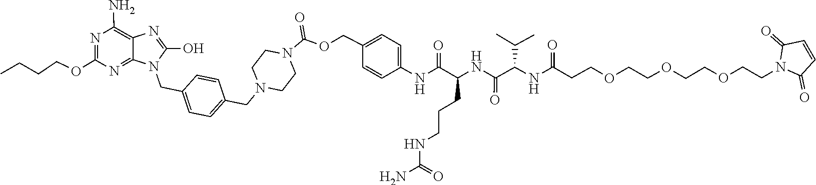 [SMILES: CCCCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3)c2n1] | MT- VC- PABC- Com- pound 166 |
| 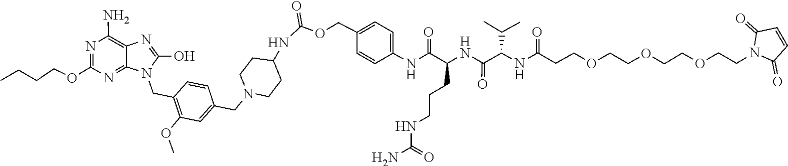 [SMILES: CCCCOc1nc(N)c2nc(O)n(Cc3ccc(CN4CCC(NC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 155 |
| 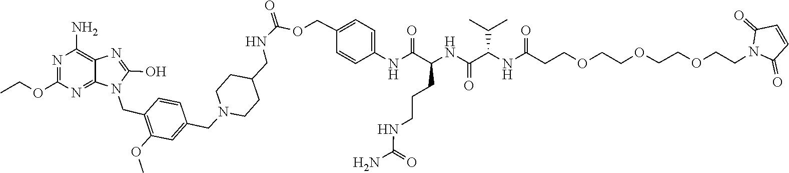 [SMILES: CCOc1nc(N)c2nc(O)n(Cc3ccc(CN4CCC(CNC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 132 |
| 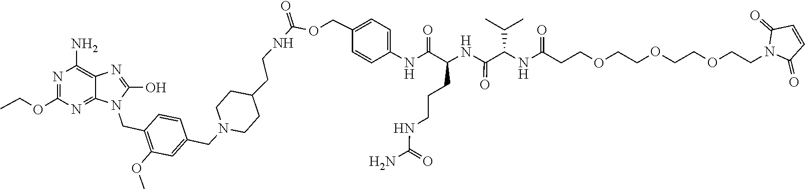 [SMILES: CCOc1nc(N)c2nc(O)n(Cc3ccc(CN4CCC(CCNC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 140 |
| 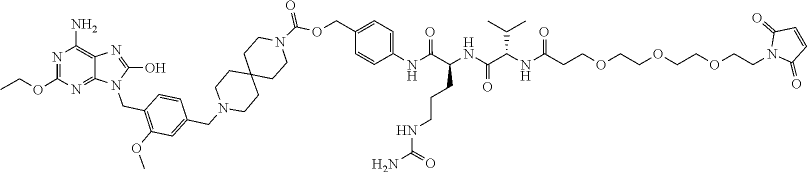 [SMILES: CCOc1nc(N)c2nc(O)n(Cc3ccc(CN4CCC5(CC4)CCN(C(=O)OCc4ccc(NC(=O)[C@H](CCCNC(N)=O)NC(=O)[C@@H](NC(=O)CCOCCOCCOCCN6C(=O)C=CC6=O)C(C)C)cc4)CC5)cc3OC)c2n1] | MT- VC- PABC- Com- pound 139 |
| 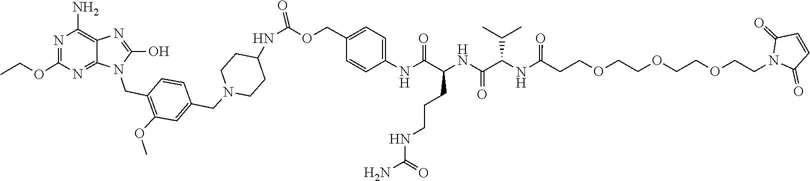 [SMILES: CCOc1nc(N)c2nc(O)n(Cc3ccc(CN4CCC(NC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 110 |
| 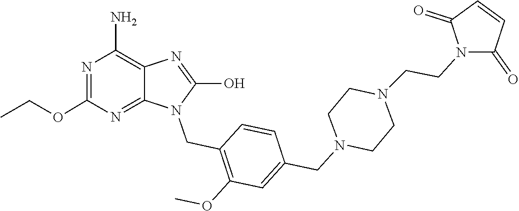 [SMILES: CCOc1nc(N)c2nc(O)n(Cc3ccc(CN4CCN(CCN5C(=O)C=CC5=O)CC4)cc3OC)c2n1] | ME2- Com- pound 100 |
| 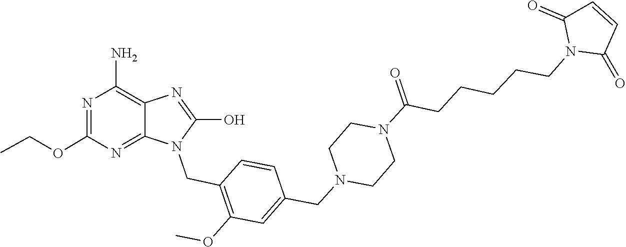 [SMILES: CCOc1nc(N)c2nc(O)n(Cc3ccc(CN4CCN(C(=O)CCCCCN5C(=O)C=CC5=O)CC4)cc3OC)c2n1] | MC- Com- pound 100 |
| 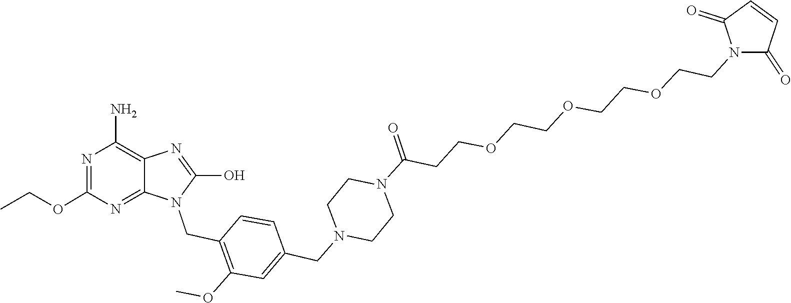 [SMILES: CCOc1nc(N)c2nc(O)n(Cc3ccc(CN4CCN(C(=O)CCOCCOCCOCCN5C(=O)C=CC5=O)CC4)cc3OC)c2n1] | MT- Com- pound 100 |
| 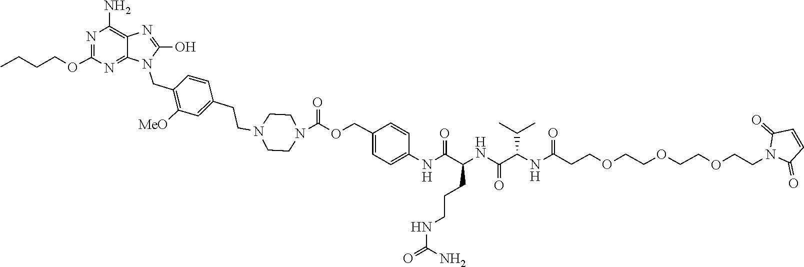 [SMILES: CCCCOc1nc(N)c2nc(O)n(Cc3ccc(CCN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 180 |
| 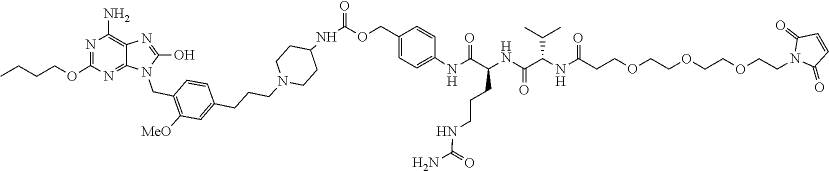 [SMILES: CCCCOc1nc(N)c2nc(O)n(Cc3ccc(CCCN4CCC(NC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 172 |
| 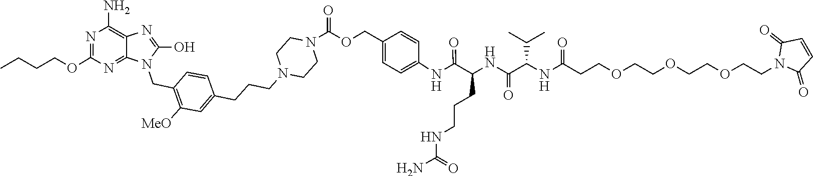 [SMILES: CCCCOc1nc(N)c2nc(O)n(Cc3ccc(CCCN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1] | MT- VC- PABC- Com- pound 173 |
| 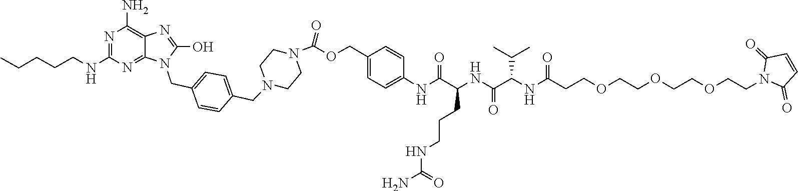 [SMILES: CCCCCNc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3)c2n1] | MT- VC- PABC- Com- pound 187 |
| 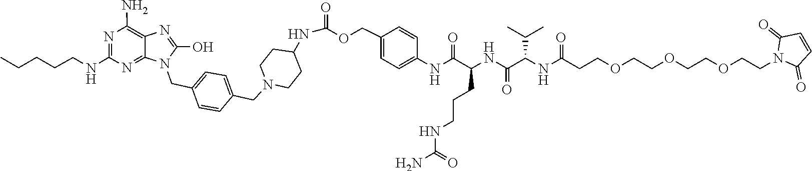 [SMILES: CCCCCNc1nc(N)c2nc(O)n(Cc3ccc(CN4CCC(NC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3)c2n1] | MT- VC- PABC- Com- pound 188 |
| 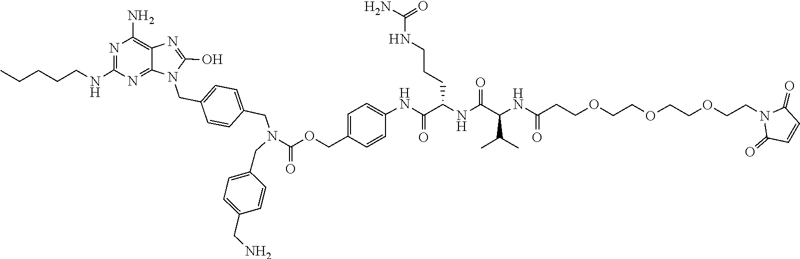 [SMILES: CCCCCNc1nc(N)c2nc(O)n(Cc3ccc(CN(Cc4ccc(CN)cc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3)c2n1] | MT- VC- PABC- Com- pound 191 |
| 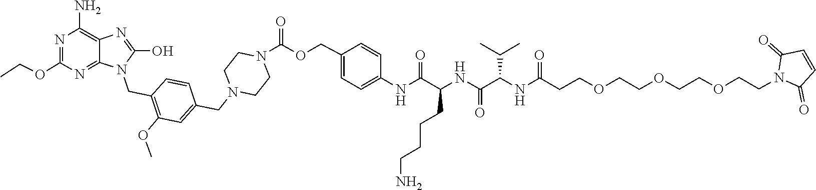 [SMILES: CCOc1nc(N)c2nc(O)n(Cc3ccc(CN4CCN(C(=O)OCc5ccc(NC(=O)[C@H](CCCCN)NC(=O)[C@@H](NC(=O)CCOCCOCCOCCN6C(=O)C=CC6=O)C(C)C)cc5)CC4)cc3OC)c2n1] | MT- VK- PABC- Com- pound 100 |
| 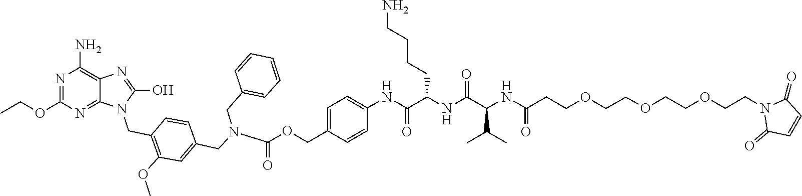 [SMILES: CCOc1nc(N)c2nc(O)n(Cc3ccc(CN(Cc4ccccc4)C(=O)OCc4ccc(NC(=O)[C@H](CCCCN)NC(=O)[C@@H](NC(=O)CCOCCOCCOCCN5C(=O)C=CC5=O)C(C)C)cc4)cc3OC)c2n1] | MT- VK- PABC- Com- pound 107 |
| 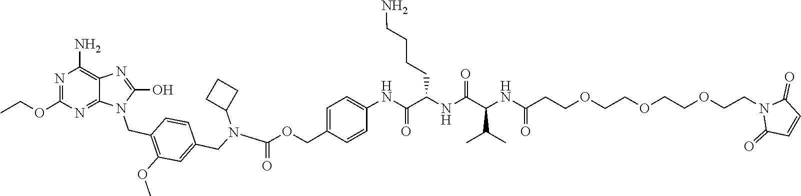 [SMILES: CCOc1nc(N)c2nc(O)n(Cc3ccc(CN(C(=O)OCc4ccc(NC(=O)[C@H](CCCCN)NC(=O)[C@@H](NC(=O)CCOCCOCCOCCN5C(=O)C=CC5=O)C(C)C)cc4)C4CCC4)cc3OC)c2n1] | MT- VK- PABC- Com- pound 108 |
| 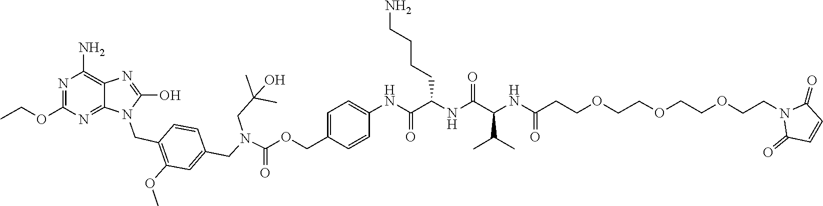 [SMILES: CCOc1nc(N)c2nc(O)n(Cc3ccc(CN(CC(C)(C)O)C(=O)OCc4ccc(NC(=O)[C@H](CCCCN)NC(=O)[C@@H](NC(=O)CCOCCOCCOCCN5C(=O)C=CC5=O)C(C)C)cc4)cc3OC)c2n1] | MT- VK- PABC- Com- pound 101 |
| 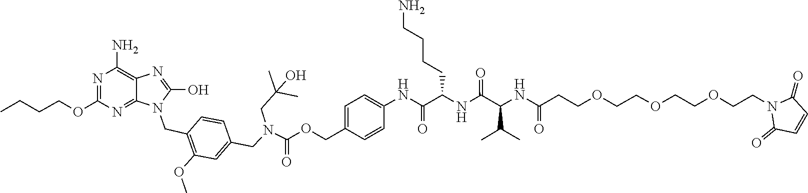 [SMILES: CCCCOc1nc(N)c2nc(O)n(Cc3ccc(CN(CC(C)(C)O)C(=O)OCc4ccc(NC(=O)[C@H](CCCCN)NC(=O)[C@@H](NC(=O)CCOCCOCCOCCN5C(=O)C=CC5=O)C(C)C)cc4)cc3OC)c2n1] | MT- VK- PABC- Com- pound 159 |
| 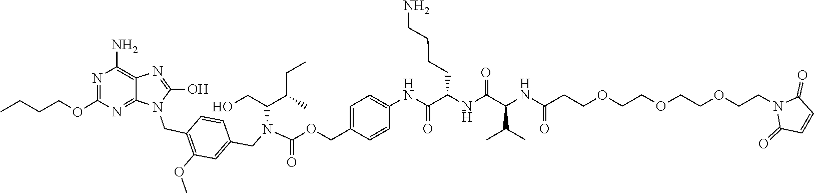 | MT- VK- PABC- Com- pound 161 |
| 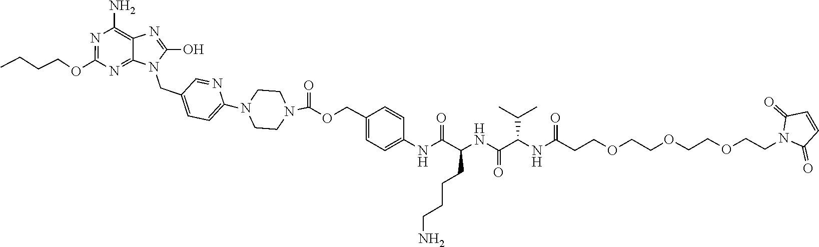 [SMILES: CCCCOc1nc(N)c2nc(O)n(Cc3ccc(N4CCN(C(=O)OCc5ccc(NC(=O)[C@H](CCCCN)NC(=O)[C@@H](NC(=O)CCOCCOCCOCCN6C(=O)C=CC6=O)C(C)C)cc5)CC4)nc3)c2n1] | MT- VK- PABC- Com- pound 167 |
| 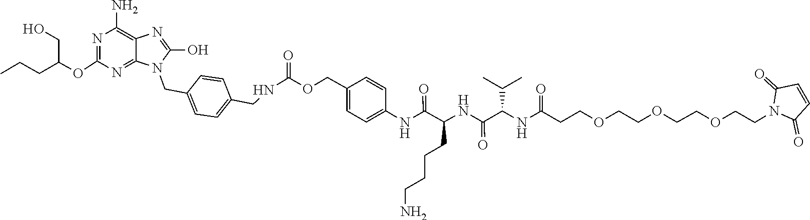 [SMILES: CCCC(CO)Oc1nc(N)c2nc(O)n(Cc3ccc(CNC(=O)OCc4ccc(NC(=O)[C@H](CCCCN)NC(=O)[C@@H](NC(=O)CCOCCOCCOCCN5C(=O)C=CC5=O)C(C)C)cc4)cc3)c2n1] | MT- VK- PABC- Com- pound 182 |
| 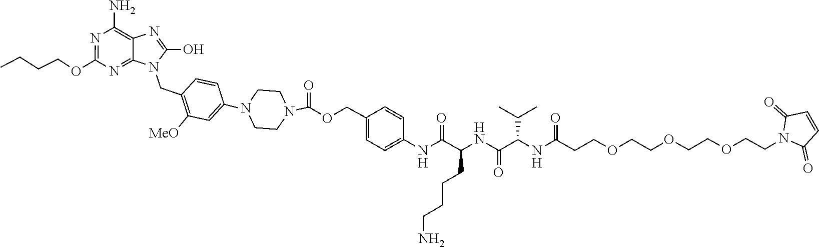 [SMILES: CCCCOc1nc(N)c2nc(O)n(Cc3ccc(N4CCN(C(=O)OCc5ccc(NC(=O)[C@H](CCCCN)NC(=O)[C@@H](NC(=O)CCOCCOCCOCCN6C(=O)C=CC6=O)C(C)C)cc5)CC4)cc3OC)c2n1] | MT- VK- PABC- Com- pound 169 |

A compound-linker construct of Formula (A) herein can be prepared using any suitable synthetic technique known in the art, e.g., as further described herein in EXAMPLE 2.

### Conjugates Comprising a Purine-Derived Compound

Further disclosed herein are conjugates (also referred to herein as “immunostimulatory antibody-drug conjugates” or “ISACs”) comprising a targeting moiety (T) coupled to one or more compounds of the present disclosure, e.g., one or more compounds according to any one or more of Formulae (I)-(IV). In various embodiments, a conjugate herein comprises one or more compound-linker construct(s) of Formula (A), L-(C)ₚ, as described herein.

In some embodiments, the conjugates of the present disclosure can comprise one or more compounds of any one of Formulae (I)-(IV) conjugated to the targeting moiety (T). In various embodiments, a conjugate of the present disclosure has a structure according to Formula (X):

T-[L-(C)ₚ]ᵣ (X)

wherein:
T is a targeting moiety;
L is a linker;
C is a compound according to any one of Formulae (I)-(IV);
p is an integer from 1 to 5; and
r has a value from about 1 to about 8.

In various embodiments of a conjugate of Formula (X) herein, L-(C)ₚ is a compound-linker construct as described herein.

As further described herein, in certain embodiments, the compounds disclosed herein can be used for the preparation of conjugates that are immunostimulatory antibody-drug conjugates (ISACs) that comprise an antibody as the targeting moiety which recognizes and binds to a tumor-associated antigen (e.g., a protein that is expressed on the surface of a tumor cell to be targeted). At a target site (e.g., tumor), the immunostimulatory compound(s) of the present disclosure that are attached to the targeting moiety can be released inside an immune cell and/or within a tumor microenvironment (TME), e.g., by using a cleavable linker, thereby causing an anti-tumor effect via activation of the immune system (e.g., via TLR7-mediated signaling and/or cytokine production).

### A. Targeting Moiety (T)

The targeting moiety, T, comprised by the conjugates of Formula (X) can be a molecule that binds, reactively associates, or complexes with a receptor, antigen or other receptive moiety associated with a given target cell population. Typically, the targeting moiety, T, functions to deliver a purine-derived compound, C, of the present disclosure to the particular target cell population with which the targeting moiety, T, reacts. Examples of targeting moieties include, but are not limited to, proteins (such as antibodies, antibody fragments and growth factors), glycoproteins, peptides (such as bombesin and gastrin-releasing peptide), lectins, vitamins (such as folic acid) and nutrient-transport molecules (such as transferrin).

Typically, the targeting moiety, T, will be bonded to linker, L, via a heteroatom of the targeting moiety, T, such as a sulfur (for example, from a sulfhydryl group), oxygen (for example, from a carbonyl, carboxyl, or hydroxyl group) or nitrogen (for example, from a primary or secondary amino group). These heteroatoms can be naturally present on targeting moiety, T, or can be introduced through engineering and/or expression, or can be introduced via chemical modification using techniques known in the art.

In some embodiments, targeting moiety, T, is an antibody. Accordingly, since the compounds of the present disclosure are capable of eliciting an immune response in a subject, various embodiments of the present disclosure relate to immunostimulatory antibody-drug conjugates (ISACs) having general Formula (X) in which the targeting moiety, T, is an antibody.

In embodiments in which the conjugate is an ISAC, the antibody included as the targeting moiety, T, can be a full-size polyclonal or monoclonal antibody, an antigen-binding antibody fragment (such as Fab, scFab, Fab′, F(ab′)₂, Fv or scFv), a domain antibody (dAb) or an antibody mimetic (such as an affibody, a DARPin, an anticalin, a versabody, a duocalin, a lipocalin or an avimer). The antibody is typically directed to a particular antigen, for example, a tumor-associated antigen (TAA).

In certain embodiments in which the conjugate (X) is an ISAC, the targeting moiety, T, is a monoclonal antibody, an antigen-binding antibody fragment thereof (such as Fab, scFab, Fab′, F(ab′)₂, Fv or scFv), or a domain antibody (dAb).

In certain embodiments, targeting moiety, T, can be a monoclonal antibody. The monoclonal antibody can be, for example, a non-human monoclonal antibody (such as a mouse or rabbit antibody), a human monoclonal antibody, a humanized monoclonal antibody, or a chimeric antibody (for example, a human-mouse antibody). In certain embodiments, the antibody included as a targeting moiety in a conjugate (X) herein can be a bispecific or multispecific antibody.

In certain embodiments, targeting moiety, T, comprised by the conjugate is an antibody or antigen-binding antibody fragment that binds to a tumor-associated antigen (TAA). In various embodiments, such TAA-binding antibody can comprise a functional Fc region capable of Fc-receptor binding.

### B. Linkers (L) Used in Conjugates

As further described herein, e.g., in the “compound-linker construct” section, a linker can couple one or more immunomodulatory compounds of the present disclosure, e.g., those of any one of compounds (I)-(IV), to the targeting moiety (T). For a detailed description of linker moieties contemplated herein, it is referred to the “compound-linker construct” section above.

As further described herein, a linker, L, of a compound-linker construct herein can comprise a functional group capable of reacting with the target group or groups on a targeting moiety, T, and at least one functional group capable of reacting with a target group on a compound of the present disclosure (e.g., a compound according to any one of Formulae (I)-(IV)), C. Suitable functional groups are known in the art and include those described herein, for example, in Bioconjugate Techniques (G. T. Hermanson, 2013, Academic Press). Functional groups on targeting moiety, T, and the purine-derived compound of the present disclosure, C, that can serve as target groups for linker attachment include, but are not limited to, thiol, hydroxyl, carboxyl, amine, aldehyde, and ketone groups.

Non-limiting examples of functional groups capable of reacting with thiols include maleimide, haloacetamide, haloacetyl, activated esters (such as succinimide esters, 4-nitrophenyl esters, pentafluorophenyl esters and tetrafluorophenyl esters), anhydrides, acid chlorides, sulfonyl chlorides, isocyanates and isothiocyanates. Also useful in this context are “self-stabilizing” maleimides as described in Lyon et al., 2014, Nat. Biotechnol., 32:1059-1062.

Non-limiting examples of functional groups capable of reacting with amines include activated esters (such as N-hydroxysuccinamide (NHS) esters and sulfo-NHS esters), imido esters (such as Traut's reagent), isothiocyanates, aldehydes and acid anhydrides (such as diethylenetriaminepentaacetic anhydride (DTPA)). Other examples include succinimido-1,1,3,3-tetra-methyluronium tetrafluoroborate (TSTU) and benzotriazol-1-yl-oxytripyrrolidino-phosphonium hexafluorophosphate (PyBOP).

Non-limiting examples of functional groups capable of reacting with an electrophilic group such as an aldehyde or ketone carbonyl group include hydrazide, oxime, amino, hydrazine, thiosemicarbazone, hydrazine carboxylate and arylhydrazide.

In certain embodiments in which targeting moiety, T, is an antibody, linker moiety, L, can include a functional group that allows for bridging of two interchain cysteines on the antibody, such as a ThioBridge™ linker (Badescu et al., 2014, Bioconjug. Chem. 25:1124-1136), a dithiomaleimide (DTM) linker (Behrens et al., 2015, Mol. Pharm. 12:3986-3998), a dithioaryl(TCEP)pyridazinedione-based linker (Lee et al., 2016, Chem. Sci., 7:799-802) or a dibromopyridazinedione-based linker (Maruani et al., 2015, Nat. Commun., 6:6645).

In other embodiments, targeting moiety, T, can be modified to include a non-natural reactive group, such as an azide, that allows for conjugation to the linker via a complementary reactive group on the linker. For example, conjugation of the linker to the targeting moiety can make use of click chemistry reactions (see, for example, Chio & Bane, 2020, Methods Mol. Biol., 2078:83-97), such as the azide-alkyne cycloaddition (AAC) reaction, which has been used successfully in the development of antibody-drug conjugates. The AAC reaction can be a copper-catalyzed AAC (CuAAC) reaction, which involves coupling of an azide with a linear alkyne, or a strain-promoted AAC (SPAAC) reaction, which involves coupling of an azide with a cyclooctyne.

In some embodiments, a (e.g., cleavable) linker is substantially stable in the extra-tumoral environment, e.g., in circulation. Such stable linker can be characterized in that at least about 90%, about 80%, about 70%, about 60%, about 50% or at least about 40% of the conjugates of Formula X is intact upon delivery to a tumor site and/or localization on a target cell surface and after a certain period of time. The intact conjugate can, for example, have an unchanged DAR (e.g., within about ±5%) compared to the conjugate at the time of administration. In other words, in some embodiments, the linker remains essentially uncleaved in the extra-tumoral environment during the time the conjugate is resident in this environment (e.g., in systemic circulation and/or a non-target tissue or organ). In some embodiments, such linker can be cleaved in the extra-tumoral environment but not to a degree that prevents a useful dosage of the intact conjugate being delivered to a target cell (e.g., a tumor cell). Whether a linker is not substantially sensitive to an extra-tumoral environment can be determined, e.g., by incubating the ISAC with plasma for a predetermined period of time (e.g., 2, 4, 8, 16, or 24 hours) and then quantitating the amount of free compound (e.g., those molecules that were cleaved from a conjugate) present in the plasma.

Various non-cleavable linkers are known in the art for linking compounds of the present disclosure to targeting moieties and can be used in the conjugates of the present disclosure in certain embodiments. Examples of non-cleavable linkers include linkers having an N-succinimidyl ester or N-sulfosuccinimidyl ester moiety for reaction with the cell binding agent, as well as a maleimido- or haloacetyl-based moiety for reaction with the compound (e.g., any one of the compounds according to Formulae (I)-(IV)), or vice versa. An example of such a non-cleavable linker is based on sulfosuccinimidyl-4-[N-maleimidomethyl]-cyclohexane-1-carboxylate (sulfo-SMCC). Sulfo-SMCC conjugation typically occurs via a maleimide group which reacts with sulfhydryls (thiols, —SH) on compound C, while the sulfo-NHS ester is reactive toward primary amines (as found in lysine and at the N-terminus of proteins or peptides) on targeting moiety T. Other non-limiting examples of such linkers include those based on N-succinimidyl 4-(maleimidomethyl)cyclohexanecarboxylate (SMCC), N-succinimidyl-4-(N-maleimidomethyl)-cyclohexane-1-carboxy-(6-amidocaproate) (“long chain” SMCC or LC-SMCC), maleimido-undecanoic acid N-succinimidyl ester (KMUA), maleimidobutyric acid N-succinimidyl ester (GMBS), maleimidocaproic acid N-hydroxysuccinimide ester (EMCS), m-maleimidobenzoyl-N-hydroxysuccinimide ester (MBS), N-(maleimidoacetoxy)-succinimide ester (AMAS), succinimidyl-6-(maleimidopropionamido)hexanoate (SMPH), N-succinimidyl 4-(p-maleimidophenyl)-butyrate (SMPB) and N-(p-maleimidophenyl)isocyanate (PMPI). Other examples include those comprising a haloacetyl-based functional group such as N-succinimidyl-4-(iodoacetyl)-aminobenzoate (SIAB), N-succinimidyl iodoacetate (SIA), N-succinimidyl bromoacetate (SBA) and N-succinimidyl 3-(bromoacetamido)propionate (SBAP).

### C. Preparation of Conjugates

Conjugates of Formula (X) herein can be prepared by standard methods known in the art (see, for example, Bioconjugate Techniques (G. T. Hermanson, 2013, Academic Press)). Various linkers and linker components are commercially available or can be prepared using standard synthetic organic chemistry techniques (see, for example, March's Advanced Organic Chemistry (Smith & March, 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press)). In addition, various antibody drug conjugation services are available commercially from companies such as Lonza Inc. (Allendale, NJ), Abzena PLC (Cambridge, UK), ADC Biotechnology (St. Asaph, UK), Baxter BioPharma Solutions (Baxter Healthcare Corporation, Deerfield, IL) and Piramel Pharma Solutions (Grangemouth, UK).

Typically, preparation of the conjugates described herein comprises first preparing a compound-linker construct of Formula (A) as described herein, C-L, comprising one or more compounds of any one of Formulae (I)-(IV) and a linker, L, and then conjugating the compound-linker construct, (C-L)ₚ, to an appropriate group on targeting moiety, T. Ligation of linker, L, to targeting moiety, T, and subsequent ligation of the targeting moiety-linker, T-L, to one or more compounds described herein, e.g., those of Formula (I), C, remains however an alternative approach that can be employed in some embodiments. Exemplary methods are described in EXAMPLE 4 herein.

Suitable groups on targeting moiety, T, for attachment of linker, L, in either of the above approaches include sulfhydryl groups (for example, on the side-chain of cysteine residues), amino groups (for example, on the side-chain of lysine residues), carboxylic acid groups (for example, on the side-chains of aspartate or glutamate residues), and carbohydrate groups (e.g., on an Fc glycan moiety). For example, targeting moiety T can comprise one or more naturally occurring sulfhydryl groups allowing targeting moiety, T, to bond to a linker, L, via the sulfur atom of a sulfhydryl group. In some embodiments, a targeting moiety, T, can comprise one or more lysine residues that can be chemically modified to introduce one or more sulfhydryl groups. Reagents that can be used to modify lysine residues include, but are not limited to, N-succinimidyl S-acetylthioacetate (SATA), N-succinimidyl-3-(2-pyridyldithio)propionate (“SPDP”) and 2-iminothiolane hydro-chloride (Traut's Reagent). In other embodiments, targeting moiety, T, can comprise one or more carbohydrate groups that can be chemically modified to include one or more sulfhydryl groups.

In some embodiments, carbohydrate groups on a targeting moiety, T, can also be oxidized to provide an aldehyde (—CHO) group (see, for example, Laguzza et al., 1989, J. Med. Chem. 32(3):548-55), which can subsequently be reacted with linker, L, for example, via a hydrazine or hydroxylamine group on linker, L.

A targeting moiety, T, can also be modified to include additional cysteine residues (see, for example, U.S. Pat. Nos. 7,521,541; 8,455,622 and 9,000,130) or non-natural amino acids that provide reactive handles, such as selenomethionine, p-acetylphenylalanine, formylglycine or p-azidomethyl-L-phenylalanine (see, for example, Hofer et al., 2009, Biochemistry, 48:12047-12057; Axup et al., 2012, PNAS, 109:16101-16106; Wu et al., 2009, PNAS, 106:3000-3005; Zimmerman et al., 2014, Bioconj. Chem., 25:351-361), to allow for site-specific conjugation. In some embodiments, targeting moiety, T, can be modified to include a non-natural reactive group, such as an azide, that allows for conjugation to the linker via a complementary reactive group on the linker, for example, by click chemistry (see, for example, Chio & Bane, 2020, Methods Mol. Biol., 2078:83-97).

Other protocols for the modification of proteins of which a targeting moiety (T) herein can comprise or consist of, to allow for the attachment or association of linker, L, are known in the art and include those described in Coligan et al., Current Protocols in Protein Science, vol. 2, John Wiley & Sons (2002).

In those embodiments in which targeting moiety, T, is an antibody, several different reactive groups on the antibody can function as a conjugation site, including E-amino groups on lysine residues, pendant carbohydrate moieties, side-chain carboxylic acid groups on aspartate or glutamate residues, cysteine-cysteine disulfide groups and cysteine thiol groups. The amino acids used for conjugation can be part of the natural sequence of the antibody, or they can be introduced by site-specific engineering techniques known in the art, as noted herein.

Once conjugation is complete, the average number of compounds of Formulae (I)-(IV) conjugated to targeting moiety, T, (i.e., the “drug-to-antibody ratio” or DAR) can be determined by standard techniques such as UV/VIS spectroscopic analysis, ELISA-based techniques, chromatography techniques such as hydrophobic interaction chromatography (HIC), UV-MALDI mass spectrometry (MS) and MALDI-TOF MS. In addition, distribution of compound-linked forms (for example, the fraction of targeting moiety, T, containing zero, one, two, three, etc. compounds of Formula (I), C) can also optionally be analyzed. Various techniques are known in the art to measure DAR distribution, including MS (with or without an accompanying chromatographic separation step), hydrophobic interaction chromatography, reverse-phase HPLC or iso-electric focusing gel electrophoresis (IEF) (see, for example, Wakankar et al., 2011, mAbs, 3:161-172).

### D. Properties of Conjugates

As described herein, the drug-to-antibody ratio (DAR) of a conjugate (or “p” in Formula X, if r=1, otherwise the product of q times r), as disclosed herein, refers to the ratio of the number of compound(s) C of Formulae (I)-(IV) conjugated to a targeting moiety, T.

As noted above and reflected by parameters p and r in Formula (X), a targeting moiety, “T,” can be conjugated to more than one compound(s) “C” of Formula (I). Those skilled in the art will appreciate that, while any particular targeting moiety T is conjugated to an integer number of compounds C, analysis of a preparation of the conjugate to determine the ratio of compound C to targeting moiety T can give a non-integer result, reflecting a statistical average. This ratio of compound C to targeting moiety T can generally be referred to as the drug-to-antibody ratio, or “DAR.” Accordingly, conjugate preparations having non-integer DARs are intended to be encompassed by Formula (X). One skilled in the art will appreciate that the term “DAR” can be employed to define conjugates comprising targeting moieties other than antibodies.

In some embodiments, the DAR of a conjugate of Formula (X) herein is obtained by any combination of linker-to-antibody ratio and/or drug-to-linker ratio (e.g., where r is the ratio of compound-linker constructs to antibody T, and p is the number of compounds, C, per linker, L). For example, where r=2 and p=2, the number of compounds C of Formulae (I)-(IV) in the respective antibody-drug conjugate is 4 (e.g., each tumor-targeting antibody T is coupled to 2 compound-linker constructs, with each linker construct comprising 2 immunostimulatory compounds of Formulae (I)-(IV)).

In some embodiments, the DAR of the conjugates of Formula (X) is from about 1 to about 32. In some embodiments, the DAR of the conjugates of Formula (X) is from about 1 to about 24, from about 1 to about 16, from about 1 to about 8, from about 3 to about 5, or from about 1 to about 4. In some embodiments, the DAR of the conjugates of Formula (X) is from about 2 to about 32, from about 2 to about 24, from about 2 to about 16, from about 2 to about 8 or from about 2 to about 4. In various embodiments, the DAR can be from about 1.5 to about 4.5.

In some embodiments, the DAR (i.e., the product of r×p in Formula (X)) of the conjugates of Formula (X) herein can have any numeric value from about 1 to about 8, and thus can have a value of about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or about 8.0. In some embodiments, the DAR of a conjugate of Formula (X) herein can have a value from about 2 to about 8, from about 2 to about 6, from about 2 to about 5, from about 3 to about 5, or from about 2 to about 4.

As further described elsewhere herein, all compounds (e.g., those of Formulae (I)-(IV)) of a conjugate of Formula (X) can be identical. In other embodiments, two or more compounds of a conjugate of Formula (X) can be different.

In some embodiments, a conjugate of Formula (X) herein can have an EC₅₀ value for agonizing TLR7 from about 10 nM to about 50 pM, from about 5 nM to about 50 pM, from about 1 nM to about 50 pM, from about 750 pM to about 50 pM, from about 600 pM to about 50 pM, from about 500 pM to about 50 pM, from about 400 pM to about 50 pM, from about 300 pM to about 50 pM, from about 200 pM to about 50 pM, or from about 100 pM to about 50 pM.

Without being bound by any theory, it is assumed that anti-tumor activity of a conjugate of Formula (X) herein can be mediated by innate immune cells. The conjugate, particularly in embodiments in which the targeting moiety is an antibody or fragment thereof, can initially bind the tumor cell via the target TAA and the immune cell via FcγR engagement. Subsequently, the conjugate can be catabolized in the phagolysosome to release the TLR7 agonist, e.g., a compound of any one of Formulae (I)-(IV). Stimulation of TLR7 in the phagolysosome can induce cytokine expression, which can drive the anti-tumor response. As a result, TLR7 agonism mediated by a compound described herein can drive anti-tumor immunity in a subject.

In various embodiments, and as further described herein, a conjugate of Formula (X) of the present disclosure that comprises one or more immunomodulatory compounds can be capable of inducing production and/or release of one or more cytokines from immune cells. Such immune cells can be human or murine immune cells, such as PBMCs or splenocytes, respectively, and can be part of a cell population further comprising other cell types, such as other immune cell, tumor cells, etc. Such induction of, e.g., pro-inflammatory cytokines, can be result of an interaction between the immunostimulatory compound of the conjugate and TLR7 of immune cells.

In various embodiments, a conjugate of Formula (X) herein can induce production of one or more cytokines when contacted with a cell population comprising immune cells. In various embodiments, such cytokine belongs to the IL6 superfamily. In various embodiments, a conjugate of Formula (X) herein can induce production of IL6. Induction of IL6 can include human IL6, murine IL6, or both.

In various embodiments, a conjugate of Formula (X) herein can have an EC₅₀ value for inducing production of a cytokine in a human or murine immune cell population of <1 nM, <500 pM, <250 pM, <100 pM, or <50 pM, or less. In some embodiments, a conjugate of Formula (X) herein can have an EC₅₀ value for inducing production of a cytokine in a human or murine immune cell population of <1 nM, <500 nM, or <250 nM. In some embodiments, a conjugate of Formula (X) herein can have an EC₅₀ value for inducing production of a cytokine in a human or murine immune cell population from about 1 nM to about 100 pM, from about 750 pM to about 50 pM, from about 500 pM to about 50 pM, from about 250 pM to about 50 pM, or from about 150 pM to about 50 pM. In various embodiments, the cytokine which production is induced by the conjugate is IL6. In some embodiments, induction of cytokine production of a conjugate can be determined via human and/or murine immune cell assays, which can comprise incubating the conjugate with a cell population comprising immune cells (e.g., ones that express TLR7) and tumor cells (e.g., those that express an antigen that the targeting moiety binds to), followed by measuring the amount of cytokine in the supernatant after a certain period of time, as further described herein in, e.g., EXAMPLE 5.

In various embodiments, a conjugate of Formula (X) that comprises a compound of any one of Formulae (I)-(IV) herein, can elicit an immune response in vivo. In various embodiments, such immune response can result in an anti-tumor activity when administered to a subject (e.g., a human, non-human primate, or rodent) suffering from a cancer. Such anti-tumor activity can be measured, e.g., by a reduction of tumor growth rate. In some embodiments, when administered to a group of tumor-bearing test subjects, the anti-tumor activity of a conjugate of Formula (X) herein is sufficient to reduce the volume of the tumor in one or more of the test subjects. In some embodiments, a conjugate of Formula (X) herein can reduce the volume of a tumor by at least about 30%, 40%, 50%, 60%, 70%, 80%, or 90%, in at least one of the test subjects.

In certain embodiments, a conjugate of Formula (X) herein can demonstrate good tolerability in vivo. In some embodiments, a conjugate of Formula (X) herein can be well tolerated in vivo at doses of at least about 1 mg/kg, at least about 3 mg/kg, at least about 15 mg/kg, or about 45 mg/kg. A conjugate herein is generally well tolerated when the total body weight loss in a test subject at the end of a study period is no more than 20% relative to the baseline body weight at the start of the study, i.e., prior to treatment. A study period can be, e.g., between 10 and 16 days. In some embodiments, a conjugate of Formula (X) is well tolerated at a dose that is effective in reducing the volume of a tumor in the test subject.

### Pharmaceutical Compositions

Compounds of Formulae (I)-(IV) and conjugates comprising compounds of Formulae (I)-(IV) can be formulated for therapeutic use. In certain embodiments, the present disclosure further discloses pharmaceutical compositions comprising a compound of the present disclosure, e.g., a compound according to any one of Formulae (I)-(IV) (see, e.g., section above “Compounds”), or a pharmaceutically acceptable salt thereof. Further disclosed herein are pharmaceutical compositions comprising a conjugate of Formula (X) (see, e.g., section above “Conjugates”). Any such pharmaceutical composition can further comprise a pharmaceutically acceptable carrier, diluent, or excipient. In some embodiments, the pharmaceutical composition is a therapeutic composition for the treatment of a disease (e.g., a cancer) in a subject in need thereof. Such pharmaceutical compositions can be prepared by known procedures using well-known and readily available ingredients.

Pharmaceutical compositions described herein can be formulated for administration to a subject by, for example, oral (including, for example, buccal or sublingual), topical, parenteral, rectal, or vaginal routes, or by inhalation or spray. The term parenteral as used herein includes subcutaneous injection, and intradermal, intra-articular, intravenous, intramuscular, intravascular, intrasternal, intrathecal injection or infusion. A pharmaceutical composition can be formulated in a format suitable for administration to the subject, for example, as a syrup, elixir, tablet, troche, lozenge, hard or soft capsule, pill, suppository, oily or aqueous suspension, dispersible powder or granule, emulsion, injectable or solution. Pharmaceutical compositions can be provided as unit dosage formulations.

Pharmaceutical compositions intended for oral use can be prepared in either solid or fluid unit dosage forms. Fluid unit dosage forms can be prepared according to procedures known in the art for the manufacture of pharmaceutical compositions and such compositions can contain one or more agents such as sweetening agents, flavouring agents, colouring agents, and preserving agents in order to provide pharmaceutically elegant and palatable preparations. An elixir can be prepared by using a hydroalcoholic (for example, ethanol) carrier with suitable sweeteners such as sugar and/or saccharin, together with an aromatic flavoring agent. Suspensions can be prepared with an aqueous carrier and a suspending agent such as acacia, tragacanth, methylcellulose, and the like.

Solid formulations, such as tablets, contain the active ingredient (e.g., a compound and/or conjugate of the present disclosure) in admixture with non-toxic pharmaceutically acceptable excipients that are suitable for the manufacture of tablets. These excipients can be for example, inert diluents, such as calcium carbonate, sodium carbonate, lactose, calcium phosphate or sodium phosphate; granulating and disintegrating agents for example, corn starch, or alginic acid; binding agents, for example starch, gelatin or acacia, and/or lubricating agents, for example magnesium stearate, stearic acid or talc, as well as other conventional ingredients such as dicalcium phosphate, magnesium aluminum silicate, calcium sulfate, starch, lactose, methylcellulose, and functionally similar materials. The tablets can be uncoated, or they can be coated by known techniques, for example, in order to delay disintegration and absorption in the gastrointestinal tract and thereby provide a sustained action over a longer period. For example, a time delay material such as glyceryl monostearate or glyceryl distearate can be employed.

Formulations for oral use can also be presented as hard gelatin capsules wherein the active ingredient (e.g., a compound and/or conjugate of the present disclosure) is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Soft gelatin capsules are typically prepared by machine encapsulation of a slurry of the active ingredient with an acceptable vegetable oil, light liquid petrolatum or other inert oil.

Aqueous suspensions can contain the active ingredient (e.g., a compound and/or conjugate of the present disclosure) in admixture with excipients suitable for the manufacture of aqueous suspensions. Such excipients include suspending agents, for example sodium carboxylmethylcellulose, methyl cellulose, hydropropylmethylcellulose, sodium alginate, polyvinylpyrrolidone, gum tragacanth and gum acacia, and dispersing or wetting agents. Dispersing and wetting agents include, for example, naturally-occurring phosphatides (e.g., lecithin), condensation products of an alkylene oxide with fatty acids (e.g., polyoxyethylene stearate), condensation products of ethylene oxide with long chain aliphatic alcohols (e.g., hepta-decaethyleneoxycetanol), condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions can also contain one or more preservatives (e.g., ethyl, or n-propyl-p-hydroxybenzoate), one or more colouring agents, one or more flavouring agents and/or one or more sweetening agents (e.g., sucrose or saccharin).

Oily suspensions can be formulated by suspending the active ingredient (e.g., a compound and/or conjugate of the present disclosure) in a vegetable oil, for example peanut oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavouring agents can be added to provide palatable oral preparations. The suspensions can optionally be preserved by the addition of an antioxidant such as ascorbic acid.

Dispersible powders and granules suitable for preparation of an aqueous suspension by the addition of water typically provide the active ingredient (e.g., a compound and/or conjugate of the present disclosure) in admixture with a dispersing or wetting agent, suspending agent and one or more preservatives. Suitable dispersing or wetting agents and suspending agents are exemplified by those already mentioned above. One or more additional excipients, for example sweetening, flavouring and/or colouring agents, can also be present.

Pharmaceutical compositions can also be in the form of oil-in-water emulsions. The oil phase can be a vegetable oil, for example olive oil or peanut oil, or a mineral oil, for example liquid paraffin, or mixtures of such oils. Suitable emulsifying agents for inclusion in oil-in-water emulsions include, for example, naturally-occurring gums (e.g., gum acacia or gum tragacanth), naturally-occurring phosphatides (e.g., soybean, lecithin), or esters or partial esters derived from fatty acids and hexitol anhydrides (e.g., sorbitan monooleate) or condensation products of such partial esters with ethylene oxide (e.g., polyoxyethylene sorbitan monooleate). The emulsions can also optionally contain sweetening and/or flavoring agents.

A pharmaceutical composition of this disclosure can be in the form of a sterile injectable aqueous or oleaginous solution or suspension. Such suspensions can be formulated using suitable dispersing or wetting agents and suspending agents such as those described above. The sterile injectable solution or suspension can comprise the active ingredient (e.g., a compound and/or conjugate of the present disclosure) in a non-toxic parentally acceptable carrier or diluent. Acceptable carriers and diluents that can be employed include, for example, 1,3-butanediol, water, Ringer's solution or isotonic sodium chloride solution. In addition, sterile, fixed oils can be employed as carriers. For this purpose, various bland fixed oils can be employed including synthetic mono- or diglycerides. In addition, fatty acids such as oleic acid find use in the preparation of injectables. Adjuvants such as local anaesthetics, preservatives and/or buffering agents can also be included in the injectable solution or suspension.

Pharmaceutical compositions can also be formulated as suppositories for rectal administration. These compositions can be prepared by mixing the active ingredient (e.g., a compound and/or conjugate of the present disclosure) with a suitable non-irritating excipient which is solid at ordinary temperatures but liquid at physiological temperature and will therefore melt in the rectum to release the drug. Such materials include cocoa butter and polyethylene glycols.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, PA (2000).

### Pharmaceutical Kits

In certain embodiments, a pharmaceutical composition comprising a compound of any one of Formulae (I)-(IV) or a conjugate of Formula (X) can be provided as part of a pharmaceutical kit or pack. Individual components of the kit can typically be packaged in separate containers. Suitable containers include, for example, bottles, blister packs, intravenous solution bags, vials, and the like, depending on the formulation of the pharmaceutical composition. In certain embodiments, the container can be in a form allowing for administration to a subject, for example, an inhaler, syringe, pipette, eye dropper, pre-soaked gauze or pad, or other such like apparatus, from which the contents can be administered to the subject.

The kit can further comprise a label or package insert on or associated with the container(s). The term “package insert” is used to refer to instructions customarily included in commercial packages of therapeutic products that contain information about the indications, usage, dosage, administration, contraindications and/or warnings concerning the use of such therapeutic products. The label or package insert can further include a notice in the form prescribed by a governmental agency regulating the manufacture, use or sale of pharmaceuticals or biological products, which notice reflects approval by the agency of manufacture, for use or sale for human or animal administration. The label or package insert typically indicates that the compound or conjugate is for use to treat the condition of choice, for example, cancer.

If appropriate, one or more components of the kit can be lyophilized or provided in a dry form, such as a powder or granules, and the kit can additionally contain a suitable solvent for reconstitution of the lyophilized or dried component(s).

### Methods of Use

Certain embodiments of the present disclosure relate to the therapeutic use of the purine-derived compounds described herein, e.g., those having a structure according to any one of Formulae (I)-(IV) and conjugates comprising these compounds, e.g., conjugates of Formula (X). Some embodiments of this disclosure relate to the use of a compounds according to any one of Formulae (I)-(IV) and/or a conjugate of Formula (X) as therapeutic agents.

The TLR7-agonizing compounds of the present disclosure (e.g., those of according to any one of Formulae (I)-(IV)) can exhibit cytotoxic activity against cancer cells, and compounds of any one of Formulae (I)-(IV) and conjugates comprising these compounds, i.e., conjugates of Formula (X), are thus useful for inhibiting abnormal cancer cell or tumor cell growth, inhibiting cancer cell or tumor cell proliferation, and/or treating cancer in a patient. In certain embodiments, compounds of any one of Formulae (I)-(IV) and conjugates of Formula (X) can be used to treat cancer in a subject in need thereof. Some embodiments of the present disclosure thus relate to the use of compounds of any one of Formulae (I)-(IV) and conjugates of general Formula (X) as anti-cancer agents.

Certain embodiments of the present disclosure relate to methods of inhibiting the proliferation of cancer or tumor cells comprising contacting the cells with a compound of any one of Formulae (I)-(IV) or a conjugate of Formula (X). Some embodiments relate to a method of killing cancer or tumor cells comprising contacting the cells with a compound of any one of Formulae (I)-(IV) or a conjugate of Formula (X).

Various embodiments of this disclosure relate to methods of agonizing a TLR7 using a compound of any one of Formulae (I)-(IV) or a conjugate of Formula (X) as described herein. In some embodiments, disclosed herein are methods of agonizing a TLR7 in vitro comprising contacting a cell that expresses TLR7 with a compound of any one of Formulae (I)-(IV) or a conjugate of Formula (X). In other embodiments, disclosed herein are methods of agonizing a TLR7 in a subject comprising administering to the subject a compound of any one of Formulae (I)-(IV), a conjugate of Formula (X), or a pharmaceutical composition comprising such compound or conjugate. Such method can further comprise contacting a cell expressing TLR7 in the subject with the compound of any one of Formulae (I)-(IV) or the conjugate of Formula (X).

Agonizing TLR7 on a cell in vitro or in vivo with a compound or conjugate of the present disclosure, e.g., a compound of any one of Formulae (I)-(IV) or the conjugate of Formula (X), can elicit an immune response by that cell or the subject. In some embodiments, disclosed herein are methods of eliciting an immune response in vitro, such method comprising contacting a cell with a compound of any one of Formulae (I)-(IV) or a conjugate of Formula (X). The cell can be a mammalian cell. The mammalian cell can be an immune cell, such as a lymphocyte (e.g., T cell) or a phagocyte (e.g., neutrophil, macrophage, dendritic cell, eosinophil, or monocyte). In yet other embodiments, disclosed herein are methods of eliciting an immune response in a subject in need thereof, comprising administering to the subject a compound of any one of Formulae (I)-(IV) or a conjugate of Formula (X). Such method can further comprise agonizing a TLR7 in the subject by the administered compound of any one of Formulae (I)-(IV) or the conjugate of Formula (X), thereby eliciting the immune response in the subject. In various embodiments, disclosed herein is a method for eliciting a local immune response in a subject, such method comprises administering a conjugate of Formula (X) herein, which comprises one or more compounds of any one of Formulae (I)-(IV), to the subject in need. Following administration (e.g., parenteral administration) of the conjugate of Formula (X) to the subject, such method can further comprise accumulation of the conjugate at a target site (e.g., a target organ or tissue) in the subject, and eliciting a local immune response at the target site. As used herein, the term “local immune response,” which stands in contrast to a systemic immune response, generally refers to an immune response that occurs in a subject at a specific location, e.g., organ, or within a certain tissue type present at one or more locations. As an example, a local anti-tumor immune response is generally understood herein to refer to an immune response that occurs at a tumor site comprising tumor tissue, or at multiple tumor sites, e.g., in the case of metastases. A local immune response can be measured or detected by a change in one or more physiological parameters, such as a local concentration of biomarkers, such as production/secretion of cytokines, small molecules, co-stimulatory molecules, and/or factors involved in inflammation cascades or regulation, and/or a change in immune cell populations.

In some embodiments, a method of agonizing TLR7 using a compound of any one of Formulae (I)-(IV) or a conjugate of Formula (X) can further comprise activating a TLR7 signaling pathway. Such activation can cause a measurable change (i.e., an increase or decrease) in the expression of one or more intermediates in the TLR7 signaling pathway. Intermediates of the TLR7 signaling pathway include, e.g., MyD88, IRAK4, IRAK1, IRAK2, TRAF6, TAK1, IKK, NF-κB, FADD, Caspase 8, Caspase 3, and/or IRF7. See, e.g., Chi H et al. Front Pharmacol. 2017; 8:304. Examples of inflammatory cytokines that can be modulated in response to incubation or contact with a compound and/or a conjugate of the present disclosure include, but are not limited to, tumor necrosis factor (TNF; also known as TNFα or cachectin), interleukin (IL)-1α, IL1β, IL2; IL5, IL6, IL8, IL15, IL18, interferon γ (IFN-γ); platelet-activating factor (PAF), thromboxane; soluble adhesion molecules; vasoactive neuropeptides; phospholipase A2; plasminogen activator inhibitor (PAI-1); free radical generation; neopterin; CD14; prostacyclin; neutrophil elastase; protein kinase; monocyte chemotactic proteins 1 and 2 (MCP-1, MCP-2); macrophage migration inhibitory factor (MIF), and high mobility group box protein 1 (HMGB-1).

Methods of assessing one or more of these physiological parameters are known in the art. For example, a cytokine can be directly detected, e.g., by ELISA. Other suitable methods include liquid chromatography and tandem mass spectrometry. Quantitative changes of the biological molecules (e.g., cytokines) can be measured in a biological sample such as organ, tissue, urine, or plasma. Detection of the biological molecules can be performed directly on a sample taken from a subject, or the sample can be treated between sample collection and analysis.

Some embodiments of this disclosure relate to methods of treating a subject having a cancer by administering to the subject a compound of Formula (I) or a conjugate of Formula (X), or a pharmaceutical composition comprising such compound or conjugate. In this context, treatment with a compound of any one of Formulae (I)-(IV) or a conjugate of Formula (X) can result in one or more of a reduction in the size of a tumor, the slowing or prevention of an increase in the size of a tumor, an increase in the disease-free survival time between the disappearance or removal of a tumor and its reappearance, prevention of a subsequent occurrence of a tumor (for example, metastasis), an increase in the time to progression, reduction of one or more adverse symptom(s) associated with a tumor, and/or an increase in the overall survival time of a subject having the cancer.

Certain embodiments relate to the use of a compound of any one of Formulae (I)-(IV) or a conjugate of Formula (X) in a method of inhibiting tumor growth in a subject. Some embodiments relate to the use of a compound of any one of Formulae (I)-(IV) or a conjugate of Formula (X) in a method of inhibiting proliferation of and/or killing cancer cells in vitro. Some embodiments relate to the use of a compound of any one of Formulae (I)-(IV) or a conjugate of Formula (X) in a method of inhibiting proliferation of and/or killing cancer cells in vivo in a subject having a cancer.

Examples of cancers which can be treated in certain embodiments of this disclosure using a compound or conjugate disclosed herein include hematologic neoplasms, including leukemias, myelomas and lymphomas; carcinomas, including adenocarcinomas and squamous cell carcinomas; melanomas and sarcomas. Carcinomas and sarcomas are also frequently referred to as “solid tumors.” Examples of commonly occurring solid tumors that can be treated in certain embodiments include, but are not limited to, brain cancer, breast cancer, cervical cancer, colon cancer, head and neck cancer, kidney cancer, lung cancer, ovarian cancer, pancreatic cancer, prostate cancer, stomach cancer, uterine cancer, non-small cell lung cancer (NSCLC) and colorectal cancer. Various forms of lymphoma also can result in the formation of a solid tumor and, therefore, can also be considered to be solid tumors in certain situations.

In other embodiments, a compound of any one of Formulae (I)-(IV), or a conjugate of Formula (X) herein, can be used as a vaccine adjuvant. In yet other embodiments, a compound of any one of Formulae (I)-(IV), or a conjugate of Formula (X) herein, can be used in a method of treating a viral infection in a subject in need thereof. Such method can comprise administering an effective amount of the compound or conjugate to the subject, thereby treating the viral infection in the subject.

### Certain Embodiments of the Disclosure

In certain embodiments, the present disclosure relates to any one or more of embodiments 1-140.

Embodiment 1. A compound of Formula (I):

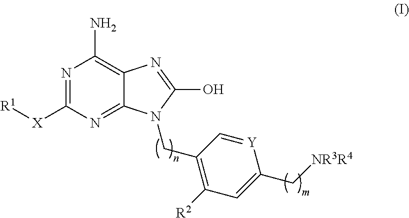

a tautomer, a protected version, and/or a pharmaceutically acceptable salt thereof, wherein:
X is O or NH;
Y is N or CH;
R¹ is optionally substituted C₂-C₆ alkyl or branched and optionally substituted C₃-C₈ hydroxyalkyl;
R² is H, halogen, optionally substituted C₁-C₆ alkyl, or optionally substituted C₁-C₆ alkoxy;
R³ and R⁴ are independently H, Q-R⁵, or R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

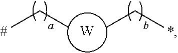

wherein * is the point of attachment to R⁵ and #is the point of attachment to N;
R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R⁶ and R⁷ are independently H, optionally substituted C₁-C₆ alkyl, optionally substituted C₁-C₆ alkoxycarbonyl, or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
n is an integer from 1 to 4;
m is an integer from 0 to 4; and
a and b are each independently integers from 0 to 4,
with the proviso that when R² is H, then:
   (i) R¹ is branched and optionally substituted C₃-C₈ hydroxyalkyl;
   (ii) X is NH and R¹ is optionally substituted C₅-C₆ alkyl; or
   (iii) m is 0 or 1 and R³ and R⁴ together with the N form an unsubstituted piperazinyl ring or an optionally substituted C₆-C₁₀-heterospirocycloalkyl ring.
      The compound of embodiment 1, wherein R² is halogen, optionally substituted C₁-C₆ alkyl, or optionally substituted C₁-C₆ alkoxy.

Embodiment 2. The compound of embodiment 1, wherein R² is halogen, optionally substituted C₁-C₆ alkyl, or optionally substituted C₁-C₆ alkoxy.

Embodiment 3. The compound of any one of embodiments 1-2, wherein R² is halogen or optionally substituted C₁-C₆ alkoxy.

Embodiment 4. The compound of any one of embodiments 1-3, wherein R² is halogen or unsubstituted C₁-C₆ alkoxy.

Embodiment 5. The compound of any one of embodiments 1-4, wherein R² is fluorine or methoxy.

Embodiment 6. The compound of any one of embodiments 1-5, wherein X is O.

Embodiment 7. The compound of any one of embodiments 1-5, wherein X is NH.

Embodiment 8. The compound of any one of embodiments 1-7, wherein R¹ is optionally substituted C₂-C₆ alkyl.

Embodiment 9. The compound of any one of embodiments 1-7, wherein R¹ is branched and optionally substituted C₃-C₈ hydroxyalkyl.

Embodiment 10. The compound of any one of embodiments 1-9, wherein R³ and R⁴ are independently H, or Q-R⁵.

Embodiment 11. The compound of any one of embodiments 1-10, wherein R³ and R⁴ are both H, or R³ and R⁴ are both Q-R⁵.

Embodiment 12. The compound of any one of embodiments 1-10, wherein R³ is H and R⁴ is Q-R⁵.

Embodiment 13. The compound of embodiment 12, wherein Q is a bond and R⁵ is optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 14. The compound of embodiment 12, wherein Q is optionally substituted C₁-C₆-alkyl and R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 15. The compound of embodiment 12, wherein Q is optionally substituted C₂-C₆-alkenyl and R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 16. The compound of any one of embodiments 1-12, wherein Q is:

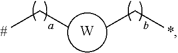

and wherein:
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl; and
a and b are each independently integers from 0 to 4.

Embodiment 17. The compound of embodiment 16, wherein Q is selected from the group consisting of:

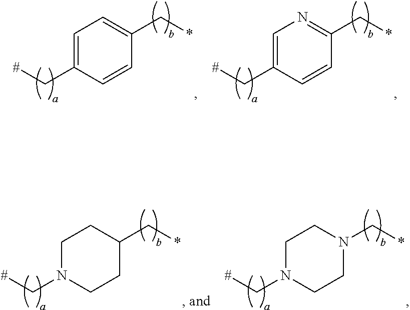

wherein a and b are each independently 1, 2, or 3.

Embodiment 18. The compound of any one of embodiments 1-12 or 14-17, wherein R⁵ is NR⁶R⁷ and R⁶ and R⁷ are both H.

Embodiment 19. The compound of any one of embodiments 1-12 or 14-17, wherein R⁵ is NR⁶R⁷, R⁶ is H and R⁷ is optionally substituted C₁-C₆ alkyl.

Embodiment 20. The compound of any one of embodiments 1-12 or 14-17, wherein R⁵ is NR⁶R⁷ and R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring.

Embodiment 21. The compound of any one of embodiments 1-9, wherein R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring.

Embodiment 22. The compound of any one of embodiments 1-21, wherein n is 1.

Embodiment 23. The compound of any one of embodiments 1-22, wherein m is an integer from 0 to 3 or from 1 to 3.

Embodiment 24. The compound of embodiment 1, wherein R² is H and R¹ is branched and optionally substituted C₃-C₈ hydroxyalkyl.

Embodiment 25. The compound of embodiment 24, wherein R¹ is branched and unsubstituted C₃-C₈ hydroxyalkyl.

Embodiment 26. The compound of embodiment 1, wherein R² is H, X is NH, and R¹ is optionally substituted C₅-C₆ alkyl.

Embodiment 27. The compound of embodiment 26, wherein R¹ is unsubstituted C₅-C₆ alkyl.

Embodiment 28. The compound of embodiment 1, wherein R² is H, m is 0 or 1, and R³ and R⁴ together with the N form an unsubstituted piperazinyl ring or an optionally substituted C₆-C₁₀-heterospirocycloalkyl ring.

Embodiment 29. The compound of embodiment 28, wherein R³ and R⁴ together with the N form an unsubstituted piperazinyl ring.

Embodiment 30. The compound of embodiment 28, wherein R³ and R⁴ together with the N form an optionally substituted C₆-C₁₀-heterospirocycloalkyl ring.

Embodiment 31. The compound of any one of embodiments 28-30, wherein m is 0.

Embodiment 32. The compound of any one of embodiments 28-30, wherein m is 1.

Embodiment 33. The compound of any one of embodiments 1-32, wherein each alkyl, alkenyl, cycloalkyl, spirocycloalkyl, heterocycloalkyl, heterospirocycloalkyl, aryl and heteroaryl group is optionally substituted —NH₂, —CO₂H, —OH, carbonyl, halogen, C₁-C₄ alkyl, C₁-C₄ hydroxyalkyl, C₁-C₄ aminoalkyl, C₁-C₄ carboxyalkyl, (C₀-C₄ alkyl)-cycloalkyl, (C₀-C₄ alkyl)-heterocycloalkyl, (C₀-C₄ alkyl)-spirocycloalkyl, (C₀-C₄ alkyl)-heterospirocycloalkyl, (C₀-C₄ alkyl)-aryl, and (C₀-C₄ alkyl)-heteroaryl, and wherein each of the alkyl, hydroxyalkyl, C₁-C₄ aminoalkyl, C₁-C₄ carboxyalkyl, cycloalkyl, heterocycloalkyl, spirocycloalkyl, heterospirocycloalkyl, aryl or heteroaryl group can itself be substituted with one or more of: —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl, or unsubstituted C₁-C₄ carboxyalkyl.

Embodiment 34. The compound of embodiment 33, wherein each alkyl, alkenyl, cycloalkyl, spirocycloalkyl, heterocycloalkyl, heterospirocycloalkyl, aryl and heteroaryl group is optionally substituted with one or more substituents selected from the group consisting of —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl and unsubstituted C₁-C₄ carboxyalkyl.

Embodiment 35. The compound of embodiment 1, wherein the compound is selected from any one of compounds 100-195 listed in TABLE 1.

Embodiment 36. The compound of embodiment 1, having the structure of Formula (II):

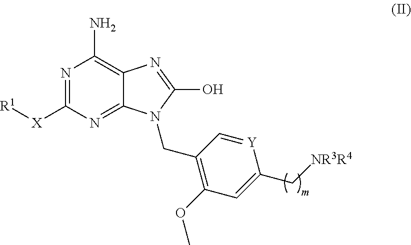

wherein:
X is O or NH;
Y is N or CH;
R¹ is optionally substituted C₂-C₆ alkyl or branched and optionally substituted C₃-C₈ hydroxyalkyl;
R³ and R⁴ are independently H, Q-R⁵, or R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

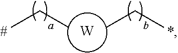

wherein * is the point of attachment to R⁵ and #is the point of attachment to N;
R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R⁶ and R⁷ are independently H, optionally substituted C₁-C₆ alkyl, optionally substituted C₁-C₆ alkoxycarbonyl, or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
m is an integer from 0 to 4; and
a and b are independently integers from 0 to 4.

Embodiment 37. The compound of embodiment 36, wherein X is O and R¹ is optionally substituted C₂-C₆ alkyl.

Embodiment 38. The compound of any one of embodiments 36-37, wherein R¹ is unsubstituted C₂-C₆ alkyl.

Embodiment 39. The compound of any one of embodiments 36-38, wherein Y is CH.

Embodiment 40. The compound of any one of embodiments 36-39, wherein m is 0 or 1, 1 or 2, or 1 or 3.

Embodiment 41. The compound of any one of embodiments 36-40, wherein R³ and R⁴ are both H.

Embodiment 42. The compound of any one of embodiments 36-40, wherein R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring.

Embodiment 43. The compound of any one of embodiments 36-40, wherein R³ and R⁴ are either both H or Q-R⁵, or R³ is H and R⁴ is Q-R⁵.

Embodiment 44. The compound of embodiment 43, wherein Q is a bond and R⁵ is optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 45. The compound of embodiment 43, wherein Q is optionally substituted C₁-C₆-alkyl and R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 46. The compound of embodiment 43, wherein Q is optionally substituted C₂-C₆-alkenyl and R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 47. The compound of any one of embodiments 36-43 or 45-46, wherein R⁵ is NR⁶R⁷ and R⁶ and R⁷ are both H.

Embodiment 48. The compound of any one of embodiments 36-43 or 45-46, wherein R⁵ is NR⁶R⁷, R⁶ is H and R⁷ is optionally substituted C₁-C₆ alkyl.

Embodiment 49. The compound of any one of embodiments 36-43 or 45-46, wherein R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring.

Embodiment 50. The compound of any one of embodiments 36-49, wherein m is 1 or 2, or 1 or 3.

Embodiment 51. The compound of embodiment 36, wherein the compound is selected from SUBTABLE 1A.

Embodiment 52. The compound of any one of embodiments 1-9, 21, or 42, wherein R³ and R⁴ together with the N form a ring selected from the group consisting of:

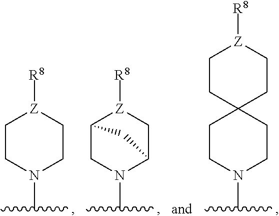

wherein:
Z is N or CH; and
R⁸ is H, NH₂, optionally substituted C₁-C₆-alkyl, optionally substituted C₁-C₆-heteroalkyl, optionally substituted C₁-C₆-aminoalkyl, or optionally substituted C₃-C₆-heterocycloalkyl.

Embodiment 53. The compound of embodiment 52, wherein R³ and R⁴ together with the N form a ring selected from the group consisting of:

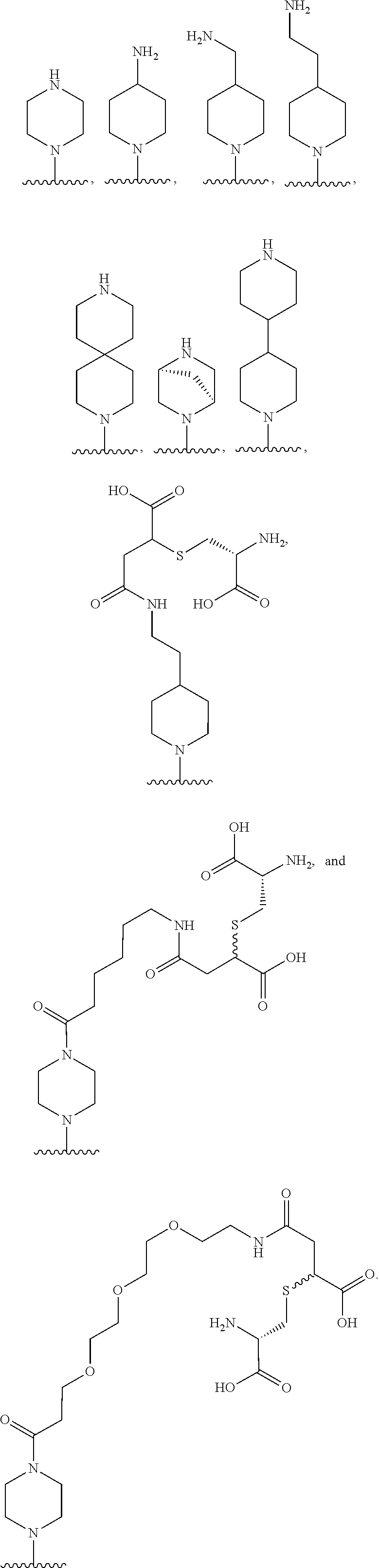

Embodiment 54. The compound of any one of embodiments 1-9 or 21, wherein the compound is selected from SUBTABLE 1B.

Embodiment 55. The compound of any one of embodiments 1-11, wherein R³ and R⁴ are both Q-R⁵.

Embodiment 56. The compound of embodiment 55, wherein the compound is:

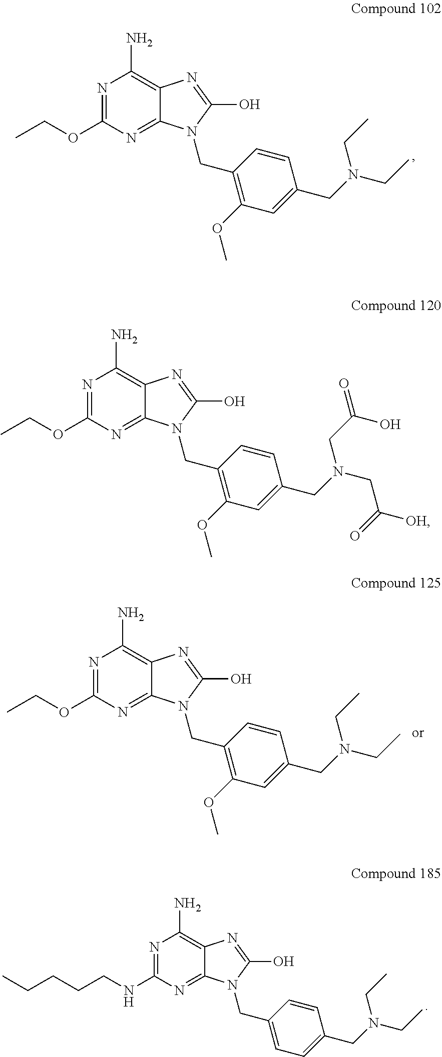

Embodiment 57. The compound of embodiment 1-11, wherein R³ and R⁴ are both H.

Embodiment 58. The compound of embodiment 57, wherein the compound is:

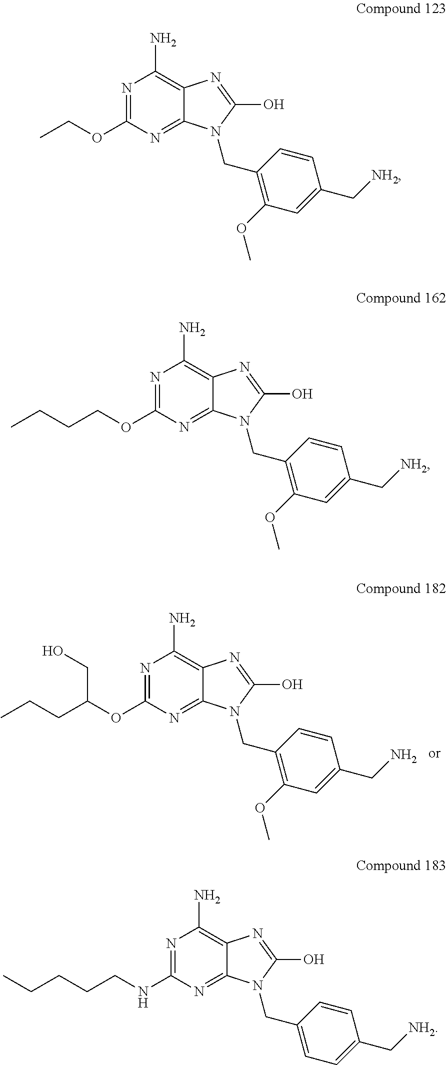 [SMILES: CCOc1nc(N)c2nc(O)n(Cc3ccc(CN)cc3OC)c2n1 ; CCCCOc1nc(N)c2nc(O)n(Cc3ccc(CN)cc3OC)c2n1 ; CCCC(CO)Oc1nc(N)c2nc(O)n(Cc3ccc(CN)cc3OC)c2n1 ; "or" ; CCCCCNc1nc(N)c2nc(O)n(Cc3ccc(CN)cc3)c2n1]

Embodiment 59. The compound of embodiment 13, wherein the compound is selected from SUBTABLE 1C.

Embodiment 60. The compound of embodiment 15 or embodiment 46, wherein the compound is:

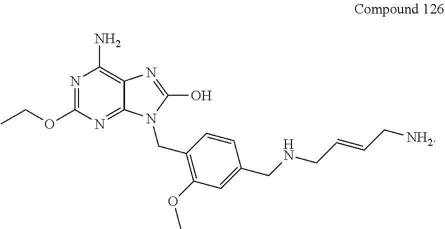 [SMILES: CCOc1nc(N)c2nc(O)n(Cc3ccc(CNC/C=C/CN)cc3OC)c2n1]

Embodiment 61. The compound of any one of embodiments 1-10 or 14, wherein the compound is selected from SUBTABLE 1D.

Embodiment 62. The compound of any one of embodiments 1-10 or 16-17, wherein R⁶ is H and R⁷ is H, optionally substituted C₁-C₆ alkyl or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring.

Embodiment 63. The compound of embodiment 62, wherein the compound is selected from SUBTABLE 1E.

Embodiment 64. The compound of any one of embodiments 1-9 or 36, wherein the compound is:

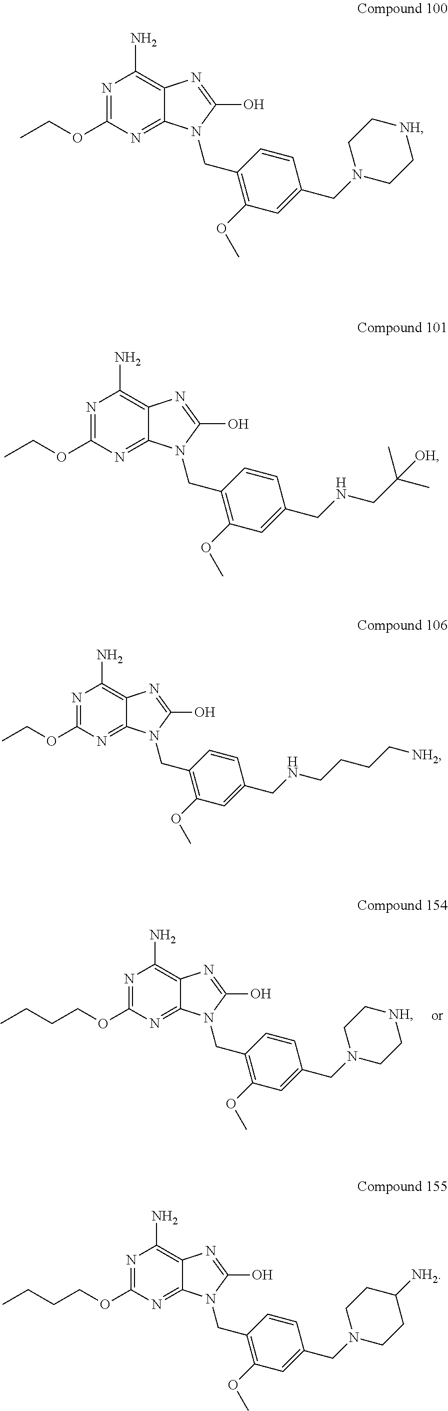

Embodiment 65. The compound of embodiments 1 or 24-25, wherein the compound is:

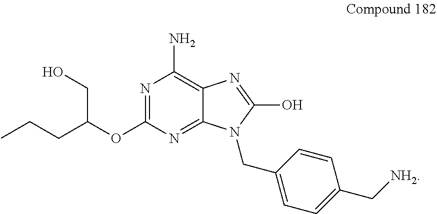 [SMILES: CCCC(CO)Oc1nc(N)c2nc(O)n(Cc3ccc(CN)cc3)c2n1]

Embodiment 66. A compound of Formula (III):

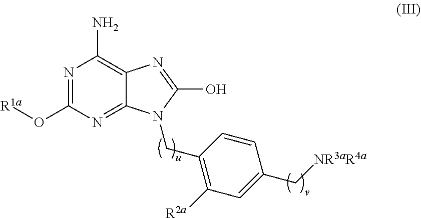

a tautomer, a protected version, and/or a pharmaceutically acceptable salt thereof, wherein:
R^{1a} is optionally substituted C₂-C₆ alkyl or branched and optionally substituted C₃-C₈ hydroxyalkyl;
R^{2a} is halogen, optionally substituted C₁-C₆ alkyl, or optionally substituted C₁-C₆ alkoxy;
R^{3a} and R^{4a} are independently H, Q-R^{5a}, or R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

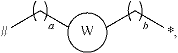

wherein * is the point of attachment to R⁵ and #is the point of attachment to N;
R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R^{6a} and R^{7a} are independently H, optionally substituted C₁-C₆ alkyl, optionally substituted C₁-C₆ alkoxycarbonyl, or R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₃-C₆-heterocycloalkyl, optionally substituted C₅-C₇-heterobicycloalkyl, optionally substituted C₆-C₁₀-heterospirocycloalkyl, optionally substituted aryl, or optionally substituted heteroaryl;
u is an integer from 1 to 3;
v is an integer from 0 to 4; and
a and b are independently integers from 0 to 4.

Embodiment 67. The compound of embodiment 66, wherein R^{1a} is optionally substituted C₂-C₆ alkyl.

Embodiment 68. The compound of any one of embodiments 66-67, wherein R^{1a} is unsubstituted C₂-C₆ alkyl.

Embodiment 69. The compound of any one of embodiments 66-68, wherein R^{2a} is halogen or optionally substituted C₁-C₆ alkoxy.

Embodiment 70. The compound of any one of embodiments 66-69, wherein R^{2a} is halogen or unsubstituted C₁-C₆ alkoxy.

Embodiment 71. The compound of any one of embodiments 66-70, wherein R^{2a} is fluorine or methoxy.

Embodiment 72. The compound of any one of embodiments 66-71, wherein R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring.

Embodiment 73. The compound of any one of embodiments 66-71, wherein R^{3a} and R^{4a} are both H.

Embodiment 74. The compound of any one of embodiments 66-71, wherein R^{3a} and R^{4a} are independently H, or Q-R⁵.

Embodiment 75. The compound of embodiment 74, wherein R^{3a} is H and R^{4a} is Q-R^{5a}.

Embodiment 76. The compound of any one of embodiments 74-75, wherein Q is a bond and R^{5a} is optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 77. The compound of any one of embodiments 74-75, wherein Q is optionally substituted C₁-C₆-alkyl and R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 78. The compound of any one of embodiments 74-75, wherein Q is optionally substituted C₂-C₆-alkenyl and R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl.

Embodiment 79. The compound of any one of embodiments 66-75, wherein Q is:

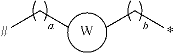

and wherein:
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl; and
a and b are independently integers from 0 to 4.

Embodiment 80. The compound of embodiment 79, wherein Q is selected from the group consisting of:

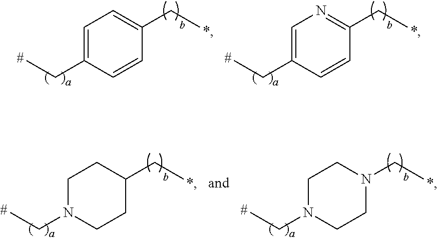

wherein a and b are independently 1, 2, or 3.

Embodiment 81. The compound of any one of embodiments 66-75 or 77-80, wherein R^{6a} and R^{7a} are both H.

Embodiment 82. The compound of any one of embodiments 66-75 or 77-80, wherein R^{6a} is H and R^{7a} is optionally substituted C₁-C₆ alkyl.

Embodiment 83. The compound of any one of embodiments 66-75 or 77-80, wherein R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring.

Embodiment 84. The compound of any one of embodiments 66-83, wherein u is 1.

Embodiment 85. The compound of any one of embodiments 66-84, wherein v is an integer from 0 to 4, from 1 to 4, 1 to 3, or from 1 to 2.

Embodiment 86. The compound of any one of embodiments 66-72, wherein R^{3a} and R^{4a} together with the N form a ring selected from the group consisting of:

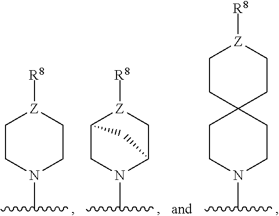

wherein:
Z is N or CH; and
R^{8a} is H, NH₂, optionally substituted C₁-C₆-alkyl, optionally substituted C₁-C₆-heteroalkyl, optionally substituted C₁-C₆-aminoalkyl, or optionally substituted C₃-C₆-heterocycloalkyl.

Embodiment 87. The compound of embodiment 86, wherein R^{3a} and R^{4a} together with the N form a ring selected from the group consisting of:

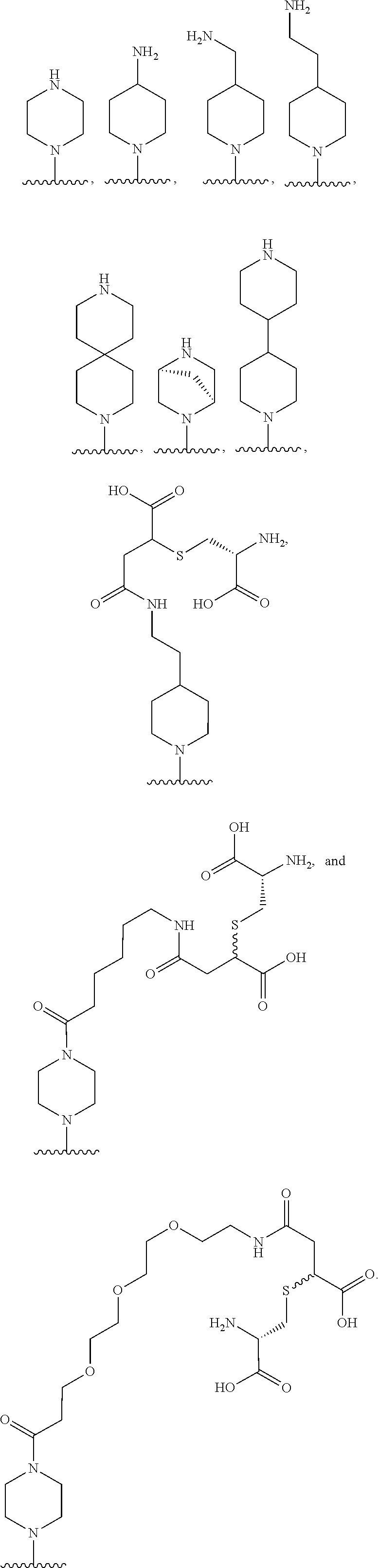

Embodiment 88. The compound of embodiment 66, having the structure of Formula (IV):

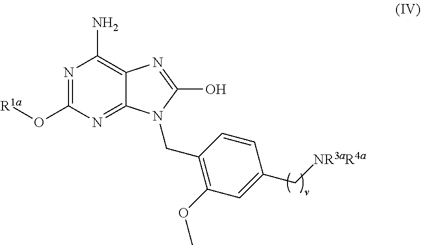

wherein:
R^{1a} is unsubstituted C₂-C₆ alkyl or branched and unsubstituted C₃-C₈ hydroxyalkyl;
R^{3a} and R^{4a} are independently H, Q-R^{5a}, or R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or

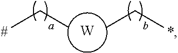

wherein * is the point of attachment to R⁵ and #is the point of attachment to N;
R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R^{6a} and R^{7a} are independently H, optionally substituted C₁-C₆ alkyl, optionally substituted C₁-C₆ alkoxycarbonyl, or R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
v is an integer from 0 to 4; and
a and b are independently integers from 0 to 4.

Embodiment 89. The compound of embodiment 66 or embodiment 88, wherein each alkyl, alkenyl, cycloalkyl, spirocycloalkyl, heterocycloalkyl, heterospirocycloalkyl, aryl and heteroaryl group is optionally substituted —NH₂, —CO₂H, —OH, carbonyl, halogen, C₁-C₄ alkyl, C₁-C₄ hydroxyalkyl, C₁-C₄ aminoalkyl, C₁-C₄ carboxyalkyl, (C₀-C₄ alkyl)-cycloalkyl, (C₀-C₄ alkyl)-heterocycloalkyl, (C₀-C₄ alkyl)-spirocycloalkyl, (C₀-C₄ alkyl)-heterospirocycloalkyl, (C₀-C₄ alkyl)-aryl, and (C₀-C₄ alkyl)-heteroaryl, and wherein each of the alkyl, hydroxyalkyl, C₁-C₄ aminoalkyl, C₁-C₄ carboxyalkyl, cycloalkyl, heterocycloalkyl, spirocycloalkyl, heterospirocycloalkyl, aryl or heteroaryl group can itself be substituted with one or more of: —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl, or unsubstituted C₁-C₄ carboxyalkyl.

Embodiment 90. The compound of embodiment 89, wherein each alkyl, alkenyl, cycloalkyl, spirocycloalkyl, heterocycloalkyl, heterospirocycloalkyl, aryl and heteroaryl group is optionally substituted with one or more substituents selected from the group consisting of —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl and unsubstituted C₁-C₄ carboxyalkyl.

Embodiment 91. The compound of embodiment 66 or embodiment 88, wherein the compound is selected from SUBTABLE 1A.

Embodiment 92. The compound of embodiment 66 or embodiment 88, wherein the compound is:

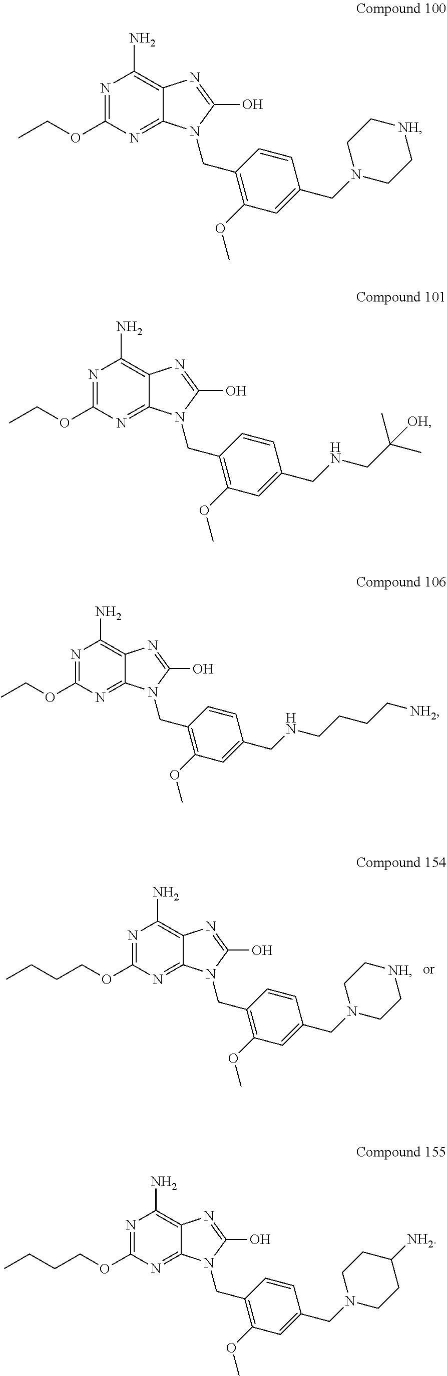

Embodiment 93. The compound of any one of embodiments 1-92, wherein the compound has an EC₅₀ value for agonizing human or murine TLR7 of <500 nM, <250 nM, or <100 nM as determined in a reporter gene assays, and/or an EC₅₀ value for inducing IL6 production in a human or murine immune cell population of <5 μM, <1 μM, <500 nM, or <100 nM, as determined in an immune cell assay.

Embodiment 94. A pharmaceutical composition comprising a compound of any one of embodiments 1-92, and a pharmaceutically acceptable carrier or diluent.

Embodiment 95. A method of agonizing TLR7, the method comprising contacting a cell that expresses TLR7 with a compound of any one of embodiments 1-92, thereby agonizing TLR7.

Embodiment 96. A method of inducing release of a cytokine from a cell, the method comprising contacting the cell with a compound of any one of embodiments 1-92, thereby inducing release of the cytokine from the cell.

Embodiment 97. A method of stimulating an immune response in a subject in need thereof, the method comprising administering to the subject an effective amount of a compound of any one of embodiments 1-92.

Embodiment 98. The method of embodiment 97, wherein the compound agonizes TLR7 in the subject, thereby stimulating the immune response in the subject.

Embodiment 99. A method of inhibiting the proliferation of cancer cells, the method comprising contacting a cell population comprising the cancer cells with an effective amount of the compound of any one of embodiments 1-92.

Embodiment 100. A method of killing cancer cells, the method comprising contacting a cell population comprising the cancer cells with an effective amount of the compound of any one of embodiments 1-92.

Embodiment 101. The method of any one of embodiments 99-100, wherein the cell population comprises immune cells.

Embodiment 102. A method of treating a cancer in a subject in need thereof, the method comprising administering to the subject an effective amount of the compound of any one of embodiments 1-92.

Embodiment 103. The method of embodiment 102, wherein the compound agonizes TLR7 in the subject, thereby treating the cancer in the subject.

Embodiment 104. A compound of any one of embodiments 1-92 for use in therapy.

Embodiment 105. A compound of any one of embodiments 1-92 for use in the treatment of cancer.

Embodiment 106. Use of a compound of any one of embodiments 1-92 in the manufacture of a medicament for the treatment of cancer.

Embodiment 107. A compound-linker construct having Formula (A):

L-(C)ₚ (A)

wherein:
L is a linker;
C is a compound of any one of embodiments 1-92; and
p is an integer from 1 to 5.

Embodiment 108. The compound-linker construct of embodiment 107, wherein L is a cleavable linker.

Embodiment 109. The compound-linker construct of embodiment 107, wherein L is a non-cleavable linker.

Embodiment 110. The compound-linker construct of any one of embodiments 107-108, wherein L comprises a dipeptide, tripeptide, tetrapeptide, or a combination thereof.

Embodiment 111. The compound-linker construct of any one of embodiments 107-110, wherein L comprises a polyethylene glycol (PEG) moiety.

Embodiment 112. The compound-linker construct of any one of embodiments 107-108, wherein L is a protease cleavable linker.

Embodiment 113. The compound-linker construct of any one of embodiments 107-112, wherein C is the compound of embodiment 36.

Embodiment 114. The compound-linker construct of any one of embodiments 107-112, wherein C is the compound of embodiment 66.

Embodiment 115. The compound-linker construct of embodiment 107, wherein the compound-linker is selected from TABLE 2B.

Embodiment 116. The compound-linker construct of any one of embodiments 107-115, wherein p is 1, 2, or 3.

Embodiment 117. A conjugate having Formula (X):

T-[L-(C)ₚ]ᵣ (X)

wherein:
T is a targeting moiety;
L is a linker;
C is a compound of any one of embodiments 1-92;
p is an integer from 1 to 5; and
r has a value from about 1 to about 8.

Embodiment 118. The conjugate of embodiment 117, wherein L-(C), is the compound-linker construct of any one of embodiments 107-116.

Embodiment 119. The conjugate of any one of embodiments 117-118, wherein r is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

Embodiment 120. The conjugate of embodiment 117, wherein C is the compound of embodiment 36.

Embodiment 121. The conjugate of embodiment 117, wherein C is the compound of embodiment 66 or embodiment 88.

Embodiment 122. The conjugate of embodiment 117, wherein C is selected from SUBTABLE 1F.

Embodiment 123. The conjugate of any one of embodiments 117-122, wherein T binds to a tumor associated antigen (TAA).

Embodiment 124. The conjugate of any one of embodiments 117-123, wherein T is an antibody or antigen-binding antibody fragment.

Embodiment 125. The conjugate of embodiment 124, wherein the antibody is a bispecific or multispecific antibody.

Embodiment 126. The conjugate of any one of embodiments 124-125, wherein the antibody or antigen-binding antibody fragment binds to a TAA.

Embodiment 127. The conjugate of any one of embodiments 117-126, wherein the conjugate has an EC₅₀ value for inducing production of a cytokine in a human or murine immune cell population of <1 nM, <500 pM, or <100 pM, as determined in a reporter gene assay.

Embodiment 128. A pharmaceutical composition comprising the conjugate of any one of embodiments 117-126, and a pharmaceutically acceptable carrier or diluent.

Embodiment 129. A method of agonizing TLR7, the method comprising contacting a cell that expresses TLR7 with a conjugate of any one of embodiments 117-126, thereby agonizing TLR7.

Embodiment 130. A method of inducing release of a cytokine from a cell, the method comprising contacting the cell with a conjugate of any one of embodiments 117-126, thereby inducing release of the cytokine from the cell.

Embodiment 131. A method of stimulating an immune response in a subject in need thereof, the method comprising administering to the subject an effective amount of a conjugate of any one of embodiments 117-126.

Embodiment 132. The method of embodiment 132, wherein the conjugate agonizes TLR7 in the subject, thereby stimulating the immune response in the subject.

Embodiment 133. A method of inhibiting the proliferation of cancer cells, the method comprising contacting a cell population comprising the cancer cells with an effective amount of the conjugate of any one of embodiments 117-126.

Embodiment 134. A method of killing cancer cells, the method comprising contacting a cell population comprising the cancer cells with an effective amount of the conjugate of any one of embodiments 117-126.

Embodiment 135. The method of any one of embodiments 133-134, wherein the cell population comprises immune cells.

Embodiment 136. A method of treating a cancer in a subject in need thereof, the method comprising administering to the subject an effective amount of the conjugate of any one of embodiments 117-126.

Embodiment 137. The method of embodiment 136, wherein the conjugate agonizes TLR7 in the subject, thereby treating the cancer in the subject.

Embodiment 138. A conjugate of any one of embodiments 117-126 for use in therapy.

Embodiment 139. A conjugate of any one of embodiments 117-126 for use in the treatment of cancer.

Embodiment 140. Use of a conjugate of any one of embodiments 117-126 in the manufacture of a medicament for the treatment of cancer.

### EXAMPLES

The following Examples are provided for illustrative purposes and are not intended to limit the scope of the invention in any way.

The following Examples provide illustrative methods of making and using compounds of the present disclosure, e.g., a compound of any one of Formulae (I)-(IV). It is understood that one skilled in the art can be able to synthesize these compounds by similar methods or by combining other methods known in the art. Preparation of other compounds of Formulae (I)-(IV) not specifically illustrated herein could be achieved by one skilled in the art using the methods described herein or similar methods with appropriate starting components and modification of the parameters of the synthesis as needed. In general, starting components and materials can be obtained from commercial sources such as Sigma Aldrich (Merck KGaA), Alfa Aesar and Maybridge (Thermo Fisher Scientific Inc.), Matrix Scientific, Tokyo Chemical Industry Ltd. (TCI) and Fluorochem Ltd., and/or synthesized according to sources known to those skilled in the art (see, for example, March's Advanced Organic Chemistry: Reactions, Mechanisms, and Structure, 7th edition, John Wiley & Sons, Inc., 2013) or prepared as described herein.

The following abbreviations are used throughout the Examples: DCM=dichloromethane; DIPEA=N,N-diisopropylethylamine; DMA=dimethylacetamide; DMF=dimethylformamide; DMSO=dimethylsulphoxide; IL6=interleukin 6; LC/MS=Liquid Chromatography/Mass Spectrometry; LC/MSD=Liquid Chromatography/Mass Selective Detector; SEC=Size-exclusion chromatography; HIC=hydrophobic interaction chromatography; RP-UPLC=reverse-phase ultra performance liquid chromatography; HPLC=high-performance liquid chromatography; MT=maleimidotriethylene glycolate; PABC=p-aminobenzyloxycarbonyl; PBMC=peripheral blood mononuclear cell; PNP=p-nitrophenol; rt=room temperature; TCEP=tris(2-carboxyethyl)phosphine; TFA=trifluoroacetic acid; TNF-α=tumor necrosis factor α; VC=valine-citrulline; UHPLC=ultra high-performance liquid chromatograph.

### General Chemistry Procedures

### General Procedure 1: Conversion of Chloride to Amine

To the chloride compound in DMF (50-100 mg/mL) was added a primary or secondary amine (2-5 eq.) followed by DIPEA (3-5 eq.), and the solution was heated to 50-75° C. with stirring. Upon completion (typically 1-18 h), the reaction mixture was adjusted to pH 2 with 6 M HCl and purified by reverse-phase HPLC to provide the desired product after lyophilization.

### General Procedure 2: Removal of Boc Protecting Group

To a stirring solution of the Boc protected amine compound in dichloromethane (0.1 M) was added TFA (10% by volume). Upon completion (typically 1 h), the reaction mixture was concentrated in vacuo and co-evaporated with DCM several times to provide a crude solid or was purified by preparative HPLC to provide the desired product after lyophilization.

### General Procedure 3: Formation of Benzyl Chlorides

The alcohol compound was dissolved 10% SOCl₂/DCM (0.05-0.1 M). The solution was stirred at room temperature until completion (typically 12-24 h), then concentrated in vacuo. The residue was co-evaporated (2×10 mL dichloromethane) to give the crude chloride compound, which was typically used without further purification.

### General Procedure 4: MT-VC-PABC Compound-Linker Construct Synthesis

To the primary or secondary amine compound in dimethylformamide (0.05-0.1 M) was added the compound of Example 2.5 (MT-VC-PABC-PNP) (1-1.1 eq.) followed by DIPEA (3 eq.). Upon completion (typically 1-4 h), the reaction mixture was acidified with aqueous HCl (1 M) then purified by reverse-phase HPLC to provide the desired drug-linker after lyophilization.

### General Procedure 5: Compound Purification

Flash Chromatography: Crude reaction products were purified with Biotage® Snap Ultra columns (10, 25, 50, or 100 g) (Biotage, Charlotte, NC), and eluting with linear gradients of ethyl acetate/hexanes or methanol/dichloromethane on a Biotage® Isolera™ automated flash system (Biotage, Charlotte, NC). Alternatively, reverse-phase flash purification was conducted using Biotage® Snap Ultra C18 columns (12, 30, 60, or 120 g) and eluting with linear gradients of CH₃CN+0.1% TFA/H₂O+0.1% TFA. Purified compounds were isolated by either removal of organic solvents by rotavap or lyophilization of acetonitrile/water mixtures.

Preparative HPLC: Reverse-phase HPLC of crude compounds was performed using a Kinetex® 5-μm EVO C18 100 Å (250×21.2 mm) column (Phenomenex) on an Agilent 1260 Infinity II preparative LC-MSD system (Agilent Technologies, Inc., Santa Clara, CA), and eluting with linear gradients of CH₃CN+0.1% TFA/H₂O+0.1% TFA. Purified compounds were isolated by lyophilization of acetonitrile/water mixtures.

### General Procedure 6: Compound Analysis

LC-MS: Reactions were monitored for completion and purified compounds were analyzed using a Kinetex® 2.6-μm C18 100 Å (30×3 mm) column (Phenomenex) on an Agilent 1290 HPLC/6120 single quad LC-MS system (Agilent Technologies, Inc., Santa Clara, CA), and eluting with a with a 10 to 100% CH₃CN+0.1% TFA/H₂O+0.1% TFA linear gradient.

NMR: ¹H NMR spectra were collected with a Bruker AVANCE III 300 Spectrometer (300 MHz) (Bruker Corporation, Billerica, MA). Chemical shifts are in parts per million (ppm).

### General Procedure 7: MT-VK-PABC Compound-Linker Construct Synthesis

To the primary or secondary amine compound in dimethylformamide (0.05-0.1 M) was added the compound of Example 2.38 (MT-VK(Boc)-PABC-PNP) (1.0-1.1 eq.) followed by DIPEA (3-5 eq.). HOBt (1.0 eq.) was used for some reactions as indicated. Upon completion (typically 1-4 h), the reaction mixture was acidified with aqueous HCl (1 M) then purified by reverse-phase HPLC to provide the Boc protected drug-linker after lyophilization. The Boc intermediate was then deprotected according to General Procedure 2. Upon completion (typically 1 h), the reaction mixture was concentrated in vacuo and co-evaporated with DCM several times to provide a crude solid or was purified by preparative HPLC to provide the desired product after lyophilization.

### General Procedure 8: Conversion of Mesylate to Amine

To the chloride compound in DMF (50-100 mg/mL) was added a primary or secondary amine (2-5 eq.) and NaI (0.1-1.0 eq.) followed by DIPEA (3-5 eq.), and the solution was heated to 80° C. with stirring. Upon completion (typically 1-18 h), the reaction mixture was adjusted to pH 2 with 6 M HCl and purified by reverse-phase HPLC to provide the desired product after lyophilization.

### General Procedure 9: Preparation of ISACs Using Maleimide-Containing Compound-Linker Constructs

The antibody (1-10 mg/mL in phosphate buffered saline, pH 7.4) was reduced with TCEP (1-10 mM in dH₂O) (1.0-3.0 eq.) in the presence of 1 mM DTPA. The solution was mixed thoroughly and incubated at 37° C. for 120 min before cooling on ice. The reduced antibody solution was then further buffer exchanged into 10 mM sodium acetate buffer, pH 4.5 by passage over a Zeba™ Spin Desalting Columns (40 KDa MWCO; Thermo Scientific™). To the reduced protein solution stored on ice was added the maleimide functionalized compound-linker construct (10 mM in DMSO) (12-20 eq.). In some embodiments, propylene glycol (10-30 percent v/v %) was added to the reduced protein solution prior to the addition of compound-linker construct. The conjugation reaction was immediately mixed thoroughly by pipetting and conjugation was allowed to proceed at room temperature for 120 to 180 min. Once complete, reaction mixture was purified by passage over Zeba™ Spin Desalting Columns (40 KDa MWCO; Pierce) pre-equilibrated with 10 mM sodium acetate, pH 4.5. The purified conjugates were stored at 4° C. and analyzed for total protein content (bicinchoninic acid assay, Pierce micro-BCA protocol, catalogue #23225), characterized by HPLC-HIC, SEC, and/or RP-UPLC-MS. The average DAR and drug distribution was derived from interpretation of HIC and/or LC-MS data. Average DAR estimates of the synthesized conjugates were in the range of about 1.5 to 5, or more specifically from 1.9 to 4.3, as shown further herein. Endotoxin levels were assessed using Endosafe® LAL test cartridges (Charles River Catalogue #PTS20005F), with a sensitivity of 0.005 EU/mL on an Endosafe® nexgen-PTS™ testing system. Residual free compound and compound-linker construct levels were assessed by RP-UPLC-MS, with a threshold set at 1% ((free compound+compound-linker construct)/(conjugated compound-linker construct)).

### Example 1: Preparation of Compounds of Formulae (I)-(IV)

### Example 1.1: 2,6-dichloro-9-(tetrahydro-2H-pyran-2-yl)-9H-purine

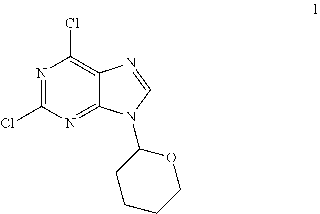 [SMILES: Clc1nc(Cl)c2ncn(C3CCCCO3)c2n1]

To a solution of 2,6-dichloro-9H-purine (11 g, 56 mmol, 1.0 eq.) in 100 mL EtOAc was added dihydropyran (7.0 g, 83 mmol, 1.5 eq.) and p-TsOH (96 mg, 0.56 mmol, 0.010 eq.). The mixture was heated to 50° C. for 3 h then concentrated in vacuo to yield the crude titled product 1 as a yellow solid (quantitative yield). LC-MS: Calc'd m/z=272.0 for C₁₀H₁₀Cl₂N₄O. found [M+H]⁺=273.0.

### Example 1.2: 2-chloro-9-(tetrahydro-2H-pyran-2-yl)-9H-purin-6-amine

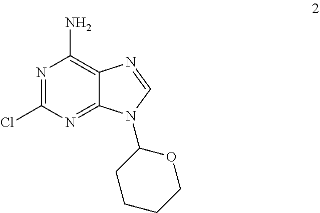 [SMILES: Nc1nc(Cl)nc2c1ncn2C1CCCCO1]

To a solution of NH₄OH in iPrOH (100 mL, 2.0 M) was added compound 1 (15 g, 55 mmol). The mixture was heated to 55° C. for 18 h then poured into H₂O (30 mL) and allowed to cool to room temperature. The resulting suspension was filtered, and the filter cake was washed with iPrOH (100 mL) to give the titled product 2 as a white solid (13 g, 51 mmol, 93%). LC-MS: Calc'd m/z=253.1 for C₁₀H₁₀ClN₅O. found [M+H]⁺=254.1.

### Example 1.3: 9-benzyl-2-chloro-9H-purin-6-amine

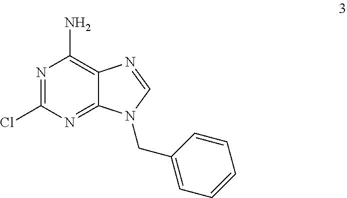 [SMILES: Nc1nc(Cl)nc2c1ncn2Cc1ccccc1]

To a solution of 2-chloroadenine (7.0 g, 41 mmol, 1.0 eq.) in DMSO (80 mL) was added K₂CO₃ (5.7 g, 41 mmol, 1.0 eq.) then benzyl bromide (7.4 g, 43 mmol, 1.1 eq.) and the resulting mixture stirred at room temperature for 18 h. The reaction was then concentrated in vacuo, the residue taken up in H₂O (100 mL) and EtOAc (50 mL) and the resulting mixture stirred vigorously for 3 h, after which the EtOAc was removed in vacuo and the suspension filtered. The filter cake was then washed with H₂O (2×50 mL) and air dried for 18 h to give the titled product 3 as a solid (7.2 g, 28 mmol, 67%). LC-MS: Calc'd m/z=259.1 for C₁₂H₁₀ClN₅. found [M+H]⁺=260.2.

### Example 1.4: 1-((tert-butyldiphenylsilyl)oxy)pentan-2-ol

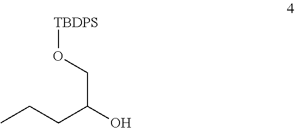

To a solution of pentane-1,2-diol (2.9 g, 28 mmol, 1.3 eq.) and imidazole (1.5 g, 21 mmol, 1.0 eq.) in DCM (10 mL) at 0° C. was added TBDPS-Cl and the mixture stirred for 1 h. The reaction was then allowed to warm to room temperature and stirred for an additional 36 h, after which it was concentrated in vacuo and the residue taken up in EtOAc (30 mL). The resulting organic solution was then washed with brine (2×10 mL), dried over MgSO₄ and concentrated in vacuo to give the titled product 4 as a clear and colourless oil (7.0 g, 20.4 mmol, 95%). ¹H NMR (300 MHz, CDCl₃) δ 7.88-7.70 (m, 5H), 7.57-7.35 (m, 5H), 3.89-3.53 (m, 3H), 3.03 (s, 1H), 1.61-1.31 (m, 4H), 1.17 (s, 9H), 1.05-0.92 (m, 3H).

### Example 1.5: 2-ethoxy-9-(tetrahydro-2H-pyran-2-yl)-9H-purin-6-amine

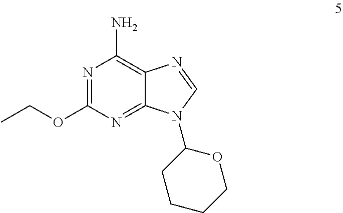 [SMILES: CCOc1nc(N)c2ncn(C3CCCCO3)c2n1]

To a solution of compound 2 (20.0 g, 78.8 mmol, 1.00 eq.) in EtOH (250 mL) was added KOtBu (17.7 g, 157 mmol, 2.0 eq.). The resulting solution was stirred at 60° C. for 18 h, after which the solvent was removed in vacuo. The resulting solid was suspended in H₂O (150 mL) and extracted with EtOAc (3×250 mL). The aqueous layer was further extracted with 10% iPrOH/EtOAc (100 mL). The pooled organics were washed with brine (50 mL) then dried over MgSO₄ and concentrated in vacuo to yield the titled compound 5 as an orange solid (20.0 g, 76.0 mmol, 96.4%). LC-MS: Calc'd m/z=263.1 for C₁₂H₁₇N₅O₂. found [M+H]⁺=264.2.

### Example 1.6: 8-bromo-2-ethoxy-9-(tetrahydro-2H-pyran-2-yl)-9H-purin-6-amine

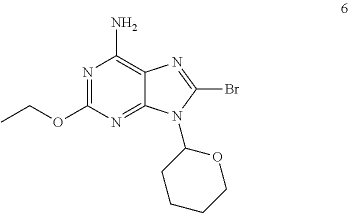 [SMILES: CCOc1nc(N)c2nc(Br)n(C3CCCCO3)c2n1]

To a suspension of compound 5 (20.0 g, 76.0 mmol, 1.00 eq.) in DCM (200 mL) was added NBS (20.3 g, 91.2 mmol, 1.20 eq.) in portions over the course of 3 mins. The resulting mixture was stirred at room temperature for 18 h, after which the reaction was quenched by the addition of 1 M NaHSO₃ (50 mL) and rapidly stirred for 30 mins. The resulting mixture was diluted with DCM (100 mL) and the separated organics washed with 1 M NaHSO₃ (2×50 mL). The pooled organics were dried over MgSO₄, filtered then concentrated in vacuo to yield the titled compound 6 as an orange solid (23.5 g, 68.7 mmol, 90.4%). LC-MS: Calc'd m/z=341.1 for C₁₂H₁₆BrN₅O₂. found [M+H]⁺=342.2.

### Example 1.7: 2-ethoxy-8-methoxy-9-tetrahydro-2H-pyran-2-yl)-9H-purin-6-amine

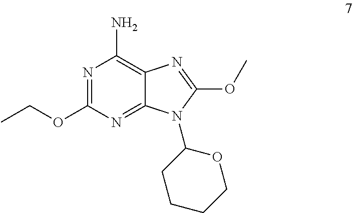 [SMILES: CCOc1nc(N)c2nc(OC)n(C3CCCCO3)c2n1]

To a solution of compound 6 (23 g, 67 mmol, 1.0 eq.) in MeOH (150 mL) was added NaOMe (9.1 g, 170 mmol, 2.5 eq.). The resulting mixture was stirred at room temperature for 72 h. Additional NaOMe (3.6 g, 66 mmol, 1.0 eq.) was added and the mixture was heated to reflux for 72 h. The solvent was removed in vacuo and crude material redissolved in EtOAc (250 mL), followed by washing with 1 M NaH₂PO₄ (150 mL). The aqueous layer was extracted with EtOAc (3×150 mL), the pooled organics dried over MgSO₄, and the solution concentrated in vacuo to yield title compound 7 as a red solid (17 g, 58 mmol, 86%). LC-MS: Calc'd m/z=293.2 for C₁₃H₁₉N₅O₃. found [M+H]⁺=294.2.

### Example 1.8: 2-ethoxy-8-methoxy-9-(tetrahydro-2H-pyran-2-yl)-9H-purin-6-amine

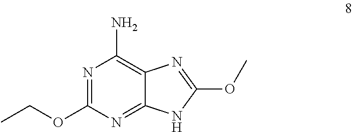

Compound 7 (13 g, 44 mmol, 1.0 eq.) was dissolved in 15% TFA/MeOH (40 mL) and the resulting solution stirred at room temperature for 72 h. The solvent was removed in vacuo to yield the titled compound 8 as a yellow solid (14 g, 40 mmol, 91%). LC-MS: Calc'd m/z=209.1 for C₈H₁₁N₅O₂. found [M+H]⁺=210.1.

### Example 1.9: Methyl 4-((6-amino-2-ethoxy-8-methoxy-9H-purin-9-yl)methyl)-3-methoxybenzoate

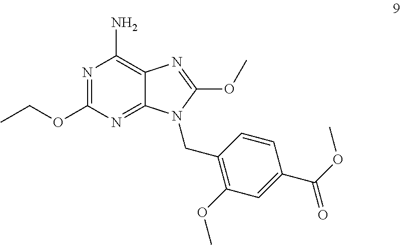 [SMILES: CCOc1nc(N)c2nc(OC)n(Cc3ccc(C(=O)OC)cc3OC)c2n1]

To a solution of compound 8 (0.70 g, 3.4 mmol, 1.0 eq.) and methyl 4-(bromomethyl)-3-methoxybenzoate (0.87 g, 3.4 mmol, 1.0 eq.) in DMF (8 mL) was added CsCO₃ (1.1 g, 3.4 mmol, 1.0 eq.). The resulting suspension was stirred at room temperature for 18 h then concentrated in vacuo. Reverse phase flash purification was accomplished as described in General Procedure 5 using a 60 g C18 column, eluting with a 10 to 60% CH₃CN+0.1% TFA/H₂O+0.1% TFA gradient. The titled compound 9 was obtained as a white solid (0.65 g, 1.7 mmol, 50%). LC-MS: Calc'd m/z=387.2 for C₁₈H₂₁N₅O₅. found [M+H]⁺=388.2.

### Example 1.10: (4-((6-amino-2-ethoxy-8-methoxy-9H-purin-9-yl)methyl)-3-methoxyphenyl)-methanol

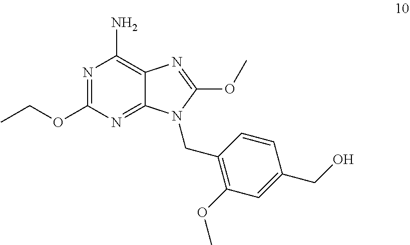 [SMILES: CCOc1nc(N)c2nc(OC)n(Cc3ccc(CO)cc3OC)c2n1]

To a stirring solution of compound 9 (250 mg, 0.65 mmol, 1.0 eq.) in anhydrous THF (5 mL) cooled to 0° C. was added lithium aluminum hydride (24 mg, 0.65 mmol, 1.0 eq.) in small portions over 5 mins. The resulting suspension was allowed to warm to room temperature and stirred for 15 mins, after which it was quenched with H₂O (1 mL) and diluted with MeOH (100 mL) then filtered through a celite plug. The filtrate was concentrated in vacuo and redissolved in 50% CH₃CN/H₂O (25 mL) to yield the titled compound 10 after lyophilization (230 mg, 0.63 mmol, 97%). LC-MS: Calc'd m/z=359.2 for C₁₇H₂₁N₅O₄. found [M+H]⁺=360.2.

### Example 1.11: 6-amino-9-(4-(chloromethyl)-2-methoxybenzyl)-2-ethoxy-9H-purin-8-ol

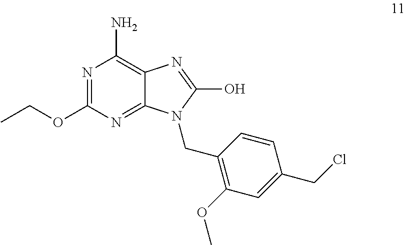 [SMILES: CCOc1nc(N)c2nc(O)n(Cc3ccc(CCl)cc3OC)c2n1]

The title compound 11 was prepared according to General Procedure 3 using compound 10 (210 mg, 0.58 mmol, 1.0 eq.) and 10% SOCl₂/DCM (10 mL). The titled compound was obtained as a yellow solid (assumed quantitative yield). LC-MS: Calc'd m/z=363.1 for C₁₆H₁₈ClN₅O₃. found [M+H]⁺=364.2.

### Example 1.12: 6-amino-2-ethoxy-9-(2-methoxy-4-(piperazin-1-ylmethyl)benzyl)-9H-purin-8-ol (Compound 100)

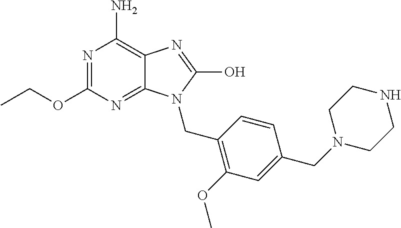 [SMILES: CCOc1nc(N)c2nc(O)n(Cc3ccc(CN4CCNCC4)cc3OC)c2n1]

The titled compound was prepared according to General Procedure 1 from compound 11 (0.040 g, 0.11 mmol, 1.0 eq.) and tert-butyl piperazine-1-carboxylate (61 mg, 0.33 mmol, 3.0 eq.). Preparative HPLC purification was accomplished as described in General Procedure 5, eluting with a 10 to 45% CH₃CN+0.1% TFA/H₂O+0.1% TFA gradient. Deprotection was accomplished according to General Procedure 2 to give the title compound 100 as a white solid (55 mg, 0.073 mmol, 66%). LC-MS: Calc'd m/z=413.2 for C₂₀H₂₇N₇O₃. found [M+H]⁺=414.3. ¹H NMR (300 MHz, MeOD) δ 7.14 (s, 1H), 7.10 (d, J=7.7 Hz, 1H), 6.98 (d, J=7.6, 1H), 5.04 (s, 2H), 4.38 (q, J=7.0 Hz, 2H), 4.17 (s, 2H), 3.89 (s, 3H), 3.50-3.42 (m, 4H), 3.33-3.26 (m, 4H), 1.34 (t, J=7.1 Hz, 3H).

### Example 1.13: 6-amino-2-ethoxy-9-(4-(((2-hydroxy-2-methylpropyl)amino)methyl)-2-methoxybenzyl)-9H-purin-8-ol (Compound 101)

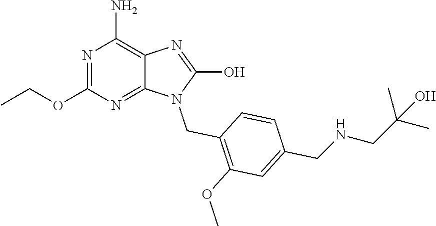 [SMILES: CCOc1nc(N)c2nc(O)n(Cc3ccc(CNCC(C)(C)O)cc3OC)c2n1]

The title compound was prepared according to General Procedure 1 from compound 11 (0.010 g, 0.028 mmol, 1.0 eq.) and 1-amino-2-methylpropan-2-ol (7.0 mg, 0.082 mmol, 3.0 eq.). Preparative HPLC purification was accomplished as described in General Procedure 5, eluting with a 10 to 45% CH₃CN+0.1% TFA/H₂O+0.1% TFA gradient to give the title compound 101 as a white solid (11.7 mg, 0.0181 mmol, 66.0%). LC-MS: Calc'd m/z=416.2 for C₂₀H₂₈N₆O₄. found [M+H]⁺=417.3. ¹H NMR (300 MHz, CD₃CN) δ 7.14 (s, 1H), 7.07 (d, J=7.8 Hz, 1H), 6.98 (d, J=7.7 1H), 4.97 (s, 2H), 4.33 (q, J=7.1 Hz, 2H), 4.18 (s, 2H), 3.86 (s, 3H), 2.89 (s, 2H), 1.29 (t, J=7.1 Hz, 3H), 1.20 (s, 6H).

### Example 1.14: 6-amino-9-(4-((diethylamino)methyl)-2-methoxybenzyl)-2-ethoxy-9H-purin-8-ol (Compound 102)

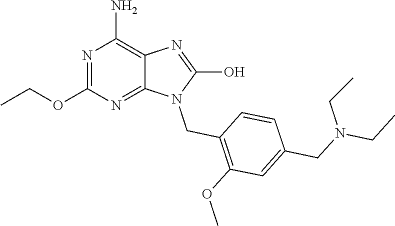 [SMILES: CCOc1nc(N)c2nc(O)n(Cc3ccc(CN(CC)CC)cc3OC)c2n1]

The title compound was prepared according to General Procedure 1 from compound 11 (0.010 g, 0.028 mmol, 1.0 eq.) and diethylamine (7.0 mg, 0.082 mmol, 3.0 eq.). Preparative HPLC purification was accomplished as described in General Procedure 5, eluting with a 10 to 45% CH₃CN+0.1% TFA/H₂O+0.1% TFA gradient to give the title compound 102 as a white solid (10 mg, 0.026 mmol, 95%). LC-MS: Calc'd m/z=400.2 for C₂₀H₂₈N₆O₃. found [M+H]⁺=401.2. ¹H NMR (300 MHz, CD₃CN) δ 7.13-7.05 (m, 2H), 6.97 (dd, J=7.7, 1.6 Hz, 1H), 4.97 (s, 2H), 4.31 (q, J=7.0 Hz, 2H), 4.19 (s, 2H), 3.87 (s, 3H), 3.11 (septet, J=7.2 Hz, 4H), 1.30-1.23 (m, 9H).

### Example 1.15: 6-amino-2-ethoxy-9-(4-(((5-hydroxypentyl)amino)methyl)-2-methoxy-benzyl)-9H-purin-8-ol (Compound 103)

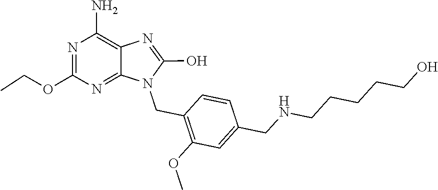 [SMILES: CCOc1nc(N)c2nc(O)n(Cc3ccc(CNCCCCCO)cc3OC)c2n1]

The title compound was prepared according to General Procedure 1 from compound 11 (0.010 g, 0.028 mmol, 1.0 eq.) and 5-aminopentan-1-ol (9.0 mg, 0.03 mmol, 3 eq.). Preparative HPLC purification was accomplished as described in General Procedure 5, eluting with a 10 to 45% CH₃CN+0.1% TFA/H₂O+0.1% TFA gradient to give the title compound 103 as a white solid (10 mg, 0.015 mmol, 95%). LC-MS: Calc'd m/z=430.2 for C₂₁H₃₀N₆O₄. found [M+H]⁺=431.2. ¹H NMR (300 MHz, CD₃CN) δ 7.11-7.01 (m, 2H), 6.94 (dd, J=7.7, 1.6 Hz, 1H), 4.97 (s, 2H), 4.32 (q, J=7.0 Hz, 2H), 4.09 (s, 2H), 3.86 (s, 3H), 3.49 (t, J=6.2 Hz, 2H), 3.01-2.90 (m, 2H), 1.66 (p, J=7.7 Hz, 2H), 1.49 (p, J=6.6 Hz, 2H), 1.43-1.30 (m, 2H), 1.29 (t, J=7.1 Hz, 3H).

### Example 1.16: 6-amino-9-(4-(((3-(4-(3-aminopropyl)piperazin-1-yl)propyl)amino)methyl)-2-methoxybenzyl)-2-ethoxy-9H-purin-8-ol (Compound 104)

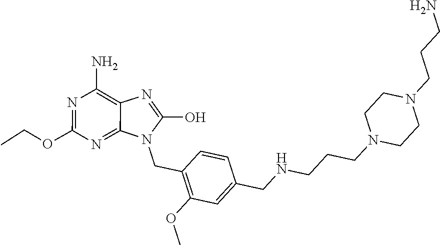 [SMILES: CCOc1nc(N)c2nc(O)n(Cc3ccc(CNCCCN4CCN(CCCN)CC4)cc3OC)c2n1]

The titled compound was prepared according to General Procedure 1 from compound 11 (0.010 g, 0.028 mmol, 1.0 eq.) and 1,4-piperazinedipropanamine (17 mg, 0.082 mmol, 3.0 eq.). Preparative HPLC purification was accomplished as described in General Procedure 5, eluting with a 10 to 45% CH₃CN+0.1% TFA/H₂O+0.1% TFA gradient to give the title compound 104 as a white solid (14.8 mg, 0.0150 mmol, 54.7%). LC-MS: Calc'd m/z=527.3 for C₂₆H₄₁N₉O₃. found [M+H]⁺=528.3. ¹H NMR (300 MHz, CD₃CN) δ 7.14-7.03 (m, 2H), 6.96 (dd, J=7.9, 1.5 Hz, 1H), 4.97 (s, 2H), 4.35 (q, J=7.1 Hz, 2H), 4.13 (s, 2H), 3.85 (s, 3H), 3.13-2.99 (m, 8H), 2.06 (qd, J=7.9, 2.8 Hz, 4H), 1.30 (t, J=7.0 Hz, 3H).

### Example 1.17: 6-amino-9-(4-(((4-(aminomethyl)benzyl)amino)methyl)-2-methoxybenzyl)-2-ethoxy-9H-purin-8-ol (Compound 105)

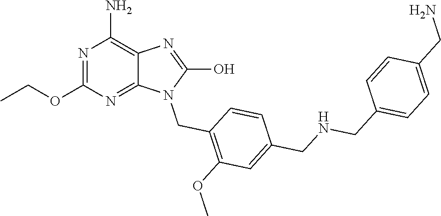 [SMILES: CCOc1nc(N)c2nc(O)n(Cc3ccc(CNCc4ccc(CN)cc4)cc3OC)c2n1]

The titled compound was prepared according to General Procedure 1 from compound 11 (0.010 g, 0.028 mmol, 1.0 eq.) and tert-butyl (4-(aminomethyl)benzyl)carbamate (0.020 g, 0.084 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 105 as a white solid (4.8 mg, 0.0059 mmol, 43%). LC-MS: Calc'd m/z=463.2 for C₂₄H₂₉N₇O₃. found [M+H]⁺=464.3. ¹H NMR (300 MHz, CD₃CN) δ 7.46 (s, 4H), 7.12-7.01 (m, 2H), 6.99-6.91 (m, 1H), 4.96 (s, 2H), 4.33 (q, J=7.1 Hz, 2H), 4.18 (s, 2H), 4.17 (s, 2H), 4.11 (s, 2H), 3.85 (s, 3H), 1.29 (t, J=7.1 Hz, 3H).

### Example 1.18: 6-amino-9-(4-(((4-aminobutyl)amino)methyl)-2-methoxybenzyl)-2-ethoxy-9H-purin-8-ol (Compound 106)

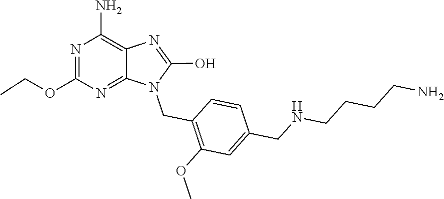 [SMILES: CCOc1nc(N)c2nc(O)n(Cc3ccc(CNCCCCN)cc3OC)c2n1]

The titled compound was prepared according to General Procedure 1 from compound 11 (0.010 g, 0.028 mmol, 1.0 eq.) and tert-butyl (4-aminobutyl)carbamate (24 mg, 0.082 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 106 as a white solid (5.6 mg, 0.0050 mmol, 36%). LC-MS: Calc'd m/z=415.2 for C₂₀H₂₉N₇O₃. found [M+H]⁺=416.3. ¹H NMR (300 MHz, CD₃CN) δ 7.13-6.99 (m, 2H), 6.95 (dd, J=7.8, 1.6 Hz, 1H), 4.96 (s, 2H), 4.29 (q, J=7.1 Hz, 2H), 4.11 (s, 2H), 3.86 (s, 3H), 3.04-2.86 (m, 4H), 1.75-1.60 (dq, J=12.5, 6.5 Hz, 4H), 1.29 (t, J=7.0 Hz, 3H).

### Example 1.19: 6-amino-9-(4-((benzylamino)methyl)-2-methoxybenzyl)-2-ethoxy-9H-purin-8-ol (Compound 107)

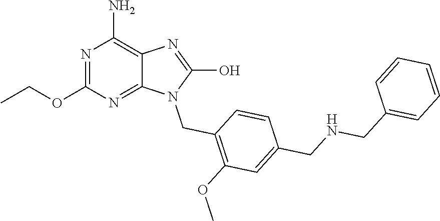 [SMILES: CCOc1nc(N)c2nc(O)n(Cc3ccc(CNCc4ccccc4)cc3OC)c2n1]

The titled compound was prepared according to General Procedure 1 from compound 11 (0.010 g, 0.028 mmol, 1.0 eq.) and benzylamine (9.0 mg, 0.082 mmol, 3.0 eq.). Preparative HPLC purification was accomplished as described in General Procedure 5, eluting with a 10 to 45% CH₃CN+0.1% TFA/H₂O+0.1% TFA gradient to give the title compound 107 as a white solid (14 mg, 0.021 mmol, 76%). LC-MS: Calc'd m/z=434.2 for C₂₃H₂₆N₆O₃. found [M+H]⁺=435.3. ¹H NMR (300 MHz, CD₃CN) δ 7.45 (s, 5H), 7.11-6.99 (m, 2H), 6.95 (dd, J=7.7, 1.6 Hz, 1H), 4.96 (s, 2H), 4.30 (q, J=7.1 Hz, 2H), 4.17 (s, 2H), 4.15 (s, 2H), 3.86 (s, 3H), 1.29 (t, J=7.0 Hz, 3H).

### Example 1.20: 6-amino-9-(4-((cyclobutylamino)methyl)-2-methoxybenzyl)-2-ethoxy-9H-purin-8-ol (Compound 193)

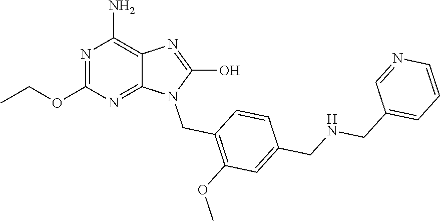 [SMILES: CCOc1nc(N)c2nc(O)n(Cc3ccc(CNCc4cccnc4)cc3OC)c2n1]

The titled compound was prepared according to General Procedure 1 from compound 11 (0.010 g, 0.028 mmol, 1.0 eq.) and picolamine (15 mg, 0.082 mmol, 3.0 eq.). Preparative HPLC purification was accomplished as described in General Procedure 5, eluting with a 10 to 45% CH₃CN+0.1% TFA/H₂O+0.1% TFA gradient to give the title compound 193 as a white solid (15 mg, 0.020 mmol, 72%). LC-MS: Calc'd m/z=435.2 for C₂₂H₂₅N₇O₃. found [M+H]⁺=436.3. ¹H NMR (300 MHz, CD₃CN) δ 8.80 (s, 1H), 8.74-8.68 (m, 1H), 8.36 (dd, J=8.1, 2.1 Hz, 1H), 7.82 (dd, J=8.1, 5.4 Hz, 1H), 7.11 (d, J=1.6 Hz, 1H), 7.05 (d, J=7.8 Hz, 1H), 6.97 (d, J=7.8, 1H), 4.96 (s, 2H), 4.37-4.29 (m, 4H), 4.24 (s, 2H), 3.84 (s, 3H), 1.29 (t, J=7.0 Hz, 3H).

### Example 1.21: 6-amino-9-(4-((cyclobutylamino)methyl)-2-methoxybenzyl)-2-ethoxy-9H-purin-8-ol (Compound 108)

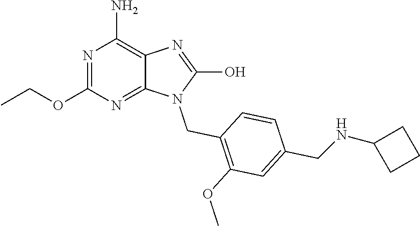 [SMILES: CCOc1nc(N)c2nc(O)n(Cc3ccc(CNC4CCC4)cc3OC)c2n1]

The titled compound was prepared according to General Procedure 1 from compound 11 (0.010 g, 0.028 mmol, 1.0 eq.) and cyclobutylamine (0.010 g, 0.082 mmol, 3.0 eq.). Preparative HPLC purification was accomplished as described in General Procedure 5, eluting with a 10 to 45% CH₃CN+0.1% TFA/H₂O+0.1% TFA gradient to give the title compound 108 as a white solid (9.0 mg, 0.014 mmol, 52%). LC-MS: Calc'd m/z=398.2 for C₂₀H₂₆N₆O₃. found [M+H]⁺=399.3. ¹H NMR (300 MHz, CD₃CN) δ 7.09-7.00 (m, 2H), 6.92 (dd, J=7.8, 1.6 Hz, 1H), 4.96 (s, 2H), 4.30 (q, J=7.1 Hz, 2H), 3.99 (s, 2H), 3.86 (s, 3H), 3.69 (p, J=8.2 Hz, 1H), 2.28-2.09 (m, 4H), 1.95-1.73 (m, 2H), 1.29 (t, J=7.0 Hz, 3H).

### Example 1.22: 6-amino-9-(4-(((3,3-difluorocyclobutyl)amino)methyl)-2-methoxybenzyl)-2-ethoxy-9H-purin-8-ol (Compound 109)

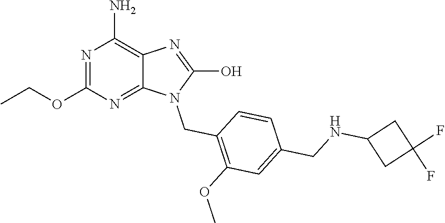 [SMILES: CCOc1nc(N)c2nc(O)n(Cc3ccc(CNC4CC(F)(F)C4)cc3OC)c2n1]

The titled compound was prepared according to General Procedure 1 from compound 11 (0.010 g, 0.028 mmol, 1.0 eq.) and 3,3-difluorocyclobutylamine (15 mg, 0.12 mmol, 3.0 eq.). Preparative HPLC purification was accomplished as described in General Procedure 5, eluting with a 10 to 45% CH₃CN+0.1% TFA/H₂O+0.1% TFA gradient to give the title compound 109 as a white solid (12 mg, 0.018 mmol, 66%). LC-MS: Calc'd m/z=434.2 for C₂₀H₂₄F₂N₆O₃. found [M+H]⁺=435.3. ¹H NMR (300 MHz, MeOD) δ 7.14 (s, 1H), 7.08 (d, J=7.6 Hz, 1H), 7.00 (d, J=8.2 Hz, 1H), 5.05 (s, 2H), 4.31 (q, J=5.2 Hz, 2H), 4.19 (s, 2H), 3.93 (s, 3H), 3.87-3.75 (m, 1H), 3.18-2.76 (m, 4H), 1.33 (t, J=7.1 Hz, 3H).

### Example 1.23: 6-amino-9-(4-((4-aminopiperidin-1-yl)methyl)-2-methoxybenzyl)-2-ethoxy-9H-purin-8-ol (Compound 110)

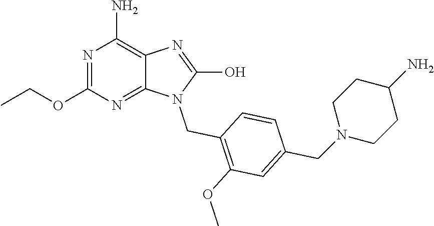 [SMILES: CCOc1nc(N)c2nc(O)n(Cc3ccc(CN4CCC(N)CC4)cc3OC)c2n1]

The titled compound was prepared according to General Procedure 1 from compound 11 (15.0 mg, 0.0412 mmol, 1.00 eq.) and 4-Boc-aminopiperidine (25 mg, 0.12 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 110 as a white solid (8.2 mg, 0.011 mmol, 52%). LC-MS: Calc'd m/z=427.2 for C₂₁H₂₉N₇O₃. found [M+H]⁺=428.3. ¹H NMR (300 MHz, CD₃CN) δ 7.14-7.11 (m, 2H), 6.97 (dd, J=7.7, 1.6 Hz, 1H), 4.99 (s, 2H), 4.37 (q, J=7.1 Hz, 2H), 4.21 (s, 2H), 3.86 (s, 3H), 3.50-3.41 (m, 4H), 3.07-2.96 (m, 2H), 2.25-2.18 (m, 2H), 1.30 (t, J=7.1 Hz, 3H).

### Example 1.24: 6-amino-9-(4-(((2-aminoethyl)amino)methyl)-2-methoxybenzyl)-2-ethoxy-9H-purin-8-ol (Compound 111)

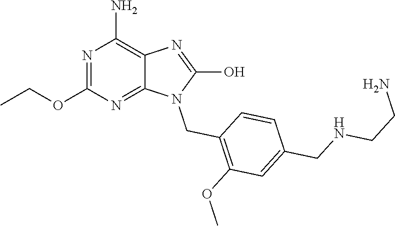 [SMILES: CCOc1nc(N)c2nc(O)n(Cc3ccc(CNCCN)cc3OC)c2n1]

The titled compound was prepared according to General Procedure 1 from compound 11 (15 mg, 0.041 mmol, 1.0 eq.) and tert-butyl (2-aminoethyl)carbamate (20 mg, 0.12 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 111 as a white solid (11.0 mg, 0.0151 mmol, 73.1%). LC-MS: Calc'd m/z=387.2 for C₁₈H₂₅N₇O₃. found [M+H]⁺=388.3. ¹H NMR (300 MHz, CD₃CN) δ 7.11 (m, 2H), 6.97 (dd, J=7.8, 1.6 Hz, 1H), 4.98 (s, 2H), 4.39 (q, J=7.0 Hz, 2H), 4.18 (s, 2H), 3.85 (s, 3H), 3.43-3.29 (m, 4H), 1.31 (t, J=7.1 Hz, 3H).

### Example 1.25: 6-amino-9-(4-(((2-aminoethyl)amino)methyl)-2-methoxybenzyl)-2-ethoxy-9H-purin-8-ol (Compound 112)

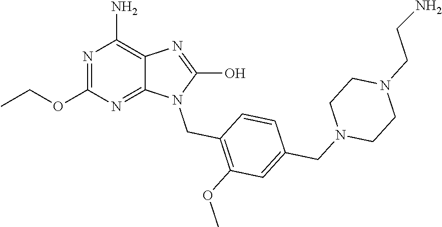 [SMILES: CCOc1nc(N)c2nc(O)n(Cc3ccc(CN4CCN(CCN)CC4)cc3OC)c2n1]

The titled compound was prepared according to General Procedure 1 from compound 11 (15 mg, 0.041 mmol, 1.0 eq.) and tert-butyl (2-(piperazin-1-yl)ethyl)carbamate (28 mg, 0.12 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 112 as a white solid (17 mg, 0.019 mmol, 91%). LC-MS: Calc'd m/z=456.2 for C₂₂H₃₂N₈O₃. found [M+H]⁺=457.3. ¹H NMR (300 MHz, CD₃CN) δ 7.20-7.07 (m, 1H), 7.12 (s, 1H), 6.99 (d, J=7.5 Hz, 1H), 4.99 (s, 2H), 4.40 (q, J=7.1, 2H), 4.24 (s, 2H), 3.84 (s, 3H), 3.42-3.35 (m, 8H), 3.07 (q, J=7.0 Hz, 2H), 2.89-2.73 (m, 2H), 1.31 (t, J=7.0 Hz, 3H).

### Example 1.26: 2-(((S)-2-amino-2-carboxyethyl)thio)-4-((2-(4-(4-((6-amino-2-ethoxy-8-hydroxy-9H-purin-9-yl)methyl)-3-methoxybenzyl)piperazin-1-yl)ethyl)amino)-4-oxobutanoic Acid (Compound 113)

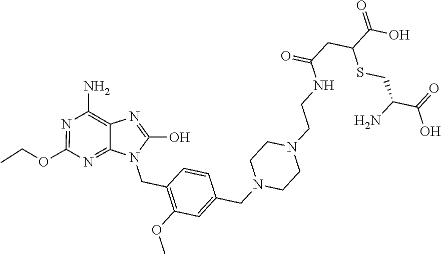 [SMILES: CCOc1nc(N)c2nc(O)n(Cc3ccc(CN4CCN(CCNC(=O)CC(SC[C@@H](N)C(=O)O)C(=O)O)CC4)cc3OC)c2n1]

To a solution of Example 2.26 (35 mg, 0.046 mmol, 1.0 eq.) in DMF (500 μL) and CH₃CN (500 μL) was added H₂O (2 mL) then L-cysteine (17 mg, 0.14 mmol, 3.0 eq.) and 0.5 M Na₂HPO₄ (500 μL). The resulting solution was stirred at room temperature for 1 h, after which 10 M NaOH was added (600 μL) and the reaction mixture stirred at room temperature for an additional 2 h. Reverse phase flash purification was accomplished as described in General Procedure 5 using a 12 g C18 column, eluting with a 10 to 50% CH₃CN+0.1% TFA/H₂O+0.1% TFA gradient to give the title compound 113 as a white solid (35 mg, 0.031 mmol, 68%). LC-MS: Calc'd m/z=675.3 for C₂₉H₁₄N₉O₈S. found [M+H]⁺=676.4. ¹H NMR (300 MHz, MeOD) δ 7.19-7.06 (m, 2H), 6.99 (d, J=7.7 Hz, 1H), 5.05 (s, 2H), 4.51-4.21 (m, 3H), 4.19 (d, J=4.0 Hz, 2H), 3.90 (s, 3H), 3.88-3.71 (m, 1H), 3.64-2.99 (m, 14H), 2.87 (dd, J=16.4, 7.1 Hz, 1H), 2.77-2.50 (m, 1H), 1.35 (t, J=7.1 Hz, 3H).

### Example 1.27: N⁶-(4-((6-amino-2-ethoxy-8-hydroxy-9H-purin-9-yl)methyl)-3-methoxybenzyl)-L-lysine (Compound 114)

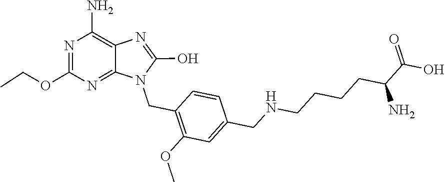 [SMILES: CCOc1nc(N)c2nc(O)n(Cc3ccc(CNCCCC[C@H](N)C(=O)O)cc3OC)c2n1]

To a solution of N_{α}-Boc-L-lysine (32 mg, 0.14 mmol, 5.0 eq.) in aqueous NaHCO₃ (0.275 mL, 1.14 M, 12.5 eq.) was added a solution of compound 11 (0.010 g, 0.027 mmol, 1.0 eq.) in 100 μL DMA. The resulting solution was heated to 50° C. for 4 h then acidified with aqueous HCl (5 M). Preparative HPLC purification was accomplished as described in General Procedure 5, eluting with a 10 to 45% CH₃CN+0.1% TFA/H₂O+0.1% TFA gradient. Deprotection was accomplished according to General Procedure 2 to give the title compound 114 as a white solid (7.9 mg, 0.010 mmol, 37%). LC-MS: Calc'd m/z=473.2 for C₂₂H₃₁N₇O₅. found [M+H]⁺=474.3. ¹H NMR (300 MHz, MeOD) δ 7.18-7.04 (m, 2H), 7.00 (d, J=7.7 Hz, 1H), 5.05 (s, 2H), 4.34 (q, J=7.1 Hz, 2H), 4.20 (s, 2H), 3.98 (t, J=6.3 Hz, 1H), 3.93 (s, 3H), 3.13-3.01 (m, 2H), 2.05-1.48 (m, 6H), 1.34 (t, J=7.1 Hz, 4H).

### Example 1.28: 6-((4-((6-amino-2-ethoxy-8-hydroxy-9H-purin-9-yl)methyl)-3-methoxybenzyl)-amino)hexanoic Acid (Compound 115)

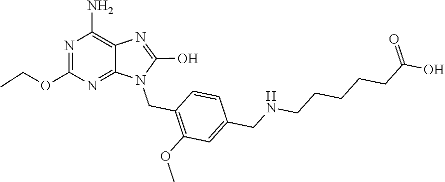 [SMILES: CCOc1nc(N)c2nc(O)n(Cc3ccc(CNCCCCCC(=O)O)cc3OC)c2n1]

To a solution of 6-aminohexanoic acid (18 mg, 0.14 mmol, 5.0 eq.) in aqueous NaHCO₃ (0.275 mL, 1.14 M, 12.5 eq.) was added a solution of compound 11 (0.010 g, 0.014 mmol, 1.0 eq.) in 100 μL DMA. The resulting solution was heated to 50° C. for 4 h then acidified with aqueous HCl (5 M). Preparative HPLC purification was accomplished as described in General Procedure 5, eluting with a 10 to 45% CH₃CN+0.1% TFA/H₂O+0.1% TFA gradient to give the title compound 115 as a white solid (6.7 mg, 0.013 mmol, 48%). LC-MS: Calc'd m/z=458.2 for C₂₂H₃₀N₆O₅. found [M+H]⁺=459.2. ¹H NMR (300 MHz, MeOD) δ 7.14 (d, J=7.5 Hz, 2H), 7.01 (dd, J=7.6, 1.6 Hz, 1H), 5.06 (s, 2H), 4.46-4.33 (m, 2H), 4.19 (s, 2H), 3.93 (s, 3H), 3.11-2.99 (m, 2H), 2.34 (t, J=7.2 Hz, 2H), 1.79-1.61 (m, 4H), 1.52-1.25 (m, 5H).

### Example 1.29: (3-((4-((6-amino-2-ethoxy-8-hydroxy-9H-purin-9-yl)methyl)-3-methoxybenzyl)-amino)-propanoyl)-L-histidine (Compound 116)

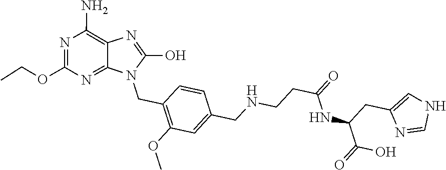 [SMILES: CCOc1nc(N)c2nc(O)n(Cc3ccc(CNCCC(=O)N[C@@H](Cc4c[nH]cn4)C(=O)O)cc3OC)c2n1]

To a solution of carnosine (31 mg, 0.14 mmol, 5.0 eq.) in aqueous NaHCO₃ (0.275 mL, 1.14 M, 12.5 eq.) was added a solution of compound 11 (0.010 g, 0.014 mmol, 1.0 eq.) in 100 μL DMA. The resulting solution was heated to 50° C. for 4 h then acidified with aqueous HCl (5 M). Preparative HPLC purification was accomplished as described in General Procedure 5, eluting with a 10 to 45% CH₃CN+0.1% TFA/H₂O+0.1% TFA gradient to give the title compound 116 as a white solid (8.5 mg, 0.0090 mmol, 35%). LC-MS: Calc'd m/z=553.2 for C₂₅H₃₁N₉O₆. found [M+H]⁺=554.3. ¹H NMR (300 MHz, MeOD) δ 8.83 (d, J=1.4 Hz, 1H), 7.37 (s, 1H), 7.18-6.98 (m, 3H), 5.06 (s, 2H), 4.80 (dd, J=8.4, 5.3 Hz, 1H), 4.38 (q, J=7.1 Hz, 2H), 4.22 (s, 2H), 3.93 (s, 3H), 3.41-3.05 (m, 4H), 2.72 (dd, J=7.6, 5.7 Hz, 2H), 1.36 (t, J=7.1 Hz, 3H).

### Example 1.30: (4-((6-amino-2-ethoxy-8-hydroxy-9H-purin-9-yl)methyl)-3-methoxybenzyl)-L-phenylalanine (Compound 117)

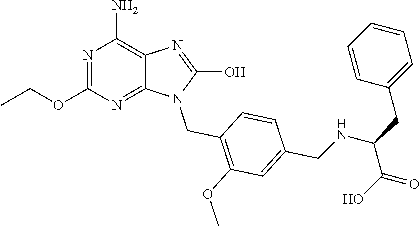 [SMILES: CCOc1nc(N)c2nc(O)n(Cc3ccc(CN[C@@H](Cc4ccccc4)C(=O)O)cc3OC)c2n1]

To a solution of L-phenylalanine (22 mg, 0.14 mmol, 5.0 eq.) in aqueous NaHCO₃ (0.275 mL, 1.14 M, 12.5 eq.) was added a solution of compound 11 (0.010 g, 0.014 mmol, 1.0 eq.) in 100 μL DMA. The resulting solution was heated to 50° C. for 4 h then acidified with aqueous HCl (5 M). Preparative HPLC purification was accomplished as described in General Procedure 5, eluting with a 10 to 45% CH₃CN+0.1% TFA/H₂O+0.1% TFA gradient to give the title compound 117 as a white solid (6.2 mg, 0.0086 mmol, 32%). LC-MS: Calc'd m/z=492.2 for C₂₅H₂₈N₆O₅. found [M+H]⁺=493.3. ¹H NMR (300 MHz, MeOD) δ 7.39-7.24 (m, 5H), 7.12-7.07 (m, 2H), 6.98 (d, J=7.7 Hz, 1H), 5.06 (s, 2H), 4.37 (q, J=7.1 Hz, 2H), 4.28-4.19 (m, 3H), 3.91 (s, 3H), 3.29-3.21 (m, 2H), 1.35 (t, J=7.2 Hz, 3H).

### Example 1.31: (S)-6-amino-2-ethoxy-9-(4-(((1-hydroxy-3-phenylpropan-2-yl)amino)methyl)-2-methoxybenzyl)-9H-purin-8-ol (Compound 118)

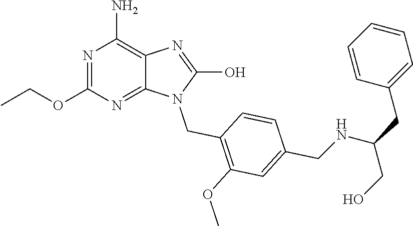 [SMILES: CCOc1nc(N)c2nc(O)n(Cc3ccc(CN[C@H](CO)Cc4ccccc4)cc3OC)c2n1]

To a solution of L-phenylalaninol (21 mg, 0.14 mmol, 5.0 eq.) in aqueous NaHCO₃ (0.275 mL, 1.14 M, 12.5 eq.) was added a solution of compound 11 (0.010 g, 0.014 mmol, 1.0 eq.) in 100 μL DMA. The resulting solution was heated to 50° C. for 4 h then acidified with aqueous HCl (5 M). Preparative HPLC purification was accomplished as described in General Procedure 5, eluting with a 10 to 45% CH₃CN+0.1% TFA/H₂O+0.1% TFA gradient to give the title compound 118 as a white solid (11 mg, 0.015 mmol, 55%). LC-MS: Calc'd m/z=478.2 for C₂₅H₃₀N₆O₄. found [M+H]⁺=479.3. ¹H NMR (300 MHz, MeOD) δ 7.41-7.11 (m, 7H), 7.04 (dd, J=7.8, 1.6 Hz, 1H), 5.07 (s, 2H), 4.45-4.22 (m, 4H), 3.93 (s, 3H), 3.78 (dd, J=12.1, 3.3 Hz, 1H), 3.58 (dd, J=12.2, 4.7 Hz, 1H), 3.50-3.35 (m, 1H), 3.11 (dd, J=13.5, 4.7 Hz, 1H), 2.99 (dd, J=13.5, 10.3 Hz, 1H), 1.36 (t, J=7.1 Hz, 3H).

### Example 1.32: (4-((6-amino-2-ethoxy-8-hydroxy-9H-purin-9-yl)methyl)-3-methoxybenzyl)-L-serine (Compound 119)

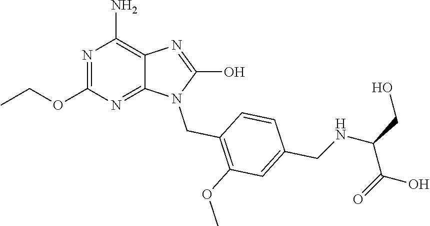 [SMILES: CCOc1nc(N)c2nc(O)n(Cc3ccc(CN[C@@H](CO)C(=O)O)cc3OC)c2n1]

To a solution of L-serine (14 mg, 0.14 mmol, 5.0 eq.) in aqueous NaHCO₃ (0.275 mL, 1.14 M, 12.5 eq.) was added a solution of compound 11 (0.010 g, 0.014 mmol, 1.0 eq.) in 100 μL DMA. The resulting solution was heated to 50° C. for 4 h then acidified with aqueous HCl (5 M). Preparative HPLC purification was accomplished as described in General Procedure 5, eluting with a 10 to 45% CH₃CN+0.1% TFA/H₂O+0.1% TFA gradient to give the title compound 119 as a white solid (8.7 mg, 0.013 mmol, 48%). LC-MS: Calc'd m/z=432.2 for C₁₉H₂₄N₆O₆. found [M+H]⁺=433.3. ¹H NMR (300 MHz, MeOD) δ 7.22-7.12 (m, 2H), 7.04 (dd, J=7.7, 1.6 Hz, 1H), 5.08 (s, 2H), 4.43 (q, J=7.1 Hz, 2H), 4.31 (s, 2H), 4.15-3.98 (m, 3H), 3.93 (s, 3H), 1.38 (t, J=7.1 Hz, 3H).

### Example 1.33: 2,2′-((4-((6-amino-2-ethoxy-8-hydroxy-9H-purin-9-yl)methyl)-3-methoxy-benzyl)azanediyl) diacetic Acid (Compound 120)

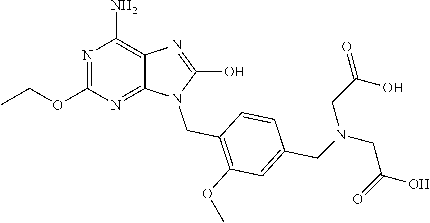 [SMILES: CCOc1nc(N)c2nc(O)n(Cc3ccc(CN(CC(=O)O)CC(=O)O)cc3OC)c2n1]

To a solution of iminodiacetic acid (18 mg, 0.14 mmol, 5.0 eq.) in aqueous NaHCO₃ (0.275 mL, 1.14 M, 12.5 eq.) was added a solution of compound 11 (0.010 g, 0.014 mmol, 1.0 eq.) in 100 μL DMA. The resulting solution was heated to 50° C. for 4 h then acidified with aqueous HCl (5 M). Preparative HPLC purification was accomplished as described in General Procedure 5, eluting with a 10 to 45% CH₃CN+0.1% TFA/H₂O+0.1% TFA gradient to give the title compound 120 as a white solid (7.9 mg, 0.011 mmol, 42%). LC-MS: Calc'd m/z=460.2 for C₂₀H₂₄N₆O₇. found [M+H]⁺=461.3. ¹H NMR (300 MHz, MeOD) δ 7.22-6.99 (m, 3H), 5.07 (s, 2H), 4.51 (s, 2H), 4.40-4.34 (m, 2H), 4.16-4.08 (m, 4H), 3.94 (s, 3H), 1.35 (t, J=7.1, 3H).

### Example 1.34: (4-((6-amino-2-ethoxy-8-hydroxy-9H-purin-9-yl)methyl)-3-methoxybenzyl)-L-isoleucine (Compound 121)

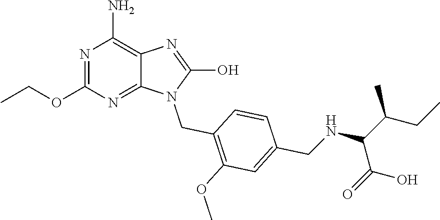

To a solution of (2S,3S)-2-amino-3-hydroxypentanoic acid (37 mg, 0.28 mmol, 10 eq.) in aqueous NaHCO₃ (0.362 mL, 1.14 M, 15.0 eq.) was added a solution of compound 11 (0.010 g, 0.014 mmol, 1.0 eq.) in 100 μL DMA. The resulting solution was heated to 50° C. for 18 h then acidified with aqueous HCl (1 M). Preparative HPLC purification was accomplished as described in General Procedure 5, eluting with a 10 to 45% CH₃CN+0.1% TFA/H₂O+0.1% TFA gradient to give the title compound 121 as a white solid (7.9 mg, 0.012 mmol, 42%). LC-MS: Calc'd m/z=458.2 for C₂₂H₃₀N₆O₅. found [M+H]⁺=459.2. ¹H NMR (300 MHz, MeOD) δ 7.19-7.13 (m, 1H), 7.12-6.96 (m, 2H), 5.05 (s, 2H), 4.40-4.17 (m, 4H), 3.96-3.85 (m, 1H), 2.10-1.93 (m, 1H), 1.72-1.53 (m, 1H), 1.53-1.38 (m, 1H), 1.34 (t, J=7.1 Hz, 3H), 1.06-0.92 (m, 6H).

### Example 1.35: 6-amino-2-ethoxy-9-(4-((((2S,3S)-1-hydroxy-3-methylpentan-2-yl)amino)-methyl)-2-methoxybenzyl)-9H-purin-8-ol (Compound 122)

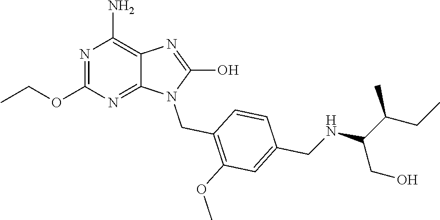

The titled compound was prepared according to General Procedure 1 from compound 11 (0.010 g, 0.027 mmol, 1.0 eq.) and (2R,3S)-2-aminopentane-1,3-diol (16 mg, 0.14 mmol, 5.0 eq.). Preparative HPLC purification was accomplished as described in General Procedure 5, eluting with a 10 to 45% CH₃CN+0.1% TFA/H₂O+0.1% TFA gradient to give the title compound 122 as a white solid (9.8 mg, 0.015 mmol, 53%). LC-MS: Calc'd m/z=444.2 for C₂₂H₃₂N₆O₄. found [M+H]⁺=445.3. ¹H NMR (300 MHz, MeOD) δ 7.18 (s, 1H), 7.12-6.98 (m, 2H), 5.04 (s, 2H), 4.41-4.22 (m, 4H), 3.94-3.81 (m, 1H), 3.75 (dd, J=12.1, 7.1 Hz, 1H), 3.19-3.08 (m, 1H), 1.94-1.80 (m, 1H), 1.54-1.18 (m, 5H), 0.98 (d, J=6.9 Hz, 3H), 0.90 (t, J=7.4 Hz, 3H).

### Example 1.36: 6-amino-9-(4-(aminomethyl)-2-methoxybenzyl)-2-ethoxy-9H-purin-8-ol (Compound 123)

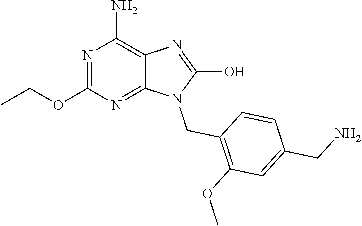 [SMILES: CCOc1nc(N)c2nc(O)n(Cc3ccc(CN)cc3OC)c2n1]

The titled compound was prepared according to General Procedure 1 from compound 11 (0.010 g, 0.027 mmol, 1.0 eq.) and ammonia/DMF (15 μL, 2 M, 5.0 eq.). Preparative HPLC purification was accomplished as described in General Procedure 5, eluting with a 10 to 45% CH₃CN+0.1% TFA/H₂O+0.1% TFA gradient to give the title compound 123 as a white solid (1.7 mg, 0.0030 mmol, 11%). LC-MS: Calc'd m/z=344.2 for C₆H₂₀N₆O₃. found [M+H]⁺=345.2. ¹H NMR (300 MHz, MeOD) δ 7.13-6.99 (m, 2H), 6.95 (d, J=7.5 Hz, 1H), 5.04 (s, 2H), 4.30 (q, J=7.1 Hz, 2H), 4.10 (s, 2H), 3.93 (s, 3H), 1.33 (t, J=7.0 Hz, 3H).

### Example 1.37: 6-amino-2-ethoxy-9-(4-((ethylamino)methyl)-2-methoxybenzyl)-9H-purin-8-ol (Compound 124)

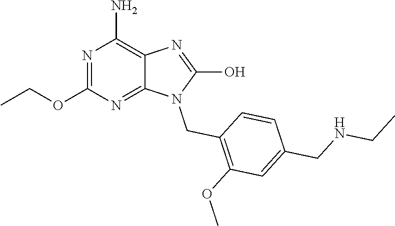 [SMILES: CCNCc1ccc(Cn2c(O)nc3c(N)nc(OCC)nc32)c(OC)c1]

The titled compound was prepared according to General Procedure 1 from compound 11 (0.010 g, 0.027 mmol, 1.0 eq.) and ethylamine (9.3 μL, 0.14 mmol, 5.0 eq.). Preparative HPLC purification was accomplished as described in General Procedure 5, eluting with a 10 to 45% CH₃CN+0.1% TFA/H₂O+0.1% TFA gradient to give the title compound 124 as a white solid (6.4 mg, 0.011 mmol, 39%). LC-MS: Calc'd m/z=372.2 for C₆H₂₀N₆O₃. found [M+H]⁺=373.2. ¹H NMR (300 MHz, MeOD) δ 7.17-7.04 (m, 2H), 7.00 (dd, J=7.7, 1.6 Hz, 1H), 5.05 (s, 2H), 4.34 (q, J=7.1 Hz, 2H), 4.18 (s, 2H), 3.93 (s, 3H), 3.12 (q, J=7.3 Hz, 2H), 1.34 (t, J=7.2 Hz, 6H).

### Example 1.38: 6-amino-9-(4-((dipropylamino)methyl)-2-methoxybenzyl)-2-ethoxy-9H-purin-8-ol (Compound 125)

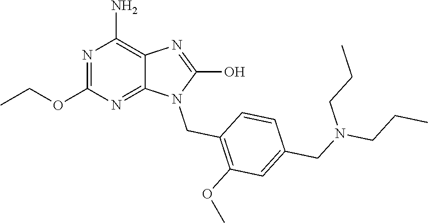 [SMILES: CCCN(CCC)Cc1ccc(Cn2c(O)nc3c(N)nc(OCC)nc32)c(OC)c1]

The titled compound was prepared according to General Procedure 1 from compound 11 (0.010 g, 0.027 mmol, 1.0 eq.) and dipropylamine (19 μL, 0.14 mmol, 5.0 eq.). Preparative HPLC purification was accomplished as described in General Procedure 5, eluting with a 10 to 45% CH₃CN+0.1% TFA/H₂O+0.1% TFA gradient to give the title compound 125 as a white solid (2.5 mg, 0.0038 mmol, 14%). LC-MS: Calc'd m/z=428.3 for C₂₂H₃₂N₆O₃. found [M+H]⁺=429.3. ¹H NMR (300 MHz, MeOD) δ 7.15 (s, 1H), 7.12-6.97 (m, 2H), 5.06 (s, 2H), 4.38-4.24 (m, 4H), 3.95 (s, 3H), 3.09 (t, J=8.5 Hz, 4H), 1.92-1.65 (m, 4H), 1.33 (t, J=7.1 Hz, 3H), 1.00 (t, J=7.3 Hz, 6H).

### Example 1.39: (E)-6-amino-9-(4-(((4-aminobut-2-en-1-yl)amino)methyl)-2-methoxybenzyl)-2-ethoxy-9H-purin-8-ol (Compound 126)

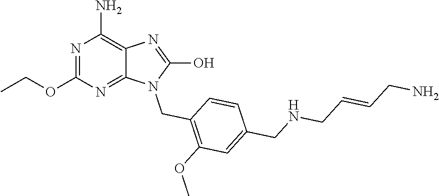 [SMILES: CCOc1nc(N)c2nc(O)n(Cc3ccc(CNC/C=C/CN)cc3OC)c2n1]

The titled compound was prepared according to General Procedure 1 from compound 11 (0.010 g, 0.028 mmol, 1.0 eq.) and tert-butyl N-[(2E)-4-aminobut-2-en-1-yl]carbamate (15 mg, 0.082 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 126 as a white solid (3.0 mg, 0.0070 mmol, 26%). LC-MS: Calc'd m/z=413.2 for C₂₀H₂₇N₇O₃. found [M+H]⁺=414.3. ¹H NMR (300 MHz, MeOD) δ 7.13 (d, J=1.6 Hz, 1H), 7.08-6.94 (m, 2H), 6.15-5.92 (m, 2H), 5.04 (s, 2H), 4.29 (q, J=7.1 Hz, 2H), 4.19 (s, 2H), 3.93 (s, 3H), 3.76 (d, J=5.8 Hz, 2H), 3.66 (d, J=5.4 Hz, 2H), 1.32 (t, J=7.1 Hz, 3H).

### Example 1.40: 6-amino-2-ethoxy-9-(2-methoxy-4-((piperidin-3-ylamino)methyl)benzyl)-9H-purin-8-ol (Compound 127)

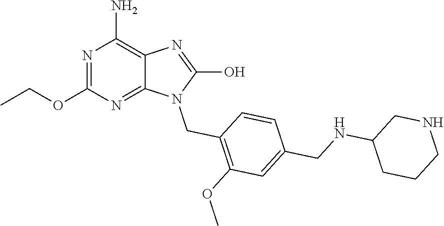 [SMILES: CCOc1nc(N)c2nc(O)n(Cc3ccc(CNC4CCCNC4)cc3OC)c2n1]

The titled compound was prepared according to General Procedure 1 from compound 11 (0.010 g, 0.028 mmol, 1.0 eq.) and tert-butyl-3-aminopiperidine-1-carboxylate (30 mg, 0.15 mmol, 5.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 127 as a white solid (3.8 mg, 0.0088 mmol, 63%). LC-MS: Calc'd m/z=427.2 for C₂₁H₂₉N₇O₃. found [M+H]⁺=428.2. ¹H NMR (300 MHz, MeOD) δ 7.18 (s, 1H), 7.10-6.97 (m, 2H), 5.04 (s, 2H), 4.38-4.22 (m, 4H), 3.94 (s, 3H), 3.92-3.70 (m, 2H), 3.62-3.38 (m, 2H), 3.15-2.91 (m, 2H), 2.45-2.35 (m, 1H), 2.21-2.10 (m, 1H), 1.88-1.55 (m, 1H), 1.33 (t, J=7.1 Hz, 3H).

### Example 1.41: 9-(4-(((1S,4S)-2,5-diazabicyclo[2.2.1]heptan-2-yl)methyl)-2-methoxybenzyl)-6-amino-2-ethoxy-9H-purin-8-ol (Compound 128)

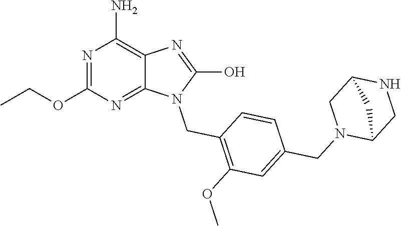 [SMILES: CCOc1nc(N)c2nc(O)n(Cc3ccc(CN4C[C@@H]5C[C@H]4CN5)cc3OC)c2n1]

The titled compound was prepared according to General Procedure 1 from compound 11 (0.010 g, 0.028 mmol, 1.0 eq.) and tert-butyl-(1S,4S)-2,5-diazabicyclo[2.2.1]heptane-2-carboxylate (27 mg, 0.15 mmol, 5.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 128 as a white solid (8.0 mg, 0.010 mmol, 71%). LC-MS: Calc'd m/z=425.2 for C₂₁H₂₇N₇O₃. found [M+H]⁺=426.3. ¹H NMR (300 MHz, MeOD) δ 7.20 (s, 1H), 7.10-6.98 (m, 2H), 5.04 (s, 2H), 4.52 (s, 1H), 4.42-4.27 (m, 4H), 4.24 (d, J=13.0 Hz, 1H), 3.93 (s, 3H), 3.75 (d, J=12.8 Hz, 1H), 3.54-3.35 (m, 3H), 2.58 (d, J=12.7 Hz, 1H), 2.16 (d, J=12.6 Hz, 1H), 1.34 (t, J=7.1 Hz, 3H).

### Example 1.42: 6-amino-9-(4-((azetidin-3-ylamino)methyl)-2-methoxybenzyl)-2-ethoxy-9H-purin-8-ol (Compound 129)

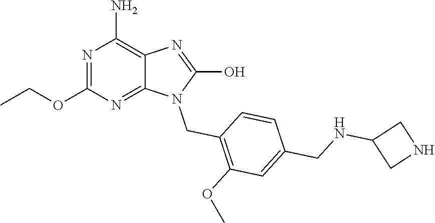 [SMILES: CCOc1nc(N)c2nc(O)n(Cc3ccc(CNC4CNC4)cc3OC)c2n1]

The titled compound was prepared according to General Procedure 1 from compound 11 (0.010 g, 0.028 mmol, 1.0 eq.) and tert-butyl 3-aminoazetidine-1-carboxylate (20 mg, 0.09 mmol, 3 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 129 as a white solid (7.0 mg, 0.0094 mmol, 67%). LC-MS: Calc'd m/z=399.2 for C₁₉H₂₅N₇O₃. found [M+H]⁺=400.3. ¹H NMR (300 MHz, MeOD) δ 7.16 (s, 1H), 7.04 (d, J=7.7 Hz, 1H), 7.01-6.93 (m, 1H), 5.04 (s, 2H), 4.37-4.20 (m, 6H), 4.15 (s, 2H), 3.94 (s, 3H), 1.44-1.28 (m, 4H).

### Example 1.43: 9-(4-(((2-azaspiro[3.3]heptan-6-yl)amino)methyl)-2-methoxybenzyl)-6-amino-2-ethoxy-9H-purin-8-ol (Compound 130)

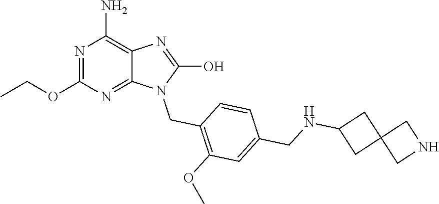 [SMILES: CCOc1nc(N)c2nc(O)n(Cc3ccc(CNC4CC5(CNC5)C4)cc3OC)c2n1]

The titled compound was prepared according to General Procedure 1 from compound 11 (0.010 g, 0.028 mmol, 1.0 eq.) and tert-butyl 6-amino-2-azaspiro[3.3]heptane-2-carboxylate (20 mg, 0.09 mmol, 3 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 130 as a white solid (7.9 mg, 0.010 mmol, 72%). LC-MS: Calc'd m/z=439.2 for C₂₂H₂₉N₇O₃. found [M+H]⁺=440.3. ¹H NMR (300 MHz, MeOD) δ 7.25-7.02 (m, 2H), 6.97 (dd, J=7.7, 1.6 Hz, 1H), 5.05 (s, 2H), 4.34 (q, J=7.1 Hz, 2H), 4.17 (s, 2H), 4.09 (d, J=3.2 Hz, 4H), 3.93 (s, 3H), 3.78-3.65 (m, 1H), 2.81-2.62 (m, 2H), 2.58-2.45 (m, 2H), 1.35 (t, J=7.1 Hz, 3H).

### Example 1.44: 6-amino-2-ethoxy-9-(2-methoxy-4-(((3-(piperazin-1-yl)propyl)amino)methyl)-benzyl)-9H-purin-8-ol (Compound 131)

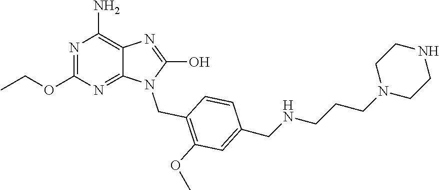 [SMILES: CCOc1nc(N)c2nc(O)n(Cc3ccc(CNCCCN4CCNCC4)cc3OC)c2n1]

The titled compound was prepared according to General Procedure 1 from compound 11 (0.010 g, 0.028 mmol, 1.0 eq.) and tert-butyl 4-(3-aminopropyl)piperazine-1-carboxylate (20 mg, 0.09 mmol, 3 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 131 as a white solid (0.010 g, 0.011 mmol, 72%). LC-MS: Calc'd m/z=470.3 for C₂₃H₃₄N₈O₃. found [M+H]⁺=471.4. ¹H NMR (300 MHz, MeOD) δ 7.28-6.97 (m, 3H), 5.06 (s, 2H), 4.45-4.28 (m, 2H), 4.22 (s, 2H), 3.93 (s, 3H), 3.32-3.23 (m, 4H), 3.27-3.10 (m, 2H), 2.97-2.82 (m, 4H), 2.71 (t, J=6.8 Hz, 2H), 2.08-1.89 (m, 2H), 1.36 (t, J=7.0 Hz, 3H).

### Example 1.45: 6-amino-9-(4-((4-(aminomethyl)piperidin-1-yl)methyl)-2-methoxybenzyl)-2-ethoxy-9H-purin-8-ol (Compound 132)

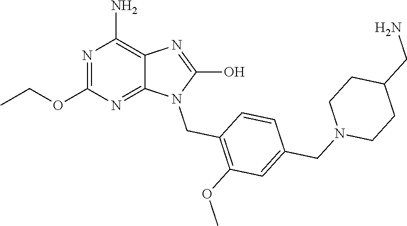 [SMILES: CCOc1nc(N)c2nc(O)n(Cc3ccc(CN4CCC(CN)CC4)cc3OC)c2n1]

The titled compound was prepared according to General Procedure 1 from compound 11 (0.010 g, 0.028 mmol, 1.0 eq.) and tert-butyl N-(piperidin-4-ylmethyl)carbamate (20 mg, 0.09 mmol, 3 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 132 as a white solid (7.9 mg, 0.010 mmol, 72%). LC-MS: Calc'd m/z=441.3 for C₂₂H₃₁N₇O₃. found [M+H]⁺=442.4. ¹H NMR (300 MHz, MeOD) δ 7.20-6.97 (m, 3H), 5.06 (s, 2H), 4.41-4.28 (m, 4H), 3.94 (s, 3H), 3.57 (d, J=12.4 Hz, 2H), 3.11-2.96 (m, 2H), 2.91 (d, J=6.7 Hz, 2H), 2.25-1.86 (m, 3H), 1.65-1.51 (m, 2H), 1.34 (t, J=7.1 Hz, 3H).

### Example 1.46: (S)-6-amino-2-ethoxy-9-(2-methoxy-4-((pyrrolidin-3-ylamino)methyl)benzyl)-9H-purin-8-ol (Compound 133)

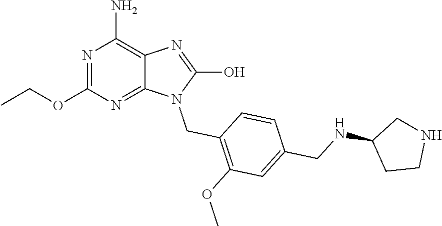 [SMILES: CCOc1nc(N)c2nc(O)n(Cc3ccc(CN[C@@H]4CCNC4)cc3OC)c2n1]

The titled compound was prepared according to General Procedure 1 from compound 11 (0.010 g, 0.028 mmol, 1.0 eq.) and tert-butyl (3R)-3-aminopyrrolidine-1-carboxylate carbamate (20 mg, 0.09 mmol, 3 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 133 as a white solid (0.010 g, 0.013 mmol, 93%). LC-MS: Calc'd m/z=413.2 for C₂₀H₂₇N₇O₃. found [M+H]⁺=414.3. ¹H NMR (300 MHz, MeOD) δ 7.21 (s, 1H), 7.15-6.99 (m, 2H), 5.06 (s, 2H), 4.41-4.27 (m, 4H), 4.14 (p, J=7.1 Hz, 1H), 3.94 (s, 3H), 3.81-3.68 (m, 1H), 3.67-3.48 (m, 2H), 3.48-3.33 (m, 1H), 2.68-2.50 (m, 1H), 2.39-2.21 (m, 1H), 1.35 (t, J=7.1 Hz, 3H).

### Example 1.47: 6-amino-2-ethoxy-9-(2-methoxy-4-(((2-(piperazin-1-yl)ethyl)amino)methyl)-benzyl)-9H-purin-8-ol (Compound 134

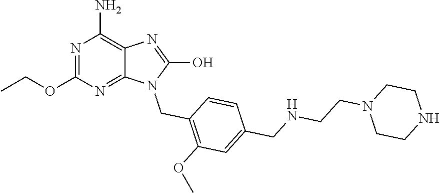 [SMILES: CCOc1nc(N)c2nc(O)n(Cc3ccc(CNCCN4CCNCC4)cc3OC)c2n1]

The titled compound was prepared according to General Procedure 1 from compound 11 (0.010 g, 0.028 mmol, 1.0 eq.) and tert-butyl 4-(2-aminoethyl)piperazine-1-carboxylate (20 mg, 0.09 mmol, 3 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 134 as a white solid (3.6 mg, 0.0039 mmol, 28%). LC-MS: Calc'd m/z=456.3 for C₂₂H₃₂N₈O₃. found [M+H]⁺=457.3. ¹H NMR (300 MHz, MeOD) δ 7.18 (d, J=1.5 Hz, 1H), 7.14-6.98 (m, 2H), 5.06 (s, 2H), 4.35 (q, J=7.1 Hz, 2H), 4.25 (s, 2H), 3.94 (s, 3H), 3.32-3.14 (m, 6H), 2.79-2.66 (m, 6H), 1.35 (t, J=7.1 Hz, 3H).

### Example 1.48: 6-amino-2-ethoxy-9-(2-methoxy-4-(((2-morpholinoethyl)amino)methyl)benzyl)-9H-purin-8-ol (Compound 135)

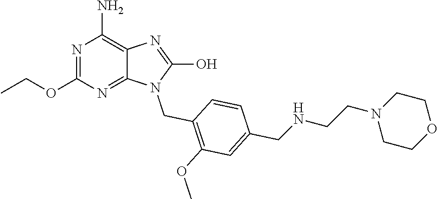 [SMILES: CCOc1nc(N)c2nc(O)n(Cc3ccc(CNCCN4CCOCC4)cc3OC)c2n1]

The titled compound was prepared according to General Procedure 1 from compound 11 (0.010 g, 0.028 mmol, 1.0 eq.) and N-aminoethylmorpholine (0.010 g, 0.09 mmol, 3 eq.). Preparative HPLC purification was accomplished as described in General Procedure 5, eluting with a 10 to 45% CH₃CN+0.1% TFA/H₂O+0.1% TFA gradient to give the title compound 135 as a white solid (3.1 mg, 0.0039 mmol, 28%). LC-MS: Calc'd m/z=457.3 for C₂₂H₃₁N₇O₄. found [M+H]⁺=458.4. ¹H NMR (300 MHz, MeOD) δ 7.16 (d, J=1.5 Hz, 1H), 7.09 (d, J=7.7 Hz, 1H), 7.02 (dd, J=7.7, 1.6 Hz, 1H), 5.05 (s, 2H), 4.32 (q, J=7.1 Hz, 2H), 4.26 (s, 2H), 3.94 (s, 3H), 3.85-3.76 (m, 4H), 3.04 (t, J=6.4 Hz, 2H), 2.90-2.84 (m, 4H), 1.48 (s, 2H), 1.34 (t, J=7.1 Hz, 3H).

### Example 1.49: 6-amino-2-ethoxy-9-(4-(((2-hydroxy-3-morpholinopropyl)amino)methyl)-2-methoxybenzyl)-9H-purin-8-ol (Compound 136)

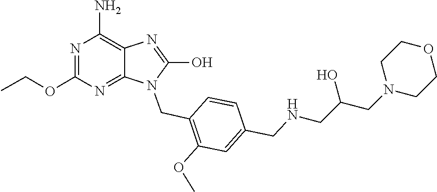 [SMILES: CCOc1nc(N)c2nc(O)n(Cc3ccc(CNCC(O)CN4CCOCC4)cc3OC)c2n1]

The titled compound was prepared according to General Procedure 1 from compound 11 (0.010 g, 0.028 mmol, 1.0 eq.) and 1-amino-3-(morpholin-4-yl)propan-2-ol (20 mg, 0.09 mmol, 3 eq.). Preparative HPLC purification was accomplished as described in General Procedure 5, eluting with a 10 to 45% CH₃CN+0.1% TFA/H₂O+0.1% TFA gradient to give the title compound 136 as a white solid (2.9 mg, 0.0036 mmol, 25%). LC-MS: Calc'd m/z=487.3 for C₂₃H₃₃N₇O₅. found [M+H]⁺=488.4. ¹H NMR (300 MHz, MeOD) δ 7.19 (d, J=1.6 Hz, 1H), 7.12-6.97 (m, 2H), 5.05 (s, 2H), 4.50-4.41 (m, 1H), 4.34 (q, J=7.1 Hz, 2H), 4.27 (s, 2H), 4.03-3.85 (m, 4H), 3.94 (s, 3H), 3.41-3.35 (m, 4H), 3.28-3.10 (m, 3H), 3.04 (dd, J=12.9, 9.1 Hz, 1H), 1.35 (t, J=7.1 Hz, 3H).

### Example 1.50: 6-amino-2-ethoxy-9-(2-methoxy-4-(((2-thiomorpholinoethyl)amino)methyl)-benzyl)-9H-purin-8-ol (Compound 137)

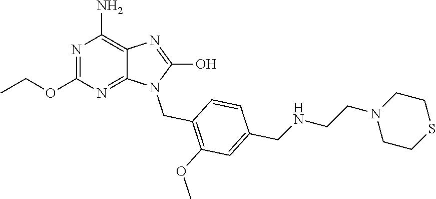 [SMILES: CCOc1nc(N)c2nc(O)n(Cc3ccc(CNCCN4CCSCC4)cc3OC)c2n1]

The titled compound was prepared according to General Procedure 1 from compound 11 (0.010 g, 0.028 mmol, 1.0 eq.) and 2-(thiomorpholin-4-yl)ethanamine (0.010 g, 0.09 mmol, 3 eq.). Preparative HPLC purification was accomplished as described in General Procedure 5, eluting with a 10 to 45% CH₃CN+0.1% TFA/H₂O+0.1% TFA gradient to give the title compound 137 as a white solid (3.5 mg, 0.0043 mmol, 31%). LC-MS: Calc'd m/z=473.2 for C₂₂H₃₁N₇O₃S. found [M+H]⁺=473.3. ¹H NMR (300 MHz, MeOD) δ 7.19-6.97 (m, 3H), 5.05 (s, 2H), 4.39-4.22 (m, 4H), 3.94 (s, 3H), 3.36-3.30 (m, 2H), 3.15-2.98 (m, 6H), 2.83 (q, J=4.6 Hz, 4H), 1.34 (t, J=7.1 Hz, 3H).

### Example 1.51: 9-(4-([4,4′-bipiperidin]-1-ylmethyl)-2-methoxybenzyl)-6-amino-2-ethoxy-9H-purin-8-ol (Compound 138)

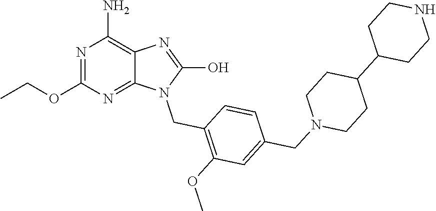 [SMILES: CCOc1nc(N)c2nc(O)n(Cc3ccc(CN4CCC(C5CCNCC5)CC4)cc3OC)c2n1]

The titled compound was prepared according to General Procedure 1 from compound 11 (12 mg, 0.033 mmol, 1.0 eq.) and tert-butyl [4,4′-bipiperidine]-1-carboxylate (35 mg, 0.13 mmol, 4.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 138 as a white solid (8.0 mg, 0.0096 mmol, 58%). LC-MS: Calc'd m/z=495.3 for C₂₆H₃₇N₇O₃. found [M+H]⁺=496.4. ¹H NMR (300 MHz, MeOD) δ 7.16 (d, J=1.5 Hz, 1H), 7.12-6.96 (m, 2H), 5.05 (s, 2H), 4.43-4.24 (m, 4H), 3.93 (s, 3H), 3.53 (d, J=12.1 Hz, 2H), 3.43 (d, J=12.6 Hz, 2H), 2.95 (q, J=10.1 Hz, 4H), 2.00 (t, J=12.4 Hz, 4H), 1.58-1.44 (m, 6H), 1.33 (t, J=7.1 Hz, 3H).

### Example 1.52: 9-(4-((3,9-diazaspiro[5.5]undecan-3-yl)methyl)-2-methoxybenzyl)-6-amino-2-ethoxy-9H-purin-8-ol (Compound 139)

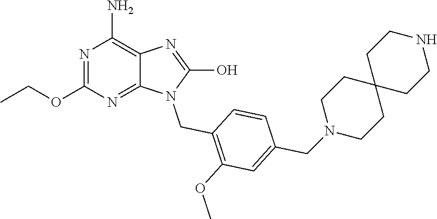 [SMILES: CCOc1nc(N)c2nc(O)n(Cc3ccc(CN4CCC5(CCNCC5)CC4)cc3OC)c2n1]

The titled compound was prepared according to General Procedure 1 from compound 11 (12 mg, 0.033 mmol, 1.0 eq.) and tert-butyl 3,9-diazaspiro[5.5]undecane-3-carboxylate (33 mg, 0.13 mmol, 4.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 139 as a white solid (7.7 mg, 0.0093 mmol, 56%). LC-MS: Calc'd m/z=481.3 for C₂₅H₃₅N₇O₃. found [M+H]⁺=482.4. ¹H NMR (300 MHz, MeOD) δ 7.16 (d, J=1.5 Hz, 1H), 7.12-6.96 (m, 2H), 5.05 (s, 2H), 4.38-4.25 (m, 4H), 3.94 (s, 3H), 3.38 (d, J=12.7 Hz, 2H), 3.24-3.18 (m, 6H), 2.08-1.96 (m, 2H), 1.96-1.90 (m, 2H), 1.72-1.66 (m, 4H), 1.33 (t, J=7.1 Hz, 3H).

### Example 1.53: 6-amino-9-(4-((4-(2-aminoethyl)piperidin-1-yl)methyl)-2-methoxybenzyl)-2-ethoxy-9H-purin-8-ol (Compound 140)

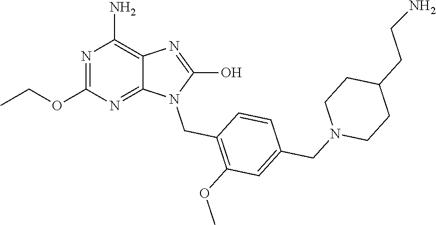 [SMILES: CCOc1nc(N)c2nc(O)n(Cc3ccc(CN4CCC(CCN)CC4)cc3OC)c2n1]

The titled compound was prepared according to General Procedure 1 from compound 11 (30 mg, 0.083 mmol, 1.0 eq.) and tert-butyl N-[2-(piperidin-4-yl)ethyl]carbamate (94 mg, 0.41 mmol, 5.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 140 as a white solid (7.7 mg, 0.0097 mmol, 23%). LC-MS: Calc'd m/z=455.3 for C₂₃H₃₃N₇O₃. found [M+H]⁺=456.3. ¹H NMR (300 MHz, MeOD) δ 7.17 (d, J=1.5 Hz, 1H), 7.09 (d, J=7.7 Hz, 1H), 7.01 (dd, J=7.7, 1.5 Hz, 1H), 5.06 (s, 2H), 4.40-4.26 (m, 4H), 3.94 (s, 3H), 3.51 (d, J=12.6 Hz, 2H), 2.97 (d, J=7.7 Hz, 5H), 2.08-1.94 (m, 2H), 1.64 (dd, J=14.4, 6.6 Hz, 2H), 1.54-1.40 (m, 2H), 1.34 (t, J=7.1 Hz, 3H).

### Example 1.54: 6-amino-2-ethoxy-9-(4-(((4-(hydroxymethyl)benzyl)amino)methyl)-2-methoxy-benzyl)-9H-purin-8-ol

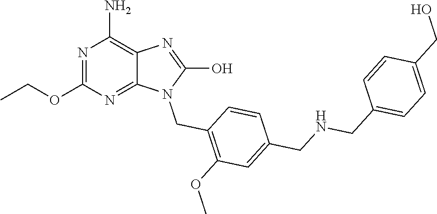 [SMILES: CCOc1nc(N)c2nc(O)n(Cc3ccc(CNCc4ccc(CO)cc4)cc3OC)c2n1]

The title compound 12 was prepared according to General Procedure 1 from compound 11 (80 mg, 0.22 mmol, 1.0 eq.) and (4-(aminomethyl)phenyl)methanol (90 mg, 0.7 mmol, 3 eq.). Preparative HPLC purification was accomplished as described in General Procedure 5, eluting with a 0 to 40% CH₃CN+0.1% TFA/H₂O+0.1% TFA gradient to give the titled compound as a white solid (75 mg, 0.16 mmol, 73%). LC-MS: Calc'd m/z=464.2 for C₂₄H₂₈N₆O₄. found [M+H]+=465.3.

### Example 1.55: 6-amino-9-(4-(((4-(chloromethyl)benzyl)amino)methyl)-2-methoxybenzyl)-2-ethoxy-9H-purin-8-ol

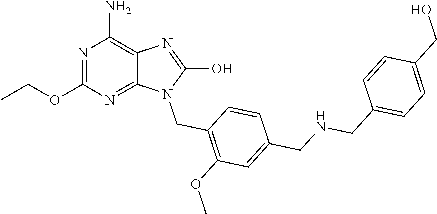 [SMILES: CCOc1nc(N)c2nc(O)n(Cc3ccc(CNCc4ccc(CO)cc4)cc3OC)c2n1]

The title compound 13 was prepared according to General Procedure 3 from compound 12 (75 mg, 0.16 mmol, 1.0 eq.) and 10% SOCl₂/DCM (4 mL). The titled compound was obtained as a yellow solid (65 mg, 0.14 mmol, 83%). LC-MS: Calc'd m/z=482.2 for C₂₄H₂₇ClN₆O₃. found [M+H]⁺=483.3.

### Example 1.56: 6-amino-9-(4-(((4-((dipropylamino)methyl)benzyl)amino)methyl)-2-methoxy-benzyl)-2-ethoxy-9H-purin-8-ol (Compound 141

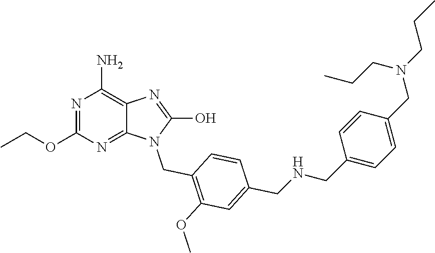 [SMILES: CCCN(CCC)Cc1ccc(CNCc2ccc(Cn3c(O)nc4c(N)nc(OCC)nc43)c(OC)c2)cc1]

The titled compound was prepared according to General Procedure 1 from compound 13 (8.0 mg, 0.017 mmol, 1.0 eq.) and dipropylamine (11 μL, 0.083 mmol, 5.0 eq.). Preparative HPLC purification was accomplished as described in General Procedure 5, eluting with a 10 to 45% CH₃CN+0.1% TFA/H₂O+0.1% TFA gradient to give the title compound 141 as a white solid (2.7 mg, 0.0030 mmol, 18%). LC-MS: Calc'd m/z=547.3 for C₃₀H₄₁N₇O₃. found [M+H]⁺=548.3. ¹H NMR (300 MHz, MeOD) δ 7.63 (s, 4H), 7.15 (d, J=1.3 Hz, 1H), 7.02 (q, J=7.8 Hz, 2H), 5.04 (s, 2H), 4.42 (s, 2H), 4.36-4.22 (m, 6H), 3.93 (s, 3H), 3.15-2.98 (m, 4H), 1.85-1.71 (m, 4H), 1.32 (t, J=7.1 Hz, 3H), 0.99 (t, J=7.3 Hz, 6H).

### Example 1.57: 6-amino-2-ethoxy-9-(2-methoxy-4-(((4-(morpholinomethyl)benzyl)amino)-methyl)benzyl)-9H-purin-8-ol (Compound 142)

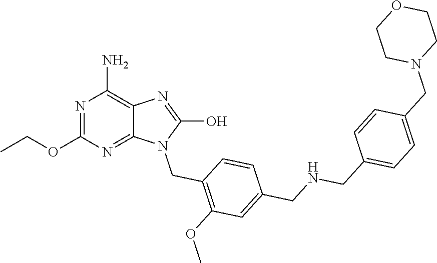 [SMILES: CCOc1nc(N)c2nc(O)n(Cc3ccc(CNCc4ccc(CN5CCOCC5)cc4)cc3OC)c2n1]

The titled compound was prepared according to General Procedure 1 from compound 13 (8.0 mg, 0.017 mmol, 1.0 eq.) and morpholine (7.2 μL, 0.083 mmol, 5.0 eq.). Preparative HPLC purification was accomplished as described in General Procedure 5, eluting with a 10 to 45% CH₃CN+0.1% TFA/H₂O+0.1% TFA gradient to give the title compound 142 as a white solid (4.1 mg, 0.0047 mmol, 28%). LC-MS: Calc'd m/z=533.3 for C₂₈H₃₅N₇O₄. found [M+H]⁺=534.3. ¹H NMR (300 MHz, MeOD) δ 7.62 (s, 4H), 7.14 (d, J=1.5 Hz, 1H), 7.11-6.95 (m, 2H), 5.04 (s, 2H), 4.42 (s, 2H), 4.39-4.23 (m, 6H), 3.92 (s, 3H), 4.14-3.67 (m, 8H), 1.33 (t, J=7.1 Hz, 3H).

### Example 1.58: tert-butyl-(1-(4-(((4-((6-amino-2-ethoxy-8-hydroxy-9H-purin-9-yl)methyl)-3-methoxybenzyl) amino)methyl)benzyl)piperidin-4-yl)carbamate (Compound 143)

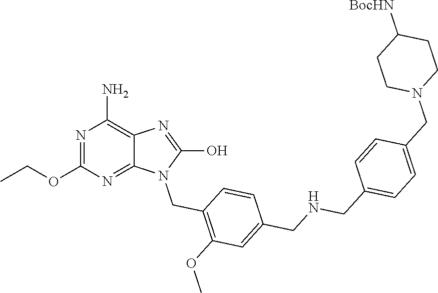 [SMILES: CCOc1nc(N)c2nc(O)n(Cc3ccc(CNCc4ccc(CN5CCC(NC(=O)OC(C)(C)C)CC5)cc4)cc3OC)c2n1]

The titled compound was prepared according to General Procedure 1 from compound 13 (8.0 mg, 0.017 mmol, 1.0 eq.) and tert-butyl-N-(piperidin-4-yl)carbamate (17 mg, 0.083 mmol, 5.0 eq.). Preparative HPLC purification was accomplished as described in General Procedure 5, eluting with a 10 to 55% CH₃CN+0.1% TFA/H₂O+0.1% TFA gradient to give the title compound 143 as a white solid (8.6 mg, 0.0083 mmol, 49%). LC-MS: Calc'd m/z=646.4 for C₃₄H₄₆N₈O₅. found [M+H]⁺=647.5. ¹H NMR (300 MHz, MeOD) δ 7.61 (s, 4H), 7.14 (s, 1H), 7.09-6.95 (m, 2H), 5.04 (s, 2H), 4.39-4.23 (m, 8H), 3.92 (s, 3H), 3.63-3.57 (m, 1H), 3.56-3.46 (m, 2H), 3.18-3.04 (m, 2H), 2.20-2.09 (m, 2H), 1.77-1.63 (m, 2H), 1.45 (s, 9H), 1.33 (t, J=7.1 Hz, 3H).

### Example 1.59: 6-amino-9-(4-(((4-((4-aminopiperidin-1-yl)methyl)benzyl)amino)methyl)-2-methoxybenzyl)-2-ethoxy-9H-purin-8-ol (Compound 144)

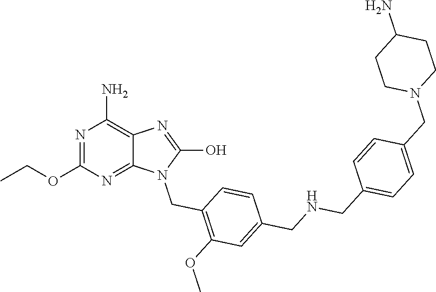 [SMILES: CCOc1nc(N)c2nc(O)n(Cc3ccc(CNCc4ccc(CN5CCC(N)CC5)cc4)cc3OC)c2n1]

Compound 143 (4.5 mg, 0.0045 mmol, 1.0 eq.) was deprotected according to General Procedure 2 to give the title compound 144 as a white solid (4.3 mg, 0.0043 mmol, 96%). LC-MS: Calc'd m/z=546.3 for C₂₉H₃₈N₈O₃. found [M+H]⁺=547.4. ¹H NMR (300 MHz, MeOD) δ 7.60 (s, 4H), 7.14 (s, 1H), 7.02 (q, J=7.8 Hz, 2H), 5.04 (s, 2H), 4.41-4.20 (m, 9H), 3.92 (s, 3H), 3.59 (d, J=12.7 Hz, 2H), 3.29-3.10 (m, 2H), 2.26 (d, J=13.9 Hz, 2H), 2.08-1.93 (m, 2H), 1.32 (t, J=7.1 Hz, 3H).

### Example 1.60: tert-butyl 4-(4-(((4-((6-amino-2-ethoxy-8-hydroxy-9H-purin-9-yl)methyl)-3-methoxybenzyl)amino)methyl)benzyl)piperazine-1-carboxylate

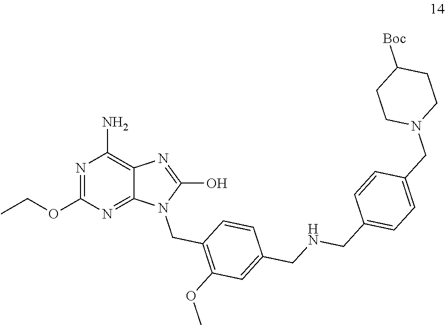 [SMILES: CCOc1nc(N)c2nc(O)n(Cc3ccc(CNCc4ccc(CN5CCC(C(=O)OC(C)(C)C)CC5)cc4)cc3OC)c2n1]

The title compound 14 was prepared according to General Procedure 1 from compound 11 (8 mg, 0.017 mmol, 1.0 eq.) and tert-butyl piperazine-1-carboxylate (15 mg, 0.083 mmol, 5.0 eq.). Preparative HPLC purification was accomplished as described in General Procedure 5, eluting with a 10 to 55% CH₃CN+0.1% TFA/H₂O+0.1% TFA gradient to give the titled compound as a white solid (9.0 mg, 0.0092 mmol, 54%). LC-MS: Calc'd m/z=632.3 for C₃₃H₄₄N₈O₅. found [M+H]⁺=633.4. ¹H NMR (300 MHz, MeOD) δ 7.62 (s, 4H), 7.17-7.11 (m, 1H), 7.10-6.95 (m, 2H), 5.04 (s, 2H), 4.41 (s, 2H), 4.38-4.23 (m, 6H), 3.92 (s, 3H), 3.72-3.66 (m, 4H), 3.30-3.24 (m, 4H), 1.49 (s, 9H), 1.33 (t, J=7.0 Hz, 3H).

### Example 1.61: 6-amino-2-ethoxy-9-(2-methoxy-4-(((4-(piperazin-1-ylmethyl)benzyl)amino)-methyl)benzyl)-9H-purin-8-ol (Compound 145)

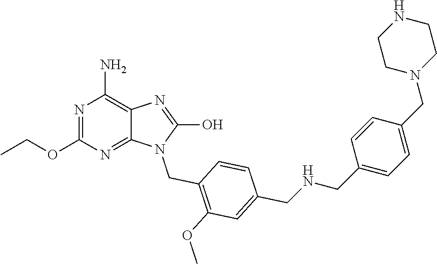 [SMILES: CCOc1nc(N)c2nc(O)n(Cc3ccc(CNCc4ccc(CN5CCNCC5)cc4)cc3OC)c2n1]

Compound 14 (5.0 mg, 0.0051 mmol, 1.0 eq.) was deprotected according to General Procedure 2 to give the title compound 145 as a white solid (4.8 mg, 0.0049 mmol, 95%). LC-MS: Calc'd m/z=532.3 for C₂₈H₃₆N₈O₃. found [M+H]⁺=533.4. ¹H NMR (300 MHz, MeOD) δ 7.49 (s, 4H), 7.16-6.95 (m, 3H), 5.04 (s, 2H), 4.32 (q, J=7.1 Hz, 2H), 4.26-4.20 (m, 4H), 3.92 (s, 3H), 3.76 (s, 2H), 3.33-3.24 (m, 4H), 2.86-2.76 (m, 4H), 1.32 (t, J=6.7 Hz, 3H).

### Example 1.62: 6-amino-9-(4-(((4-(((4-(aminomethyl)benzyl)amino)methyl)benzyl)amino)-methyl)-2-methoxybenzyl)-2-ethoxy-9H-purin-8-ol (Compound 146)

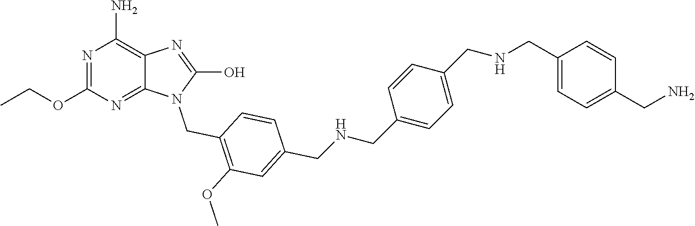 [SMILES: CCOc1nc(N)c2nc(O)n(Cc3ccc(CNCc4ccc(CNCc5ccc(CN)cc5)cc4)cc3OC)c2n1]

The titled compound was prepared according to General Procedure 1 from compound 13 (8.0 mg, 0.017 mmol, 1.0 eq.) and tert-butyl (4-(aminomethyl)benzyl)carbamate (20 mg, 0.083 mmol, 5.0 eq.). Preparative HPLC purification was accomplished as described in General Procedure 5, eluting with a 10 to 55% CH₃CN+0.1% TFA/H₂O+0.1% TFA gradient. Half of the resulting Boc intermediate was reserved and deprotection of the remaining material was accomplished according to General Procedure 2 to give the title compound 146 as a white solid (5.4 mg, 0.0052 mmol, 61%). LC-MS: Calc'd m/z=582.3 for C₃₂H₃₈N₈O₃. found [M+H]⁺=583.4. ¹H NMR (300 MHz, MeOD) δ 7.58 (d, J=4.0 Hz, 8H), 7.13 (s, 1H), 7.07-6.94 (m, 2H), 5.03 (s, 2H), 4.36-4.21 (m, 10H), 4.18 (s, 2H), 3.92 (s, 3H), 1.32 (t, J=7.1 Hz, 3H).

### Example 1.63: 6-amino-9-(4-(((4-((benzylamino)methyl)benzyl)amino)methyl)-2-methoxy-benzyl)-2-ethoxy-9H-purin-8-ol (Compound 147)

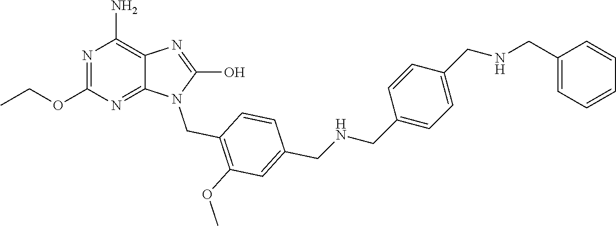 [SMILES: CCOc1nc(N)c2nc(O)n(Cc3ccc(CNCc4ccc(CNCc5ccccc5)cc4)cc3OC)c2n1]

The titled compound was prepared according to General Procedure 1 from compound 13 (8.0 mg, 0.017 mmol, 1.0 eq.) and benzylamine (9.1 μL, 0.083 mmol, 5.0 eq.). Preparative HPLC purification was accomplished as described in General Procedure 5, eluting with a 10 to 55% CH₃CN+0.1% TFA/H₂O+0.1% TFA gradient to give the title compound 147 as a white solid (2.7 mg, 0.0030 mmol, 18%). LC-MS: Calc'd m/z=553.3 for C₃₁H₃₅N₇O₃. found [M+H]⁺=554.4. ¹H NMR (300 MHz, MeOD) δ 7.59 (s, 4H), 7.49 (s, 5H), 7.13 (d, J=1.3 Hz, 1H), 7.08-6.94 (m, 2H), 5.03 (s, 2H), 4.37-4.22 (m, 10H), 3.92 (s, 3H), 1.32 (t, J=7.1 Hz, 3H).

### Example 1.64: 2,2′-((4-(((4-((6-amino-2-ethoxy-8-hydroxy-9H-purin-9-yl)methyl)-3-methoxy-benzyl)amino) methyl)benzyl)azanediyl)diacetic Acid (Compound 151)

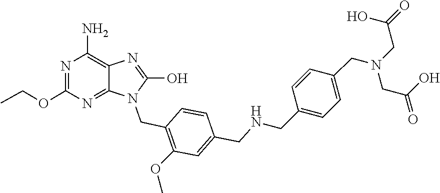 [SMILES: CCOc1nc(N)c2nc(O)n(Cc3ccc(CNCc4ccc(CN(CC(=O)O)CC(=O)O)cc4)cc3OC)c2n1]

To a solution of iminodiacetic acid (14 mg, 0.10 mmol, 5.0 eq.) in aqueous NaHCO₃ (0.272 mL, 1.14 M, 15.0 eq.) was added a solution of compound 11 (0.010 g, 0.021 mmol, 1.0 eq.) in 100 μL DMF. The resulting solution was heated to 60° C. for 18 h. Preparative HPLC purification was accomplished as described in General Procedure 5, eluting with a 10 to 45% CH₃CN+0.1% TFA/H₂O+0.1% TFA gradient to give the title compound 151 as a white solid (1.5 mg, 0.0026 mmol, 26%). LC-MS: Calc'd m/z=579.2 for C₂₈H₃₃N₇O₇. found [M+H]⁺=580.3. ¹H NMR (300 MHz, MeOD) δ 7.63-7.47 (m, 4H), 7.13 (s, 1H), 7.10-6.95 (m, 2H), 5.04 (s, 2H), 4.37-4.21 (m, 8H), 3.93 (s, 3H), 3.79 (s, 4H), 1.33 (t, J=7.1 Hz, 3H).

### Example 1.65: 2-(((S)-2-amino-2-carboxyethyl)thio)-4-((6-(4-(4-((6-amino-2-ethoxy-8-hydroxy-9H-purin-9-yl)methyl)-3-methoxybenzyl)piperazin-1-yl)-6-oxohexyl)amino)-4-oxobutanoic Acid (Compound 148)

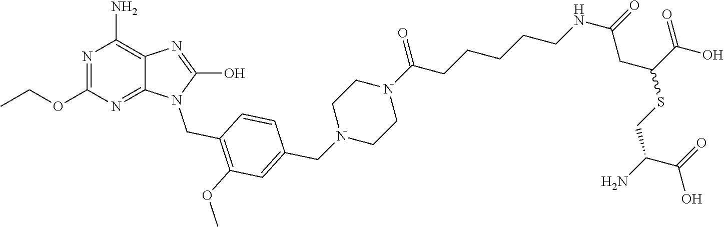 [SMILES: CCOc1nc(N)c2nc(O)n(Cc3ccc(CN4CCN(C(=O)CCCCCNC(=O)CC(SC[C@@H](N)C(=O)O)C(=O)O)CC4)cc3OC)c2n1]

To a solution of compound 100 (0.020 g, 0.048 mmol, 1.0 eq.) in DMF (500 μL) was added 2,5-dioxopyrrolidin-1-yl 6-(2,5-dioxopyrrol-1-yl)hexanoate (16 mg, 0.053 mmol, 1.1 eq.) then DIPEA (42 μL, 0.24 mmol, 5.0 eq.). The resulting solution was stirred at room temperature for 18 h, after which L-cysteine (12 mg, 0.096 mmol, 2.0 eq.) was added and the resulting solution was stirred at room temperature for 1 h. H₂O (200 μL) was then added and the reaction mixture stirred at room temperature for an additional 2 h. Preparative HPLC purification was accomplished as described in General Procedure 5, eluting with a 5 to 45% CH₃CN+0.1% TFA/H₂O+0.1% TFA gradient to give the title compound 148 as a white solid (5.1 mg, 0.0047 mmol, 9.8%). LC-MS: Calc'd m/z=745.3 for C₃₃H₄₇N₉O₉S. found [M+H]⁺=746.4.

### Example 1.66: (19S)-19-amino-1-(4-(4-((6-amino-2-ethoxy-8-hydroxy-9H-purin-9-yl)methyl)-3-methoxybenzyl)piperazin-1-yl)-16-carboxy-1,14-dioxo-4,7,10-trioxa-17-thia-13-azaicosan-20-oic Acid (Compound 149)

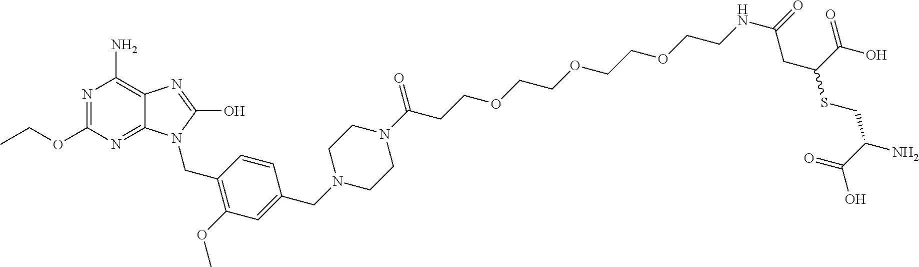 [SMILES: CCOc1nc(N)c2nc(O)n(Cc3ccc(CN4CCN(C(=O)CCOCCOCCOCCNC(=O)CC(SC[C@H](N)C(=O)O)C(=O)O)CC4)cc3OC)c2n1]

To a solution of compound 100 (0.020 g, 0.048 mmol, 1.0 eq.) in DMF (500 μL) was added compound 56 (16 mg, 0.053 mmol, 1.1 eq.) then DIPEA (42 μL, 0.24 mmol, 5.0 eq.). The resulting solution was stirred at room temperature for 18 h, after which L-cysteine (12 mg, 0.096 mmol, 2.0 eq.) was added and the resulting solution was stirred at room temperature for 1 h. H₂O (200 μL) was then added and the reaction mixture stirred at room temperature for an additional 2 h. Preparative HPLC purification was accomplished as described in General Procedure 5, eluting with a 5 to 45% CH₃CN+0.1% TFA/H₂O+0.1% TFA gradient to give the title compound 149 as a white solid (0.5 mg, 0.0004 mmol, 1%). LC-MS: Calc'd m/z=835.4 for C₃₆H₅₃N₉O₁₂S. found [M+H]⁺=836.5.

### Example 1.67: Methyl 4-((6-amino-2-ethoxy-8-methoxy-9H-purin-9-yl)methyl)benzoate

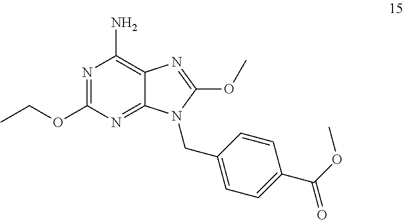 [SMILES: CCOc1nc(N)c2nc(OC)n(Cc3ccc(C(=O)OC)cc3)c2n1]

To a solution of compound 8 (0.75 g, 1.7 mmol, 1.0 eq.) and methyl 4-(bromomethyl)benzoate (0.39 g, 1.7 mmol, 1.0 eq.) in DMF (8 mL) was added CsCO₃ (0.56 g, 1.7 mmol, 1.0 eq.). The resulting suspension was stirred at room temperature for 18 h then concentrated in vacuo. Reverse phase flash purification was accomplished as described in General Procedure 5 using a 60 g C18 column, eluting with a 10 to 50% CH₃CN+0.1% TFA/H₂O+0.1% TFA gradient. The title compound 15 was obtained as a white solid (0.65 g, 1.4 mmol, 81%). LC-MS: Calc'd m/z=357.1 for C₁₇H₁₉N₅O₄. found [M+H]⁺=358.2.

### Example 1.68: (4-((6-amino-2-ethoxy-8-methoxy-9H-purin-9-yl)methyl)phenyl)methanol

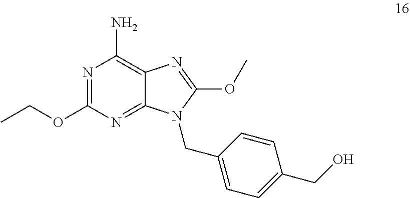 [SMILES: CCOc1nc(N)c2nc(OC)n(Cc3ccc(CO)cc3)c2n1]

To a stirring solution of compound 15 (0.20 g, 0.56 mmol, 1.0 eq.) in anhydrous THF (15 mL) cooled to 0° C. was added lithium aluminum hydride (64 mg, 1.7 mmol, 3.0 eq.) in small portions over 5 mins. The resulting suspension was allowed to warm to room temperature and stirred for 15 mins, after which it was quenched with H₂O (10 mL) and diluted with MeOH (50 mL) then filtered through a celite plug. Reverse phase flash purification was accomplished as described in General Procedure 5 using a 60 g C18 column, eluting with a 20 to 65% CH₃CN+0.1% TFA/H₂O+0.1% TFA gradient to give the title product 16 as a white solid (0.12 g, 0.36 mmol, 65%). LC-MS: Calc'd m/z=329.2 for C₁₁H₁₉N₅O₃. found [M+H]⁺=330.2.

### Example 1.69: 6-amino-9-(3-(chloromethyl)benzyl)-2-ethoxy-9H-purin-8-ol

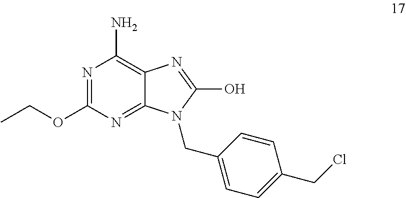 [SMILES: CCOc1nc(N)c2nc(O)n(Cc3ccc(CCl)cc3)c2n1]

The title compound 17 was prepared according to General Procedure 3 from compound 16 (0.10 g, 0.30 mmol, 1.0 eq.) and 10% SOCl₂/DCM (5 mL) and obtained as a yellow solid (assumed quantitative yield). LC-MS: Calc'd m/z=333.1 for C₁₅H₁₆ClN₅O₂. found [M+H]⁺=333.2.

### Example 1.70: 6-amino-2-ethoxy-9-(4-(piperazin-1-ylmethyl)benzyl)-9H-purin-8-ol (Compound 150)

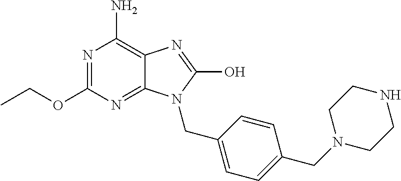 [SMILES: CCOc1nc(N)c2nc(O)n(Cc3ccc(CN4CCNCC4)cc3)c2n1]

The titled compound was prepared according to General Procedure 1 from compound 17 (16 mg, 0.048 mmol, 1.0 eq.) and tert-butyl piperazine-1-carboxylate (28 mg, 0.14 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 150 as a white solid (5.1 mg, 0.0070 mmol, 30%). LC-MS: Calc'd m/z=383.2 for C₁₉H₂₅N₇O₂. found [M+H]⁺=383.3. ¹H NMR (300 MHz, MeOD) δ 7.45 (d, J=8.1 Hz, 2H), 7.37 (d, J=8.1 Hz, 2H), 5.02 (s, 2H), 4.39 (q, J=7.1 Hz, 2H), 3.80 (s, 2H), 3.31-3.25 (m, 4H), 2.91-2.82 (m, 4H), 1.38 (t, J=7.1 Hz, 3H).

### Example 1.71: Methyl-4-((6-amino-2-ethoxy-8-methoxy-9H-purin-9-yl)methyl)-3-fluorobenzoate

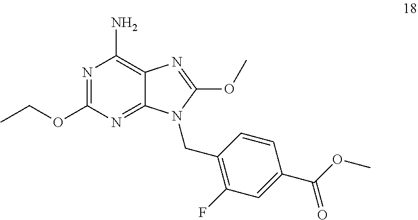 [SMILES: CCOc1nc(N)c2nc(OC)n(Cc3ccc(C(=O)OC)cc3F)c2n1]

To a solution of compound 8 (430 mg, 2.0 mmol, 1.0 eq.) and methyl 4-(bromomethyl)-3-fluorobenzoate (0.50 g, 2.0 mmol, 1.0 eq.) in DMF (5 mL) was added CsCO₃ (660 mg, 2.0 mmol, 1.0 eq.). The resulting suspension was stirred at room temperature for 18 h then concentrated in vacuo. Reverse phase flash purification was accomplished as described in General Procedure 5 using a 60 g C18 column, eluting with a 10 to 50% CH₃CN+0.1% TFA/H₂O+0.1% TFA gradient. The title compound 18 was obtained as a white solid (0.50 g, 1.3 mmol, 66%). LC-MS: Calc'd m/z=375.1 for C₁₇H₁₈FN₅O₄. found [M+H]⁺=376.2.

### Example 1.72: (4-((6-amino-2-ethoxy-8-methoxy-9H-purin-9-yl)methyl)-3-fluorophenyl)-methanol

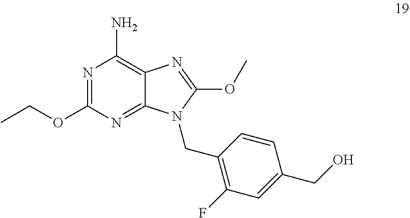 [SMILES: CCOc1nc(N)c2nc(OC)n(Cc3ccc(CO)cc3F)c2n1]

To a stirring solution of compound 18 (0.50 g, 1.3 mmol, 1.0 eq.) in anhydrous THF (4 mL) cooled to 0° C. was added lithium aluminum hydride (51 mg, 1.3 mmol, 1.0 eq.). The resulting suspension was allowed to warm to room temperature and stirred for 15 mins, after which it was diluted with MeOH (50 mL) then filtered through a celite plug. Reverse phase flash purification was accomplished as described in General Procedure 5 using a 60 g C18 column, eluting with a 10 to 40% CH₃CN+0.1% TFA/H₂O+0.1% TFA gradient to give the title product 19 as a white solid (0.40 g, 1.2 mmol, 86%). LC-MS: Calc'd m/z=347.1 for C₁₆H₁₈FN₅O₃. found [M+H]⁺=348.2.

### Example 1.73: 6-amino-9-(4-(chloromethyl)-2-fluorobenzyl)-2-ethoxy-9H-purin-8-ol

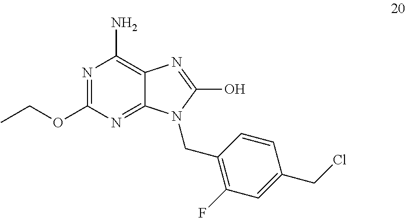 [SMILES: CCOc1nc(N)c2nc(O)n(Cc3ccc(CCl)cc3F)c2n1]

The title compound 20 was prepared according to General Procedure 3 from compound 19 (0.20 g, 0.58 mmol, 1.0 eq.) and 10% SOCl₂/DCM (5 mL) and obtained as a yellow solid (assumed quantitative yield). LC-MS: Calc'd m/z=351.1 for C₁₅H₁₅ClFN₅O₂. found [M+H]⁺=352.2.

### Example 1.74: 6-amino-2-ethoxy-9-(2-fluoro-4-(piperazin-1-ylmethyl)benzyl)-9H-purin-8-ol (Compound 152)

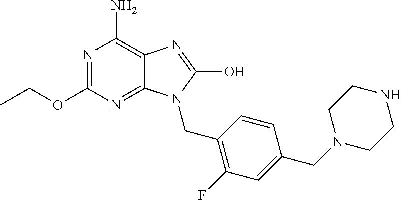 [SMILES: CCOc1nc(N)c2nc(O)n(Cc3ccc(CN4CCNCC4)cc3F)c2n1]

The titled compound was prepared according to General Procedure 1 from compound 20 (0.020 g, 0.057 mmol, 1.0 eq.) and tert-butyl piperazine-1-carboxylate (32 mg, 0.17 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 152 as a white solid (12 mg, 0.016 mmol, 57%). LC-MS: Calc'd m/z=401.2 for C₁₉H₂₄FN₇O₂. found [M+H]⁺=402.3. ¹H NMR (300 MHz, MeOD) δ 7.34 (t, J=7.7 Hz, 1H), 7.28-7.14 (m, 2H), 5.10 (s, 2H), 4.41 (q, J=7.1 Hz, 2H), 3.80 (s, 2H), 3.30 (d, J=5.3 Hz, 4H), 2.88 (t, J=5.3 Hz, 4H), 1.38 (t, J=7.1 Hz, 3H).

### Example 1.75: 6-amino-9-(4-((4-aminopiperidin-1-yl)methyl)-2-fluorobenzyl)-2-ethoxy-9H-purin-8-ol (Compound 153)

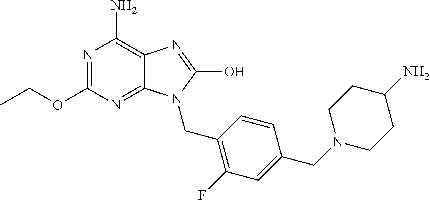 [SMILES: CCOc1nc(N)c2nc(O)n(Cc3ccc(CN4CCC(N)CC4)cc3F)c2n1]

The titled compound was prepared according to General Procedure 1 from compound 20 (0.020 g, 0.057 mmol, 1.0 eq.) and tert-butyl N-(4-aminobutyl)carbamate (34 mg, 0.17 mmol, 3.0 eq.). Preparative HPLC purification was accomplished as described in General Procedure 5, eluting with a 10 to 45% CH₃CN+0.1% TFA/H₂O+0.1% TFA gradient. Half of the resulting Boc intermediate was reserved and deprotection of the remaining material was accomplished according to General Procedure 2 to give the title compound 153 as a white solid (0.010 g, 0.013 mmol, 46%). LC-MS: Calc'd m/z=415.2 for C₁₉H₂₄FN₇O₂. found [M+H]⁺=416.3. ¹H NMR (300 MHz, MeOD) δ 7.48-7.27 (m, 3H), 5.13 (s, 2H), 4.43-4.30 (m, 4H), 3.65-3.54 (m, 2H), 3.49-3.39 (m, 1H), 3.16 (t, J=12.6 Hz, 2H), 2.32-2.21 (m, 2H), 2.05-1.88 (m, 2H), 1.36 (t, J=7.1 Hz, 3H).

### Example 1.76: 2-butoxy-9-(tetrahydro-2H-pyran-2-yl)-9H-purin-6-amine

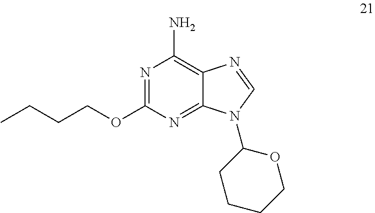 [SMILES: CCCCOc1nc(N)c2ncn(C3CCCCO3)c2n1]

To a solution of compound 2 (9.8 g, 39 mmol, 1.0 eq.) in nBuOH (100 mL) was added KOtBu (8.7 g, 77 mmol, 2.0 eq.). The resulting solution was stirred at 100° C. for 18 h, after which the solvent was removed in vacuo. The resulting solid was dissolved in EtOH (100 mL) and H₂O (20 mL) then extracted with EtOAc (2×50 mL). The pooled organics were washed with brine (5 mL) then dried over MgSO₄ and concentrated in vacuo to yield the title compound 21 as an orange solid (assumed quantitative yield). LC-MS: Calc'd m/z=291.2 for C₁₄H₂₁N₅O₂. found [M+H]⁺=292.2.

### Example 1.77: 8-bromo-2-butoxy-9-(tetrahydro-2H-pyran-2-yl)-9H-purin-6-amine

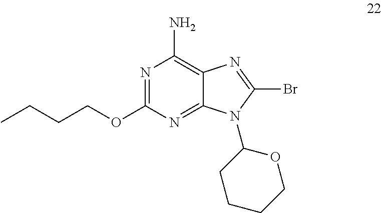 [SMILES: CCCCOc1nc(N)c2nc(Br)n(C3CCCCO3)c2n1]

To a suspension of compound 21 (11 g, 39 mmol, 1.0 eq.) in DCM (50 mL) was added NBS (6.9 g, 39 mmol, 1.0 eq.). The resulting mixture was stirred at room temperature for 18 h, after which the reaction was quenched by the addition of 1 M NaHSO₃ (50 mL) and rapidly stirred for 30 mins. The resulting mixture was diluted with DCM (100 mL) and the separated organics washed with 1 M NaHSO₃ (2×50 mL). The pooled organics were dried over MgSO₄, filtered then concentrated in vacuo to yield the title compound 22 as an orange solid (13 g, 34 mmol, 88%). LC-MS: Calc'd m/z=369.1 for C₁₄H₂₀BrN₅O₂. found [M+H]⁺=370.2.

### Example 1.78: 2-butoxy-8-methoxy-9-(tetrahydro-2H-pyran-2-yl)-9H-purin-6-amine

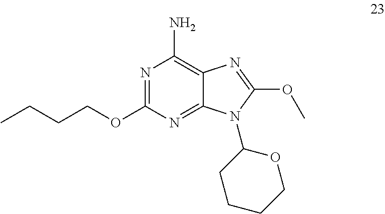 [SMILES: CCCCOc1nc(N)c2nc(OC)n(C3CCCCO3)c2n1]

To a solution of compound 22 (13 g, 34 mmol, 1.0 eq.) in MeOH (100 mL) was added NaOMe (5.47 g, 101 mmol, 3.00 eq.). The resulting mixture was stirred at room temperature for 72 h. Additional NaOMe (3.6 g, 66 mmol, 1.0 eq.) was added and the mixture was heated to reflux for 72 h. The solvent was removed in vacuo and crude material redissolved in EtOAc (250 mL) then washed with 1 M NaH₂PO₄ (150 mL). The aqueous layer was then extracted with EtOAc (3×150 mL), the pooled organics dried over MgSO₄, and the solution concentrated in vacuo to yield the title compound 23 as a red solid (11 g, 34 mmol, 100%). LC-MS: Calc'd m/z=321.2 for C₁₅H₂₃N₅O₃. found [M+H]⁺=322.3.

### Example 1.79: 2-butoxy-8-methoxy-9-(tetrahydro-H-pyran-2-yl)-9H-purin-6-amine

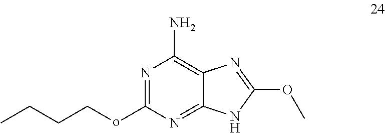

Compound 23 (11 g, 34 mmol, 1.0 eq.) was dissolved in 10% TFA/MeOH (50 mL) and the resulting solution stirred at room temperature for 2 weeks. The solvent was removed in vacuo and the residue co-evaporated with MeOH (2×20 mL) to yield the title compound 24 as an orange solid (9.8 g, 28 mmol, 83%). LC-MS: Calc'd m/z=237.1 for C₁₀H₁₅N₅O₂. found [M+H]⁺=238.2.

### Example 1.80: Methyl-4-((6-amino-2-butoxy-8-methoxy-9H-purin-9-yl)methyl)-3-methoxybenzoate

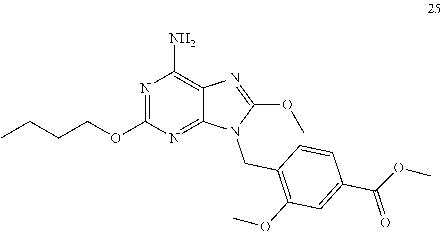 [SMILES: CCCCOc1nc(N)c2nc(OC)n(Cc3ccc(C(=O)OC)cc3OC)c2n1]

To a solution of compound 24 (570 mg, 2.4 mmol, 1.0 eq.) and methyl 4-(bromomethyl)-3-methoxybenzoate (620 mg, 2.4 mmol, 1.00 eq.) in DMF (5 mL) was added CsCO₃ (620 mg, 2.4 mmol, 1.0 eq.). The resulting suspension was stirred at room temperature for 18 h then concentrated in vacuo. Reverse phase flash purification was accomplished as described in General Procedure 5 using a 60 g C18 column, eluting with a 10 to 60% CH₃CN+0.1% TFA/H₂O+0.1% TFA gradient. The title compound 25 was obtained as a white solid (240 mg, 0.58 mmol, 24%). LC-MS: Calc'd m/z=415.2 for C₂₀H₂₅N₅O₅. found [M+H]⁺=416.3.

### Example 1.81: (4-((6-amino-2-butoxy-8-methoxy-9H-purin-9-yl)methyl)-3-methoxyphenyl)-methanol

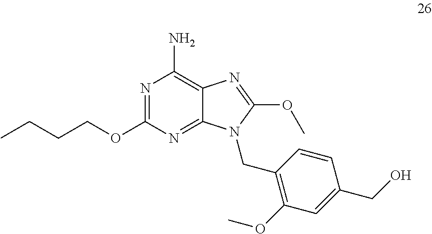 [SMILES: CCCCOc1nc(N)c2nc(OC)n(Cc3ccc(CO)cc3OC)c2n1]

To a stirring solution of compound 25 (240 mg, 0.58 mmol, 1.0 eq.) in anhydrous THF (5 mL) cooled to 0° C. was added lithium aluminum hydride (22 mg, 0.58 mmol, 1.0 eq.). The resulting suspension was allowed to warm to room temperature and stirred for 15 mins, after which it was diluted with MeOH (25 mL) then filtered through a celite plug. The filtrate was concentrated in vacuo to yield the title compound 26 (220 mg, 0.57 mmol, 98%). LC-MS: Calc'd m/z=387.2 for C₁₉H₂₅N₅O₄. found [M+H]⁺=388.3.

### Example 1.82: 6-amino-2-butoxy-9-(4-(chloromethyl)-2-methoxybenzyl)-9H-purin-8-ol

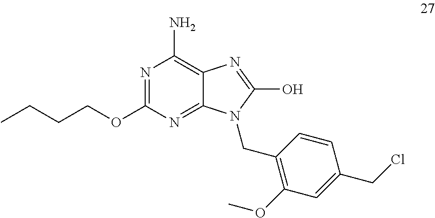 [SMILES: CCCCOc1nc(N)c2nc(O)n(Cc3ccc(CCl)cc3OC)c2n1]

The title compound 27 was prepared according to General Procedure 3 from compound 26 (220 mg, 0.57 mmol, 1.0 eq.) and 15% SOCl₂/DCM (5 mL). The titled compound was obtained as a yellow solid (210 mg, 0.577 mmol, 98.8%). LC-MS: Calc'd m/z=391.1 for C₁₈H₂₂ClN₅O₃. found [M+H]⁺=392.2.

### Example 1.83: 6-amino-2-butoxy-9-(2-methoxy-4-(piperazin-1-ylmethyl)benzyl)-9H-purin-8-ol (Compound 154)

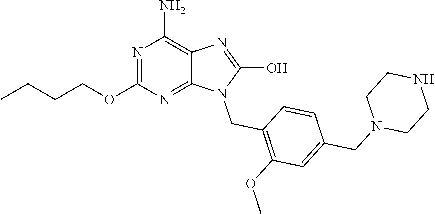 [SMILES: CCCCOc1nc(N)c2nc(O)n(Cc3ccc(CN4CCNCC4)cc3OC)c2n1]

The titled compound was prepared according to General Procedure 1 from compound 27 (28 mg, 0.071 mmol, 1.0 eq.) and tert-butyl piperazine-1-carboxylate (53 mg, 0.28 mmol, 4.0 eq.). Preparative HPLC purification was accomplished as described in General Procedure 5, eluting with a 10 to 55% CH₃CN+0.1% TFA/H₂O+0.1% TFA gradient. Half of the resulting Boc intermediate was reserved and deprotection of the remaining material was accomplished according to General Procedure 2 to give the title compound 154 as a white solid (6.8 mg, 0.0087 mmol, 24%). LC-MS: Calc'd m/z=441.2 for C₂₂H₃₁N₇O₃. found [M+H]⁺=442.3. ¹H NMR (300 MHz, MeOD) δ 7.07-6.94 (m, 2H), 6.90 (d, J=7.7 Hz, 1H), 5.03 (s, 2H), 4.28 (t, J=6.6 Hz, 2H), 3.90 (s, 3H), 3.76 (s, 2H), 3.34-3.24 (m, 4H), 2.84 (s, 4H), 1.72 (p, J=6.7 Hz, 2H), 1.46 (h, J=7.4 Hz, 2H), 0.97 (t, J=7.4 Hz, 3H).

### Example 1.84: 6-amino-9-(4-((4-aminopiperidin-1-yl)methyl)-2-methoxybenzyl)-2-butoxy-9H-purin-8-ol (Compound 155)

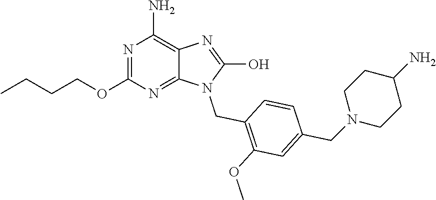 [SMILES: CCCCOc1nc(N)c2nc(O)n(Cc3ccc(CN4CCC(N)CC4)cc3OC)c2n1]

The titled compound was prepared according to General Procedure 1 from compound 27 (25 mg, 0.064 mmol, 1.0 eq.) and tert-butyl N-(4-aminobutyl)carbamate (51 mg, 0.26 mmol, 4.0 eq.). Preparative HPLC purification was accomplished as described in General Procedure 5, eluting with a 10 to 45% CH₃CN+0.1% TFA/H₂O+0.1% TFA gradient. Deprotection was accomplished according to General Procedure 2 to give the title compound 155 as a white solid (6.1 mg, 0.0076 mmol, 24%). LC-MS: Calc'd m/z=455.3 for C₂₃H₃₃N₇O₃. found [M+H]⁺=456.3. ¹H NMR (300 MHz, MeOD) δ 7.15 (s, 1H), 7.03 (q, J=7.9 Hz, 2H), 5.05 (s, 2H), 4.35-4.20 (m, 4H), 3.94 (s, 3H), 3.61 (d, J=12.9 Hz, 2H), 3.45 (s, 1H), 3.14 (t, J=13.1 Hz, 2H), 2.27 (d, J=14.7 Hz, 2H), 1.93 (d, J=13.3 Hz, 2H), 1.78-1.63 (m, 2H), 1.46 (h, J=7.4 Hz, 2H), 0.98 (t, J=7.4 Hz, 2H).

### Example 1.85: 6-amino-9-(4-((4-(aminomethyl)piperidin-1-yl)methyl)-2-methoxybenzyl)-2-butoxy-9H-purin-8-ol (Compound 156)

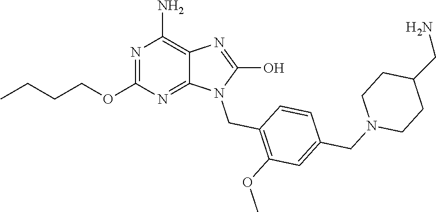 [SMILES: CCCCOc1nc(N)c2nc(O)n(Cc3ccc(CN4CCC(CN)CC4)cc3OC)c2n1]

The titled compound was prepared according to General Procedure 1 from compound 27 (30 mg, 0.077 mmol, 1.0 eq.) and tert-butyl N-(piperidin-4-ylmethyl)carbamate (49 mg, 0.23 mmol, 3.0 eq.). Preparative HPLC purification was accomplished as described in General Procedure 5, eluting with a 10 to 55% CH₃CN+0.1% TFA/H₂O+0.1% TFA gradient. Deprotection was accomplished according to General Procedure 2 to give the title compound 156 as a white solid (8.0 mg, 0.0099 mmol, 13%). LC-MS: Calc'd m/z=469.3 for C₂₄H₃₅N₇O₃, found [M+H]⁺=470.4. ¹H NMR (300 MHz, MeOD) δ 7.16 (s, 1H), 7.10-6.96 (m, 2H), 5.05 (s, 2H), 4.34-4.20 (m, 4H), 3.94 (s, 3H), 3.62-3.56 (m, 2H), 3.12-2.96 (m, 2H), 2.95-2.87 (m, 2H), 2.14-1.87 (m, 4H), 1.79-1.63 (m, 2H), 1.60-1.35 (m, 3H), 0.96 (t, J=7.4 Hz, 3H).

### Example 1.86: 9-(4-((3,9-diazaspiro[5.5]undecan-3-yl)methyl)-2-methoxybenzyl)-6-amino-2-butoxy-9H-purin-8-ol (Compound 157)

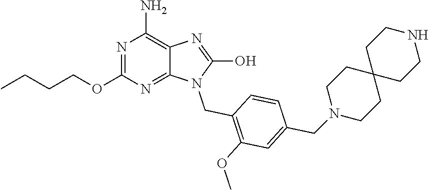 [SMILES: CCCCOc1nc(N)c2nc(O)n(Cc3ccc(CN4CCC5(CCNCC5)CC4)cc3OC)c2n1]

The titled compound was prepared according to General Procedure 1 from compound 27 (30 mg, 0.077 mmol, 1.0 eq.) and tert-butyl 3,9-diazaspiro[5.5]undecane-3-carboxylate (58 mg, 0.23 mmol, 3.0 eq.). Preparative HPLC purification was accomplished as described in General Procedure 5, eluting with a 10 to 55% CH₃CN+0.1% TFA/H₂O+0.1% TFA gradient. Deprotection was accomplished according to General Procedure 2 to give the title compound 157 as a white solid (0.010 g, 0.012 mmol, 15%). LC-MS: Calc'd m/z=509.3 for C₂₇H₃₉N₇O₃. found [M+H]⁺=510.3. ¹H NMR (300 MHz, MeOD) δ 7.15 (s, 1H), 7.03 (q, J=7.7 Hz, 2H), 5.05 (s, 2H), 4.32 (s, 2H), 4.25 (t, J=6.6 Hz, 2H), 3.95 (s, 3H), 3.24-3.19 (m, 8H), 3.19-3.08 (m, 2H), 2.08-1.97 (m, 2H), 1.96-1.90 (m, 2H), 1.69 (s, 6H), 1.55-1.35 (m, 2H), 0.96 (t, J=7.4 Hz, 3H).

### Example 1.87: 6-amino-9-(4-((4-(2-aminoethyl)piperidin-1-yl)methyl)-2-methoxybenzyl)-2-butoxy-9H-purin-8-ol (Compound 158)

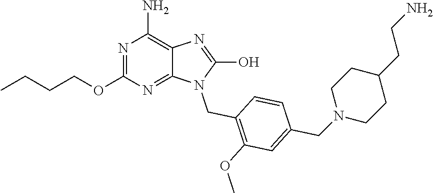 [SMILES: CCCCOc1nc(N)c2nc(O)n(Cc3ccc(CN4CCC(CCN)CC4)cc3OC)c2n1]

The titled compound was prepared according to General Procedure 1 from compound 27 (30 mg, 0.077 mmol, 1.0 eq.) and tert-butyl N-[2-(piperidin-4-yl)ethyl]carbamate (52 mg, 0.23 mmol, 3.0 eq.). Preparative HPLC purification was accomplished as described in General Procedure 5, eluting with a 10 to 55% CH₃CN+0.1% TFA/H₂O+0.1% TFA gradient. Deprotection was accomplished according to General Procedure 2 to give the title compound 158 as a white solid (9.0 mg, 0.011 mmol, 14%). LC-MS: Calc'd m/z=483.3 for C₂₅H₃₇N₇O₃. found [M+H]⁺=484.3. ¹H NMR (300 MHz, MeOD) δ 7.15 (s, 1H), 7.02 (q, J=7.9 Hz, 2H), 5.05 (s, 2H), 4.32-4.20 (m, 4H), 3.95 (s, 3H), 3.50 (s, 2H), 3.05-2.93 (m, 6H), 2.06-1.96 (m, 2H), 1.78-1.60 (m, 4H), 1.53-1.35 (m, 3H), 0.96 (t, J=7.4 Hz, 3H).

### Example 1.88: 6-amino-2-butoxy-9-(4-(((2-hydroxy-2-methylpropyl)amino)methyl)-2-methoxybenzyl)-9H-purin-8-ol (Compound 159)

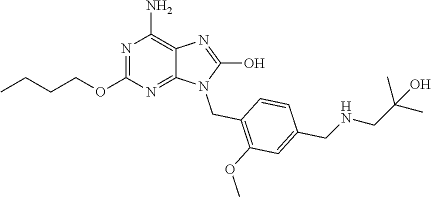 [SMILES: CCCCOc1nc(N)c2nc(O)n(Cc3ccc(CNCC(C)(C)O)cc3OC)c2n1]

The titled compound was prepared according to General Procedure 1 from compound 27 (30 mg, 0.077 mmol, 1.0 eq.) and 1-amino-2-methylpropan-2-ol (0.020 g, 0.23 mmol, 3.0 eq.). Preparative HPLC purification was accomplished as described in General Procedure 5, eluting with a 10 to 45% CH₃CN+0.1% TFA/H₂O+0.1% TFA gradient to give the title compound 159 as a white solid (0.010 g, 0.030 mmol, 39%). LC-MS: Calc'd m/z=444.2 for C₂₂H₃₂N₆O₄. found [M+H]⁺=445.3. ¹H NMR (300 MHz, MeOD) δ 7.18 (s, 1H), 7.08-6.96 (m, 2H), 5.04 (s, 2H), 4.30-4.19 (m, 4H), 3.94 (s, 3H), 2.93-2.83 (m, 2H), 1.78-1.63 (m, 2H), 1.55-1.33 (m, 2H), 1.29 (s, 6H), 0.96 (t, J=7.4 Hz, 3H).

### Example 1.89: 6-amino-9-(4-(((4-aminobutyl)amino)methyl)-2-methoxybenzyl)-2-butoxy-9H-purin-8-ol (Compound 160)

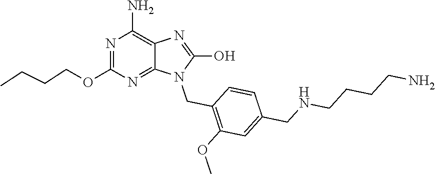 [SMILES: CCCCOc1nc(N)c2nc(O)n(Cc3ccc(CNCCCCN)cc3OC)c2n1]

The titled compound was prepared according to General Procedure 1 from compound 27 (30 mg, 0.077 mmol, 1.0 eq.) and tert-butyl N-(4-aminobutyl)carbamate (43 mg, 0.23 mmol, 3.0 eq.). Preparative HPLC purification was accomplished as described in General Procedure 5, eluting with a 10 to 55% CH₃CN+0.1% TFA/H₂O+0.1% TFA gradient. Deprotection was accomplished according to General Procedure 2 to give the title compound 160 as a white solid (0.010 g, 0.013 mmol, 17%). LC-MS: Calc'd m/z=443.3 for C₂₂H₃₃N₇O₃. found [M+H]⁺=444.3. ¹H NMR (300 MHz, MeOD) δ 7.14 (s, 1H), 7.08-6.94 (m, 2H), 5.04 (s, 2H), 4.30-4.17 (m, 4H), 3.94 (s, 3H), 3.10 (t, J=7.6 Hz, 2H), 2.98 (t, J=7.3 Hz, 2H), 1.89-1.62 (m, 8H), 1.55-1.37 (m, 2H), 0.97 (t, J=7.4 Hz, 3H).

### Example 1.90: 6-amino-2-butoxy-9-(4-((((2R,3S)-1-hydroxy-3-methylpentan-2-yl)amino)-methyl)-2-methoxybenzyl)-9H-purin-8-ol (Compound 161)

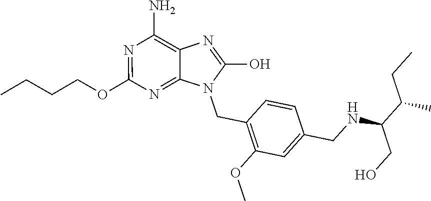 [SMILES: CCCCOc1nc(N)c2nc(O)n(Cc3ccc(CN[C@H](CO)[C@@H](C)CC)cc3OC)c2n1]

The titled compound was prepared according to General Procedure 1 from compound 27 (30 mg, 0.077 mmol, 1.0 eq.) and (2R,3S)-2-aminopentane-1,3-diol (27 mg, 0.23 mmol, 3.0 eq.). Preparative HPLC purification was accomplished as described in General Procedure 5, eluting with a 10 to 45% CH₃CN+0.1% TFA/H₂O+0.1% TFA gradient to give the title compound 161 as a white solid (1.9 mg, 0.0027 mmol, 3.5%). LC-MS: Calc'd m/z=472.3 for C₂₄H₃₆N₆O₄. found [M+H]⁺=473.4. ¹H NMR (300 MHz, MeOD) δ 7.19 (s, 1H), 7.04 (s, 2H), 5.05 (s, 2H), 4.42-4.19 (m, 4H), 3.94 (s, 3H), 3.88 (dd, J=12.1, 3.9 Hz, 1H), 3.76 (dd, J=12.1, 7.2 Hz, 1H), 3.15 (dt, J=7.3, 4.2 Hz, 1H), 1.88 (s, 1H), 1.78-1.63 (m, 2H), 1.56-1.38 (m, 3H), 1.38-1.25 (m, 1H), 1.04-0.87 (m, 9H).

### Example 1.91: 6-amino-9-(4-(aminomethyl)-2-methoxybenzyl)-2-butoxy-9H-purin-8-ol (Compound 162)

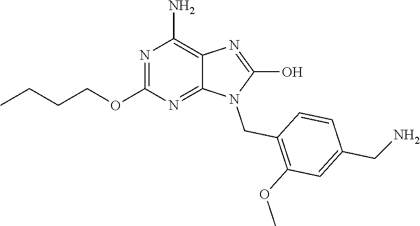 [SMILES: CCCCOc1nc(N)c2nc(O)n(Cc3ccc(CN)cc3OC)c2n1]

The titled compound was prepared according to General Procedure 1 from compound 27 (13 mg, 0.033 mmol, 1.0 eq.) and ammonia/DMF (83 μL, 2 M, 5.0 eq.). Preparative HPLC purification was accomplished as described in General Procedure 5, eluting with a 10 to 45% CH₃CN+0.1% TFA/H₂O+0.1% TFA gradient to give the title compound 162 as a white solid (1.6 mg, 0.0027 mmol, 8.1%). LC-MS: Calc'd m/z=372.2 for C₁₈H₂₄N₆O₃. found [M+H]⁺=373.3. ¹H NMR (300 MHz, MeOD) δ 7.10 (d, J=1.6 Hz, 1H), 7.03 (d, J=7.8 Hz, 1H), 6.95 (dd, J=7.7, 1.6 Hz, 1H), 5.04 (s, 2H), 4.25 (t, J=6.5 Hz, 2H), 4.10 (s, 2H), 3.93 (s, 3H), 1.79-1.63 (m, 2H), 1.58-1.37 (m, 2H), 0.97 (t, J=7.4 Hz, 3H).

### Example 1.92: 6-amino-2-butoxy-9-(2-methoxy-4-((methylamino)methyl)benzyl)-9H-purin-8-ol (Compound 163)

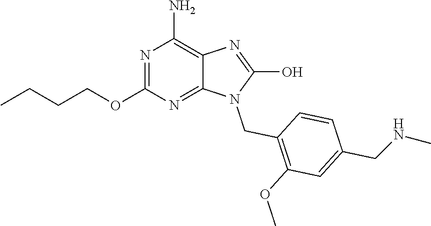 [SMILES: CCCCOc1nc(N)c2nc(O)n(Cc3ccc(CNC)cc3OC)c2n1]

The titled compound was prepared according to General Procedure 1 from compound 27 (13 mg, 0.033 mmol, 1.0 eq.) and methylamine hydrochloride (11.2 mg, 0.17 mmol, 5.0 eq.). Preparative HPLC purification was accomplished as described in General Procedure 5, eluting with a 10 to 45% CH₃CN+0.1% TFA/H₂O+0.1% TFA gradient to give the title compound 163 as a white solid (2.6 mg, 0.0042 mmol, 13%). LC-MS: Calc'd m/z=386.2 for C₁₉H₂₆N₆O₃. found [M+H]⁺=387.3. ¹H NMR (300 MHz, MeOD) δ 7.11 (s, 1H), 7.08-6.93 (m, 2H), 5.04 (s, 2H), 4.24 (t, J=6.5 Hz, 2H), 4.17 (s, 2H), 3.94 (s, 3H), 2.73 (s, 3H), 1.70 (p, J=6.7 Hz, 2H), 1.46 (dq, J=14.5, 7.3 Hz, 2H), 0.96 (t, J=7.4 Hz, 3H).

### Example 1.93: 6-amino-2-butoxy-9-(4-((cyclobutylamino)methyl)-2-methoxybenzyl)-9H-purin-8-ol (Compound 164)

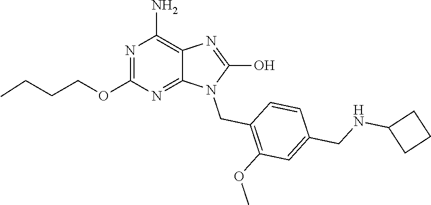 [SMILES: CCCCOc1nc(N)c2nc(O)n(Cc3ccc(CNC4CCC4)cc3OC)c2n1]

The titled compound was prepared according to General Procedure 1 from compound 27 (13 mg, 0.033 mmol, 1.0 eq.) and cyclobutylamine (14 μL, 0.17 mmol, 5.0 eq.). Preparative HPLC purification was accomplished as described in General Procedure 5, eluting with a 10 to 45% CH₃CN+0.1% TFA/H₂O+0.1% TFA gradient to give the title compound 164 as a white solid (5.3 mg, 0.0081 mmol, 25%). LC-MS: Calc'd m/z=426.2 for C₂₂H₃₀N₆O₃. found [M+H]⁺=427.3. ¹H NMR (300 MHz, MeOD) δ 7.19-6.92 (m, 3H), 5.05 (s, 2H), 4.26 (t, J=6.5 Hz, 2H), 4.07 (s, 2H), 3.94 (s, 3H), 3.80 (p, J=8.0 Hz, 1H), 2.50-2.02 (m, 4H), 2.03-1.77 (m, 2H), 1.78-1.61 (m, 2H), 1.60-1.37 (m, 2H), 0.97 (t, J=7.4 Hz, 3H).

### Example 1.94: 6-amino-9-(4-((benzylamino)methyl)-2-methoxybenzyl)-2-butoxy-9H-purin-8-ol (Compound 165)

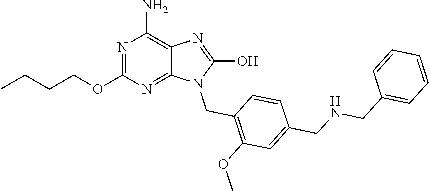 [SMILES: CCCCOc1nc(N)c2nc(O)n(Cc3ccc(CNCc4ccccc4)cc3OC)c2n1]

The titled compound was prepared according to General Procedure 1 from compound 27 (13 mg, 0.033 mmol, 1.0 eq.) and benzylamine (18 μL, 0.17 mmol, 5.0 eq.). Preparative HPLC purification was accomplished as described in General Procedure 5, eluting with a 10 to 45% CH₃CN+0.1% TFA/H₂O+0.1% TFA gradient to give the title compound 165 as a white solid (7.8 mg, 0.011 mmol, 34%). LC-MS: Calc'd m/z=462.2 for C₂₅H₃₀N₆O₃. found [M+H]⁺=463.3. ¹H NMR (300 MHz, MeOD) δ 7.48 (s, 5H), 7.13 (d, J=1.5 Hz, 1H), 7.10-6.94 (m, 2H), 5.04 (s, 2H), 4.32-4.16 (m, 6H), 3.93 (s, 3H), 1.86-1.62 (m, 2H), 1.57-1.36 (m, 2H), 0.96 (t, J=7.4 Hz, 3H).

### Example 1.95: Methyl-4-((6-amino-2-butoxy-8-methoxy-9H-purin-9-yl)methyl)benzoate

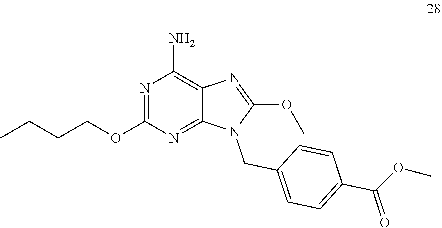 [SMILES: CCCCOc1nc(N)c2nc(OC)n(Cc3ccc(C(=O)OC)cc3)c2n1]

To a solution of compound 24 (0.90 g, 3.8 mmol, 1.0 eq.) and methyl 4-(bromomethyl)-3-methoxybenzoate (0.87 g, 3.8 mmol, 1.0 eq.) in DMF (8 mL) was added CsCO₃ (1.3 g, 3.8 mmol, 1.0 eq.). The resulting suspension was stirred at room temperature for 18 h then concentrated in vacuo. Reverse phase flash purification was accomplished as described in General Procedure 5 using a 60 g C18 column, eluting with a 10 to 60% CH₃CN+0.1% TFA/H₂O+0.1% TFA gradient. The title compound 28 was obtained as a white solid (0.23 g, 0.60 mmol, 16%). LC-MS: Calc'd m/z=385.2 for C₁₉H₂₃N₅O₄. found [M+H]⁺=386.3.

### Example 1.96: (4-((6-amino-2-butoxy-8-methoxy-9H-purin-9-yl)methyl)phenyl)methanol

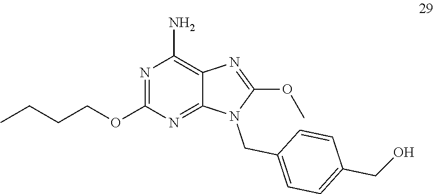 [SMILES: CCCCOc1nc(N)c2nc(OC)n(Cc3ccc(CO)cc3)c2n1]

To a stirring solution of compound 28 (0.23 g, 0.60 mmol, 1.0 eq.) in anhydrous THF (8 mL) cooled to 0° C. was added lithium aluminum hydride (23 mg, 0.60 mmol, 1.0 eq.). The resulting suspension was stirred for 5 mins then allowed to warm to room temperature and stirred for a further 15 mins, after which it was diluted with MeOH (50 mL) then filtered through a celite plug. The filtrate was concentrated in vacuo to yield the title compound 29 as a yellow solid (0.20 g, 0.56 mmol, 94%). LC-MS: Calc'd m/z=357.2 for C₁₈H₂₃N₅O₃. found [M+H]⁺=358.3.

### Example 1.97: 6-amino-2-butoxy-9-(4-(chloromethyl)benzyl)-9H-purin-8-ol

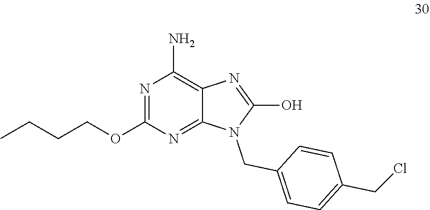 [SMILES: CCCCOc1nc(N)c2nc(O)n(Cc3ccc(CCl)cc3)c2n1]

The title compound 30 was prepared according to General Procedure 3 from compound 29 (200 mg, 0.56 mmol, 1.0 eq.) and 10% SOCl₂/DCM (10 mL). The titled compound was obtained as a yellow solid (assumed quantitative yield). LC-MS: Calc'd m/z=361.1 for C₇H₂₀ClN₅O₂. found [M+H]⁺=396.2.

### Example 1.98: 6-amino-2-butoxy-9-(4-(piperazin-1-ylmethyl)benzyl)-9H-purin-8-ol (Compound 166)

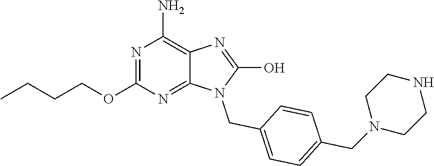 [SMILES: CCCCOc1nc(N)c2nc(O)n(Cc3ccc(CN4CCNCC4)cc3)c2n1]

The titled compound was prepared according to General Procedure 1 from compound 30 (30 mg, 0.083 mmol, 1.0 eq.) and tert-butyl piperazine-1-carboxylate (77 mg, 0.42 mmol, 5.0 eq.). Preparative HPLC purification was accomplished as described in General Procedure 5, eluting with a 10 to 55% CH₃CN+0.1% TFA/H₂O+0.1% TFA gradient. Half of the resulting Boc intermediate was reserved and deprotection of the remaining material was accomplished according to General Procedure 2 to give the title compound 166 as a white solid (0.010 g, 0.014 mmol, 33%). LC-MS: Calc'd m/z=411.2 for C₂₁H₂₉N₇O₂. found [M+H]⁺=412.3. ¹H NMR (300 MHz, MeOD) δ 7.07-6.94 (m, 2H), 6.90 (d, J=7.7 Hz, 1H), 5.03 (s, 2H), 4.28 (t, J=6.6 Hz, 2H), 3.90 (s, 3H), 3.76 (s, 2H), 3.34-3.24 (m, 4H), 2.84 (s, 4H), 1.72 (p, J=6.7 Hz, 2H), 1.46 (h, J=7.4 Hz, 2H), 0.97 (t, J=7.4 Hz, 3H).

### Example 1.99: 2-butoxy-9-((6-chloropyridin-3-yl)methyl)-8-methoxy-9H-purin-6-amine

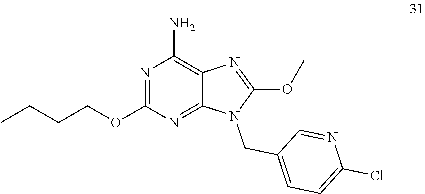 [SMILES: CCCCOc1nc(N)c2nc(OC)n(Cc3ccc(Cl)nc3)c2n1]

To a solution of compound 24 (0.70 g, 3.0 mmol, 1.0 eq.) and 2-chloro-5-(chloromethyl)pyridine (0.48 g, 3.0 mmol, 1.0 eq.) in DMF (5 mL) was added CsCO₃ (0.96 g, 3.0 mmol, 1.0 eq.). The resulting suspension was stirred at room temperature for 18 h then concentrated in vacuo. Reverse phase flash purification was accomplished as described in General Procedure 5 using a 60 g C18 column, eluting with a 10 to 60% CH₃CN+0.1% TFA/H₂O+0.1% TFA gradient. The title compound 31 was obtained as a white solid (0.65 g, 1.8 mmol, 61%). LC-MS: Calc'd m/z=362.1 for C₁₆H₁₉ClN₆O₂. found [M+H]⁺=363.2.

### Example 1.100: 6-amino-2-butoxy-9-((6-(piperazin-1-yl)pyridin-3-yl)methyl)-9H-purin-8-ol (Compound 167)

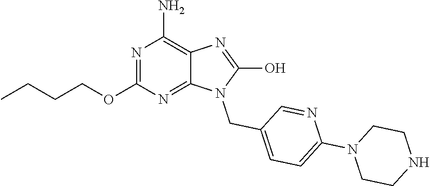 [SMILES: CCCCOc1nc(N)c2nc(O)n(Cc3ccc(N4CCNCC4)nc3)c2n1]

A DMF (0.40 mL) solution of compound 31 (15 mg, 0.041 mmol, 1.0 eq.), tert-butyl piperazine-1-carboxylate (9.2 mg, 0.050 mmol, 1.2 eq.), KOtBu (7.0 mg, 0.062 mmol, 1.5 eq.) and Pd-PEPPSI™-iPr in a crimp-sealable microwave vial was thoroughly degassed and sealed under N₂ then heated to 85° C. for 2 h. The reaction mixture was then diluted with CH₃CN/H₂O (1 mL) and reverse phase flash purification was accomplished as described in General Procedure 5 using a 12 g C18 column, eluting with a 10 to 60% CH₃CN+0.1% TFA/H₂O+0.1% TFA gradient to give the title compound 167 as a white solid (0.010 g, 0.025 mmol, 60%). LC-MS: Calc'd m/z=398.2 for C₁₉H₂₆N₈O₂. found [M+H]⁺=399.3. ¹H NMR (300 MHz, MeOD) δ 8.31 (d, J=2.3 Hz, 1H), 7.79 (dd, J=8.8, 2.4 Hz, 1H), 6.95 (d, J=8.8 Hz, 1H), 4.94 (s, 2H), 4.36 (t, J=6.5 Hz, 2H), 3.86-3.76 (m, 4H), 3.33-3.27 (m, 4H), 1.85-1.70 (m, 2H), 1.52 (dq, J=14.5, 7.4 Hz, 2H), 1.01 (t, J=7.4 Hz, 3H).

### Example 1.101: 9-((6-(3,9-diazaspiro[5.5]undecan-3-yl)pyridin-3-yl)methyl)-6-amino-2-butoxy-9H-purin-8-ol (Compound 168)

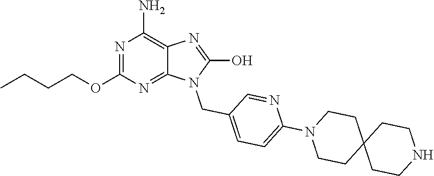 [SMILES: CCCCOc1nc(N)c2nc(O)n(Cc3ccc(N4CCC5(CCNCC5)CC4)nc3)c2n1]

To a solution of tert-butyl 3,9-diazaspiro[5.5]undecane-3-carboxylate (280 mg, 1.1 mmol, 10 eq.) in DMF (0.50 mL) and DIPEA (50 μL) was added compound 31 (0.040 g, 0.11 mmol, 1.0 eq.) and the resulting mixture heated to 140° C. for 18 h. The reaction was then adjusted to pH 1 with 6 M HCl and preparative HPLC purification was accomplished as described in General Procedure 5, eluting with a 10 to 65% CH₃CN+0.1% TFA/H₂O+0.1% TFA gradient to give the title compound 168 as a white solid (0.7 mg, 0.0009 mmol, 0.8%). LC-MS: Calc'd m/z=466.3 for C₂₄H₃₄N₈O₂. found [M+H]⁺=467.4. ¹H NMR (300 MHz, MeOD) δ 8.09 (s, 1H), 8.01 (d, J=8.2 Hz, 1H), 7.25 (d, J=9.4 Hz, 1H), 4.93 (s, 2H), 4.32 (t, J=6.5 Hz, 2H), 4.12 (q, J=7.0 Hz, 4H), 3.67 (t, J=5.9 Hz, 6H), 3.23 (t, J=5.9 Hz, 6H), 1.78-1.62 (m, 2H), 1.58-1.35 (m, 2H), 1.00 (t, J=7.4 Hz, 3H).

### Example 1.102: 9-(4-bromo-2-methoxybenzyl)-2-butoxy-8-methoxy-9H-purin-6-amine

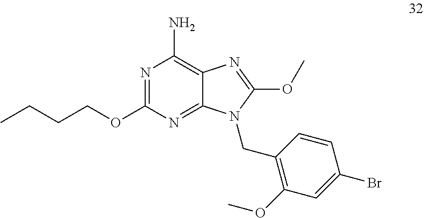 [SMILES: CCCCOc1nc(N)c2nc(OC)n(Cc3ccc(Br)cc3OC)c2n1]

To a solution of compound 24 (580 mg, 2.4 mmol, 1.0 eq.) and 4-bromo-1-(bromomethyl)-2-methoxybenzene (680 mg, 2.4 mmol, 1.0 eq.) in DMF (8 mL) was added CsCO₃ (0.80 g, 2.4 mmol, 1.0 eq.). The resulting suspension was stirred at room temperature for 18 h then concentrated in vacuo. Reverse phase flash purification was accomplished as described in General Procedure 5 using a 60 g C18 column, eluting with a 10 to 60% CH₃CN+0.1% TFA/H₂O+0.1% TFA gradient. The title compound 32 was obtained as a white solid (0.50 g, 1.1 mmol, 47%). LC-MS: Calc'd m/z=435.1 for C₁₈H₂₂BrN₅O₃. found [M+H]⁺=436.2.

### Example 1.103: 6-amino-2-butoxy-9-(2-methoxy-4-(piperazin-1-yl)benzyl)-9H-purin-8-ol (Compound 169)

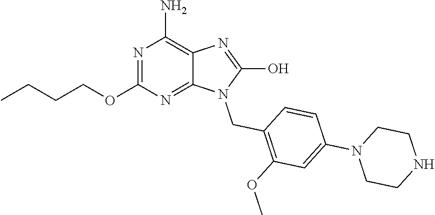 [SMILES: CCCCOc1nc(N)c2nc(O)n(Cc3ccc(N4CCNCC4)cc3OC)c2n1]

A DMF (0.50 mL) solution of compound 32 (0.020 g, 0.046 mmol, 1.0 eq.), tert-butyl piperazine-1-carboxylate (11 mg, 0.060 mmol, 1.3 eq.), KOtBu (18 mg, 0.16 mmol, 3.5 eq.) and Pd₂dba₃ (4.2 mg, 0.0046 mmol, 0.10 eq.) and RuPhos (11 mg, 0.023 mmol, 0.50 eq.) in a crimp-sealable microwave vial was thoroughly degassed and sealed under N₂ then heated to 100° C. for 18 h. The reaction mixture was then diluted with EtOAc (25 mL), filtered through celite and concentrated in vacuo. Preparative HPLC purification was accomplished as described in General Procedure 5, eluting with a 10 to 60% CH₃CN+0.1% TFA/H₂O+0.1% TFA gradient to give the Boc/8-MeO intermediate. This intermediate was then dissolved in CH₃CN (500 μL), H₂O (500 μL) and 6 M HCl (100 μL) and the mixture heated to 55° C. for 3 h. The reaction mixture was cooled to room temperature to give the title product 169 after lyophilization (0.010 g, 0.015 mmol, 33%). LC-MS: Calc'd m/z=427.2 for C₂₁H₂₉N₇O₃. found [M+H]⁺=428.3. ¹H NMR (300 MHz, MeOD) δ 6.98 (d, J=8.3 Hz, 1H), 6.64 (d, J=2.1 Hz, 1H), 6.57-6.44 (m, 1H), 4.94 (s, 2H), 4.30 (t, J=6.5 Hz, 2H), 3.88 (s, 3H), 3.39 (q, J=6.1 Hz, 8H), 1.80-1.69 (m, 2H), 1.56-1.36 (m, 2H), 0.96 (t, J=7.4 Hz, 3H).

### Example 1.104: 6-amino-9-(4-(4-aminopiperidin-1-yl)-2-methoxybenzyl)-2-butoxy-9H-purin-8-ol (Compound 170)

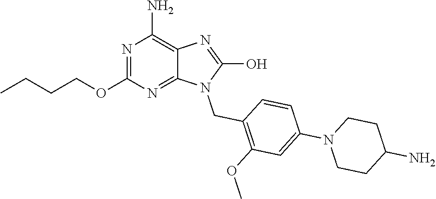 [SMILES: CCCCOc1nc(N)c2nc(O)n(Cc3ccc(N4CCC(N)CC4)cc3OC)c2n1]

A DMF (0.50 mL) solution of compound 32 (15 mg, 0.034 mmol, 1.0 eq.), 4-Boc-aminopiperidine (10 mg, 0.052 mmol, 1.5 eq.), KOtBu (15 mg, 0.14 mmol, 4.0 eq.) and Pd₂dba₃ (6.3 mg, 0.0068 mmol, 0.20 eq.) and RuPhos (12 mg, 0.028 mmol, 0.80 eq.) in a crimp-sealable microwave vial was thoroughly degassed and sealed under N₂ then heated to 100° C. for 18 h. The reaction mixture was then diluted with EtOAc (25 mL), filtered through celite and concentrated in vacuo. Preparative HPLC purification was accomplished as described in General Procedure 5, eluting with a 10 to 60% CH₃CN+0.1% TFA/H₂O+0.1% TFA gradient to give the Boc/8-MeO intermediate. This intermediate was then dissolved in CH₃CN (500 μL) and 6 M HCl (250 μL) and the mixture heated to 55° C. for 3 h. The reaction mixture was cooled to room temperature to give the title product 170 after lyophilization (1.8 mg, 0.0027 mmol, 7.9%). LC-MS: Calc'd m/z=441.3 for C₂₂H₃₁N₇O₃. found [M+H]⁺=442.4. ¹H NMR (300 MHz, MeOD) δ 6.96 (d, J=8.3 Hz, 1H), 6.62 (s, 1H), 6.52 (dd, J=8.4, 2.2 Hz, 1H), 4.96 (s, 2H), 4.31 (t, J=6.5 Hz, 2H), 3.84 (s, 3H), 3.90-3.75 (m, 3H), 2.86 (t, J=12.4 Hz, 2H), 2.12-2.07 (m, 2H), 1.84-1.65 (m, 4H), 1.56-1.38 (m, 2H), 0.97 (t, J=7.4 Hz, 3H).

### Example 1.105: 6-amino-2-butoxy-9-(2-methoxy-4-(3,9-diazaspiro[5.5]undecan-3-yl)benzyl)-9H-purin-8-ol (Compound 171)

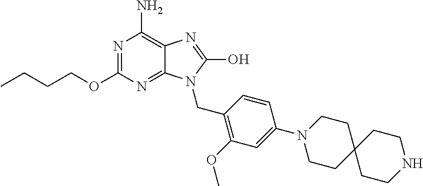 [SMILES: CCCCOc1nc(N)c2nc(O)n(Cc3ccc(N4CCC5(CCNCC5)CC4)cc3OC)c2n1]

A DMF (0.50 mL) solution of compound 32 (13 mg, 0.030 mmol, 1.0 eq.), tert-butyl 3,9-diazaspiro[5.5]undecane-3-carboxylate (12 mg, 0.046 mmol, 1.5 eq.), KOtBu (14 mg, 0.12 mmol, 4.0 eq.) and Pd₂dba₃ (5.9 mg, 0.0060 mmol, 0.20 eq.) and RuPhos (11 mg, 0.024 mmol, 0.80 eq.) in a crimp-sealable microwave vial was thoroughly degassed and sealed under N₂ then heated to 100° C. for 18 h. The reaction mixture was then diluted with EtOAc (25 mL), filtered through celite and concentrated in vacuo. Preparative HPLC purification was accomplished as described in General Procedure 5, eluting with a 10 to 60% CH₃CN+0.1% TFA/H₂O+0.1% TFA gradient to give the Boc/8-MeO intermediate. This intermediate was then dissolved in CH₃CN (500 μL) and 6 M HCl (250 μL) and the mixture heated to 55° C. for 3 h. The reaction mixture was cooled to room temperature to give the title product 171 after lyophilization (6.4 mg, 0.0088 mmol, 29%). LC-MS: Calc'd m/z=495.3 for C₂₆H₃₇N₇O₃. found [M+H]⁺=496.4. ¹H NMR (300 MHz, MeOD) δ 7.20-7.10 (m, 2H), 7.02 (dd, J=8.2, 2.2 Hz, 1H), 5.03 (s, 2H), 4.31 (t, J=6.5 Hz, 2H), 3.94 (s, 3H), 3.56 (t, J=5.7 Hz, 4H), 3.29-3.23 (m, 4H), 2.08-1.85 (m, 8H), 1.81-1.65 (m, 2H), 1.56-1.38 (m, 2H), 0.97 (t, J=7.4 Hz, 3H).

### Example 1.106: 3-(4-((6-amino-2-butoxy-8-methoxy-9H-purin-9-yl)methyl)-3-methoxy-phenyl)prop-2-yn-1-ol

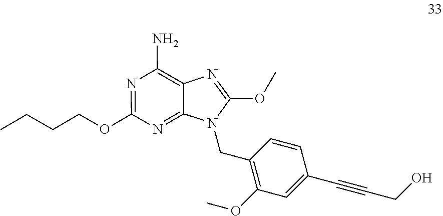 [SMILES: CCCCOc1nc(N)c2nc(OC)n(Cc3ccc(C#CCO)cc3OC)c2n1]

A solution of compound 32 (380 mg, 0.87 mmol, 1.0 eq.), Pd(OAc)₂ (20 mg, 0.087 mmol, 0.10 eq.), triphenylphosphine (110 mg, 0.44 mmol, 0.50 eq.), DIPEA (0.91 mL, 5.2 mmol, 6.0 eq.) and CuI (33 mg, 0.17 mmol, 0.20 eq.) in anhydrous DMF (4 mL) was sealed in a round-bottomed flask with a rubber septum and thoroughly degassed with N₂. Anhydrous propargyl alcohol (75 μL, 1.3 mmol, 1.5 eq.) was added via syringe and the reaction mixture heated to 75° C. for 18 h under N₂, after which it was diluted with H₂O (50 mL) and extracted with EtOAc (3×50 mL). The pooled organics were then washed with brine (1×50 mL), dried over Na₂SO₄ and concentrated in vacuo to give the crude product. Reverse phase flash purification was accomplished as described in General Procedure 5 using a 60 g C18 column, eluting with a 10 to 75% CH₃CN+0.1% TFA/H₂O+0.1% TFA gradient to give the title product 33 as a deep red solid (130 mg, 0.32 mmol, 36%). LC-MS: Calc'd m/z=411.2 for C₂₁H₂₅N₅O₄. found [M+H]⁺=412.3.

### Example 1.107: 3-(4-((6-amino-2-butoxy-8-methoxy-9H-purin-9-yl)methyl)-3-methoxy-phenyl)propan-1-ol

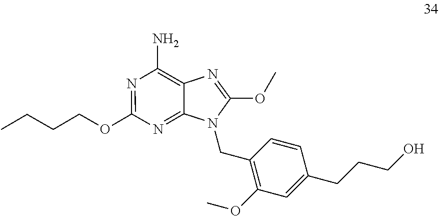 [SMILES: CCCCOc1nc(N)c2nc(OC)n(Cc3ccc(CCCO)cc3OC)c2n1]

A solution of compound 33 (150 mg, 0.37 mmol, 1.0 eq.) in MeOH (5 mL) was degassed with N₂, after which 10% Pd/C (39 mg, 0.037 mmol, 0.10 eq.) was added and the reaction vessel sealed and purged with additional N₂. The reaction vessel was then purged with H2 and the mixture stirred under H₂ at room temperature for 18 h. The suspension was filtered through celite and the filtrate concentrated in vacuo to give the title product 34 as a red oily solid, which was carried forward without additional purification (130 mg, 0.30 mmol, 83%; carried). LC-MS: Calc'd m/z=415.2 for C₂₁H₂₉N₅O₄. found [M+H]⁺=416.3.

### Example 1.108: 3-(4-((6-amino-2-butoxy-8-hydroxy-9H-purin-9-yl)methyl)-3-methoxyphenyl)-propyl Methanesulfonate

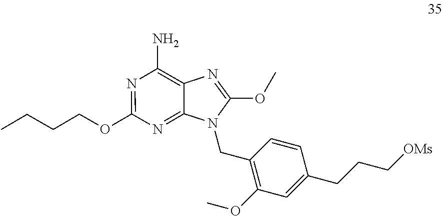 [SMILES: CCCCOc1nc(N)c2nc(OC)n(Cc3ccc(CCCOS(C)(=O)=O)cc3OC)c2n1]

To a solution of compound 34 (0.050 g, 0.12 mmol, 1.0 eq.) dissolved in anhydrous DCM (5 mL) was added DIPEA (63 μL, 0.36 mmol, 3.0 eq.) then methanesulfonyl chloride (17 mg, 0.14 mmol, 1.2 eq.) and the resulting solution stirred at room temperature for 1 h. The solvent was removed in vacuo to give the titled product 35, which was carried forward without additional purification (assumed quantitative yield). LC-MS: Calc'd m/z=479.2 for C₂₁H₂₉N₅O₆S. found [M+H]⁺=480.3.

### Example 1.109: 6-amino-2-butoxy-9-(2-methoxy-4-(3-(piperazin-1-yl)propyl)benzyl)-9H-purin-8-ol (Compound 173)

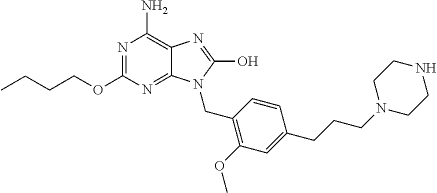 [SMILES: CCCCOc1nc(N)c2nc(O)n(Cc3ccc(CCCN4CCNCC4)cc3OC)c2n1]

The titled compound was prepared according to General Procedure 8 from compound 35 (0.010 g, 0.020 mmol, 1.0 eq.) and tert-butyl piperazine-1-carboxylate (15 mg, 0.081 mmol, 4.0 eq.). Preparative HPLC purification was accomplished as described in General Procedure 5, eluting with a 10 to 60% CH₃CN+0.1% TFA/H₂O+0.1% TFA gradient to give the Boc/8-MeO intermediate. This intermediate was then dissolved in CH₃CN (400 μL) and 6 M HCl (200 μL) and the mixture heated to 55° C. for 3 h. The reaction mixture was cooled to room temperature to give the title product 173 after lyophilization (4.6 mg, 0.0057 mmol, 28%). LC-MS: Calc'd m/z=469.3 for C₂₄H₃₅N₇O₃. found [M+H]⁺=470.4. ¹H NMR (300 MHz, MeOD) δ 7.02-6.85 (m, 2H), 6.83-6.71 (m, 1H), 5.01 (s, 2H), 4.30 (t, J=6.5 Hz, 2H), 4.16 (s, 2H), 3.86 (s, 3H), 3.56-3.43 (m, 4H), 3.06-2.93 (m, 2H), 2.71 (t, J=7.4 Hz, 2H), 2.10-1.94 (m, 2H), 1.77-1.64 (m, 2H), 1.63-1.28 (m, 4H), 0.97 (t, J=7.4 Hz, 3H).

### Example 1.110: 6-amino-9-(4-(3-(4-aminopiperidin-1-yl)propyl)-2-methoxybenzyl)-2-butoxy-9H-purin-8-ol (Compound 172)

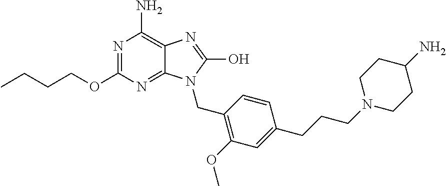 [SMILES: CCCCOc1nc(N)c2nc(O)n(Cc3ccc(CCCN4CCC(N)CC4)cc3OC)c2n1]

The titled compound was prepared according to General Procedure 8 from compound 35 (5.1 mg, 0.010 mmol, 1.0 eq.) and 4-Boc-aminopiperidine (8.3 mg, 0.042 mmol, 4.0 eq.). Preparative HPLC purification was accomplished as described in General Procedure 5, eluting with a 10 to 60% CH₃CN+0.1% TFA/H₂O+0.1% TFA gradient to give the Boc/8-MeO intermediate. This intermediate was then dissolved in CH₃CN (400 μL) and 6 M HCl (200 μL) and the mixture heated to 55° C. for 3 h. The reaction mixture was cooled to room temperature to give the title product 172 after lyophilization (1.0 mg, 0.0012 mmol, 12%). LC-MS: Calc'd m/z=483.3 for C₂₅H₃₇N₇O₃. found [M+H]⁺=484.4. ¹H NMR (300 MHz, MeOD) δ 6.96-6.85 (m, 2H), 6.76 (d, J=7.7 Hz, 1H), 5.00 (s, 2H), 4.27 (t, J=6.5 Hz, 2H), 3.88 (s, 3H), 3.75-3.65 (m, 2H), 3.47-3.41 (m, 1H), 3.19-3.01 (m, 2H), 2.77-2.66 (m, 2H), 2.33-2.22 (m, 2H), 2.11-2.05 (m, 2H), 1.98-1.88 (m, 2H), 1.79-1.63 (m, 2H), 1.48-1.34 (m, 4H), 0.97 (t, J=7.4 Hz, 3H).

### Example 1.111: 6-amino-9-(4-(3-(4-aminopiperidin-1-yl)propyl)-2-methoxybenzyl)-2-butoxy-9H-purin-8-ol (Compound 174)

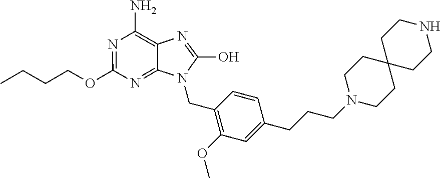 [SMILES: CCCCOc1nc(N)c2nc(O)n(Cc3ccc(CCCN4CCC5(CCNCC5)CC4)cc3OC)c2n1]

The titled compound was prepared according to General Procedure 8 from compound 35 (0.010 g, 0.020 mmol, 1.0 eq.) and tert-butyl 3,9-diazaspiro[5.5]undecane-3-carboxylate (0.020 g, 0.081 mmol, 4.0 eq.). Preparative HPLC purification was accomplished as described in General Procedure 5, eluting with a 10 to 60% CH₃CN+0.1% TFA/H₂O+0.1% TFA gradient to give the Boc/8-MeO intermediate. This intermediate was then dissolved in CH₃CN (400 μL) and 6 M HCl (200 μL) and the mixture heated to 55° C. for 3 h. The reaction mixture was cooled to room temperature to give the title product 174 after lyophilization (4.6 mg, 0.0052 mmol, 26%). LC-MS: Calc'd m/z=537.3 for C₂₉H₄₃N₇O₃. found [M+H]⁺=538.4. ¹H NMR (300 MHz, MeOD) δ 6.95-6.85 (m, 2H), 6.76 (d, J=7.8 Hz, 1H), 5.00 (s, 2H), 4.27 (t, J=6.6 Hz, 2H), 3.88 (s, 3H), 3.51-3.41 (m, 2H), 3.28-3.18 (m, 4H), 3.15-3.09 (m, 2H), 2.77-2.66 (m, 2H), 2.13-1.93 (m, 6H), 1.93-1.87 (m, 4H), 1.72-1.66 (m, 4H), 1.56-1.37 (m, 2H), 0.95 (q, J=9.9 Hz, 3H).

### Example 1.112: 6-amino-9-(4-(3-(4-(2-aminoethyl)piperidin-1-yl)propyl)-2-methoxybenzyl)-2-butoxy-9H-purin-8-ol (Compound 175)

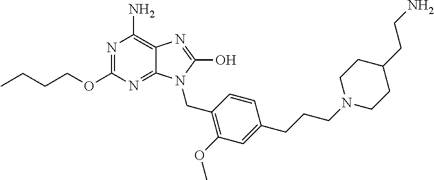 [SMILES: CCCCOc1nc(N)c2nc(O)n(Cc3ccc(CCCN4CCC(CCN)CC4)cc3OC)c2n1]

The titled compound was prepared according to General Procedure 8 from compound 35 (0.010 g, 0.020 mmol, 1.0 eq.) and tert-butyl N-[2-(piperidin-4-yl)ethyl]carbamate (19 mg, 0.081 mmol, 4.0 eq.). Preparative HPLC purification was accomplished as described in General Procedure 5, eluting with a 10 to 60% CH₃CN+0.1% TFA/H₂O+0.1% TFA gradient to give the Boc/8-MeO intermediate. This intermediate was then dissolved in CH₃CN (400 μL) and 6 M HCl (200 μL) and the mixture heated to 55° C. for 3 h. The reaction mixture was cooled to room temperature to give the title product 175 after lyophilization (1.6 mg, 0.0019 mmol, 9.4%). LC-MS: Calc'd m/z=511.3 for C₂₇H₄₁N₇O₃. found [M+H]⁺=512.4. ¹H NMR (300 MHz, MeOD) δ 6.94-6.85 (m, 2H), 6.76 (d, J=7.7 Hz, 1H), 5.00 (s, 2H), 4.25 (t, J=6.6 Hz, 2H), 3.88 (s, 3H), 3.64-3.58 (m, 2H), 3.21-2.85 (m, 6H), 2.74-2.66 (m, 2H), 2.21-1.93 (m, 4H), 1.80-1.57 (m, 5H), 1.53-1.39 (m, 2H), 1.36-1.28 (m, 2H), 0.96 (t, J=7.4 Hz, 3H).

### Example 1.113: 6-amino-2-butoxy-9-(2-methoxy-4-(3-(piperidin-4-ylamino)propyl)benzyl)-9H-purin-8-ol (Compound 176)

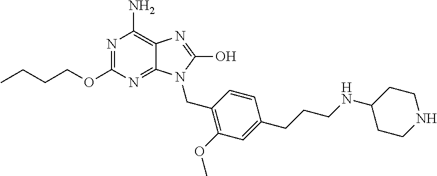 [SMILES: CCCCOc1nc(N)c2nc(O)n(Cc3ccc(CCCNC4CCNCC4)cc3OC)c2n1]

The titled compound was prepared according to General Procedure 8 from compound 35 (0.010 g, 0.020 mmol, 1.0 eq.) and 1-Boc-aminopiperidine carbamate (19 mg, 0.081 mmol, 4.0 eq.). Preparative HPLC purification was accomplished as described in General Procedure 5, eluting with a 10 to 60% CH₃CN+0.1% TFA/H₂O+0.1% TFA gradient to give the Boc/8-MeO intermediate. This intermediate was then dissolved in CH₃CN (400 μL) and 6 M HCl (200 μL) and the mixture heated to 55° C. for 3 h. The reaction mixture was cooled to room temperature to give the title product 176 after lyophilization (1.5 mg, 0.0018 mmol, 9.1%). LC-MS: Calc'd m/z=483.3 for C₂₅H₃₇N₇O₃. found [M+H]⁺=484.4. ¹H NMR (300 MHz, MeOD) δ 7.02-6.84 (m, 2H), 6.76 (dd, J=7.8, 1.5 Hz, 1H), 5.01 (s, 2H), 4.30 (t, J=6.5 Hz, 2H), 3.87 (s, 3H), 3.56-3.41 (m, 4H), 3.31-3.25 (m, 5H), 3.06-2.92 (m, 2H), 2.71 (t, J=7.4 Hz, 2H), 2.09-1.93 (m, 2H), 1.84-1.64 (m, 2H), 1.56-1.28 (m, 2H), 0.97 (t, J=7.4 Hz, 3H).

### Example 1.114: Ethyl-2-(3-methoxy-4-methylphenyl)acetate

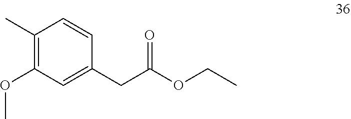

To a solution of (3-methoxy-4-methylphenyl)acetic acid (5.0 g, 28 mmol, 1.0 eq.) in EtOH (30 mL) was added concentrated H₂SO₄ (0.3 mL). The resulting mixture was heated to reflux for 1.5 h then concentrated in vacuo and redissolved in EtOAc (50 mL) The resulting organic solution was extracted with H₂O (1×20 mL) then brine (1×10 mL), dried over MgSO₄ and concentrated in vacuo to yield the crude title product 36 as a yellow oil, which was carried forward without additional purification (assumed quantitative yield). LC-MS: Calc'd m/z=208.1 for C₁₂H₁₆O₃. found [M+H]⁺=209.2.

### Example 1.115: Ethyl-2-(4-(bromomethyl)-3-methoxyphenyl)acetate

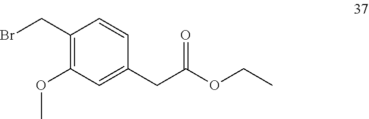 [SMILES: CCOC(=O)Cc1ccc(CBr)c(OC)c1]

To a solution of compound 36 (2.7 g, 13 mmol, 1.0 eq.) in DCM (10 mL) was added NBS (2.5 g, 14 mmol, 1.0 eq.) followed by 30% H₂O₂ (1.5 g, 13 mmol, 1.0 eq.). The resulting solution was stirred at room temperature for 18 h, concentrated in vacuo and redissolved in CH₃CN (5 mL) and H2O (1 mL). Reverse phase flash purification was accomplished as described in General Procedure 5 using a 60 g C18 column, eluting with a 20 to 60% CH₃CN+0.1% TFA/H₂O+0.1% TFA gradient, followed by normal phase flash purification as described in General Procedure 5 using a 50 g silica column, eluting with a 10 to 100% EtOAc/hexanes gradient to give the title compound 37 (350 mg, 1.2 mmol, 9.4%). LC-MS: Calc'd m/z=286.0 for C₁₂H₁₅BrO₃, found [M+H]⁺=287.0.

### Example 1.116: Ethyl-2-(4-((6-amino-2-butoxy-8-methoxy-9H-purin-9-yl)methyl)-3-methoxy-phenyl)acetate

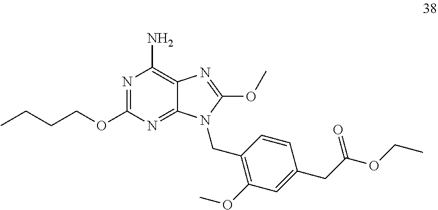 [SMILES: CCCCOc1nc(N)c2nc(OC)n(Cc3ccc(CC(=O)OCC)cc3OC)c2n1]

To a solution of compound 24 (470 mg, 2.0 mmol, 1.0 eq.) and compound 37 (560 mg, 2.0 mmol, 1.0 eq.) in DMF (3 mL) was added CsCO₃ (640 mg, 2.0 mmol, 1.0 eq.). The resulting suspension was stirred at room temperature for 18 h then concentrated in vacuo. Reverse phase flash purification was accomplished as described in General Procedure 5 using a 60 g C18 column, eluting with a 10 to 50% CH₃CN+0.1% TFA/H₂O+0.1% TFA gradient. The title compound 38 was obtained as a white solid (160 mg, 0.35 mmol, 18%). LC-MS: Calc'd m/z=443.2 for C₂₂H₂₉N₅O₅. found [M+H]⁺=444.3.

### Example 1.117: 2-(4-((6-amino-2-butoxy-8-methoxy-9H-purin-9-yl)methyl)-3-methoxy-phenyl)ethan-1-ol

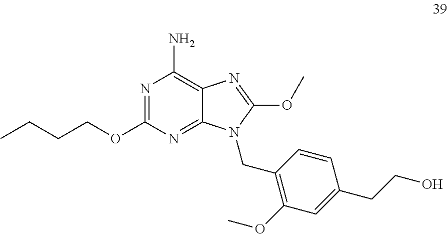 [SMILES: CCCCOc1nc(N)c2nc(OC)n(Cc3ccc(CCO)cc3OC)c2n1]

To a stirring solution of compound 38 (380 mg, 0.68 mmol, 1.0 eq.) in anhydrous THF (2 mL) cooled to 0° C. was added lithium aluminum hydride (86 mg, 1.4 mmol, 2.0 eq.). The resulting suspension was allowed to warm to room temperature and stirred for 15 mins, after which it was diluted with MeOH (5 mL) and H₂O (1 mL) then filtered through a celite plug. The filtrate was then concentrated in vacuo and reverse phase flash purification was accomplished as described in General Procedure 5 using a 30 g C18 column, eluting with a 10 to 50% CH₃CN+0.1% TFA/H₂O+0.1% TFA gradient to give the title product 39 as a white solid (0.20 g, 0.39 mmol, 57%). LC-MS: Calc'd m/z=401.2 for C₂₀H₂₇N₅O₄. found [M+H]⁺=402.3.

### Example 1.118: 6-amino-2-butoxy-9-(4-(2-chloroethyl)-2-methoxybenzyl)-9H-purin-8-ol

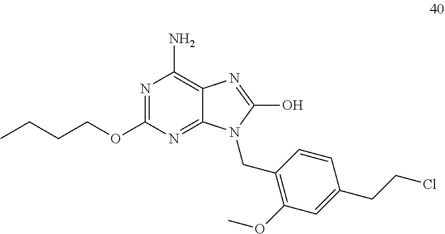 [SMILES: CCCCOc1nc(N)c2nc(O)n(Cc3ccc(CCCl)cc3OC)c2n1]

The title compound 40 was prepared according to General Procedure 3 from compound 39 (0.10 g, 0.19 mmol, 1.0 eq.) and 10% SOCl₂/DCM (10 mL). The titled compound was obtained as a yellow solid (72 mg, 0.16 mmol, 84%). LC-MS: Calc'd m/z=405.2 for C₁₉H₂₄ClN₅O₃. found [M+H]⁺=406.3.

### Example 1.119: 6-amino-2-butoxy-9-(2-methoxy-4-(2-(piperazin-1-yl)ethyl)benzyl)-9H-purin-8-ol (Compound 180)

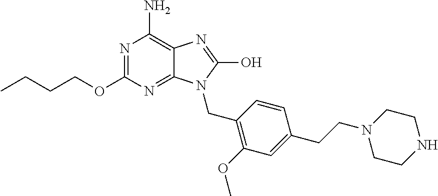 [SMILES: CCCCOc1nc(N)c2nc(O)n(Cc3ccc(CCN4CCNCC4)cc3OC)c2n1]

The titled compound was prepared according to General Procedure 1 from compound 40 (9.7 mg, 0.024 mmol, 1.0 eq.), NaI (0.5 mg, 0.003 mmol, 0.1 eq.) and tert-butyl piperazine-1-carboxylate (9.0 mg, 0.048 mmol, 2.0 eq.). Preparative HPLC purification was accomplished as described in General Procedure 5, eluting with a 10 to 60% CH₃CN+0.1% TFA/H₂O+0.1% TFA gradient. Deprotection was accomplished according to General Procedure 2 to give the title compound 180 as a white solid (7.0 mg, 0.010 mmol, 43%). LC-MS: Calc'd m/z=455.3 for C₂₃H₃₃N₇O₃. found [M+H]⁺=456.4. ¹H NMR (300 MHz, MeOD) δ 7.23 (dd, J=8.5, 2.2 Hz, 1H), 7.03-6.97 (m, 2H), 5.04 (s, 2H), 4.35 (td, J=6.5, 4.1 Hz, 2H), 3.86 (d, J=2.0 Hz, 3H), 3.58-3.43 (m, 6H), 3.32-3.19 (m, 4H), 2.96 (dd, J=10.0, 6.1 Hz, 2H), 1.74 (dq, J=8.5, 6.6 Hz, 2H), 1.56-1.38 (m, 2H), 0.97 (t, J=7.4 Hz, 3H).

### Example 1.120: 6-amino-9-(4-(2-(4-aminopiperidin-1-yl)ethyl)-2-methoxybenzyl)-2-butoxy-9H-purin-8-ol (Compound 181)

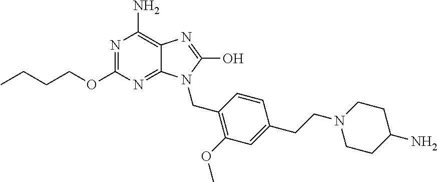 [SMILES: CCCCOc1nc(N)c2nc(O)n(Cc3ccc(CCN4CCC(N)CC4)cc3OC)c2n1]

The titled compound was prepared according to General Procedure 1 from compound 40 (9.7 mg, 0.024 mmol, 1.0 eq.), NaI (0.5 mg, 0.003 mmol, 0.1 eq.) and 4-Boc-aminopiperidine (0.010 g, 0.048 mmol, 2.0 eq.). Preparative HPLC purification was accomplished as described in General Procedure 5, eluting with a 10 to 60% CH₃CN+0.1% TFA/H₂O+0.1% TFA gradient. Deprotection was accomplished according to General Procedure 2 to give the title compound 181 as a white solid (6.1 mg, 0.0087 mmol, 37%). LC-MS: Calc'd m/z=469.3 for C₂₄H₃₅N₇O₃. found [M+H]⁺=470.4. ¹H NMR (300 MHz, MeOD) δ 7.23 (dd, J=8.3, 2.3 Hz, 1H), 7.04-6.93 (m, 2H), 5.04 (s, 2H), 4.35 (t, J=2.2 Hz, 2H), 3.85 (s, 3H), 3.79-3.39 (m, 3H), 3.33-3.05 (m, 4H), 2.98 (dd, J=10.3, 6.4 Hz, 2H), 2.38-1.89 (m, 4H), 1.82-1.58 (m, 2H), 1.56-1.38 (m, 2H), 0.97 (t, J=7.4 Hz, 3H).

### Example 1.121: 9-(4-(2-(3,9-diazaspiro[5.5]undecan-3-yl)ethyl)-2-methoxybenzyl)-6-amino-2-butoxy-9H-purin-8-ol (Compound 177)

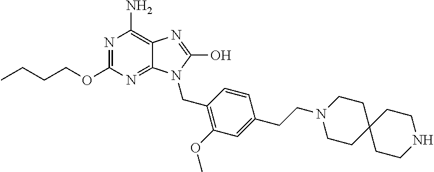 [SMILES: CCCCOc1nc(N)c2nc(O)n(Cc3ccc(CCN4CCC5(CCNCC5)CC4)cc3OC)c2n1]

The titled compound was prepared according to General Procedure 1 from compound 40 (9.7 mg, 0.024 mmol, 1.0 eq.), NaI (4 mg, 0.02 mmol, 1 eq.) and tert-butyl 3,9-diazaspiro[5.5]undecane-3-carboxylate (18 mg, 0.071 mmol, 3.0 eq.). Preparative HPLC purification was accomplished as described in General Procedure 5, eluting with a 10 to 60% CH₃CN+0.1% TFA/H₂O+0.1% TFA gradient. Deprotection was accomplished according to General Procedure 2 to give the title compound 177 as a white solid (11 mg, 0.015 mmol, 63%). LC-MS: Calc'd m/z=523.3 for C₂₈H₄₁N₇O₃. found [M+H]⁺=524.4. ¹H NMR (300 MHz, MeOD) δ 7.23 (dd, J=8.4, 2.2 Hz, 1H), 7.04-6.96 (m, 2H), 5.04 (s, 2H), 4.37 (t, J=6.5 Hz, 2H), 3.86 (s, 3H), 3.55-3.45 (m, 2H), 3.34-3.05 (m, 8H), 3.03-2.91 (m, 2H), 2.08-1.86 (m, 4H), 1.83-1.64 (m, 6H), 1.60-1.29 (m, 2H), 0.97 (t, J=7.4 Hz, 3H).

### Example 1.122: 6-amino-9-(4-(2-(4-(2-aminoethyl)piperidin-1-yl)ethyl)-2-methoxybenzyl)-2-butoxy-9H-purin-8-ol (Compound 178)

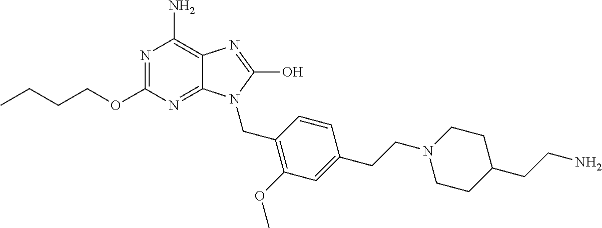 [SMILES: CCCCOc1nc(N)c2nc(O)n(Cc3ccc(CCN4CCC(CCN)CC4)cc3OC)c2n1]

The titled compound was prepared according to General Procedure 1 from compound 40 (9.7 mg, 0.024 mmol, 1.0 eq.), NaI (4 mg, 0.02 mmol, 1 eq.) and tert-butyl N-[2-(piperidin-4-yl)ethyl]carbamate (16 mg, 0.071 mmol, 3.0 eq.). Preparative HPLC purification was accomplished as described in General Procedure 5, eluting with a 10 to 60% CH₃CN+0.1% TFA/H₂O+0.1% TFA gradient. Deprotection was accomplished according to General Procedure 2 to give the title compound 178 as a white solid (13 mg, 0.018 mmol, 76%). LC-MS: Calc'd m/z=497.3 for C₂₆H₃₉N₇O₃. found [M+H]⁺=498.4. ¹H NMR (300 MHz, MeOD) δ 7.26-7.10 (m, 1H), 7.04-6.88 (m, 1H), 6.94 (s, 1H), 5.03 (t, J=2.4 Hz, 2H), 4.40-4.24 (m, 2H), 3.83 (s, 3H), 3.72-3.58 (m, 2H), 3.26 (dd, J=10.4, 6.3 Hz, 2H), 2.97 (dt, J=18.0, 7.2 Hz, 4H), 2.71 (t, J=7.1 Hz, 2H), 2.02 (d, J=14.1 Hz, 1H), 1.81-1.59 (m, 6H), 1.45 (p, J=7.5 Hz, 2H), 1.34-1.28 (m, 2H), 0.97 (t, J=7.4 Hz, 3H).

### Example 1.123: 6-amino-2-butoxy-9-(2-methoxy-4-(2-(piperidin-4-ylamino)ethyl)benzyl)-9H-purin-8-ol (Compound 179)

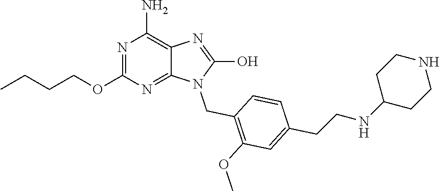 [SMILES: CCCCOc1nc(N)c2nc(O)n(Cc3ccc(CCNC4CCNCC4)cc3OC)c2n1]

The titled compound was prepared according to General Procedure 1 from compound 40 (9.7 mg, 0.024 mmol, 1.0 eq.), NaI (4 mg, 0.02 mmol, 1 eq.) and 1-Boc-aminopiperidine (0.010 g, 0.048 mmol, 2.0 eq.). Preparative HPLC purification was accomplished as described in General Procedure 5, eluting with a 10 to 60% CH₃CN+0.1% TFA/H₂O+0.1% TFA gradient. Deprotection was accomplished according to General Procedure 2 to give the title compound 179 as a white solid (12 mg, 0.015 mmol, 62%). LC-MS: Calc'd m/z=469.3 for C₂₄H₃₅N₇O₃. found [M+H]⁺=470.4. ¹H NMR (300 MHz, MeOD) δ 7.23 (dd, J=8.4, 2.2 Hz, 1H), 7.00 (dd, J=5.3, 3.1 Hz, 2H), 5.04 (s, 2H), 4.36 (t, J=6.5 Hz, 2H), 3.85 (s, 3H), 3.61-3.34 (m, 3H), 3.24 (dd, J=9.2, 6.6 Hz, 2H), 3.08 (td, J=13.2, 2.8 Hz, 2H), 2.91 (dd, J=9.2, 6.5 Hz, 2H), 2.33 (d, J=13.6 Hz, 2H), 1.99-1.67 (m, 4H), 1.56-1.38 (m, 2H), 1.33 (s, 3H).

### Example 1.124: 2-((1-((tert-butyldiphenylsilyl)oxy)pentan-2-yl)oxy)-9-(tetrahydro-2H-pyran-2-yl)-9H-purin-6-amine

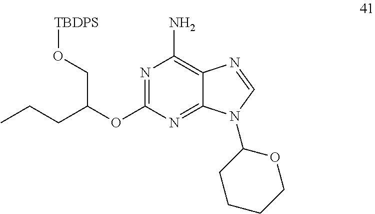

A mixture of compound 2 (5.0 g, 19 mmol, 1.0 eq.), compound 4 (20.3 g, 59.1 mmol, 3.0 eq.) and CsCO₃ (6.4 g, 19 mmol, 1.0 eq.) was heated at 170° C. for 18 h. K₂CO₃ (20 mg) was then added, and the reaction stirred at 170° C. for an additional 72 h. The reaction was diluted with DCM (1000 mL), filtered, and concentrated in vacuo to give the crude product as a brown solid. Normal phase flash purification was accomplished as described in General Procedure 5 using a 100 g silica column, eluting with a 0 to 100% EtOAc/hexanes gradient, followed by reverse phase flash purification was accomplished as described in General Procedure 5 using a 60 g C18 column, eluting with a 10 to 100% CH₃CN+0.1% TFA/H₂O+0.1% TFA gradient to give the title product 41 as a white solid (790 mg, 1.4 mmol, 7.2%). LC-MS: Calc'd m/z=559.3 for C₃₁H₄₁N₅O₃Si, found [M+H]⁺=560.4.

### Example 1.125: 2-((1-((tert-butyldiphenylsilyl)oxy)pentan-2-yl)oxy)-9H-purin-6-amine

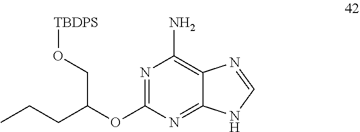

Compound 41 (210 mg, 0.38 mmol, 1.0 eq.) was dissolved in 15% TFA/MeOH (5 mL) and the resulting solution stirred at room temperature for 18 h, after which the solvent was removed in vacuo. Reverse phase flash purification was accomplished as described in General Procedure 5 using a 60 g C18 column, eluting with a 10 to 50% CH₃CN+0.1% TFA/H₂O+0.1% TFA gradient to give the title product 42 as a solid (57 mg, 0.097 mmol, 26%). LC-MS: Calc'd m/z=475.3 for C₂₆H₃₃N₅O₂Si. found [M+H]⁺=476.4.

### Example 1.126: tert-butyl (4-((6-amino-2-((1-((tert-butyldiphenylsilyl)oxy)pentan-2-yl)oxy)-9H-purin-9-yl)methyl)benzyl)carbamate

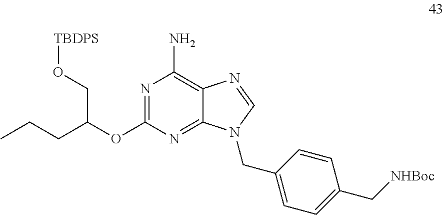

To a solution of compound 42 (41 mg, 0.14 mmol, 1.0 eq.) and tert-butyl (4-(aminomethyl)benzyl)carbamate (0.080 g, 0.14 mmol, 1.0 eq.) in DMF (2 mL) was added CsCO₃ (44 mg, 0.14 mmol, 1.0 eq.). The resulting suspension was stirred at room temperature for 18 h then concentrated in vacuo. Reverse phase flash purification was accomplished as described in General Procedure 5 using a 12 g C18 column, eluting with a 10 to 50% CH₃CN+0.1% TFA/H₂O+0.1% TFA gradient. The title compound 43 was obtained as a white solid (120 mg, 0.13 mmol, 91%). LC-MS: Calc'd m/z=694.4 for C₃₉H₅₀N₆O₄Si. found [M+H]⁺=695.5.

### Example 1.127: tert-butyl (4-((6-amino-2-((1-hydroxypentan-2-yl)oxy)-9H-purin-9-yl)-methyl)benzyl)carbamate

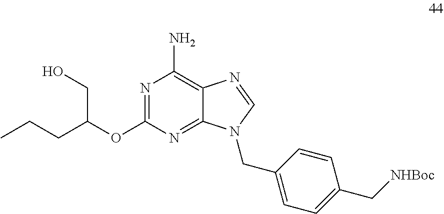 [SMILES: CCCC(CO)Oc1nc(N)c2ncn(Cc3ccc(CNC(=O)OC(C)(C)C)cc3)c2n1]

A solution of compound 43 (120 mg, 0.17 mmol, 1.0 eq.) and TBAF (0.70 mL, 1 M in THF, 1.0 eq.) was stirred at 40° C. for 18 h. Reverse phase flash purification was accomplished as described in General Procedure 5 using a 12 g C18 column, eluting with a 10 to 50% CH₃CN+0.1% TFA/H₂O+0.1% TFA gradient. The title compound 44 was obtained as a white solid (65 mg, 0.14 mmol, 84%). LC-MS: Calc'd m/z=456.3 for C₂₃H₃₂N₆O₄. found [M+H]⁺=457.4.

### Example 1.128: tert-butyl (4-((6-amino-8-bromo-2-((1-hydroxypentan-2-yl)oxy)-9H-purin-9-yl)methyl)benzyl)carbamate

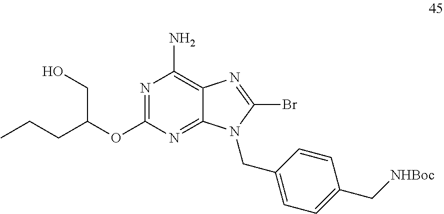 [SMILES: CCCC(CO)Oc1nc(N)c2nc(Br)n(Cc3ccc(CNC(=O)OC(C)(C)C)cc3)c2n1]

To a suspension of compound 44 (52 mg, 0.11 mmol, 1.0 eq.) and CsCO3 (37 mg, 0.11 mmol, 1.0 eq.) in DCM (3 mL) was added NBS (0.020 g, 0.11 mmol, 1.0 eq.) and the resulting mixture stirred at room temperature for 2 h. The reaction mixture was then quenched with 1 M NaHSO₃ (0.50 mL), diluted with H₂O (5 mL) and extracted with EtOAc (2×10 mL). The pooled organics were then dried over MgSO₄ and concentrated in vacuo to yield the crude product as an orange solid. Reverse phase flash purification was accomplished as described in General Procedure 5 using a 12 g C18 column, eluting with a 10 to 100% CH₃CN+0.1% TFA/H₂O+0.1% TFA gradient. The title compound 45 was obtained as a solid (17 mg, 0.032 mmol, 28%). LC-MS: Calc'd m/z=534.2 for C₂₃H₃₁BrN₆O₄. found [M+H]⁺=535.2.

### Example 1.129: 6-amino-9-(4-(aminomethyl)benzyl)-2-((1-hydroxypentan-2-yl)oxy)-9H-purin-8-ol (Compound 182)

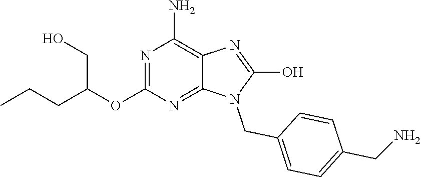 [SMILES: CCCC(CO)Oc1nc(N)c2nc(O)n(Cc3ccc(CN)cc3)c2n1]

Compound 45 (7.0 mg, 0.013 mmol, 1.0 eq.) was dissolved in MeOH (5 mL) followed by the addition of NaOMe (2.8 mg, 0.052 mmol, 4.0 eq.) and the resulting mixture heated at 80° C. for 36 h, after which the solvent was removed in vacuo to give the crude intermediate. This intermediate was then dissolved in 3 M HCl (3 mL) and heated to 60° C. for 3 h. Preparative HPLC purification was accomplished as described in General Procedure 5, eluting with a 10 to 50% CH₃CN+0.1% TFA/H₂O+0.1% TFA gradient to give the title compound 182 as a white solid (4.1 mg, 0.011 mmol, 85%). LC-MS: Calc'd m/z=372.2 for C₁₈H₂₄N₆O₃. found [M+H]⁺=373.3. ¹H NMR (300 MHz, MeOD) δ 7.54-7.38 (m, 4H), 5.03 (s, 2H), 4.14-4.07 (m, 3H), 3.70 (d, J=5.0 Hz, 2H), 1.76-1.63 (m, 2H), 1.59-1.37 (m, 2H), 0.96 (t, J=7.3 Hz, 3H).

### Example 1.130: N²-pentyl-9-(tetrahydro-2H-pyran-2-yl)-9H-purine-2,6-diamine

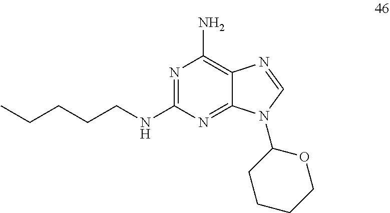 [SMILES: CCCCCNc1nc(N)c2ncn(C3CCCCO3)c2n1]

To a solution of compound 2 (2.0 g, 7.9 mmol, 1.0 eq.) in ethylene glycol (10 mL) was added amylamine (3.6 mL, 32 mmol, 4.0 eq.). The resulting solution was stirred at 120° C. for 18 h, after which it was cooled and taken up in EtOAc (100 mL). The resulting organic solution was washed with H₂O (2×50 mL), dried over MgSO₄, and concentrated in vacuo to yield the title compound 46 as a viscous brown oil (2.4 g, 7.9 mmol, 100%). LC-MS: Calc'd m/z=304.2 for C₁₅H₂₄N₆O. found [M+H]⁺=305.2.

### Example 1.131: 8-bromo-N²-pentyl-9-(tetrahydro-2H-pyran-2-yl)-9H-purine-2,6-diamine

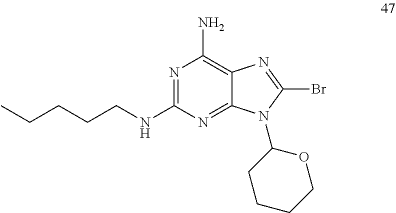 [SMILES: CCCCCNc1nc(N)c2nc(Br)n(C3CCCCO3)c2n1]

To a suspension of compound 46 (2.4 g, 7.9 mmol, 1.0 eq.) in DCM (50 mL) at 0° C. was added NBS (1.5 g, 8.3 mmol, 1.1 eq.). The resulting mixture was allowed to warm to room temperature and stirred for 30 mins. The reaction mixture was then washed with H₂O (2×50 mL) and the remaining organics dried over MgSO₄ then concentrated in vacuo to yield the title product 47 (2.9 g, 7.6 mmol, 96%). LC-MS: Calc'd m/z=382.1 for C₁₅H₂₃BrN₆O. found [M+H]⁺=383.2.

### Example 1.132: 8-methoxy-N²-pentyl-9H-purine-2,6-diamine

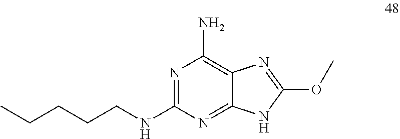 [SMILES: CCCCCNc1nc(N)c2nc(OC)[nH]c2n1]

To a solution of compound 47 (2.9 g, 7.6 mmol, 1.0 eq.) in MeOH (50 mL) was added NaOMe (4.1 g, 76 mmol, 10 eq.) and the resulting mixture heated at 70° C. for 36 h, after which the solvent was removed in vacuo and the residue taken up in EtOAc (50 mL). The resulting organic solution was washed with H₂O (2×50 mL) and then concentrated in vacuo to give the crude THP-protected 8-methoxy intermediate. This intermediate was then dissolved in 15% TFA/MeOH (50 mL) and the resulting solution stirred at room temperature for 72 h. The solvent was removed in vacuo to yield the title compound 48 (2.2 g, 6.0 mmol, 80%). LC-MS: Calc'd m/z=250.2 for C₁₁H₁₆N₆O. found [M+H]⁺=251.3.

### Example 1.133: Methyl-4-((6-amino-8-methoxy-2-(pentylamino)-9H-purin-9-yl)methyl)-benzoate

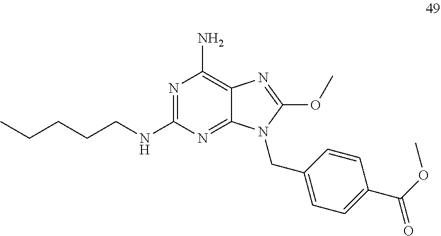 [SMILES: CCCCCNc1nc(N)c2nc(OC)n(Cc3ccc(C(=O)OC)cc3)c2n1]

To a solution of compound 48 (2.2 g, 6.0 mmol, 1.0 eq.) and methyl-4-(bromomethyl)benzoate (1.4 g, 6.0 mmol, 1.0 eq.) in DMF (50 mL) was added CsCO₃ (5.9 g, 18 mmol, 3.0 eq.). The resulting suspension was stirred at room temperature for 18 h then concentrated in vacuo. Reverse phase flash purification was accomplished as described in General Procedure 5 using a 30 g C18 column, eluting with a 10 to 50% CH₃CN+0.1% TFA/H₂O+0.1% TFA gradient. The title compound 49 was obtained as a white solid (910 mg, 2.3 mmol, 38%). LC-MS: Calc'd m/z=398.2 for C₂₀H₂₆N₆O₃. found [M+H]⁺=399.3.

### Example 1.134: (4-((6-amino-8-methoxy-2-(pentylamino)-9H-purin-9-yl)methyl)phenyl)-methanol

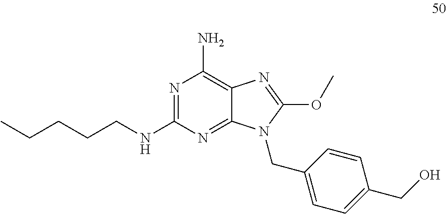 [SMILES: CCCCCNc1nc(N)c2nc(OC)n(Cc3ccc(CO)cc3)c2n1]

To a stirring solution of compound 49 (910 mg, 2.3 mmol, 1.0 eq.) in anhydrous THF (20 mL) cooled to 0° C. was added lithium aluminum hydride (4.5 mL, 1 M in THF, 2.0 eq.). The resulting suspension was allowed to warm to room temperature and stirred for 1 h, after which it was quenched with wetted Na₂SO₄ (1 g) and H₂O (2 mL) then filtered through a celite plug. The celite plug was washed with MeOH (50 mL) and the combined filtrate concentrated in vacuo. Reverse phase flash purification was accomplished as described in General Procedure 5 using a 60 g C18 column, eluting with a 10 to 50% CH₃CN+0.1% TFA/H₂O+0.1% TFA gradient to give the title product 50 as a white solid (480 mg, 1.3 mmol, 57%). LC-MS: Calc'd m/z=370.2 for C₁₉H₂₆N₆O₂. found [M+H]⁺=371.3.

### Example 1.135: 6-amino-9-(4-(chloromethyl)benzyl)-2-(pentylamino)-9H-purin-8-ol

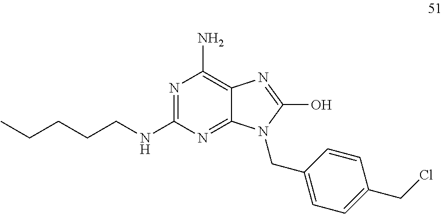 [SMILES: CCCCCNc1nc(N)c2nc(O)n(Cc3ccc(CCl)cc3)c2n1]

To a solution of compound 50 (0.10 g, 0.27 mmol, 1.0 eq.) in DCM (5 mL) was added SOCl₂ (0.10 mL, 1.4 mmol, 5.0 eq.) and the resulting solution stirred at room temperature for 3 h. The solvent was then removed in vacuo and the residue co-evaporated with toluene (2×10 mL) to give the title compound 51 as a yellow solid (assumed quantitative yield). LC-MS: Calc'd m/z=374.2 for C₁₈H₂₃ClN₆O. found [M+H]⁺=375.3.

### Example 1.136: 6-amino-9-(4-(aminomethyl)benzyl)-2-(pentylamino)-9H-purin-8-ol (Compound 183)

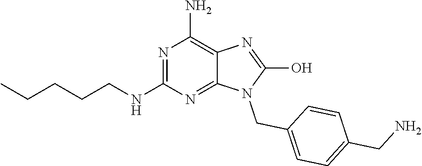 [SMILES: CCCCCNc1nc(N)c2nc(O)n(Cc3ccc(CN)cc3)c2n1]

To compound 51 (0.010 g, 0.027 mmol, 1.0 eq.) was added NH₄OH (1 mL, 1 M in MeOH) and the resulting solution heated to 60° C. for 18 h, after which the solvent was removed in vacuo. Reverse phase flash purification was accomplished as described in General Procedure 5 using a 60 g C18, eluting with a 10 to 40% CH₃CN+0.1% TFA/H₂O+0.1% TFA gradient to give the title product 183 as a white solid (2.1 mg, 0.0036 mmol, 13%). LC-MS: Calc'd m/z=355.2 for C₁₈H₂₅N₇O. found [M+H]⁺=356.3. ¹H NMR (300 MHz, MeOD) δ 7.55-7.38 (m, 4H), 5.02 (s, 2H), 4.11 (s, 2H), 3.41 (t, J=7.1 Hz, 2H), 1.64 (t, J=7.1 Hz, 2H), 1.44-1.28 (m, 4H), 1.00-0.90 (m, 3H).

### Example 1.137: 6-amino-9-(4-(((2-hydroxy-2-methylpropyl)amino)methyl)benzyl)-2-(pentyl-amino)-9H-purin-8-ol (Compound 184)

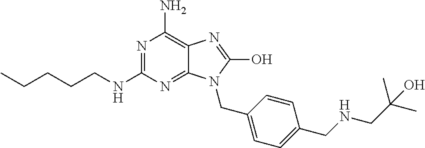 [SMILES: CCCCCNc1nc(N)c2nc(O)n(Cc3ccc(CNCC(C)(C)O)cc3)c2n1]

The titled compound was prepared according to General Procedure 1 from compound 51 (0.010 g, 0.027 mmol, 1.0 eq.) and 1-amino-2-methylpropan-2-ol (20 mg, 0.23 mmol, 8.5 eq.). Preparative HPLC purification was accomplished as described in General Procedure 5, eluting with a 10 to 50% CH₃CN+0.1% TFA/H₂O+0.1% TFA gradient to give the title compound 184 as a white solid (8.5 mg, 0.013 mmol, 49%). LC-MS: Calc'd m/z=427.3 for C₂₂H₃₃N₇O₂. found [M+H]⁺=428.4. ¹H NMR (300 MHz, MeOD) δ 7.52 (d, J=2.0 Hz, 4H), 5.04 (s, 2H), 4.24 (s, 2H), 3.42 (t, J=7.1 Hz, 2H), 2.90 (s, 2H), 1.65 (t, J=7.1 Hz, 2H), 1.39 (h, J=3.3 Hz, 4H), 1.26 (s, 6H), 1.00-0.89 (m, 3H).

### Example 1.138: 6-amino-9-(4-((diethylamino)methyl)benzyl)-2-(pentylamino)-9H-purin-8-ol (Compound 185)

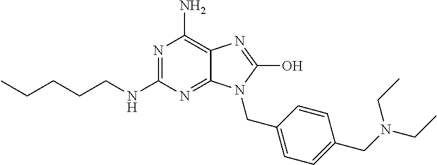 [SMILES: CCCCCNc1nc(N)c2nc(O)n(Cc3ccc(CN(CC)CC)cc3)c2n1]

The titled compound was prepared according to General Procedure 1 from compound 51 (0.010 g, 0.027 mmol, 1.0 eq.) and diethylamine (28 μL, 0.27 mmol, 10 eq.). Preparative HPLC purification was accomplished as described in General Procedure 5, eluting with a 10 to 50% CH₃CN+0.1% TFA/H₂O+0.1% TFA gradient to give the title compound 185 as a white solid (8.3 mg, 0.013 mmol, 49%). LC-MS: Calc'd m/z=411.3 for C₂₂H₃₃N₇O. found [M+H]⁺=412.4. ¹H NMR (300 MHz, MeOD) δ 7.60-7.46 (m, 4H), 5.05 (s, 2H), 4.34 (s, 2H), 3.42 (t, J=7.1 Hz, 2H), 3.28-3.13 (m, 4H), 1.70-1.57 (m, 2H), 1.44-1.29 (m, 10H), 0.95 (td, J=5.7, 3.0 Hz, 3H).

### Example 1.139: 6-amino-9-(4-(((3,3-difluorocyclobutyl)amino)methyl)benzyl)-2-(pentyl-amino)-9H-purin-8-ol (Compound 186)

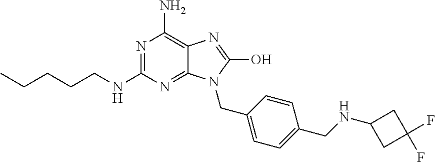 [SMILES: CCCCCNc1nc(N)c2nc(O)n(Cc3ccc(CNC4CC(F)(F)C4)cc3)c2n1]

The titled compound was prepared according to General Procedure 1 from compound 51 (0.010 g, 0.027 mmol, 1.0 eq.) and 3,3-difluorocyclobutylamine (20 mg, 0.18 mmol, 6.7 eq.). Preparative HPLC purification was accomplished as described in General Procedure 5, eluting with a 10 to 50% CH₃CN+0.1% TFA/H₂O+0.1% TFA gradient to give the title compound 186 as a white solid (6.4 mg, 0.0095 mmol, 35%). LC-MS: Calc'd m/z=445.2 for C₂₂H₂₉F₂N₇O. found [M+H]⁺=446.3. ¹H NMR (300 MHz, MeOD) δ 7.51 (q, J=8.3 Hz, 4H), 5.04 (s, 2H), 4.21 (s, 2H), 3.79 (dt, J=14.6, 7.5 Hz, 1H), 3.41 (t, J=7.1 Hz, 2H), 3.14-2.75 (m, 4H), 1.72-1.59 (m, 2H), 1.45-1.32 (m, 4H), 1.00-0.89 (m, 3H).

### Example 1.140: 6-amino-2-(pentylamino)-9-(4-(piperazin-1-ylmethyl)benzyl)-9H-purin-8-ol (Compound 187)

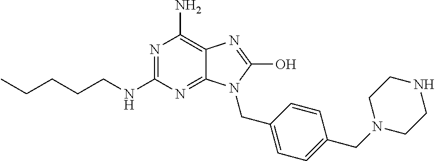 [SMILES: CCCCCNc1nc(N)c2nc(O)n(Cc3ccc(CN4CCNCC4)cc3)c2n1]

The titled compound was prepared according to General Procedure 1 from compound 51 (0.010 g, 0.027 mmol, 1.0 eq.) and tert-butyl piperazine-1-carboxylate (20 mg, 0.10 mmol, 3.7 eq.). Preparative HPLC purification was accomplished as described in General Procedure 5, eluting with a 10 to 50% CH₃CN+0.1% TFA/H₂O+0.1% TFA gradient. Half of the resulting Boc intermediate was reserved and deprotection of the remaining material was accomplished according to General Procedure 2 to give the title compound 186 as a yellow solid (3.8 mg, 0.0050 mmol, 37%). LC-MS: Calc'd m/z=424.3 for C₂₂H₃₂N₈O. found [M+H]⁺=425.4. ¹H NMR (300 MHz, MeOD) δ 7.48 (d, J=8.0 Hz, 2H), 7.41 (d, J=7.9 Hz, 2H), 5.01 (s, 2H), 3.91 (s, 2H), 3.44 (t, J=7.1 Hz, 2H), 3.39-3.31 (m, 4H), 3.04-2.94 (m, 4H), 1.71-1.60 (m, 2H), 1.44-1.36 (m, 4H), 1.00-0.90 (m, 3H).

### Example 1.141: 6-amino-9-(4-((4-aminopiperidin-1-yl)methyl)benzyl)-2-(pentylamino)-9H-purin-8-ol (Compound 188)

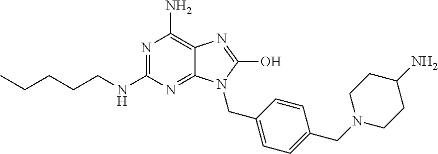 [SMILES: CCCCCNc1nc(N)c2nc(O)n(Cc3ccc(CN4CCC(N)CC4)cc3)c2n1]

The titled compound was prepared according to General Procedure 1 from compound 51 (0.010 g, 0.027 mmol, 1.0 eq.) and 4-Boc-aminopiperidine (20 mg, 0.096 mmol, 3.6 eq.). Preparative HPLC purification was accomplished as described in General Procedure 5, eluting with a 10 to 50% CH₃CN+0.1% TFA/H₂O+0.1% TFA gradient. Half of the resulting Boc intermediate was reserved and deprotection of the remaining material was accomplished according to General Procedure 2 to give the title compound 188 as a white solid (4.1 mg, 0.0053 mmol, 39%). LC-MS: Calc'd m/z=438.3 for C₂₃H₃₄N₈O. found [M+H]⁺=439.4. ¹H NMR (300 MHz, MeOD) δ 7.53 (q, J=8.1 Hz, 4H), 5.05 (s, 2H), 4.34 (s, 2H), 3.59 (d, J=13.0 Hz, 2H), 3.48-3.38 (m, 3H), 3.15 (t, J=13.2 Hz, 2H), 2.30-2.20 (m, 2H), 2.04-1.90 (m, 2H), 1.71-1.60 (m, 2H), 1.44-1.28 (m, 4H), 1.00-0.89 (m, 3H).

### Example 1.142: 6-amino-2-(pentylamino)-9-(4-((piperidin-4-ylamino)methyl)benzyl)-9H-purin-8-ol (Compound 194)

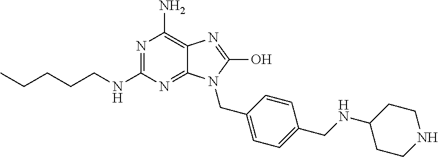 [SMILES: CCCCCNc1nc(N)c2nc(O)n(Cc3ccc(CNC4CCNCC4)cc3)c2n1]

The titled compound was prepared according to General Procedure 1 from compound 51 (0.010 g, 0.027 mmol, 1.0 eq.) and 1-Boc-aminopiperidine (20 mg, 0.096 mmol, 3.6 eq.). Preparative HPLC purification was accomplished as described in General Procedure 5, eluting with a 10 to 50% CH₃CN+0.1% TFA/H₂O+0.1% TFA gradient. Half of the resulting Boc intermediate was reserved and deprotection of the remaining material was accomplished according to General Procedure 2 to give the title compound 194 as a white solid (6.0 mg, 0.0077 mmol, 57%). LC-MS: Calc'd m/z=438.3 for C₂₃H₃₄N₈O. found [M+H]⁺=439.4. ¹H NMR (300 MHz, MeOD) δ 7.52 (d, J=1.2 Hz, 4H), 5.04 (s, 2H), 4.29 (s, 2H), 3.63-3.37 (m, 5H), 3.12 (td, J=13.2, 2.9 Hz, 2H), 2.43 (d, J=13.7 Hz, 2H), 1.94 (qd, J=13.2, 4.2 Hz, 2H), 1.71-1.60 (m, 2H), 1.44-1.28 (m, 4H), 1.01-0.90 (m, 3H).

### Example 1.143: 6-amino-9-(4-(((2-aminoethyl)amino)methyl)benzyl)-2-(pentylamino)-9H-purin-8-ol (Compound 189)

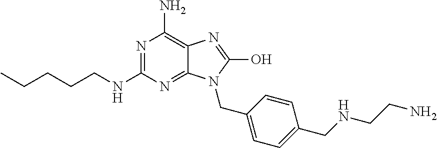 [SMILES: CCCCCNc1nc(N)c2nc(O)n(Cc3ccc(CNCCN)cc3)c2n1]

The titled compound was prepared according to General Procedure 1 from compound 51 (0.010 g, 0.027 mmol, 1.0 eq.) and tert-butyl (2-aminoethyl)carbamate (20 mg, 0.12 mmol, 4.6 eq.). Preparative HPLC purification was accomplished as described in General Procedure 5, eluting with a 10 to 50% CH₃CN+0.1% TFA/H₂O+0.1% TFA gradient. Half of the resulting Boc intermediate was reserved and deprotection of the remaining material was accomplished according to General Procedure 2 to give the title compound 189 as a white solid (4.4 mg, 0.0059 mmol, 44%). LC-MS: Calc'd m/z=398.3 for C₂₀H₃₀N₈O. found [M+H]⁺=399.4. ¹H NMR (300 MHz, MeOD) δ 7.53 (d, J=2.3 Hz, 4H), 5.04 (s, 2H), 4.30 (s, 2H), 3.48-3.35 (m, 4H), 3.40-3.34 (m, 2H), 1.66 (t, J=7.1 Hz, 2H), 1.44-1.34 (m, 4H), 1.00-0.90 (m, 3H).

### Example 1.144: 6-amino-9-(4-(((4-aminobutyl)amino)methyl)benzyl)-2-(pentylamino)-9H-purin-8-ol (Compound 190)

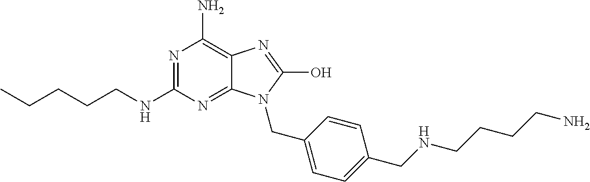 [SMILES: CCCCCNc1nc(N)c2nc(O)n(Cc3ccc(CNCCCCN)cc3)c2n1]

The titled compound was prepared according to General Procedure 1 from compound 51 (0.010 g, 0.027 mmol, 1.0 eq.) and tert-butyl (4-aminobutyl)carbamate (20 mg, 0.11 mmol, 3.9 eq.). Preparative HPLC purification was accomplished as described in General Procedure 5, eluting with a 10 to 50% CH₃CN+0.1% TFA/H₂O+0.1% TFA gradient. Half of the resulting Boc intermediate was reserved and deprotection of the remaining material was accomplished according to General Procedure 2 to give the title compound 190 as a white solid (4.1 mg, 0.0053 mmol, 40%). LC-MS: Calc'd m/z=426.3 for C₂₂H₃₄N₈O. found [M+H]⁺=427.4. ¹H NMR (300 MHz, MeOD) δ 7.57-7.47 (m, 4H), 5.01 (d, J=13.8 Hz, 2H), 4.21 (s, 2H), 3.42 (t, J=7.1 Hz, 2H), 3.10 (t, J=7.5 Hz, 2H), 2.98 (t, J=7.2 Hz, 2H), 1.88-1.56 (m, 6H), 1.44-1.28 (m, 4H), 1.00-0.89 (m, 3H).

### Example 1.145: 6-amino-9-(4-(((4-(aminomethyl)benzyl)amino)methyl)benzyl)-2-(pentyl-amino)-9H-purin-8-ol (Compound 191)

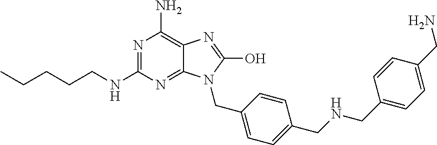 [SMILES: CCCCCNc1nc(N)c2nc(O)n(Cc3ccc(CNCc4ccc(CN)cc4)cc3)c2n1]

The titled compound was prepared according to General Procedure 1 from compound 51 (0.010 g, 0.027 mmol, 1.0 eq.) and tert-butyl (4-(aminomethyl)benzyl)carbamate (20 mg, 0.084 mmol, 3.1 eq.). Preparative HPLC purification was accomplished as described in General Procedure 5, eluting with a 10 to 50% CH₃CN+0.1% TFA/H₂O+0.1% TFA gradient. Half of the resulting Boc intermediate was reserved and deprotection of the remaining material was accomplished according to General Procedure 2 to give the title compound 191 as a white solid (4.3 mg, 0.0053 mmol, 39%). LC-MS: Calc'd m/z=474.3 for C₂₆H₃₄N₈O. found [M+H]⁺=475.4. ¹H NMR (300 MHz, MeOD) δ 7.63-7.44 (m, 8H), 5.03 (s, 2H), 4.31-4.23 (m, 4H), 4.17 (s, 2H), 3.42 (t, J=7.1 Hz, 2H), 1.65 (t, J=7.1 Hz, 2H), 1.43-1.28 (m, 4H), 0.99-0.89 (m, 3H).

### Example 1.146: 6-amino-2-(pentylamino)-9-(4-(((2-(piperazin-1-yl)ethyl)amino)methyl)-benzyl)-9H-purin-8-ol (Compound 195)

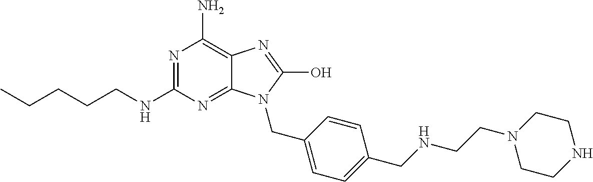 [SMILES: CCCCCNc1nc(N)c2nc(O)n(Cc3ccc(CNCCN4CCNCC4)cc3)c2n1]

The titled compound was prepared according to General Procedure 1 from compound 51 (0.010 g, 0.027 mmol, 1.0 eq.) and tert-butyl 4-(2-aminoethyl)piperazine-1-carboxylate (20 mg, 0.087 mmol, 3.2 eq.). Preparative HPLC purification was accomplished as described in General Procedure 5, eluting with a 10 to 50% CH₃CN+0.1% TFA/H₂O+0.1% TFA gradient. Half of the resulting Boc intermediate was reserved and deprotection of the remaining material was accomplished according to General Procedure 2 to give the titled compound as a white solid (4.5 mg, 0.0056 mmol, 41%). LC-MS: Calc'd m/z=467.3 for C₂₄H₃₇N₉O. found [M+H]⁺=468.4. ¹H NMR (300 MHz, MeOD) δ 7.52 (s, 4H), 5.04 (s, 2H), 4.26 (s, 2H), 3.43 (t, J=7.1 Hz, 2H), 3.26 (dt, J=10.1, 5.1 Hz, 6H), 3.19 (t, J=5.8 Hz, 2H), 2.79-2.69 (m, 4H), 1.71-1.60 (m, 2H), 1.44-1.28 (m, 4H), 1.00-0.89 (m, 3H).

### Example 1.146: Methyl 5-(bromomethyl)picolinate

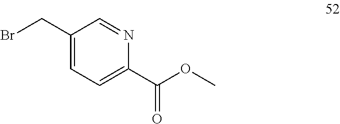

To a solution of methyl 5-methylpyridine-2-carboxylate (750 mg, 5.0 mmol, 1.0 eq.) and AIBN (81 mg, 0.50 mmol, 0.10 eq.) in CCl₄ (5 mL) at 55° C. was added NBS (970 mg, 5.5 mmol, 1.1 eq.) dissolved in CCl₄ (5 mL). The resulting mixture was heated to reflux for 18 h then concentrated in vacuo and redissolved in DCM (150 mL). The resulting solution was extracted with H₂O (1×100 mL) and brine (1×50 mL) then dried over MgSO₄ and concentrated in vacuo to give the crude product. Normal phase flash purification was accomplished as described in General Procedure 5 using a 50 g silica column, eluting with a 0 to 50% hexanes/EtOAc gradient to give the title product 52 as an off-white solid (0.35 g, 1.5 mmol, 31%). LC-MS: Calc'd m/z=229.0 for C₉H₁₄O₅S. found [M+H]⁺=230.0. ¹H NMR (300 MHz, CDCl₃) δ 8.77 (dd, J=2.2, 0.8 Hz, 1H), 8.15 (dd, J=8.0, 0.9 Hz, 1H), 7.91 (dd, J=8.1, 2.3 Hz, 1H), 4.53 (s, 2H), 4.04 (s, 3H).

### Example 1.147: Methyl 5-((6-amino-2-ethoxy-8-methoxy-9H-purin-9-yl)methyl)picolinate

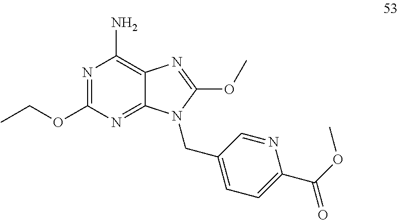 [SMILES: CCOc1nc(N)c2nc(OC)n(Cc3ccc(C(=O)OC)nc3)c2n1]

To a solution of compound 8 (310 mg, 1.5 mmol, 1.0 eq.) and compound 52 (340 mg, 1.5 mmol, 1.0 eq.) in DMF (8 mL) was added CsCO₃ (480 mg, 1.5 mmol, 1.0 eq.). The resulting suspension was stirred at room temperature for 18 h and then concentrated in vacuo. Reverse phase flash purification was accomplished as described in General Procedure 5 using a 60 g C18 column, eluting with a 10 to 60% CH₃CN+0.1% TFA/H₂O+0.1% TFA gradient. The title compound 53 was obtained as a white solid (230 mg, 0.64 mmol, 43%). LC-MS: Calc'd m/z=358.1 for C₁₈H₂₂N₆O₄. found [M+H]⁺=359.2.

### Example 1.148: (5-((6-amino-2-ethoxy-8-methoxy-9H-purin-9-yl)methyl)pyridin-2-yl)-methanol

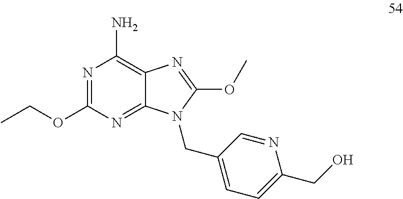 [SMILES: CCOc1nc(N)c2nc(OC)n(Cc3ccc(CO)nc3)c2n1]

To a solution of compound 53 (230 mg, 0.64 mmol, 1.0 eq.) in anhydrous THF (8 mL) cooled to 0° C. was added lithium aluminum hydride (24 mg, 0.64 mmol, 1.0 eq.) over 5 mins. The resulting suspension was allowed to warm to room temperature and stirred for 15 mins, after which it was diluted with MeOH (50 mL) then filtered through a celite plug. The filtrate was concentrated in vacuo to yield the title compound 54, which was carried forward without additional purification (0.20 g, 0.61 mmol, 94%). LC-MS: Calc'd m/z=330.1 for C₁₅H₁₈N₆O₃. found [M+H]⁺=331.2.

### Example 1.149: 6-amino-2-ethoxy-9-((6-(chloromethyl)pyridin-3-yl)methyl)-9H-purin-8-ol

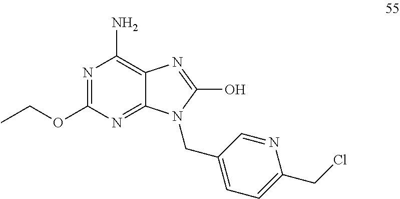 [SMILES: CCOc1nc(N)c2nc(O)n(Cc3ccc(CCl)nc3)c2n1]

The title compound 55 was prepared according to General Procedure 3 from compound 54 (0.20 g, 0.61 mmol, 1.0 eq.) and 15% SOCl₂/DCM (5 mL). The titled compound was obtained as a yellow solid (assumed quantitative yield). LC-MS: Calc'd m/z=334.1 for C₁₄H₁₅ClN₆O₂. found [M+H]⁺=335.2.

### Example 1.150: 6-amino-2-ethoxy-9-((6-(piperazin-1-ylmethyl)pyridin-3-yl)methyl)-9H-purin-8-ol (Compound 192)

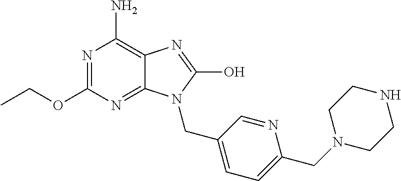 [SMILES: CCOc1nc(N)c2nc(O)n(Cc3ccc(CN4CCNCC4)nc3)c2n1]

The titled compound was prepared according to General Procedure 1 from compound 55 (70 mg, 0.21 mmol, 1.0 eq.) and tert-butyl piperazine-1-carboxylate (0.20 g, 1.0 mmol, 5.0 eq.). Preparative HPLC purification was accomplished as described in General Procedure 5, eluting with a 10 to 55% CH₃CN+0.1% TFA/H₂O+0.1% TFA gradient. Half of the resulting Boc intermediate was reserved and deprotection of the remaining material was accomplished according to General Procedure 2 to give the title compound 192 as a yellow solid (3.1 mg, 0.0065 mmol, 6.2%). LC-MS: Calc'd m/z=484.2 for C₁₈H₂₄N₈O₂. found [M+H]⁺=385.3. ¹H NMR (300 MHz, MeOD) δ 8.73 (s, 1H), 8.10 (dd, J=8.1, 2.3 Hz, 1H), 7.67 (d, J=8.1 Hz, 1H), 5.11 (s, 2H), 4.38 (q, J=7.1 Hz, 2H), 3.88 (s, 2H), 3.33-3.23 (m, 4H), 2.82 (t, J=5.2 Hz, 4H), 1.38 (t, J=7.1 Hz, 3H).

### Example 2: Preparation of Compound-Linker Constructs

### Example 2.1: 2,3,5,6-tetrafluorophenyl 3-(2-(2-(2-(2,5-dioxo-2,5-dihydro-1H-pyrrol-1-yl)-ethoxy) ethoxy)ethoxy)propanoate (MT-TFP)

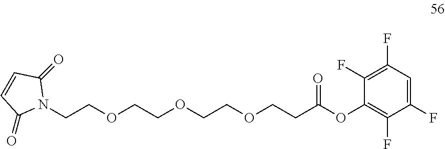 [SMILES: O=C(CCOCCOCCOCCN1C(=O)C=CC1=O)Oc1c(F)c(F)cc(F)c1F]

Title compound 56 was prepared according to the procedure described in International Patent Publication No. WO2017/054080. LC-MS: Calc'd m/z=449.4 for C₁₉H₁₉F₄O₇. found [M+H]⁺=450.4. ¹H NMR (300 MHz, MeOD) δ 7.42 (tt, J=10.5, 7.2 Hz, 1H), 6.81 (s, 2H), 3.87 (t, J=6.0 Hz, 2H), 3.73-3.56 (m, 12H), 2.98 (t, J=6.0 Hz, 2H).

### Example 2.2: (S)-2-((S)-2-amino-3-methylbutanamido)-N-(4-(hydroxymethyl)phenyl)-5-ureidopentanamide (VC-PAB-OH)

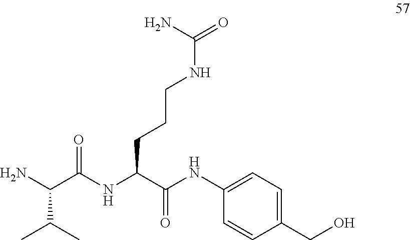 [SMILES: CC(C)[C@H](N)C(=O)N[C@@H](CCCNC(N)=O)C(=O)Nc1ccc(CO)cc1]

Title compound 57 was prepared according to the procedure described in International Patent Publication No. WO 2005/112919. LC-MS: Calc'd m/z=379.2 for C₁₈H₂₉N₅O₄. found [M+H]⁺=380.2.

### Example 2.3: (S)-2-((S)-1-(2,5-dioxo-2,5-dihydro-1H-pyrrol-1-yl)-14-isopropyl-12-oxo-3,6,9-trioxa-13-azapentadecan-15-amido)-N-(4-(hydroxymethyl)phenyl)-5-ureidopentanamide (MT-VC-PAB-OH)

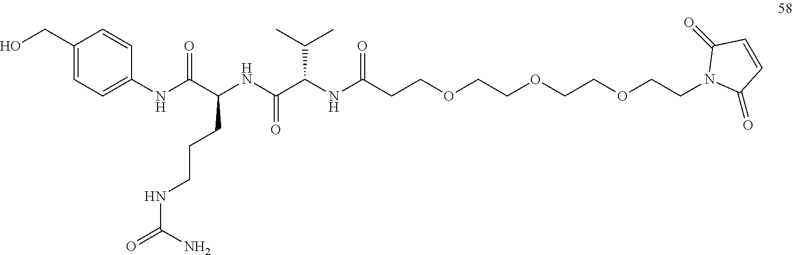 [SMILES: CC(C)[C@H](NC(=O)CCOCCOCCOCCN1C(=O)C=CC1=O)C(=O)N[C@@H](CCCNC(N)=O)C(=O)Nc1ccc(CO)cc1]

To a solution of compound 57 (2.37 g, 6.26 mmol, 1.00 eq.) in DMF (15 mL) was added compound 56 (2.81 g, 6.26 mmol, 1.00 eq.) followed by DIPEA (2.73 mL, 15.6 mmol, 2.50 eq.). The resulting mixture stirred at room temperature for 18 h, after which the reaction was adjusted to pH 1 with 1 M HCl. Reverse phase flash purification was accomplished as described in General Procedure 5 using a 120 g C18 column, eluting with a 10 to 50% CH₃CN+0.1% TFA/H₂O+0.1% TFA gradient to give the title compound 58 as a white solid (4.00 g, 6.04 mmol, 96.5%). LC-MS: Calc'd m/z=662.3 for C₃₁H₄₆N₆O₁₀. found [M+H]⁺=663.2.

### Example 2.4: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (4-nitrophenyl) carbonate (MT-VC-PABC-PNP)

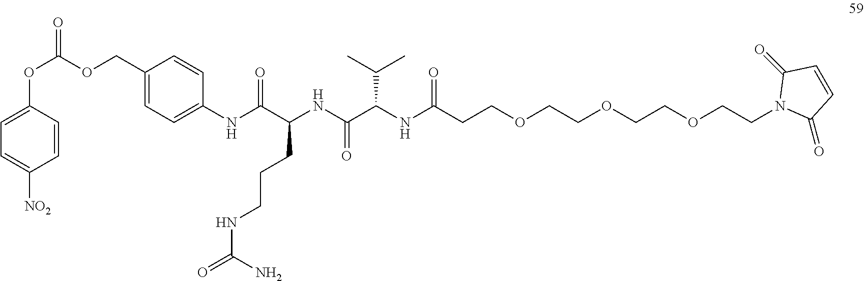 [SMILES: CC(C)[C@H](NC(=O)CCOCCOCCOCCN1C(=O)C=CC1=O)C(=O)N[C@@H](CCCNC(N)=O)C(=O)Nc1ccc(COC(=O)Oc2ccc([N+](=O)[O-])cc2)cc1]

To a solution of compound 58 (2.50 g, 3.77 mmol, 1.00 eq.) in DMF (5 mL) was added bis(4-nitrophenyl) carbonate (1.26 g, 4.15 mmol, 1.50 eq.) followed by DIPEA (1.98 mL, 11.3 mmol, 3.00 eq.). The resulting mixture stirred at room temperature for 18 h, after which the reaction was adjusted to pH 1 with 1 M HCl. Reverse phase flash purification was accomplished as described in General Procedure 5 using a 120 g C18 column, eluting with a 20 to 65% CH₃CN+0.1% TFA/H₂O+0.1% TFA gradient to give the title compound 59 as a white solid (2.50 g, 3.02 mmol, 80.1%). LC-MS: Calc'd m/z=827.3 for C₃₈H₄₉N₇O₁₄. found [M+H]⁺=828.4. ¹H NMR (300 MHz, MeOD) δ 8.39-8.29 (m, 2H), 7.68 (d, J=8.1 Hz, 2H), 7.54-7.39 (m, 4H), 6.83 (s, 2H), 5.28 (s, 2H), 4.54 (dd, J=9.0, 4.7 Hz, 1H), 4.22 (d, J=6.9 Hz, 1H), 3.82-3.51 (m, 12H), 3.28-3.08 (m, 5H), 2.57 (t, J=6.0 Hz, 2H), 2.21-1.49 (m, 4H), 1.01 (dd, J=6.8, 4.9 Hz, 6H).

### Example 2.5: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl-4-(4-((6-amino-2-ethoxy-8-hydroxy-9H-purin-9-yl)methyl)-3-methoxybenzyl)piperazine-1-carboxylate (MT-VC-PABC-Compound 100)

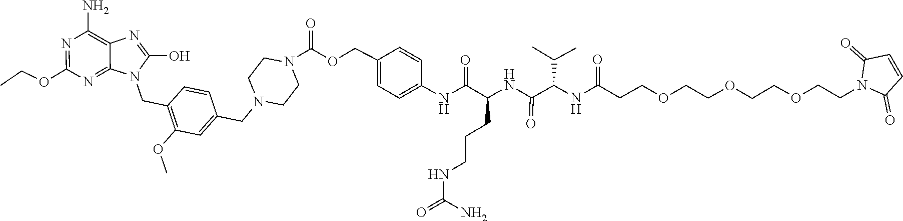 [SMILES: CCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 100 (26.5 mg, 0.0640 mmol, 1.00 eq.) and compound 59 (59 mg. 0.071 mmol, 1.1 eq.). Preparative HPLC purification was accomplished as described in General Procedure 5, eluting with a 10 to 55% CH₃CN+0.1% TFA/H₂O+0.1% TFA gradient to give the titled compound as a white solid (49.0 mg, 0.0443 mmol, 69.2%). LC-MS: Calc'd m/z=1101.5 for C₅₂H₇₁N₁₃O₁₄. found [M+H]⁺=1102.8. ¹H NMR (300 MHz, MeOD) δ 7.69-7.55 (m, 2H), 7.39-7.30 (m, 2H), 7.19-7.10 (m, 2H), 7.02 (dd, J=7.7, 1.5 Hz, 1H), 6.82 (s, 2H), 5.12 (s, 2H), 5.06 (s, 2H), 4.54-4.48 (m J=9.2, 4.9 Hz, 1H), 4.38 (q, J=7.1 Hz, 2H), 4.34 (s, 2H), 4.22 (d, J=6.9 Hz, 1H), 3.92 (s, 3H), 3.82-3.67 (m, 2H), 3.72-3.59 (m, 4H), 3.57 (d, J=2.4 Hz, 8H), 3.29-3.08 (m, 1H), 2.57 (t, J=6.0 Hz, 2H), 2.13 (h, J=6.7 Hz, 1H), 1.94 (dd, J=12.9, 5.6 Hz, 1H), 1.84-1.69 (m, 1H), 1.60 (h, J=7.2 Hz, 2H), 1.35 (t, J=7.1 Hz, 3H), 1.00 (dd, J=6.8, 4.0 Hz, 6H).

### Example 2.6: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl(4-((6-amino-2-ethoxy-8-hydroxy-9H-purin-9-yl)methyl)-3-methoxybenzyl)(benzyl)carbamate (MT-VC-PABC-Compound 107)

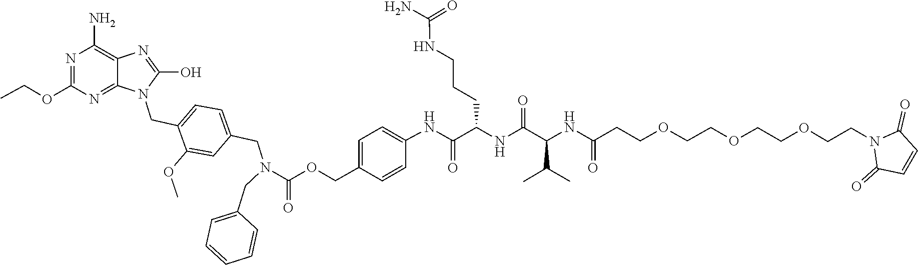 [SMILES: CCOc1nc(N)c2nc(O)n(Cc3ccc(CN(Cc4ccccc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1]

The titled compound was prepared according to General Procedure 4 from compound 107 (8.8 mg, 0.013 mmol, 1.0 eq.) and compound 59 (12 mg. 0.015 mmol, 1.1 eq.). Preparative HPLC purification was accomplished as described in General Procedure 5, eluting with a 40 to 52% CH₃CN+0.1% TFA/H₂O+0.1% TFA gradient to give the titled compound as a white solid (2.7 mg, 0.0022 mmol, 17%). LC-MS: Calc'd m/z=1122.5 for C₅₅H₇₀N₁₂O₁₄. found [M+H]⁺=1123.7. ¹H NMR (300 MHz, MeOD) δ 7.71-6.95 (m, 12H), 6.81 (s, 2H), 5.26-5.06 (m, 2H), 5.04-4.98 (m, 2H), 4.62-4.30 (m, 7H), 4.22 (d, J=6.8 Hz, 1H), 3.85-3.49 (m, 17H), 3.29-2.89 (m, 2H), 2.59 (t, 2H), 2.31-1.50 (m, 5H), 1.46-1.25 (m, 3H), 1.12-0.89 (m, 6H).

### Example 2.7: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl(4-((6-amino-2-ethoxy-8-hydroxy-9H-purin-9-yl)methyl)-3-methoxybenzyl)(4-(aminomethyl)benzyl)carbamate (MT-VC-PABC-Compound 105)

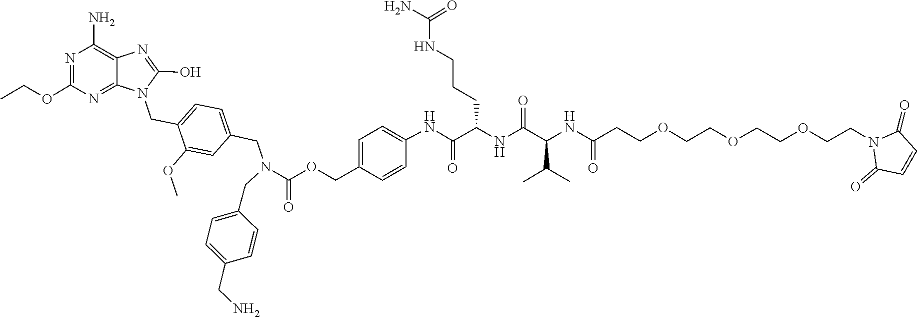 [SMILES: CCOc1nc(N)c2nc(O)n(Cc3ccc(CN(Cc4ccc(CN)cc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1]

The titled compound was prepared according to General Procedure 4 from the Boc intermediate of compound 105 (17 mg, 0.021 mmol, 1.0 eq.) and compound 59 (19 mg. 0.023 mmol, 1.1 eq.) followed by deprotection of the crude intermediate according to General Procedure 3. Preparative HPLC purification was accomplished as described in General Procedure 5, eluting with a 10 to 45% CH₃CN+0.1% TFA/H₂O+0.1% TFA gradient to give the titled compound as a white solid (8.7 mg, 0.0063 mmol, 30%). LC-MS: Calc'd m/z=1151.5 for C₅₆H₇₃N₁₃O₁₄. found [M+H]⁺=1152.7. ¹H NMR (300 MHz, MeOD) δ 7.58-7.52 (m, 2H), 7.41-7.04 (m, 9H), 6.81 (s, 2H), 5.16 (s, 2H), 4.98 (s, 2H), 4.55-4.40 (m, 3H), 4.36-4.17 (m, 3H), 4.10 (s, 2H), 3.86-3.50 (m, 19H), 3.26-3.09 (m, 2H), 2.59 (t, J=6.0 Hz, 2H), 2.21-2.09 (m, 1H), 1.97-1.91 (m, 1H), 1.83-1.74 (m, 1H), 1.66-1.51 (m, 2H), 1.31 (t, J=7.1 Hz, 3H), 1.07-0.97 (m, 6H).

### Example 2.8: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl(4-((6-amino-2-ethoxy-8-hydroxy-9H-purin-9-yl)methyl)-3-methoxybenzyl)(cyclobutyl)carbamate (MT-VC-PABC-Compound 108)

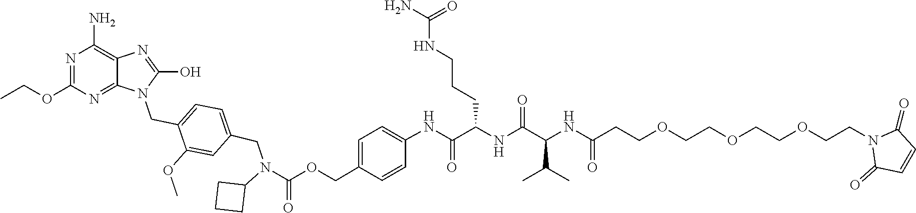 [SMILES: CCOc1nc(N)c2nc(O)n(Cc3ccc(CN(C(=O)OCc4ccc(NC(=O)[C@H](CCCNC(N)=O)NC(=O)[C@@H](NC(=O)CCOCCOCCOCCN5C(=O)C=CC5=O)C(C)C)cc4)C4CCC4)cc3OC)c2n1]

The titled compound was prepared according to General Procedure 4 from compound 108 (15 mg, 0.024 mmol, 1.0 eq.) and compound 59 (0.020 g. 0.024 mmol, 1.0 eq.). Preparative HPLC purification was accomplished as described in General Procedure 5, eluting with a 10 to 45% CH₃CN+0.1% TFA/H₂O+0.1% TFA gradient to give the titled compound as a white solid (14 mg, 0.012 mmol, 51%). LC-MS: Calc'd m/z=1086.5 for C₅₂H₇₀N₁₂O₁₄. found [M+H]⁺=1087.7. ¹H NMR (300 MHz, MeOD) δ 7.49-7.43 (m, 4H), 7.05 (d, J=7.6 Hz, 1H), 6.81 (s, 2H), 6.73-6.64 (m, 2H), 5.02 (s, 4H), 4.54 (s, 3H), 4.36 (q, J=7.1 Hz, 2H), 4.22 (d, J=6.9 Hz, 1H), 3.88-3.47 (m, 19H), 3.35-3.07 (m, 2H), 2.59 (t, J=6.0 Hz, 2H), 2.11 (s, 4H), 2.01-1.71 (m, 2H), 1.60 (dt, J=15.2, 7.7 Hz, 4H), 1.32 (t, J=7.0 Hz, 3H), 1.02 (dd, J=6.8, 4.3 Hz, 6H).

### Example 2.9: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl(4-((6-amino-2-ethoxy-8-hydroxy-9H-purin-9-yl)methyl)-3-methoxybenzyl)(4-((dipropylamino)methyl)benzyl)-carbamate (MT-VC-PABC-Compound 141)

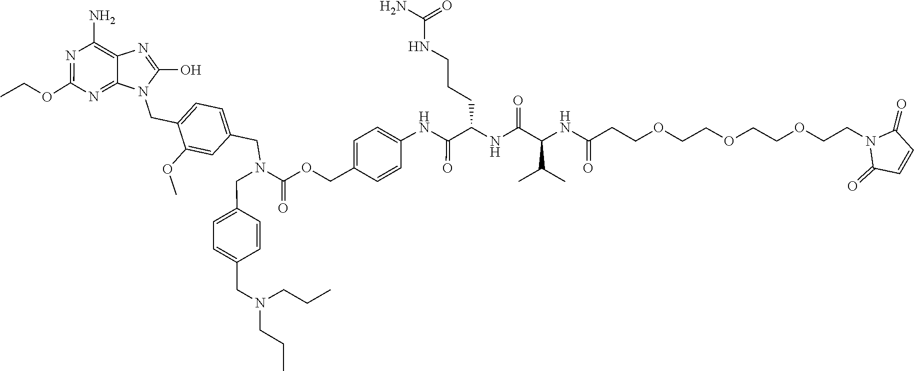 [SMILES: CCCN(CCC)Cc1ccc(CN(Cc2ccc(Cn3c(O)nc4c(N)nc(OCC)nc43)c(OC)c2)C(=O)OCc2ccc(NC(=O)[C@H](CCCNC(N)=O)NC(=O)[C@@H](NC(=O)CCOCCOCCOCCN3C(=O)C=CC3=O)C(C)C)cc2)cc1]

The titled compound was prepared according to General Procedure 4 from compound 141 (15 mg, 0.017 mmol, 1.0 eq.) and compound 59 (15 mg. 0.018 mmol, 1.1 eq.). Preparative HPLC purification was accomplished as described in General Procedure 5, eluting with a 30 to 55% CH₃CN+0.1% TFA/H₂O+0.1% TFA gradient to give the titled compound as a white solid (5.0 mg, 0.0032 mmol, 19%). LC-MS: Calc'd m/z=1235.6 for C₆₂H₈₅N₁₃O₁₄. found [M+H]⁺=1236.8. ¹H NMR (300 MHz, MeOD) δ 7.66-7.11 (m, 9H), 6.94-6.88 (m, 1H), 6.81 (s, 2H), 6.68-6.62 (m, 1H), 5.16 (s, 2H), 4.99 (s, 2H), 4.54 (d, J=20.3 Hz, 5H), 4.34 (s, 4H), 4.21 (d, J=6.8 Hz, 1H), 3.90-3.49 (m, 19H), 3.29-2.98 (m, 6H), 2.59 (t, J=6.0 Hz, 2H), 2.14 (p, J=6.8 Hz, 1H), 2.04-1.49 (m, 4H), 1.32 (t, 3H), 1.07-0.94 (m, 14H).

### Example 2.10: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl(4-((6-amino-2-ethoxy-8-hydroxy-9H-purin-9-yl)methyl)-3-methoxybenzyl)(4-(morpholinomethyl)benzyl)-carbamate (MT-VC-PABC-Compound 142)

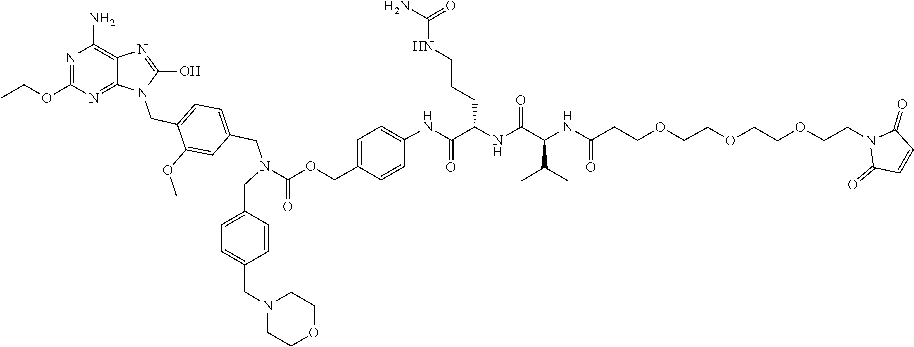 [SMILES: CCOc1nc(N)c2nc(O)n(Cc3ccc(CN(Cc4ccc(CN5CCOCC5)cc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1]

The titled compound was prepared according to General Procedure 4 from compound 142 (15 mg, 0.017 mmol, 1.0 eq.) and compound 59 (16 mg. 0.019 mmol, 1.1 eq.). Preparative HPLC purification was accomplished as described in General Procedure 5, eluting with a 30 to 55% CH₃CN+0.1% TFA/H₂O+0.1% TFA gradient to give the titled compound as a white solid (5.0 mg, 0.0032 mmol, 19%). LC-MS: Calc'd m/z=1221.6 for C₆₀H₇₉N₁₃O₁₅. found [M+H]⁺=1222.8. ¹H NMR (300 MHz, MeOD) δ 7.59-7.53 (m, 2H), 7.48-7.04 (m, 8H), 6.95-6.89 (m, 1H), 6.81 (s, 2H), 5.16 (s, 2H), 4.98 (s, 2H), 4.50 (s, 4H), 4.32 (q, J=6.9 Hz, 3H), 4.21 (d, J=6.7 Hz, 1H), 4.07 (s, 2H), 3.89-3.45 (m, 17H), 3.29-3.10 (m, 2H), 3.04-2.98 (m, 4H), 2.91-2.85 (m, 4H), 2.59 (t, J=6.0 Hz, 2H), 2.22-2.09 (m, 1H), 2.01-1.70 (m, 2H), 1.66-1.55 (m, 2H), 1.31 (t, J=7.1 Hz, 3H), 1.03 (dd, J=6.8, 4.1 Hz, 6H).

### Example 2.11: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl(4-((6-amino-2-ethoxy-8-hydroxy-9H-purin-9-yl)methyl)-3-methoxybenzyl)(4-((4-((tert-butoxycarbonyl)-amino)piperidin-1-yl)methyl)benzyl)carbamate (MT-VC-PABC-Compound 143)

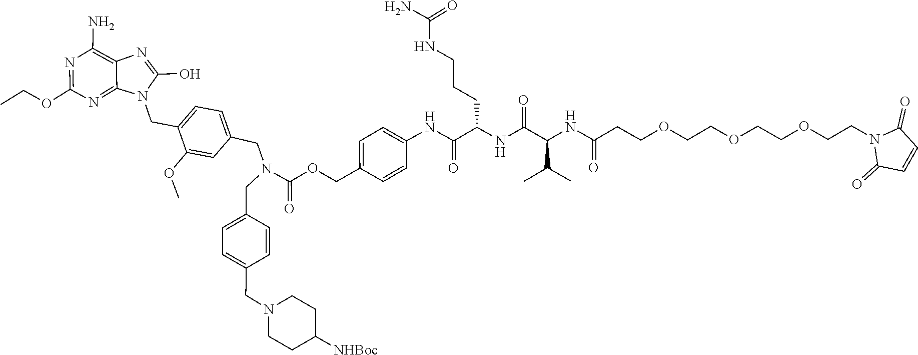 [SMILES: CCOc1nc(N)c2nc(O)n(Cc3ccc(CN(Cc4ccc(CN5CCC(NC(=O)OC(C)(C)C)CC5)cc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3OC)c2n1]

The titled compound was prepared according to General Procedure 4 from compound 143 (15 mg, 0.015 mmol, 1.0 eq.) and compound 59 (14 mg. 0.017 mmol, 1.1 eq.). Preparative HPLC purification was accomplished as described in General Procedure 5, eluting with a 30 to 55% CH₃CN+0.1% TFA/H₂O+0.1% TFA gradient to give the titled compound as a white solid (7.6 mg, 0.0045 mmol, 30%). LC-MS: Calc'd m/z=1334.7 for C₆₆H₉₀N₁₄O₁₆. found [M+H]⁺=1335.8. ¹H NMR (300 MHz, MeOD) δ 7.59-7.53 (m, 2H), 7.45-7.04 (m, 8H), 6.99-6.91 (m, 1H), 6.81 (s, 2H), 5.16 (s, 2H), 4.99 (s, 2H), 4.53-4.47 (m, 4H), 4.42-4.15 (m, 5H), 3.87-3.42 (m, 20H), 3.29-2.98 (m, 7H), 2.59 (t, J=6.0 Hz, 2H), 2.25-1.53 (m, 7H), 1.45 (s, 9H), 1.32 (t, J=7.1 Hz, 3H), 1.03 (dd, J=6.8, 4.1 Hz, 6H).

### Example 2.12: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (2-(4-(4-((6-amino-2-ethoxy-8-hydroxy-9H-purin-9-yl)methyl)-3-methoxybenzyl)piperazin-1-yl)ethyl)carbamate (MT-VC-PABC-Compound 112)

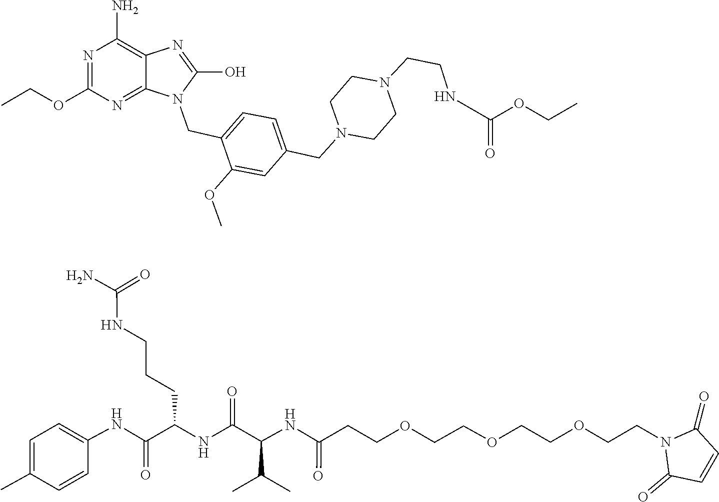

The titled compound was prepared according to General Procedure 4 from compound 112 (15 mg, 0.033 mmol, 1.0 eq.) and compound 59 (0.030 g. 0.036 mmol, 1.1 eq.). Preparative HPLC purification was accomplished as described in General Procedure 5, eluting with a 10 to 55% CH₃CN+0.1% TFA/H₂O+0.1% TFA gradient to give the titled compound as a white solid (2.0 mg, 0.0013 mmol, 4.1%). LC-MS: Calc'd m/z=1144.6 for C₅₄H₇₆N₁₄O₁₄. found [M+H]⁺=1147.8. ¹H NMR (300 MHz, MeOD) δ 7.63 (d, J=8.1 Hz, 2H), 7.34 (d, J=8.1 Hz, 2H), 7.11-6.99 (m, 2H), 6.95 (d, J=5.3 Hz, 1H), 6.83 (s, 2H), 5.09-5.01 (m, 4H), 4.50 (s, 1H), 4.32 (q, J=7.1 Hz, 2H), 4.19 (s, 1H), 3.99 (s, 2H), 3.92 (s, 3H), 3.85-3.50 (m, 18H), 3.37 (s, 2H), 3.23-2.80 (m, 8H), 2.57 (t, J=6.0 Hz, 2H), 2.17-1.47 (m, 5H), 1.33 (t, J=7.0 Hz, 3H), 0.99 (dd, J=6.9, 2.8 Hz, 6H).

### Example 2.13: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(4-((6-amino-2-ethoxy-8-hydroxy-9H-purin-9-yl)methyl)benzyl)piperazine-1-carboxylate (MT-VC-PABC-Compound 150)

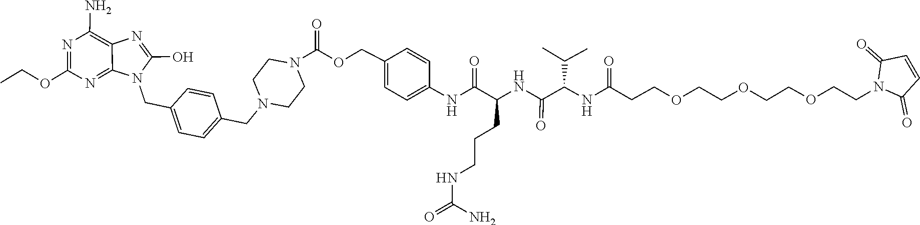 [SMILES: CCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3)c2n1]

The titled compound was prepared according to General Procedure 4 from compound 150 (15 mg, 0.039 mmol, 1.0 eq.) and compound 59 (36 mg. 0.043 mmol, 1.1 eq.). Preparative HPLC purification was accomplished as described in General Procedure 5, eluting with a 10 to 55% CH₃CN+0.1% TFA/H₂O+0.1% TFA gradient to give the titled compound as a white solid (8.7 mg, 0.0080 mmol, 20%). LC-MS: Calc'd m/z=1071.5 for C₅₁H₆₉N₁₃O₁₃. found [M+H]⁺=1072.7. ¹H NMR (300 MHz, MeOD) δ 7.65-7.57 (m, 2H), 7.53 (d, J=8.2 Hz, 2H), 7.46 (d, J=8.2 Hz, 2H), 7.32 (d, J=8.5 Hz, 2H), 6.80 (s, 2H), 5.10 (s, 2H), 5.04 (s, 2H), 4.51 (dd, J=9.1, 4.8 Hz, 1H), 4.36 (dd, J=13.9, 6.8 Hz, 4H), 4.27-4.15 (m, 1H), 3.80-3.50 (m, 18H), 3.27-3.04 (m, 6H), 2.55 (t, J=6.0 Hz, 2H), 2.20-2.00 (m, 1H), 1.96-1.83 (m, 1H), 1.82-1.68 (m, 1H), 1.67-1.52 (m, 2H), 1.36 (t, J=7.1 Hz, 3H), 0.98 (dd, J=6.8, 4.0 Hz, 6H).

### Example 2.14: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (4-((4-((6-amino-2-ethoxy-8-hydroxy-9H-purin-9-yl)methyl)-3-methoxybenzyl)amino)butyl)carbamate (MT-VC-PABC-Compound 106)

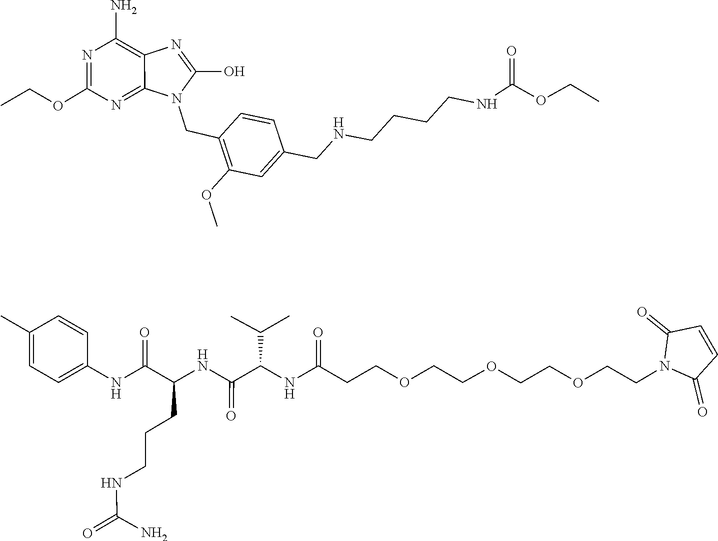

The titled compound was prepared according to General Procedure 4 from compound 106 (15 mg, 0.036 mmol, 1.0 eq.) and compound 59 (33 mg. 0.040 mmol, 1.1 eq.). Preparative HPLC purification was accomplished as described in General Procedure 5, eluting with a 10 to 55% CH₃CN+0.1% TFA/H₂O+0.1% TFA gradient to give the titled compound as a white solid (3.2 mg, 0.0024 mmol, 6.7%). LC-MS: Calc'd m/z=1103.5 for C₅₂H₇₃N₁₃O₁₄. found [M+H]⁺=1104.7. ¹H NMR (300 MHz, MeOD) δ 7.59 (d, J=8.2 Hz, 2H), 7.31 (d, J=8.3 Hz, 2H), 7.14-7.02 (m, 2H), 6.83 (s, 2H), 5.08-5.01 (m, 4H), 4.54-4.48 (m, 1H), 4.31 (q, J=7.1 Hz, 2H), 4.21 (d, J=6.9 Hz, 1H), 4.14 (s, 2H), 3.94 (s, 3H), 3.80-3.49 (m, 14H), 3.24-3.11 (m, 3H), 3.08-3.02 (m, 3H), 2.57 (t, J=6.0 Hz, 2H), 2.19-2.07 (m, 1H), 1.95-1.89 (m, 1H), 1.74-1.68 (m, 3H), 1.61-1.55 (m, 5H), 1.33 (t, J=7.1 Hz, 3H), 1.00 (dd, J=6.8, 3.6 Hz, 6H).

### Example 2.15: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (2-((4-((6-amino-2-ethoxy-8-hydroxy-9H-purin-9-yl)methyl)-3-methoxybenzyl)amino)ethyl)carbamate (MT-VC-PABC-Compound 111)

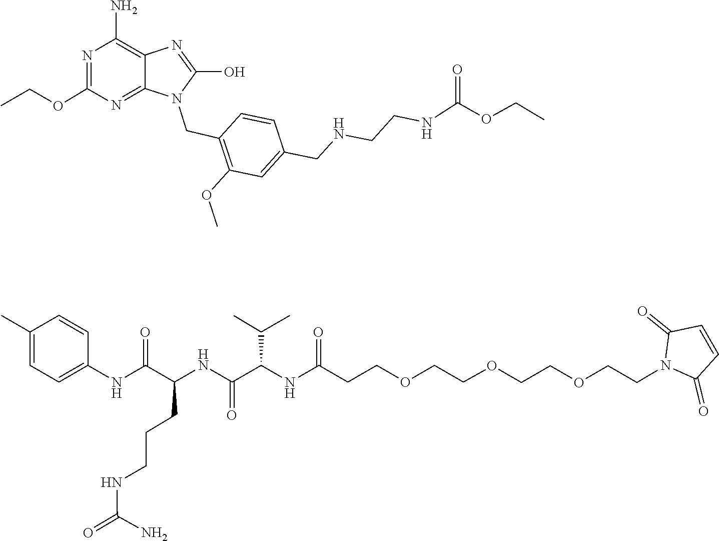

The titled compound was prepared according to General Procedure 4 from compound 111 (15 mg, 0.039 mmol, 1.0 eq.) and compound 59 (35 mg. 0.043 mmol, 1.1 eq.). Preparative HPLC purification was accomplished as described in General Procedure 5, eluting with a 10 to 55% CH₃CN+0.1% TFA/H₂O+0.1% TFA gradient to give the titled compound as a white solid (4.8 mg, 0.0037 mmol, 9.4%). LC-MS: Calc'd m/z=1075.5 for C₅₀H₆₉N₁₃O₁₄. found [M+H]⁺=1076.7. ¹H NMR (300 MHz, MeOD) δ 7.60 (d, J=8.1 Hz, 2H), 7.32 (d, J=8.2 Hz, 2H), 7.17-7.04 (m, 2H), 6.98 (d, J=7.8 Hz, 1H), 6.82 (s, 2H), 5.10-5.03 (m, 4H), 4.56-4.48 (m, 1H), 4.33 (q, J=7.1 Hz, 2H), 4.25-4.19 (m, 3H), 3.93 (s, 3H), 3.85-3.41 (m, 16H), 3.27-3.09 (m, 4H), 2.57 (t, J=6.0 Hz, 2H), 2.14 (dt, J=13.6, 6.8 Hz, 1H), 1.96-1.90 (m, 1H), 1.82-1.69 (m, 1H), 1.67-1.54 (m, 2H), 1.34 (t, J=7.1 Hz, 3H), 1.01 (dd, J=6.8, 3.8 Hz, 6H).

### Example 2.16: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (1-(4-(((4-((6-amino-2-ethoxy-8-hydroxy-9H-purin-9-yl)methyl)-3-methoxybenzyl)amino)methyl)benzyl)-piperidin-4-yl)carbamate (MT-VC-PABC-Compound 144)

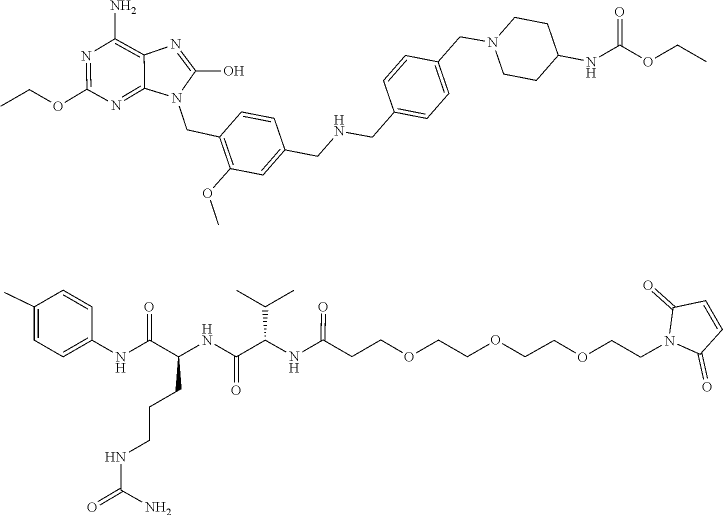

The titled compound was prepared according to General Procedure 4 from compound 144 (0.010 g, 0.011 mmol, 1.0 eq.) and compound 59 (8.3 mg. 0.010 mmol, 0.90 eq.). Preparative HPLC purification was accomplished as described in General Procedure 5, eluting with a 10 to 55% CH₃CN+0.1% TFA/H₂O+0.1% TFA gradient to give the titled compound as a white solid (4.9 mg, 0.0031 mmol, 31%). LC-MS: Calc'd m/z=1234.6 for C₆₁H₈₂N₁₄O₁₄. found [M+H]⁺=1235.7. ¹H NMR (300 MHz, MeOD) δ 7.64-7.58 (m, 6H), 7.32 (d, J=8.2 Hz, 2H), 7.14 (s, 1H), 7.11-6.96 (m, 2H), 6.83 (s, 2H), 5.04 (s, 4H), 4.51 (s, 1H), 4.42-4.12 (m, 9H), 3.92 (s, 3H), 3.79-3.45 (m, 18H), 3.16 (ddt, J=20.8, 13.8, 7.4 Hz, 4H), 2.57 (t, J=6.0 Hz, 2H), 2.22-2.02 (m, 3H), 1.94 (d, J=7.5 Hz, 1H), 1.83-1.56 (m, 4H), 1.33 (t, J=7.1 Hz, 3H), 1.00 (dd, J=6.8, 3.8 Hz, 6H).

### Example 2.17: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(4-(((4-((6-amino-2-ethoxy-8-hydroxy-9H-purin-9-yl)methyl)-3-methoxybenzyl)amino)methyl)benzyl)-piperazine-1-carboxylate (MT-VC-PABC-Compound 145)

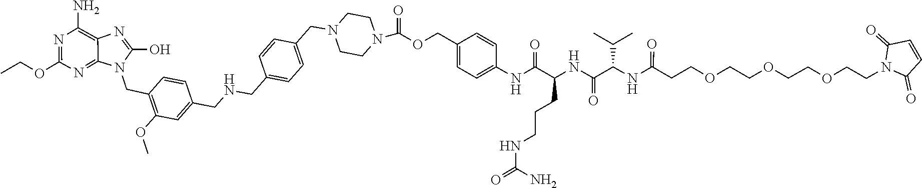 [SMILES: CCOc1nc(N)c2nc(O)n(Cc3ccc(CNCc4ccc(CN5CCN(C(=O)OCc6ccc(NC(=O)[C@H](CCCNC(N)=O)NC(=O)[C@@H](NC(=O)CCOCCOCCOCCN7C(=O)C=CC7=O)C(C)C)cc6)CC5)cc4)cc3OC)c2n1]

The titled compound was prepared according to General Procedure 4 from compound 145 (0.010 g, 0.011 mmol, 1.0 eq.) and compound 59 (8.5 mg. 0.010 mmol, 0.90 eq.). Preparative HPLC purification was accomplished as described in General Procedure 5, eluting with a 40 to 52% CH₃CN+0.1% TFA/H₂O+0.1% TFA gradient to give the titled compound as a white solid (4.7 mg, 0.0030 mmol, 29%). LC-MS: Calc'd m/z=1220.6 for C₆₀H₈₀N₁₄O₁₄. found [M+H]⁺=1221.7. ¹H NMR (300 MHz, MeOD) δ 7.72-7.52 (m, 6H), 7.35 (d, J=8.5 Hz, 2H), 7.12 (s, 1H), 7.09-6.95 (m, 2H), 6.83 (s, 2H), 5.13 (s, 2H), 5.04 (s, 2H), 4.57-4.47 (m, 1H), 4.40 (s, 2H), 4.38-4.17 (m, 7H), 3.92 (s, 3H), 3.82-3.52 (m, 14H), 3.29-3.04 (m, 10H), 2.57 (t, J=6.0 Hz, 2H), 2.11 (td, J=13.5, 6.7 Hz, 1H), 1.93 (s, 1H), 1.83-1.68 (m, 1H), 1.67-1.54 (m, 2H), 1.33 (t, J=7.1 Hz, 3H), 1.01 (dd, J=6.8, 3.7 Hz, 6H).

### Example 2.18: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(4-((6-amino-2-ethoxy-8-hydroxy-9H-purin-9-yl)methyl)-3-fluorobenzyl)piperazine-1-carboxylate (MT-VC-PABC-Compound 152)

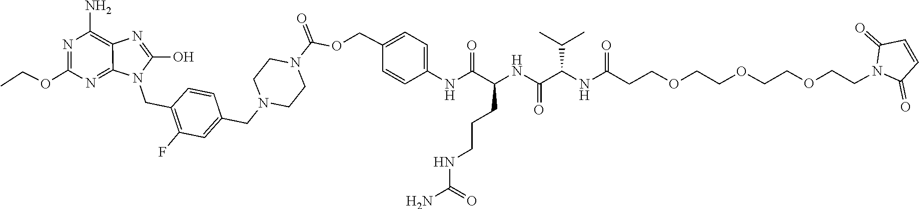 [SMILES: CCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3F)c2n1]

The titled compound was prepared according to General Procedure 4 from compound 152 (8.4 mg, 0.013 mmol, 1.0 eq.) and compound 59 (11 mg. 0.013 mmol, 1.0 eq.). Preparative HPLC purification was accomplished as described in General Procedure 5, eluting with a 10 to 50% CH₃CN+0.1% TFA/H₂O+0.1% TFA gradient to give the titled compound as a white solid (0.010 g, 0.00076 mmol, 57%). LC-MS: Calc'd m/z=1089.5 for C₅₁H₆₈FN₁₃O₁₃. found [M+H]+=1090.7. ¹H NMR (300 MHz, MeOD) δ 7.69-7.57 (m, 2H), 7.51-7.32 (m, 1H), 7.38-7.25 (m, 4H), 6.82 (s, 2H), 5.17-5.09 (m, 4H), 4.59-4.31 (m, 5H), 4.22 (d, J=6.9 Hz, 1H), 3.86-3.48 (m, 14H), 3.31-3.02 (m, 10H), 2.57 (t, J=6.1 Hz, 2H), 2.12 (hept, J=6.6 Hz, 1H), 1.94 (td, J=13.0, 7.8 Hz, 1H), 1.77 (dtd, J=14.0, 9.3, 5.1 Hz, 1H), 1.59 (qd, J=13.9, 6.9 Hz, 2H), 1.37 (t, J=7.1 Hz, 3H), 1.00 (dd, J=6.8, 4.0 Hz, 6H).

### Example 2.19: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(4-((6-amino-2-butoxy-8-hydroxy-9H-purin-9-yl)methyl)-3-methoxybenzyl)piperazine-1-carboxylate (MT-VC-PABC-Compound 154)

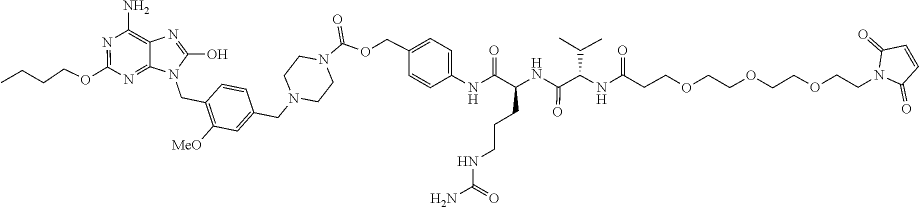 [SMILES: CCCCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 154 (2.2 mg, 0.0057 mmol, 1.0 eq.) and compound 59 (4.7 mg. 0.0057 mmol, 1.0 eq.). Preparative HPLC purification was accomplished as described in General Procedure 5, eluting with a 10 to 50% CH₃CN+0.1% TFA/H₂O+0.1% TFA gradient to give the titled compound as a white solid (2.3 mg, 0.0020 mmol, 36%). LC-MS: Calc'd m/z=1129.6 for C₅₄H₇₅N₁₃O₁₄. found [M+H]⁺=1130.7. ¹H NMR (300 MHz, MeOD) δ 7.67-7.58 (m, 2H), 7.35 (d, J=8.5 Hz, 2H), 7.18-6.96 (m, 3H), 6.82 (s, 2H), 5.13 (s, 2H), 5.05 (s, 2H), 4.52 (dt, J=9.9, 5.2 Hz, 1H), 4.37-4.17 (m, 5H), 3.94 (s, 3H), 3.85-3.47 (m, 14H), 3.27-3.08 (m, 10H), 2.57 (t, J=6.1 Hz, 2H), 2.13 (h, J=6.7 Hz, 1H), 1.92 (dd, J=13.7, 8.4 Hz, 1H), 1.86-1.37 (m, 7H), 1.08-0.91 (m, 9H).

### Example 2.20: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(4-((6-amino-2-butoxy-8-hydroxy-9H-purin-9-yl)methyl)benzyl)piperazine-1-carboxylate (MT-VC-PABC-Compound 166)

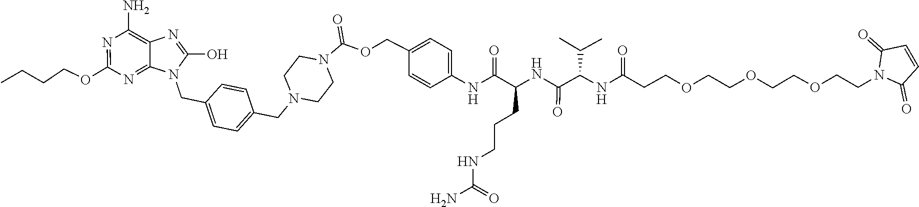 [SMILES: CCCCOc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3)c2n1]

The titled compound was prepared according to General Procedure 4 from compound 166 (2.2 mg, 0.0034 mmol, 1.0 eq.) and compound 59 (2.8 mg. 0.0034 mmol, 1.0 eq.). Preparative HPLC purification was accomplished as described in General Procedure 5, eluting with a 10 to 50% CH₃CN+0.1% TFA/H₂O+0.1% TFA gradient to give the titled compound as a white solid (2.3 mg, 0.00017 mmol, 50%). LC-MS: Calc'd m/z=1099.6 for C₅₃H₇₃N₁₃O₁₃. found [M+H]⁺=1100.7. ¹H NMR (300 MHz, MeOD) δ 8.24 (d, J=7.6 Hz, OH), 7.67-7.44 (m, 7H), 7.34 (d, J=8.5 Hz, 2H), 6.82 (s, 2H), 5.12 (s, 2H), 5.06 (s, 2H), 4.56-4.46 (m, 1H), 4.38-4.27 (m, 4H), 4.21 (d, J=6.9 Hz, 1H), 3.87-3.49 (m, 14H), 3.29-3.08 (m, 10H), 2.57 (t, J=6.0 Hz, 2H), 2.22-2.05 (m, 1H), 1.98-1.85 (m, 1H), 1.83-1.67 (m, 3H), 1.67-1.57 (m, 1H), 1.57-1.40 (m, 2H), 1.05-0.94 (m, 9H).

### Example 2.21: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (1-(4-((6-amino-2-butoxy-8-hydroxy-9H-purin-9-yl)methyl)-3-methoxybenzyl)piperidin-4-yl)carbamate (MT-VC-PABC-Compound 155)

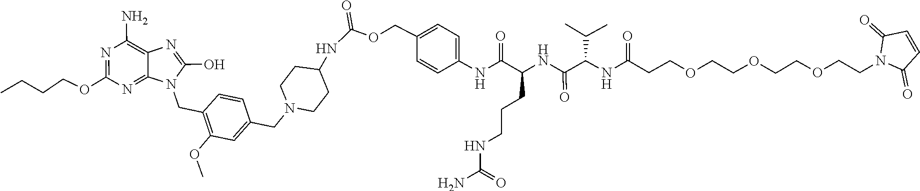 [SMILES: CCCCOc1nc(N)c2nc(O)n(Cc3ccc(CN4CCC(NC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 155 (3.4 mg, 0.0050 mmol, 1.0 eq.) and compound 59 (4.1 mg. 0.0050 mmol, 1.0 eq.). Preparative HPLC purification was accomplished as described in General Procedure 5, eluting with a 10 to 50% CH₃CN+0.1% TFA/H₂O+0.1% TFA gradient to give the titled compound as a white solid (2.6 mg, 0.0026 mmol, 38%). LC-MS: Calc'd m/z=1143.6 for C₅₅H₇₇N₁₃O₁₄. found [M+H]⁺=1144.7. ¹H NMR (300 MHz, MeOD) δ 7.61 (d, J=8.4 Hz, 2H), 7.32 (d, J=8.5 Hz, 2H), 7.17-6.96 (m, 3H), 6.83 (s, 2H), 5.06 (s, 4H), 4.56-4.45 (m, 1H), 4.38-4.16 (m, 5H), 3.95 (s, 3H), 3.81-3.44 (m, 18H), 3.27-3.03 (m, 7H), 2.57 (t, J=6.1 Hz, 2H), 2.30-1.36 (m, 9H), 1.05-0.91 (m, 9H).

### Example 2.22: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(4-((6-amino-2-butoxy-8-hydroxy-9H-purin-9-yl)methyl)-3-methoxybenzyl)piperazine-1-carboxylate (MT-VC-PABC-Compound 132)

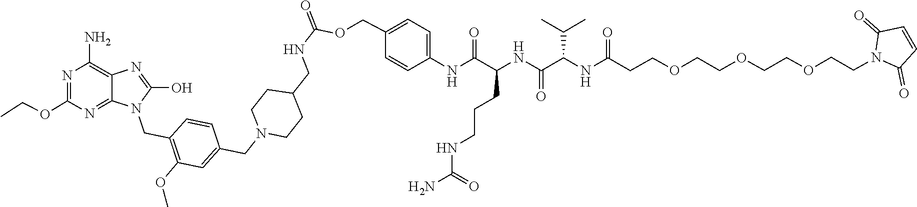 [SMILES: CCOc1nc(N)c2nc(O)n(Cc3ccc(CN4CCC(CNC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 132 (4.4 mg, 0.0066 mmol, 1.0 eq.) and compound 59 (5.5 mg. 0.0066 mmol, 1.0 eq.). Preparative HPLC purification was accomplished as described in General Procedure 5, eluting with a 10 to 50% CH₃CN+0.1% TFA/H₂O+0.1% TFA gradient to give the titled compound as a white solid (5.0 mg, 0.0040 mmol, 61%). LC-MS: Calc'd m/z=1129.6 for C₅₄H₇₅N₁₃O₁₄. found [M+H]⁺=1130.7. ¹H NMR (300 MHz, MeOD) δ 7.68-7.56 (m, 2H), 7.31 (d, J=8.4 Hz, 2H), 7.17-7.06 (m, 2H), 7.05-6.96 (m, 1H), 6.83 (s, 2H), 5.09-5.00 (m, 4H), 4.52 (dd, J=9.1, 5.0 Hz, 1H), 4.42-4.17 (m, 5H), 3.94 (s, 3H), 3.81-3.41 (m, 18H), 3.29-2.86 (m, 7H), 2.57 (t, J=6.0 Hz, 2H), 2.23-1.26 (m, 10H), 1.00 (dd, J=6.8, 3.7 Hz, 6H).

### Example 2.23: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (2-(1-(4-((6-amino-2-ethoxy-8-hydroxy-9H-purin-9-yl)methyl)-3-methoxybenzyl)piperidin-4-yl)ethyl)-carbamate (MT-VC-PABC-Compound 140)

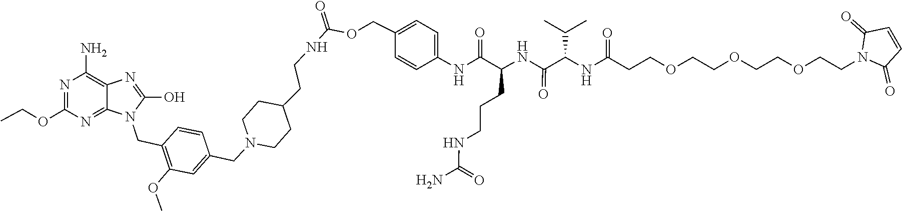 [SMILES: CCOc1nc(N)c2nc(O)n(Cc3ccc(CN4CCC(CCNC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 140 (4.5 mg, 0.0066 mmol, 1.0 eq.) and compound 59 (5.5 mg. 0.0066 mmol, 1.0 eq.). Preparative HPLC purification was accomplished as described in General Procedure 5, eluting with a 10 to 50% CH₃CN+0.1% TFA/H₂O+0.1% TFA gradient to give the titled compound as a white solid (5.3 mg, 0.0039 mmol, 59%). LC-MS: Calc'd m/z=1143.6 for C₅₅H₇₇N₁₃O₁₄. found [M+H]⁺=1144.7. ¹H NMR (300 MHz, MeOD) δ 7.62 (d, J=8.2 Hz, 2H), 7.33 (d, J=8.4 Hz, 2H), 7.17-7.06 (m, 2H), 7.05-6.97 (m, 1H), 6.83 (s, 2H), 5.09-5.00 (m, 4H), 4.50 (dd, J=9.1, 4.9 Hz, 1H), 4.40-4.14 (m, 5H), 3.94 (s, 3H), 3.85-3.39 (m, 18H), 3.15 (dt, J=16.9, 6.8 Hz, 8H), 2.88 (t, J=12.6 Hz, 1H), 2.57 (t, J=6.0 Hz, 2H), 2.20-1.39 (m, 7H), 1.34 (t, J=7.1 Hz, 3H), 0.99 (dd, J=6.9, 2.7 Hz, 6H).

### Example 2.24: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 9-(4-((6-amino-2-ethoxy-8-hydroxy-9H-purin-9-yl)methyl)-3-methoxybenzyl)-3,9-diazaspiro[5.5]undecane-3-carboxylate (MT-VC-PABC-Compound 139)

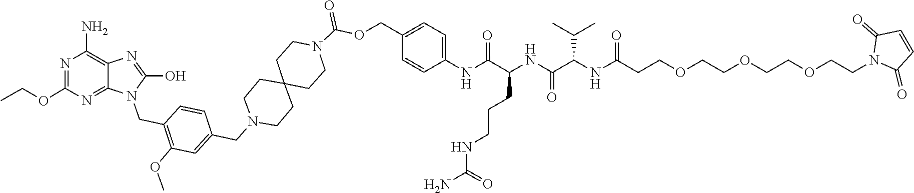 [SMILES: CCOc1nc(N)c2nc(O)n(Cc3ccc(CN4CCC5(CC4)CCN(C(=O)OCc4ccc(NC(=O)[C@H](CCCNC(N)=O)NC(=O)[C@@H](NC(=O)CCOCCOCCOCCN6C(=O)C=CC6=O)C(C)C)cc4)CC5)cc3OC)c2n1]

The titled compound was prepared according to General Procedure 4 from compound 139 (6.2 mg, 0.0087 mmol, 1.0 eq.) and compound 59 (7.2 mg. 0.0087 mmol, 1.0 eq.). Preparative HPLC purification was accomplished as described in General Procedure 5, eluting with a 10 to 50% CH₃CN+0.1% TFA/H₂O+0.1% TFA gradient to give the titled compound as a white solid (6.1 mg, 0.0044 mmol, 50%). LC-MS: Calc'd m/z=1169.6 for C₅₇H₇₉N₁₃O₁₄. found [M+H]⁺=1170.7. ¹H NMR (300 MHz, MeOD) δ 7.66-7.57 (m, 2H), 7.33 (d, J=8.5 Hz, 2H), 7.18-7.07 (m, 2H), 7.01 (dd, J=7.7, 1.5 Hz, 1H), 6.83 (s, 2H), 5.11-5.03 (m, 4H), 4.51 (dd, J=9.1, 5.0 Hz, 1H), 4.41-4.27 (m, 4H), 4.21 (d, J=6.9 Hz, 1H), 3.94 (s, 3H), 3.83-3.42 (m, 18H), 3.27-3.08 (m, 8H), 2.57 (t, J=6.0 Hz, 2H), 2.22-2.04 (m, 1H), 1.97 (d, J=14.7 Hz, 4H), 1.89-1.47 (m, 4H), 1.45-1.29 (m, 5H), 1.00 (dd, J=6.8, 4.1 Hz, 6H).

### Example 2.25: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (1-(4-((6-amino-2-ethoxy-8-hydroxy-9H-purin-9-yl)methyl)-3-methoxybenzyl)piperidin-4-yl)carbamate (MT-VC-PABC-Compound 110)

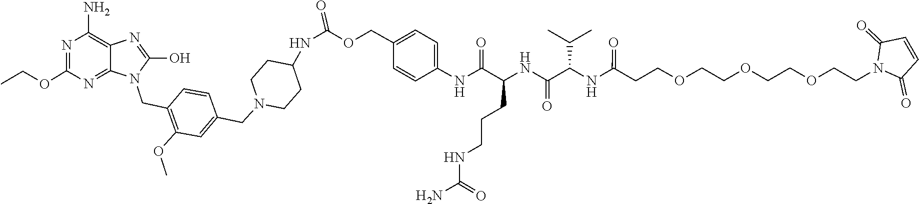 [SMILES: CCOc1nc(N)c2nc(O)n(Cc3ccc(CN4CCC(NC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 110 (18 mg, 0.027 mmol, 1.0 eq.) and compound 59 (23 mg. 0.027 mmol, 1.0 eq.). Preparative HPLC purification was accomplished as described in General Procedure 5, eluting with a 10 to 50% CH₃CN+0.1% TFA/H₂O+0.1% TFA gradient to give the titled compound as a white solid (13 mg, 0.010 mmol, 35%). LC-MS: Calc'd m/z=1115.5 for C₅₃H₇₃N₁₃O₁₄. found [M+H]⁺=1116.7. ¹H NMR (300 MHz, MeOD) δ 7.60 (d, J=8.1 Hz, 2H), 7.31 (d, J=8.2 Hz, 2H), 7.18-7.07 (m, 2H), 7.01 (dd, J=7.7, 1.5 Hz, 1H), 6.82 (s, 2H), 5.09-5.01 (m, 4H), 4.58-4.43 (m, 1H), 4.44-4.18 (m, 5H), 3.93 (s, 3H), 3.81-3.45 (m, 18H), 3.29-3.01 (m, 7H), 2.57 (t, J=6.0 Hz, 2H), 2.24-1.50 (m, 5H), 1.35 (t, J=7.1 Hz, 3H), 1.00 (dd, J=6.8, 4.0 Hz, 6H).

### Example 2.26: 1-(2-(4-(4-((6-amino-2-ethoxy-8-hydroxy-9H-purin-9-yl)methyl)-3-methoxy-benzyl)piperazin-1-yl)ethyl)-1H-pyrrole-2,5-dione (ME2-Compound 100)

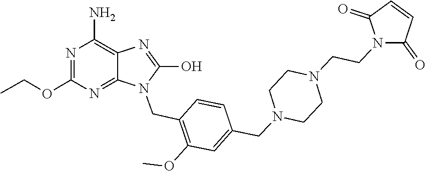 [SMILES: CCOc1nc(N)c2nc(O)n(Cc3ccc(CN4CCN(CCN5C(=O)C=CC5=O)CC4)cc3OC)c2n1]

To a suspension of 1-(2-hydroxyethyl)pyrrole-2,5-dione (0.10 g, 0.71 mmol, 15 eq.) in DCM (10 mL) was added Dess-Martin periodinane (0.30 g, 0.71 mmol, 15 eq.). The resulting mixture was stirred at room temperature for 18 h then filtered through a celite plug. The filtrate was concentrated in vacuo to give the crude intermediate aldehyde which was carried forward without additional purification. The aldehyde was then dissolved in 3:1 EtOAc/AcOH (10 mL) along with Compound 100 (35 mg, 0.046 mmol, 1.0 eq.) followed by the addition of Na(OAc)₃BH (98 mg, 0.46 mmol, 10 eq.). The resulting mixture was stirred at room temperature for 3 h then concentrated in vacuo to give the crude product. Preparative HPLC purification was accomplished as described in General Procedure 5, eluting with a 10 to 50% CH₃CN+0.1% TFA/H₂O+0.1% TFA gradient to give the titled compound as a white solid (16 mg, 0.021 mmol, 46%). LC-MS: Calc'd m/z=536.3 for C₂₆H₃₂N₈O₅. found [M+H]⁺=537.4. ¹H NMR (300 MHz, MeOD) δ 7.16 (d, J=8.6 Hz, 2H), 7.01 (d, J=7.7 Hz, 1H), 6.85 (s, 2H), 5.07 (s, 2H), 4.42 (q, J=7.1 Hz, 2H), 4.23 (s, 2H), 3.92 (s, 3H), 3.79-3.69 (m, 2H), 3.26-3.20 (m, 4H), 3.05-2.99 (m, 4H), 2.90-2.84 (m, 2H), 1.37 (t, J=7.1 Hz, 3H).

### Example 2.27: 1-(6-(4-(4-((6-amino-2-ethoxy-8-hydroxy-9H-purin-9-yl)methyl)-3-methoxy-benzyl)piperazin-1-yl)-6-oxohexyl)-1H-pyrrole-2,5-dione (MC-Compound 100)

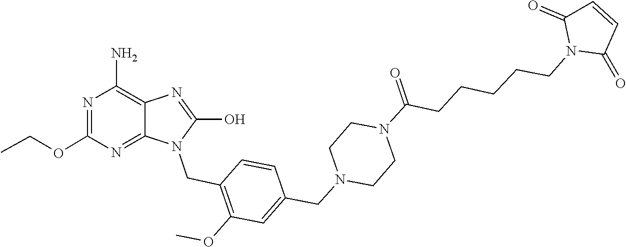 [SMILES: CCOc1nc(N)c2nc(O)n(Cc3ccc(CN4CCN(C(=O)CCCCCN5C(=O)C=CC5=O)CC4)cc3OC)c2n1]

To a solution of compound 100 (7.0 mg, 0.0093 mmol, 1.0 eq.) in DMF (300 μL) was added 2,5-dioxopyrrolidin-1-yl 6-(2,5-dioxopyrrol-1-yl)hexanoate (3.5 mg, 0.011 mmol, 1.2 eq.) then DIPEA (4.7 μL, 0.027 mmol, 3.0 eq.) and the resulting solution stirred at room temperature for 1 h. Preparative HPLC purification was accomplished as described in General Procedure 5, eluting with a 10 to 50% CH₃CN+0.1% TFA/H₂O+0.1% TFA gradient to give the titled compound as a white solid (4.6 mg, 0.0055 mmol, 59%). LC-MS: Calc'd m/z=606.3 for C₃₀H₃₈N₈O₆. found [M+H]⁺=607.4. ¹H NMR (300 MHz, MeOD) δ 7.17 (d, J=1.5 Hz, 1H), 7.11 (d, J=7.7 Hz, 1H), 7.03 (dd, J=7.7, 1.5 Hz, 1H), 6.82 (s, 2H), 5.06 (s, 2H), 4.33 (dd, J=14.3, 7.2 Hz, 4H), 3.95 (s, 3H), 3.52 (t, J=6.9 Hz, 2H), 3.47-2.97 (m, 8H), 2.44 (t, J=7.4 Hz, 3H), 1.73-1.54 (m, 6H), 1.34 (t, J=7.1 Hz, 2H).

### Example 2.28: 1-(2-(2-(2-(3-(4-(4-((6-amino-2-ethoxy-8-hydroxy-9H-purin-9-yl)methyl)-3-methoxybenzyl)piperazin-1-yl)-3-oxopropoxy)ethoxy)ethoxy)ethyl)-1H-pyrrole-2,5-dione (MT-Compound 100)

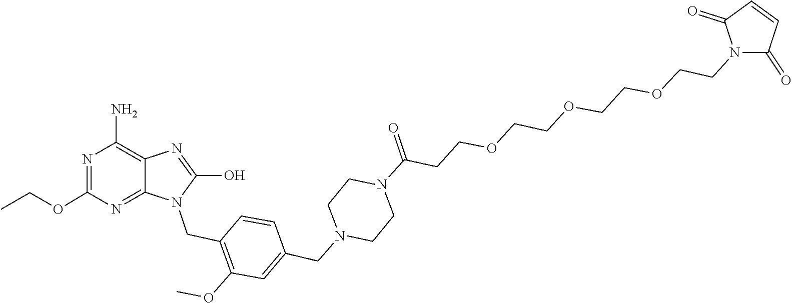 [SMILES: CCOc1nc(N)c2nc(O)n(Cc3ccc(CN4CCN(C(=O)CCOCCOCCOCCN5C(=O)C=CC5=O)CC4)cc3OC)c2n1]

To a solution of compound 100 (0.010 g, 0.012 mmol, 1.0 eq.) and compound 56 (5.4 mg, 0.012 mmol, 1.0 eq.) in DMF (400 μL) was added DIPEA (6.1 μL, 0.035 mmol, 3.0 eq.) and the resulting solution stirred at room temperature for 1 h. Preparative HPLC purification was accomplished as described in General Procedure 5, eluting with a 10 to 50% CH₃CN+0.1% TFA/H₂O+0.1% TFA gradient to give the titled compound as a white solid (6.3 mg, 0.0068 mmol, 57%). LC-MS: Calc'd m/z=696.3 for C₃₃H₄₄N₈O₉. found [M+H]⁺=697.4. ¹H NMR (300 MHz, MeOD) δ 7.21-7.08 (m, 2H), 7.04 (dd, J=7.7, 1.5 Hz, 1H), 6.80 (s, 2H), 5.06 (s, 2H), 4.42-4.29 (m, 4H), 3.94 (s, 3H), 3.81-3.54 (m, 18H), 3.49-3.03 (m, 4H), 2.71 (s, 2H), 1.35 (t, J=7.1 Hz, 3H).

### Example 2.29: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(4-((6-amino-2-butoxy-8-hydroxy-9H-purin-9-yl)methyl)-3-methoxyphenethyl)piperazine-1-carboxylate (MT-VC-PABC-Compound 180)

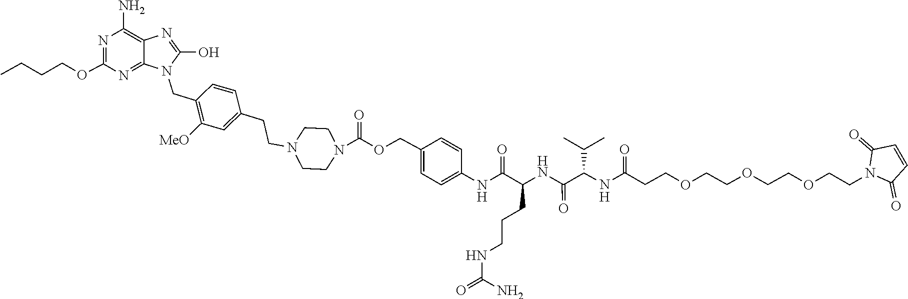 [SMILES: CCCCOc1nc(N)c2nc(O)n(Cc3ccc(CCN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 178 (3.4 mg, 0.0050 mmol, 1.0 eq.) and compound 59 (4.2 mg. 0.0050 mmol, 1.0 eq.). Preparative HPLC purification was accomplished as described in General Procedure 5, eluting with a 20 to 60% CH₃CN+0.1% TFA/H₂O+0.1% TFA gradient to give the titled compound as a white solid (2.8 mg, 0.0020 mmol, 41%). LC-MS: Calc'd m/z=1143.6 for C₅₅H₇₇N₁₃O₁₄. found [M+H]⁺=1144.8.

### Example 2.30: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (1-(3-(4-((6-amino-2-butoxy-8-hydroxy-9H-purin-9-yl)methyl)-3-methoxyphenyl)propyl)piperidin-4-yl)carbamate (MT-VC-PABC-Compound 172)

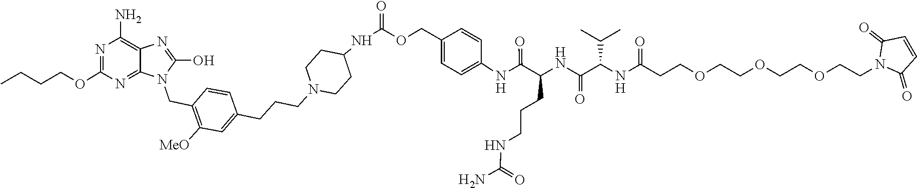 [SMILES: CCCCOc1nc(N)c2nc(O)n(Cc3ccc(CCCN4CCC(NC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 172 (5.0 mg, 0.0070 mmol, 1.0 eq.) and compound 59 (5.8 mg. 0.0050 mmol, 1.0 eq.). Preparative HPLC purification was accomplished as described in General Procedure 5, eluting with a 20 to 60% CH₃CN+0.1% TFA/H₂O+0.1% TFA gradient to give the titled compound as a white solid (1.2 mg, 0.00086 mmol, 12%). LC-MS: Calc'd m/z=1171.6 for C₅₇H₈₁N₁₃O₁₄. found [M+H]⁺=1172.8.

### Example 2.31: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(3-(4-((6-amino-2-butoxy-8-hydroxy-9H-purin-9-yl)methyl)-3-methoxyphenyl)propyl)piperazine-1-carboxylate (MT-VC-PABC-Compound 173)

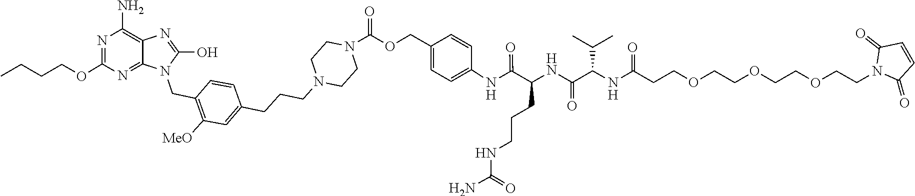 [SMILES: CCCCOc1nc(N)c2nc(O)n(Cc3ccc(CCCN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3OC)c2n1]

The titled compound was prepared according to General Procedure 4 from compound 174 (5.0 mg, 0.0072 mmol, 1.0 eq.) and compound 59 (5.9 mg. 0.0072 mmol, 1.0 eq.). Preparative HPLC purification was accomplished as described in General Procedure 5, eluting with a 20 to 60% CH₃CN+0.1% TFA/H₂O+0.1% TFA gradient to give the titled compound as a white solid (3.3 mg, 0.0024 mmol, 33%). LC-MS: Calc'd m/z=1157.6 for C₅₆H₇₉N₁₃O₁₄. found [M+H]⁺=1158.8.

### Example 2.32: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl 4-(4-((6-amino-8-hydroxy-2-(pentylamino)-9H-purin-9-yl)methyl)benzyl)piperazine-1-carboxylate (MT-VC-PABC-Compound 187)

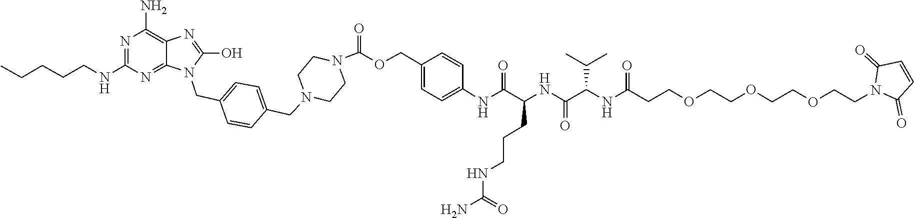 [SMILES: CCCCCNc1nc(N)c2nc(O)n(Cc3ccc(CN4CCN(C(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3)c2n1]

The titled compound was prepared according to General Procedure 4 from compound 187 (20 mg, 0.048 mmol, 1.0 eq.) and compound 59 (44 mg. 0.053 mmol, 1.1 eq.). Preparative HPLC purification was accomplished as described in General Procedure 5, eluting with a 10 to 55% CH₃CN+0.1% TFA/H₂O+0.1% TFA gradient to give the titled compound as a white solid (24.4 mg, 0.018 mmol, 38%). LC-MS: Calc'd m/z=1112.6 for C₅₄H₇₆N₁₄O₁₂. found [M+H]⁺=1113.8. ¹H NMR (300 MHz, MeOD) δ 7.67-7.46 (m, 6H), 7.34 (d, J=8.5 Hz, 2H), 6.82 (s, 2H), 5.12 (s, 2H), 5.05 (s, 2H), 4.58-4.47 (m, 1H), 4.36 (s, 2H), 4.26-4.16 (m, 1H), 3.85-3.52 (m, 18H), 3.42 (t, J=7.1 Hz, 2H), 3.18 (dtd, J=20.4, 13.5, 6.7 Hz, 6H), 2.57 (t, J=6.0 Hz, 2H), 2.20-2.02 (m, 1H), 1.98-1.88 (m, 1H), 1.83-1.73 (m, 1H), 1.70-1.56 (m, 4H), 1.43-1.32 (m, 4H), 1.05-0.89 (m, 9H).

### Example 2.33: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (1-(4-((6-amino-8-hydroxy-2-(pentylamino)-9H-purin-9-yl)methyl)benzyl)piperidin-4-yl)carbamate (MT-VC-PABC-Compound 188)

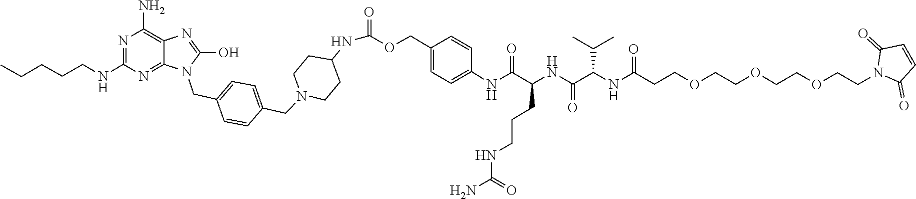 [SMILES: CCCCCNc1nc(N)c2nc(O)n(Cc3ccc(CN4CCC(NC(=O)OCc5ccc(NC(=O)[C@H](CCCNC(N)=O)NC(=O)[C@@H](NC(=O)CCOCCOCCOCCN6C(=O)C=CC6=O)C(C)C)cc5)CC4)cc3)c2n1]

The titled compound was prepared according to General Procedure 4 from compound 188 (25 mg, 0.057 mmol, 1.0 eq.) and compound 59 (52 mg. 0.063 mmol, 1.1 eq.). Preparative HPLC purification was accomplished as described in General Procedure 5, eluting with a 10 to 55% CH₃CN+0.1% TFA/H₂O+0.1% TFA gradient to give the titled compound as a white solid (21 mg, 0.015 mmol, 27%). LC-MS: Calc'd m/z=1126.6 for C₅₅H₇₈N₁₄O₁₂. found [M+H]⁺=1127.8. ¹H NMR (300 MHz, MeOD) δ 7.64-7.45 (m, 6H), 7.31 (d, J=8.2 Hz, 2H), 6.82 (s, 2H), 5.04 (s, 4H), 4.54-4.48 (m, 1H), 4.30 (s, 2H), 4.21 (dd, J=7.0, 2.9 Hz, 1H), 3.84-3.38 (m, 21H), 3.29-3.01 (m, 6H), 2.57 (t, J=6.0 Hz, 2H), 2.26-1.86 (m, 3H), 1.84-1.52 (m, 4H), 1.43-1.32 (m, 4H), 1.08-0.89 (m, 9H).

### Example 2.34: 4-((14S,17S)-1-(2,5-dioxo-2,5-dihydro-1H-pyrrol-1-yl)-14-isopropyl-12,15-dioxo-17-(3-ureidopropyl)-3,6,9-trioxa-13,16-diazaoctadecan-18-amido)benzyl (4-((6-amino-8-hydroxy-2-(pentylamino)-9H-purin-9-yl)methyl)benzyl)(4-(aminomethyl)benzyl)carbamate (MT-VC-PABC-Compound 191)

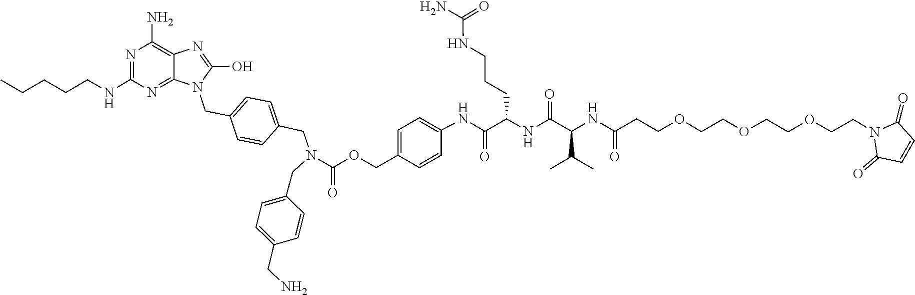 [SMILES: CCCCCNc1nc(N)c2nc(O)n(Cc3ccc(CN(Cc4ccc(CN)cc4)C(=O)OCc4ccc(NC(=O)[C@H](CCCNC(N)=O)NC(=O)[C@@H](NC(=O)CCOCCOCCOCCN5C(=O)C=CC5=O)C(C)C)cc4)cc3)c2n1]

The titled compound was prepared according to General Procedure 4 from the Boc intermediate of compound 191 (37 mg, 0.046 mmol, 1.0 eq.) and compound 59 (42 mg. 0.051 mmol, 1.1 eq.). Preparative HPLC purification was accomplished as described in General Procedure 5, eluting with a 30 to 60% CH₃CN+0.1% TFA/H₂O+0.1% TFA gradient. Half of the resulting Boc intermediate was reserved and deprotection of the remaining material was accomplished according to General Procedure 2. Final preparative HPLC purification was accomplished as described in General Procedure 5, eluting with a 10 to 55% CH₃CN+0.1% TFA/H₂O+0.1% TFA gradient. to give the titled compound as a white solid (10 mg, 0.0072 mmol, 31%). LC-MS: Calc'd m/z=1162.6 for C₅₈H₇₈N₁₄O₁₂. found [M+H]⁺=1163.8. ¹H NMR (300 MHz, MeOD) δ 7.60-7.07 (m, 12H), 6.81 (s, 2H), 5.14 (s, 2H), 4.99 (s, 2H), 4.62-4.39 (m, 4H), 4.19 (d, J=6.8 Hz, 1H), 4.11 (s, 2H), 3.77 (q, J=5.5 Hz, 1H), 3.70-3.49 (m, 14H), 3.44-3.34 (m, 2H), 3.27-3.09 (m, 4H), 2.60 (t, J=6.1 Hz, 2H), 2.22-2.09 (m, 1H), 1.99-1.93 (m, 1H), 1.85-1.75 (m, 1H), 1.66-1.58 (m, 4H), 1.41-1.28 (m, 4H), 1.03 (dd, J=6.8, 3.3 Hz, 4H), 0.95-0.89 (m, 3H).

### Example 2.35: N²-((((9H-fluoren-9-yl)methoxy)carbonyl)-L-valyl)-N⁶-(tert-butoxycarbonyl)-L-lysine (Fmoc-VK(Boc)-OH)

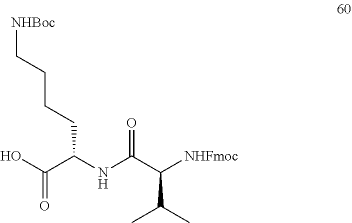

To a solution of 2,5-dioxopyrrolidin-1-yl-(((9H-fluoren-9-yl)methoxy)carbonyl)-L-valinate (14 g, 33 mmol, 1.0 eq.) and N⁶-(tert-butoxycarbonyl)-L-lysine (8.9 g, 36 mmol, 1.1 eq.) in THF (100 mL) was added saturated NaHCO₃ solution (43 mL, 49 mmol, 1.5 eq.) and H₂O (50 mL) and the resulting mixture stirred at room temperature for 18 h. The reaction mixture was concentrated in vacuo, redissolved in 5:1 toluene/iPrOH (300 mL) and heated to reflux for 30 mins then cooled to room temperature. The reaction mixture was then dried over MgSO₄, filtered, and concentrated in vacuo to give the crude product as an oily foam solid, which was then suspended in 3:1 EtOAc/hexanes (100 mL). The resulting suspension was sonicated for 20 mins then stirred at room temperature for 18 h, after which the title product 60 was isolated as a solid cake after filtration (12 g, 21 mmol, 63%). LC-MS: Calc'd m/z=567.3 for C₃₁H₄₁N₃O₇. found [M+H]⁺=568.4.

### Example 2.36: (9H-fluoren-9-yl)methyl ((S)-1-(((S)-6-((tert-butoxycarbonyl)amino)-1-((4-(hydroxymethyl)phenyl)amino)-1-oxohexan-2-yl)amino)-3-methyl-1-oxobutan-2-yl)carbamate (Fmoc-VK(Boc)-PAB-OH)

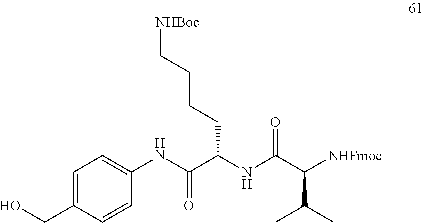

A mixture of compound 60 (1.1 g, 2.0 mmol, 1.0 eq.), EEDQ (0.98 g, 3.9 mmol, 2.0 eq.) and P-aminobenzylalcohol (0.34 g, 2.8 mmol, 1.4 eq.) in THF (25 mL) was stirred at room temperature for 18 h in a tinfoil wrapped flask. The reaction mixture was then concentrated in vacuo and normal phase flash purification was accomplished as described in General Procedure 5 using a 50 g silica column, eluting with a 0 to 10% DCM/MeOH gradient to give the title compound 61 as a yellow solid (1.2 g, 1.8 mmol, 90%). LC-MS: Calc'd m/z=672.4 for C₃₈H₄₈N₄O₇. found [M+H]⁺=673.5.

### Example 2.37: tert-butyl ((14S,17S)-1-(2,5-dioxo-2,5-dihydro-1H-pyrrol-1-yl)-14-isopropyl-17-((4-((((4-nitrophenoxy)carbonyl)oxy)methyl)phenyl)carbamoyl)-12,15-dioxo-3,6,9-trioxa-13,16-diazahenicosan-21-yl)carbamate (MT-VK(Boc)-PABC-PNP)

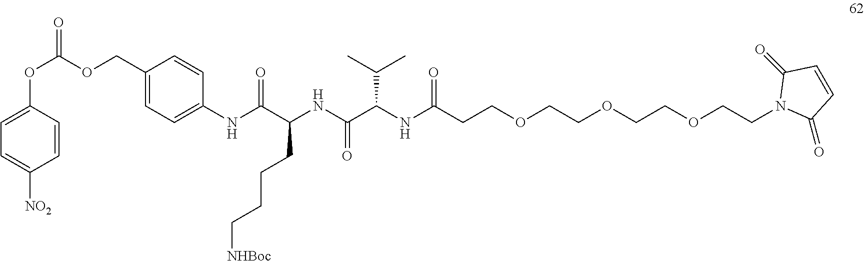 [SMILES: CC(C)[C@H](NC(=O)CCOCCOCCOCCN1C(=O)C=CC1=O)C(=O)N[C@@H](CCCCNC(=O)OC(C)(C)C)C(=O)Nc1ccc(COC(=O)Oc2ccc([N+](=O)[O-])cc2)cc1]

Compound 61 (0.080 g, 1.8 mmol, 1.0 eq.) was dissolved in 20% piperidine/DMF (2 mL) and the resulting solution stirred for 30 mins, after which the solvent was removed in vacuo and the residue co-evaporated with DMF (1×3 mL) to give the crude intermediate. A mixture of the crude intermediate and compound 56 (0.080 g, 0.18 mmol, 1.0 eq.) was then dissolved in DMF (1 mL), followed by the addition of DIPEA (93 mL, 0.54 mmol, 3.0 eq.). The resulting solution was stirred at room temperature for 1 h, after which bis(4-nitrophenyl) carbonate (0.11 g, 0.36 mmol, 2.0 eq.) was added. The resulting mixture was stirred at room temperature for an additional 30 mins, after which the reaction was adjusted to pH 1 with 1 M HCl. Reverse phase flash purification was accomplished as described in General Procedure 5 using a 60 g C18 column, eluting with a 10 to 90% CH₃CN+0.1% TFA/H₂O+0.1% TFA gradient to give the title compound 62 as a white solid (140 mg, 0.16 mmol, 87%). LC-MS: Calc'd m/z=898.4 for C₄₃H₅₆N₆O₁₅. found [M+H-Boc]+=799.5.

### Example 2.38: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl 4-(4-((6-amino-2-ethoxy-8-hydroxy-9H-purin-9-yl)methyl)-3-methoxybenzyl)piperazine-1-carboxylate (MT-VK-PABC-Compound 100)

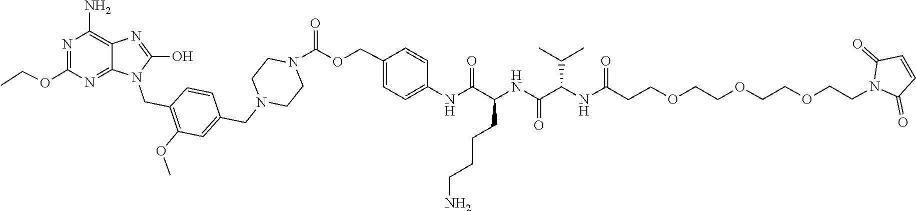 [SMILES: CCOc1nc(N)c2nc(O)n(Cc3ccc(CN4CCN(C(=O)OCc5ccc(NC(=O)[C@H](CCCCN)NC(=O)[C@@H](NC(=O)CCOCCOCCOCCN6C(=O)C=CC6=O)C(C)C)cc5)CC4)cc3OC)c2n1]

The titled compound was prepared according to General Procedure 7 from compound 100 (0.010 g, 0.024 mmol, 1.0 eq.) and compound 62 (24 mg. 0.027 mmol, 1.1 eq.). Preparative HPLC purification of the Boc intermediate was accomplished as described in General Procedure 5, eluting with a 20 to 65% CH₃CN+0.1% TFA/H₂O+0.1% TFA gradient, followed by deprotection as described in General Procedure 2. Final preparative HPLC purification was accomplished as described in General Procedure 5, eluting with a 20 to 65% CH₃CN+0.1% TFA/H₂O+0.1% TFA gradient to give the titled product as a white solid (11 mg, 0.0075 mmol, 31%). LC-MS: Calc'd m/z=1072.5 for C₅₂H₇₂N₁₂O₃. found [M+H]⁺=1073.7. ¹H NMR (300 MHz, MeOD) δ 7.64-7.54 (m, 2H), 7.38-7.29 (m, 2H), 7.17-7.03 (m, 2H), 6.99 (dd, J=7.7, 1.5 Hz, 1H), 6.82 (s, 2H), 5.11 (s, 2H), 5.03 (s, 2H), 4.53 (td, J=8.9, 4.6 Hz, 1H), 4.32 (s, 2H), 4.30 (q, J=7.1 Hz, 2H), 4.14-4.03 (m, 1H), 3.92 (s, 3H), 3.85-3.50 (m, 18H), 3.26 (s, 4H), 2.95 (t, J=7.3 Hz, 2H), 2.55 (t, J=6.0 Hz, 2H), 2.16-1.98 (m, 1H), 2.02-1.87 (m, 1H), 1.90-1.76 (m, 1H), 1.73-1.67 (m, 2H), 1.62-1.50 (m, 2H), 1.31 (t, J=7.1 Hz, 3H), 1.00 (dd, J=6.8, 3.6 Hz, 6H).

### Example 2.39: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl (4-((6-amino-2-ethoxy-8-hydroxy-9H-purin-9-yl)methyl)-3-methoxybenzyl)(benzyl)carbamate (MT-VK-PABC-Compound 107)

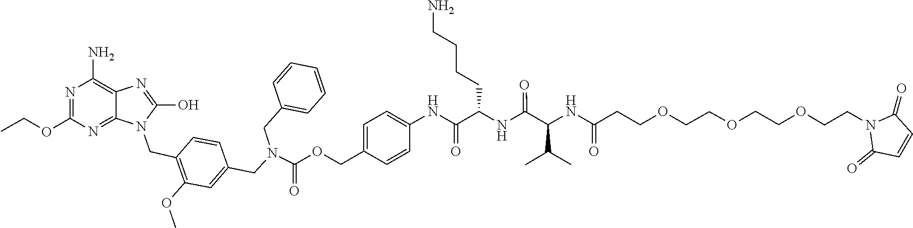 [SMILES: CCOc1nc(N)c2nc(O)n(Cc3ccc(CN(Cc4ccccc4)C(=O)OCc4ccc(NC(=O)[C@H](CCCCN)NC(=O)[C@@H](NC(=O)CCOCCOCCOCCN5C(=O)C=CC5=O)C(C)C)cc4)cc3OC)c2n1]

The titled compound was prepared according to General Procedure 7 from compound 107 (7.0 mg, 0.018 mmol, 1.0 eq.) and compound 62 (17 mg. 0.019 mmol, 1.1 eq.). Preparative HPLC purification of the Boc intermediate was accomplished as described in General Procedure 5, eluting with a 20 to 65% CH₃CN+0.1% TFA/H₂O+0.1% TFA gradient, followed by deprotection as described in General Procedure 2. Final preparative HPLC purification was accomplished as described in General Procedure 5, eluting with a 10 to 55% CH₃CN+0.1% TFA/H₂O+0.1% TFA gradient to give the titled product as a white solid (3.1 mg, 0.0023 mmol, 13%). LC-MS: Calc'd m/z=1093.5 for C₅₅H₇₁N₁₁O₁₃. found [M+H]⁺=1094.7. ¹H NMR (300 MHz, MeOD) δ 7.54 (s, 2H), 7.27 (s, 7H), 7.17 (s, 2H), 6.98 (s, 1H), 6.81 (s, 2H), 5.15 (s, 2H), 5.01 (s, 2H), 4.43 (t, J=23.4 Hz, 7H), 4.22 (d, J=6.8 Hz, 1H), 3.81-3.52 (m, 17H), 3.05 (t, J=7.3 Hz, 2H), 2.58 (t, J=6.0 Hz, 2H), 2.21-2.08 (m, 1H), 1.98-1.89 (m, 1H), 1.79-1.73 (m, 1H), 1.55-1.49 (m, 2H), 1.45-1.40 (m, 2H), 1.40-1.28 (m, 3H), 1.02 (dd, J=6.7, 4.7 Hz, 6H).

### Example 2.40: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl (4-((6-amino-2-ethoxy-8-hydroxy-9H-purin-9-yl)methyl)-3-methoxybenzyl)(cyclobutyl)carbamate (MT-VK-PABC-Compound 108)

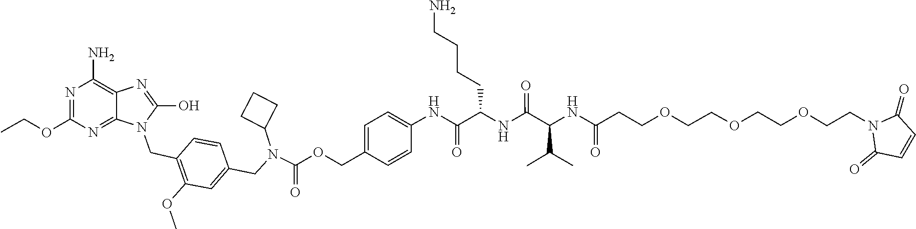 [SMILES: CCOc1nc(N)c2nc(O)n(Cc3ccc(CN(C(=O)OCc4ccc(NC(=O)[C@H](CCCCN)NC(=O)[C@@H](NC(=O)CCOCCOCCOCCN5C(=O)C=CC5=O)C(C)C)cc4)C4CCC4)cc3OC)c2n1]

The titled compound was prepared according to General Procedure 7 from compound 108 (7.0 mg, 0.018 mmol, 1.0 eq.) and compound 62 (17 mg. 0.019 mmol, 1.1 eq.). Preparative HPLC purification of the Boc intermediate was accomplished as described in General Procedure 5, eluting with a 20 to 65% CH₃CN+0.1% TFA/H₂O+0.1% TFA gradient, followed by deprotection as described in General Procedure 2. Final preparative HPLC purification was accomplished as described in General Procedure 5, eluting with a 10 to 50% CH₃CN+0.1% TFA/H₂O+0.1% TFA gradient to give the titled product as a white solid (3.1 mg, 0.0024 mmol, 13%). LC-MS: Calc'd m/z=1057.5 for C₅₂H₇₁N₁₁O₁₃. found [M+H]⁺=1058.7. ¹H NMR (300 MHz, MeOD) δ 7.62-6.97 (m, 6H), 6.89 (s, 1H), 6.83 (s, 1H), 5.07 (s, 2H), 5.00 (s, 2H), 4.61-4.52 (m, 3H), 4.30 (q, J=7.1 Hz, 2H), 4.18-4.07 (m, 1H), 3.90-3.46 (m, 18H), 2.98 (t, J=7.3 Hz, 2H), 2.58 (t, J=5.9 Hz, 2H), 2.21-1.47 (m, 14H), 1.30 (t, J=7.0 Hz, 3H), 1.03 (dd, J=6.7, 2.6 Hz, 6H).

### Example 2.41: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl (4-((6-amino-2-ethoxy-8-hydroxy-9H-purin-9-yl)methyl)-3-methoxybenzyl)(2-hydroxy-2-methylpropyl)carbamate (MT-VK-PABC-Compound 101)

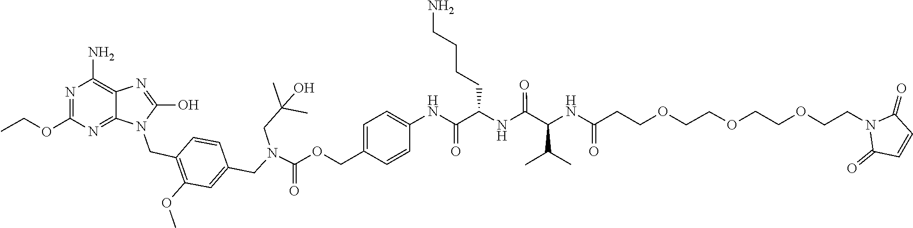 [SMILES: CCOc1nc(N)c2nc(O)n(Cc3ccc(CN(CC(C)(C)O)C(=O)OCc4ccc(NC(=O)[C@H](CCCCN)NC(=O)[C@@H](NC(=O)CCOCCOCCOCCN5C(=O)C=CC5=O)C(C)C)cc4)cc3OC)c2n1]

The titled compound was prepared according to General Procedure 7 from compound 101 (20 mg, 0.049 mmol, 1.0 eq.), compound 62 (48 mg. 0.054 mmol, 1.1 eq.) and HOBt (7 mg, 0.05 mmol, 1 eq.). Preparative HPLC purification of the Boc intermediate was accomplished as described in General Procedure 5, eluting with a 20 to 65% CH₃CN+0.1% TFA/H₂O+0.1% TFA gradient, followed by deprotection as described in General Procedure 2. Final preparative HPLC purification was accomplished as described in General Procedure 5, eluting with a 10 to 55% CH₃CN+0.1% TFA/H₂O+0.1% TFA gradient to give the titled product as a white solid (19.2 mg, 0.015 mmol, 30%). LC-MS: Calc'd m/z=1075.5 for C₅₂H₇₃N₁₁O₁₄. found [M+H]⁺=1076.7. ¹H NMR (300 MHz, MeOD) δ 7.60 (d, J=8.3 Hz, 1H), 7.48 (d, J=8.2 Hz, 2H), 7.37 (d, J=8.2 Hz, 1H), 7.10 (d, J=8.2 Hz, 2H), 6.96 (d, J=8.0 Hz, 1H), 6.83 (s, 2H), 5.09-4.99 (m, 4H), 4.67 (s, 2H), 4.63-4.51 (m, 1H), 4.41-4.28 (m, 3H), 4.18-4.08 (m, 2H), 3.86-3.50 (m, 17H), 2.97 (t, J=7.3 Hz, 2H), 2.58 (t, J=5.9 Hz, 2H), 2.19-2.04 (m, 1H), 2.04-1.90 (m, 1H), 1.93-1.78 (m, 1H), 1.78-1.65 (m, 2H), 1.61-1.55 (m, 2H), 1.31 (t, J=6.9 Hz, 3H), 1.18 (s, 6H), 1.02 (dd, J=6.7, 1.8 Hz, 6H).

### Example 2.42: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl (4-((6-amino-2-butoxy-8-hydroxy-9H-purin-9-yl)methyl)-3-methoxybenzyl)(2-hydroxy-2-methylpropyl)carbamate (MT-VK-PABC-Compound 159)

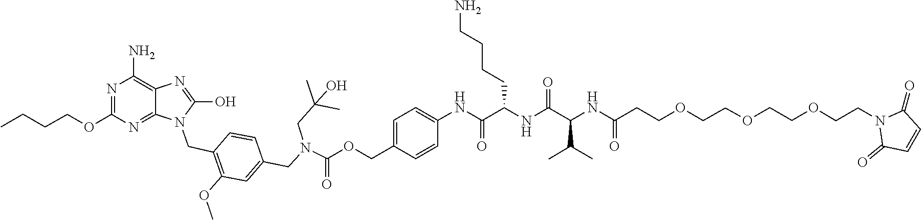 [SMILES: CCCCOc1nc(N)c2nc(O)n(Cc3ccc(CN(CC(C)(C)O)C(=O)OCc4ccc(NC(=O)[C@H](CCCCN)NC(=O)[C@@H](NC(=O)CCOCCOCCOCCN5C(=O)C=CC5=O)C(C)C)cc4)cc3OC)c2n1]

The titled compound was prepared according to General Procedure 7 from compound 159 (5.0 mg, 0.011 mmol, 1.0 eq.), compound 62 (9.0 mg. 0.011 mmol, 1.0 eq.) and HOBt (2 mg, 0.01 mmol, 1 eq.). Preparative HPLC purification of the Boc intermediate was accomplished as described in General Procedure 5, eluting with a 10 to 60% CH₃CN+0.1% TFA/H₂O+0.1% TFA gradient, followed by deprotection as described in General Procedure 2 to give the titled product as a white solid (1.0 mg, 0.00075 mmol, 6.7%). LC-MS: Calc'd m/z=1103.6 for C₅₄H₇₇N₁₁O₁₄. found [M+H]⁺=1104.8. ¹H NMR (300 MHz, MeOD) δ 7.63 (dd, J=20.2, 8.5 Hz, 2H), 7.49-7.31 (m, 2H), 7.10 (d, J=8.1 Hz, 1H), 6.97-6.89 (m, 2H), 6.84 (s, 2H), 5.08 (s, 2H), 5.01 (s, 2H), 4.61-4.55 (m, 2H), 4.24 (d, J=8.8 Hz, 1H), 4.10 (t, J=7.2 Hz, 3H), 3.90-3.49 (m, 17H), 3.02-2.89 (m, 4H), 2.57 (t, J=5.9 Hz, 2H), 1.94-1.50 (m, 7H), 1.46-1.25 (m, 4H), 1.25-1.12 (m, 3H), 1.08-0.84 (m, 12H).

### Example 2.43: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl (4-((6-amino-2-butoxy-8-hydroxy-9H-purin-9-yl)methyl)-3-methoxybenzyl(2S,3S)-1-hydroxy-3-methylpentan-2-yl)carbamate (MT-VK-PABC-Compound 161)

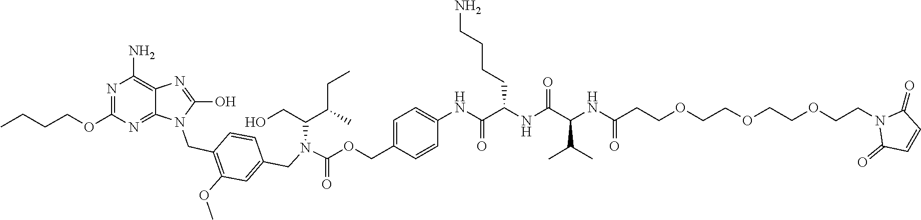

The titled compound was prepared according to General Procedure 7 from compound 161 (7.5 mg, 0.016 mmol, 1.0 eq.), compound 62 (14 mg. 0.016 mmol, 1.0 eq.) and HOBt (2 mg, 0.02 mmol, 1 eq.). Preparative HPLC purification of the Roe intermediate was accomplished as described in General Procedure 5, eluting with a 10 to 60% CH₃CN+0.1% TFA/H₂O++0.1% TFA gradient, followed by deprotection as described in General Procedure 2 to give the titled product as a white solid (1.2 mg, 0.00082 mmol, 5.6%). LC-MS: Calc'd m/z=1131.6 for C₅₆H₈₁N₁₁O₁₄. found [M+H]⁺=1132.8.

### Example 2.44: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl 4-(5-((6-amino-2-butoxy-8-hydroxy-9H-purin-9-yl)methyl)pyridin-2-yl)piperazine-1-carboxylate (MT-VK-PABC-Compound 167)

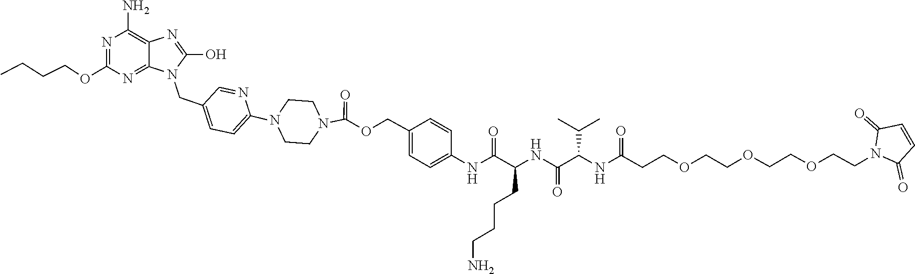 [SMILES: CCCCOc1nc(N)c2nc(O)n(Cc3ccc(N4CCN(C(=O)OCc5ccc(NC(=O)[C@H](CCCCN)NC(=O)[C@@H](NC(=O)CCOCCOCCOCCN6C(=O)C=CC6=O)C(C)C)cc5)CC4)nc3)c2n1]

The titled compound was prepared according to General Procedure 7 from compound 167 (2.8 mg, 0.0070 mmol, 1.0 eq.), compound 62 (6.3 mg. 0.0070 mmol, 1.0 eq.) and HOBt (1 mg, 0.007 mmol, 1 eq.). Preparative HPLC purification of the Boc intermediate was accomplished as described in General Procedure 5, eluting with a 10 to 60% CH₃CN+0.1% TFA/H₂O+0.1% TFA gradient, followed by deprotection as described in General Procedure 2 to give the titled product as a white solid (3.4 mg, 0.0026 mmol, 38%). LC-MS: Calc'd m/z=1057.5 for C₅₁H₇₁N₁₃O₁₂. found [M+H]⁺=1058.7. ¹H NMR (300 MHz, MeOD) δ 8.19-8.05 (m, 2H), 7.71-7.57 (m, 2H), 7.47-7.28 (m, 3H), 6.84 (s, 2H), 5.14 (s, 2H), 4.98 (s, 2H), 4.55 (dd, J=9.1, 4.2 Hz, 1H), 4.36 (t, J=6.5 Hz, 2H), 4.15-4.06 (m, 1H), 3.88-3.51 (m, 22H), 2.97 (t, J=7.3 Hz, 2H), 2.57 (t, J=5.8 Hz, 2H), 2.26-1.42 (m, 11H), 1.06-0.95 (m, 9H).

### Example 2.45: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl (4-((6-amino-8-hydroxy-2-((1-hydroxypentan-2-yl)oxy)-9H-purin-9-yl)methyl)benzyl)carbamate (MT-VK-PABC-Compound 182)

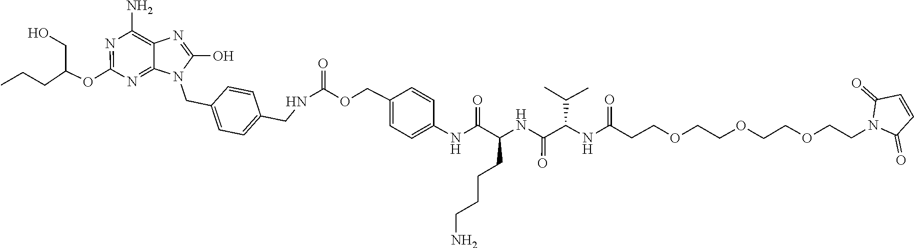 [SMILES: CCCC(CO)Oc1nc(N)c2nc(O)n(Cc3ccc(CNC(=O)OCc4ccc(NC(=O)[C@H](CCCCN)NC(=O)[C@@H](NC(=O)CCOCCOCCOCCN5C(=O)C=CC5=O)C(C)C)cc4)cc3)c2n1]

The titled compound was prepared according to General Procedure 8 from compound 182 (1.5 mg, 0.0040 mmol, 1.0 eq.) and compound 62 (3.6 mg. 0.0040 mmol, 1.0 eq.). Preparative HPLC purification of the Boc intermediate was accomplished as described in General Procedure 5, eluting with a 10 to 60% CH₃CN+0.1% TFA/H₂O+0.1% TFA gradient, followed by deprotection as described in General Procedure 2 to give the titled product as a white solid (2.5 mg, 0.0020 mmol, 50%). LC-MS: Calc'd m/z=1031.5 for C₅₀H₆₉N₁₁O₁₃. found [M+H]⁺=1032.7. ¹H NMR (300 MHz, MeOD) δ 7.57 (d, J=8.2 Hz, 2H), 7.41-7.20 (m, 6H), 6.83 (s, 2H), 5.22-5.12 (m, 1H), 5.05 (s, 2H), 4.99 (s, 2H), 4.57 (s, 1H), 4.27 (s, 2H), 4.11 (t, J=7.2 Hz, 1H), 3.87-3.46 (m, 16H), 2.97 (t, J=7.3 Hz, 2H), 2.57 (t, J=5.9 Hz, 2H), 2.20-1.25 (m, 11H), 1.06-0.88 (m, 9H).

### Example 2.46: 4-((14S,17S)-17-(4-aminobutyl)-1-(2,5-dioxo-2,5-dihydro-1H-pyrrol-1-yl)-14-isopropyl-12,15-dioxo-3,6,9-trioxa-13,16-diazaoctadecan-18-amido)benzyl 4-(4-((6-amino-2-butoxy-8-hydroxy-9H-purin-9-yl)methyl)-3-methoxyphenyl)piperazine-1-carboxylate (MT-VK-PABC-Compound 169)

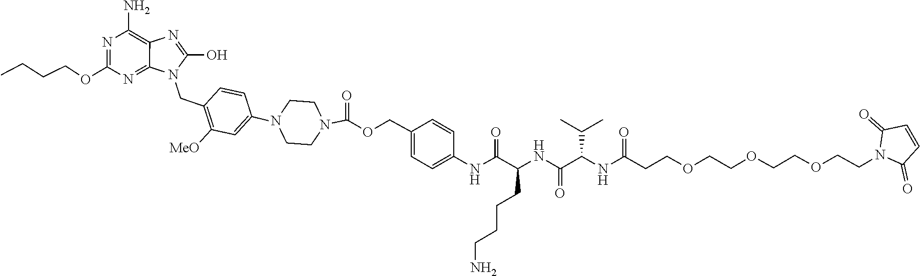 [SMILES: CCCCOc1nc(N)c2nc(O)n(Cc3ccc(N4CCN(C(=O)OCc5ccc(NC(=O)[C@H](CCCCN)NC(=O)[C@@H](NC(=O)CCOCCOCCOCCN6C(=O)C=CC6=O)C(C)C)cc5)CC4)cc3OC)c2n1]

The titled compound was prepared according to General Procedure 8 from compound 169 (7.0 mg, 0.016 mmol, 1.0 eq.) and compound 62 (16 mg. 0.018 mmol, 1.1 eq.). Preparative HPLC purification of the Boc intermediate was accomplished as described in General Procedure 5, eluting with a 10 to 60% CH₃CN+0.1% TFA/H₂O+0.1% TFA gradient, followed by deprotection as described in General Procedure 2. Final preparative HPLC purification was accomplished as described in General Procedure 5, eluting with a 10 to 55% CH₃CN+0.1% TFA/H₂O+0.1% TFA gradient to give the titled product as a white solid (2.3 mg, 0.0017 mmol, 11%). LC-MS: Calc'd m/z=1086.55 for C₅₃H₇₄N₁₂O₁₃. found [M+H]⁺=1087.7. ¹H NMR (300 MHz, MeOD) δ 7.61 (d, J=8.3 Hz, 2H), 7.37 (d, J=8.5 Hz, 2H), 6.98 (d, J=8.4 Hz, 1H), 6.83 (s, 2H), 6.61 (d, J=2.1 Hz, 1H), 6.50 (dd, J=8.4, 2.2 Hz, 1H), 5.13 (s, 2H), 4.96 (s, 2H), 4.63-4.51 (m, 1H), 4.32 (t, J=6.5 Hz, 2H), 4.16-4.05 (m, 1H), 3.91-3.51 (m, 21H), 3.20-3.14 (m, 4H), 2.97 (t, J=7.3 Hz, 2H), 2.57 (t, J=5.9 Hz, 2H), 2.19-1.38 (m, 11H), 1.06-0.91 (m, 9H).

### Example 3: In Vitro Agonism of TLR7

The ability of immunostimulatory compounds of Formulae (I)-(IV) herein to agonize human and mouse TLR7 and initiate downstream immunomodulatory activity, e.g., cytokine production, was assessed by reporter gene assays (RGAs) and cytokine release assays from primary immune cells, respectively.

### Methods

### Reporter Gene Assay

1.5×10⁴ HEK-Blue™ hTLR7 (Invivogen, San Diego, CA) or HEK-Blue™ mTLR7 (Invivogen, San Diego, CA) reporter cells were treated with a titration series of test compound (4-fold titration from 15 pM, 11 points). Compounds were initially diluted to 1 mM in DMSO and subsequently serial-diluted in assay media (HEK-Blue™ Detection; Invivogen, San Diego, CA). Cells with test compound were incubated overnight at 37° C.+5% CO₂, and the amount of secreted embryonic alkaline phosphatase (SEAP) was determined by measuring absorbance (620 nm) using a Synergy™ H1 microplate reader. Dose response curves were plotted on PRISM (GraphPad Software, San Diego, CA) and EC₅₀ values were calculated as the concentration of the compound required to produce 50% maximal effect.

### Human PBMC Assay

Human peripheral blood mononuclear cells (PBMCs) were isolated from fresh Leukopak using magnetic separation. Briefly, erythrocytes and granulocytes were magnetically labeled with Erythrocyte Depletion MicroBeads (Miltenyi Biotec) and Granulocyte Depletion MicroBeads (Miltenyi Biotec), respectively. The cell suspension was loaded onto a Multi-24 Column Block (Miltenyi Biotec) and placed in the magnetic field of a MultiMACS Cell24 Separator Plus (Miltenyi Biotec). The unlabelled PBMCs were collected in the run through. PBMCs were resuspended in CryoStor® CS10 (STEMCELL) and frozen. On the day before the assay, PBMCs were thawed in warm assay buffer (RPMI+10% FBS) and resuspended in assay buffer+10 ng/ml hIL3 (R&D Systems). Cells were rested at 37° C.+5% CO₂ overnight. The following day, cells were spun down and resuspended in fresh assay buffer. 300,000 cells were added to each well of plates containing test compound. For test compound preparation, each compound was initially diluted to 1 mM in DMSO and further diluted in assay buffer. A 4-fold 11-point titration starting from 15 μM was performed. Cells were incubated with test compound at 37° C.+5% CO₂ for 24 hours before supernatant was harvested. Human interleukin 6 (hIL6), human tumor necrosis factor alpha (hTNFα), and human interferon alpha (hIFNα) were quantified in the supernatant using homogeneous time resolved fluorescence (Cisbio). Dose response curves were plotted on PRISM (GraphPad Software, San Diego, CA) and EC₅₀ values were calculated as the concentration of the compound required to produce 50% maximal effect.

### Mouse Splenocyte Assay

Mouse splenocytes were isolated from fresh mouse spleens. Briefly, spleens were harvested from female balb/c nude mice and transferred to a 70 μm cell strainer. The cell strainer was subsequently placed in a 6-well suspension culture plate containing 5 mL media (RPMI+10% FBS). The spleens were crushed against the cell strainer using the flat end of a 10 mL syringe plunger and the cell strainer was washed with 5 mL media. The cell strainer was discarded, and the cell suspension was washed in PBS. Red blood cells were removed using PharmaLyse (BD). The cell suspension was washed in media, resuspended in CryoStor® CS10 (STEMCELL) and frozen. On assay day, mouse splenocytes were thawed in warm assay buffer (RPMI+10% FBS). Cells were spun down and resuspended in fresh assay buffer. 100,000 cells were added to each well of plates containing test compound. For test compound preparation, each compound was initially diluted to 1 mM in DMSO and further diluted in assay buffer. A 4-fold 11-point titration starting from 15 μM was performed. Cells were incubated with test compound at 37° C.+5% CO₂ for 24 hours before supernatant was harvested. Murine IL6 (mIL6) and murine TNFα (mTNFα) were quantified in the supernatant using homogeneous time resolved fluorescence (Cisbio). Dose response curves were plotted on PRISM (GraphPad Software, San Diego, CA) and EC₅₀ values were calculated as the concentration of the compound required to produce 50% maximal effect.

### Controls and Analyses

Each assay included a positive control compound (referred to herein as “benchmark,” see, e.g., TABLE 3 below, and taken from International Patent Publication No. WO2017/072662, structure shown below) and a negative control (growth medium). Dose response curves were plotted on PRISM (GraphPad Software, San Diego, CA) and EC₅₀ values were calculated as the concentration of the compound required to produce 50% maximal effect (IL6 induction or NF-κB activation measured by SEAP for PBMC and HEK Blue hTLR7 assays, respectively). The structure of the benchmark compound from International Patent Publication No. WO 2017/072662 is as follows:

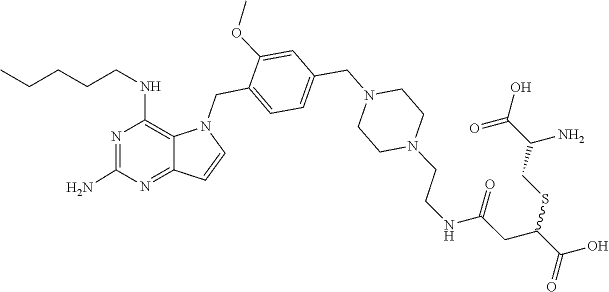

### Results

The results of the Reporter Gene Assay (RGA, for human (h) and mouse (in) TLR7) experiments as well as the compounds' ability to induce production of cytokines in PBMCs and mouse splenocytes are shown and summarized below in TABLE 3.

TABLE 3
In Vitro Immunomodulatory Properties of Tested Compounds

| Compound | hTLR7 RGA | mTLR7 RGA | PBMC IL6 | PBMC IFNa | Splenocyte |
|---|---|---|---|---|---|
| # | EC₅₀ (nM) | EC₅₀ (nM) | EC₅₀ (nM) | EC₅₀¹ (nM) | IL6 EC₅₀ (nM) |
| Benchmark | 27 | 95.4 | 206.8 | 10 − 1 | 222 |
| 100 | 6.3 | 77.9 | 239.9 | 10 − 1 | 2942 |
| 101 | 57 | 47.7 | 127 | 1 − 0.1 | 153.5 |
| 102 | 2 | 5 | 69.1 | <0.1 | 99.7 |
| 103 | 138 | 27.4 | 9268 | 1 − 0.1 | 1333 |
| 104 | 1308 | 1035 | 598 | − | 1362 |
| 105 | 7 | 2.4 | 393.8 | <0.1 | 26.2 |
| 106 | 348 | 94.1 | 688.1 | 10 − 1 | 2059 |
| 107 | 13 | 0.3 | 2718 | 1 − 0.1 | 33.3 |
| 108 | 6 | 5.0 | 563.9 | <0.1 | 71.9 |
| 109 | 56 | 13.1 | 456.1 | 10 − 1 | 166.5 |
| 110 | 112 | 184.1 | 937.1 | 10 − 1 | ≥10³ |
| 111 | 102 | 86.9 | 530.9 | 10 − 1 | 20.8 |
| 112 | 122 | 93.9 | 232.9 | 10 − 1 | 242.9 |
| 113 | 187.4 | 662.5 | 1359 | − | 1175 |
| 114 | 261.4 | 1523 | 1754 | − | 900.7 |
| 115 | 277.1 | 1596 | 2810 | − | 3236 |
| 116 | 184.5 | 1024 | 3082 | − | 2178 |
| 117 | 12972 | − | inactive | − | inactive |
| 118 | 2.04 | 11.4 | 1398 | 1 − 0.1 | 638.7 |
| 119 | 259 | 1963.0 | 3913 | − | ≥10³ |
| 120 | 33.2 | 636.7 | 524.5 | − | 881.4 |
| 121 | 342.7 | 771.6 | 13338 | − | ≥10³ |
| 122 | 9 | 10.2 | 279.1 | 1 − 0.1 | 66.1 |
| 123 | 30.7 | 146.1 | 683.9 | 10 − 1 | 145.4 |
| 124 | 5.4 | 33.5 | 674.4 | 1 − 0.1 | 743.5 |
| 125 | 0.5 | 4.3 | 2229 | 10 − 1 | 170.6 |
| 126 | 33 | 659.3 | − | − | − |
| 127 | 175 | 1033 | − | − | − |
| 128 | 18.2 | 443.1 | 167.1 | 10 − 1 | inactive |
| 129 | 81.1 | 541.2 | − | − | − |
| 130 | 201.8 | 1267 | − | − | − |
| 131 | 86.1 | 675.9 | − | − | − |
| 132 | 5.8 | 65.1 | 116.4 | − | 233.7 |
| 133 | 177.9 | 858.9 | − | − | − |
| 134 | 363.5 | 1647 | − | − | − |
| 135 | 13.3 | 156.9 | 42.3 | 1 − 0.1 | 136.6 |
| 136 | 223.2 | 1710 | − | − | − |
| 137 | 22.2 | 210.2 | 39.4 | 1 − 0.1 | 230.5 |
| 138 | 17.2 | 242.7 | 129.7 | − | 186.4 |
| 139 | 6.5 | 96.5 | 115.1 | − | 170.4 |
| 140 | 6.8 | 34.8 | 53.6 | − | 179.5 |
| 141 | 0.1 | 1 | 9294 | <0.1 | 1.3 |
| 142 | 2.6 | 14.6 | 13.6 | <0.1 | 9.9 |
| 143 | 0.2 | 15.1 | 27.1 | <0.1 | 9.6 |
| 144 | 79.5 | 1326 | 190.8 | − | 372.4 |
| 145 | 69.0 | 142.2 | 125.8 | + | 337.5 |
| 146 | 5.8 | 28.7 | 16.7 | <0.1 | 12.9 |
| 147 | 0.9 | 16.9 | 38.8 | <0.1 | 13.2 |
| 148 | 167 | 1261 | − | − | − |
| 149 | 520 | 1260 | − | − | − |
| 150 | 6.9 | 83.8 | 322.4 | 1 − 0.1 | 2133 |
| 151 | 12.1 | 125.2 | 59.2 | − | 68.8 |
| 152 | 1.3 | 31.7 | 17 | 1 − 0.1 | inactive |
| 153 | 6.1 | 262.8 | 39 | − | inactive |
| 154 | 0.2 | 2.9 | 1972 | − | 216.6 |
| 155 | 0.2 | 9.2 | 877.6 | − | 148.2 |
| 156 | 0.3 | 0.3 | − | − | 2.5 |
| 157 | 0.9 | 3.7 | 472.8 | − | 9.4 |
| 158 | 0.1 | 0.1 | − | 10 − 1 | 3.2 |
| 159 | 1.6 | 2.9 | − | <0.1 | 2 |
| 160 | 7.2 | 10 | 1076 | <0.1 | 6.6 |
| 161 | <0.1 | <0.1 | 16.7 | <0.1 | 0.3 |
| 162 | 1.7 | 2.3 | − | 10 − 1 | 1.3 |
| 163 | 0.7 | 3.3 | − | <0.1 | 2.9 |
| 164 | <0.1 | <0.1 | 2535 | <0.1 | 0.3 |
| 165 | <0.1 | <0.1 | 6987 | <0.1 | − |
| 166 | <0.1 | 11 | 1570 | − | 150.2 |
| 167 | 0.5 | 5.3 | 2030 | − | 18 |
| 168 | 0.1 | 5.7 | inactive | − | 35.7 |
| 169 | 1.1 | 3 | − | 10 − 1 | 23.2 |
| 170 | 1 | 2 | 1466 | − | 51.7 |
| 171 | <0.1 | 1.1 | 1553 | − | 8.9 |
| 172 | <0.1 | <0.1 | − | − | 2.1 |
| 173 | 0.2 | 0.6 | 1799 | 10 − 1 | 5 |
| 174 | 0.2 | <0.1 | 917.9 | − | 5.4 |
| 175 | <0.1 | <0.1 | inactive | − | 6 |
| 176 | <0.1 | <0.1 | inactive | − | 5.8 |
| 177 | 411 | 15454 | − | − | − |
| 178 | 144 | − | 284.5 | 10 − 1 | 245 |
| 179 | 181 | 421.5 | − | − | − |
| 180 | 34 | 79.9 | inactive | − | 872.8 |
| 181 | 4.5 | − | inactive | − | 234.6 |
| 182 | 2.1 | 5.9 | 5222 | − | 2.4 |
| 183 | 145 | 60.6 | 291.4 | − | 235 |
| 184 | 278 | 20.4 | 29.2 | 1 − 0.1 | 144.2 |
| 185 | 10 | 1 | 12.6 | 10 − 1 | 20.4 |
| 186 | 452 | 11.1 | inactive | − | inactive |
| 187 | 94 | 9 | 9 | 1 − 0.1 | 130.8 |
| 188 | 351 | 26.2 | 19.6 | 10 − 1 | 198.2 |
| 189 | 698 | 41 | 151 | 10 − 1 | 80.6 |
| 190 | 1417 | 80.8 | 131.2 | 10 − 1 | 106 |
| 191 | 29 | 0.5 | − | <0.1 | 45.9 |
| 192 | 51.42 | 680.9 | 202.2 | − | inactive |
| 193 | 60 | 5.9 | 109.2 | 1 − 0.1 | 48.23 |
| 194 | 311 | 35.9 | 1095 | 10 − 1 | 1394 |
| 195 | 1748 | 127.4 | 790.6 | 100 − 10 | 1005 |

¹For PBMC IFNα, EC₅₀ values were approximated and divided into the following groups: (i) “−” = >10 nM, (ii) 10 − 1 nM, (iii) 1 − 0.1 nM, or (iv) <0.1 nM.

The results demonstrate that the majority of the tested immunostimulatory compounds have TLR7 agonist activity and/or are capable of inducing cytokine release in these assays. Notably, many of the compounds comprising a substituted (e.g., with methoxy, fluorine, etc.) aryl moiety attached to the N⁹ atom of the purine moiety, showed double or single digit nanomolar EC₅₀ values for targeting human and/or mouse TLR7, and for inducing cytokine production in human PBMCs and/or mouse splenocytes.

### Example 4: Preparation of Immunostimulatory Antibody-Drug Conjugates (ISACS) Comprising a Compound of the Present Disclosure

This example describes the preparation of immunostimulatory antibody conjugates comprising an immunostimulatory compound of the present disclosure.

### Example 4.1: Trastuzumab-MT-VC-PABC-Compound 105

This conjugate was obtained following the General Procedure 9, using Trastuzumab as the antibody reduced with 2.2 equivalents of TCEP. The conjugation was performed in the presence of 30 percent v/v propylene glycol with 17.4 equivalents of compound-linker construct (MT-VC-PABC-Compound 105) with an incubation time of 180 min. The average DAR and drug distribution was derived from interpretation of HPLC-HIC data and is shown in TABLE 4 herein.

### Example 4.2: Trastuzumab-ME2-Compound 100

This conjugate was obtained following the General Procedure 9, using Trastuzumab as the antibody reduced with 2.15 equivalents of TCEP. The conjugation was performed with 20 equivalents of compound-linker construct (ME2-Compound 100) with an incubation time of 180 min. The average DAR and drug distribution was derived from interpretation of LC-MS data and is shown in TABLE 4 herein.

### Example 4.3: Trastuzumab-MT-VK-PABC-Compound 107

This conjugate was obtained following the General Procedure 9, using Trastuzumab as the antibody reduced with 2.15 equivalents of TCEP. The conjugation was performed in the presence of 30 percent v/v propylene glycol with 20 equivalents of compound-linker construct (MT-VK-PABC-Compound 107) with an incubation time of 180 min. The average DAR and drug distribution was derived from interpretation of HPLC-HIC data and is shown in TABLE 4 herein.

### Example 4.4: Trastuzumab-MT-VK-PABC-Compound 108

This conjugate was obtained following the General Procedure 9, using Trastuzumab as the antibody reduced with 2.15 equivalents of TCEP. The conjugation was performed with 20 equivalents of compound-linker construct (MT-VK-PABC-Compound 108) with an incubation time of 180 min. The average DAR and drug distribution was derived from interpretation of HPLC-HIC data and is shown in TABLE 4 herein.

### Example 4.5: Trastuzumab-MT-VK-PABC-Compound 101

This conjugate was obtained following the General Procedure 9, using Trastuzumab as the antibody reduced with 2.15 equivalents of TCEP. The conjugation was performed with 20 equivalents of compound-linker construct (MT-VK-PABC-Compound 101) with an incubation time of 180 min. The average DAR and drug distribution was derived from interpretation of HPLC-HIC data and is shown in TABLE 4 herein.

### Example 4.6: Trastuzumab-MT-VC-PABC-Compound 112

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 112) with an incubation time of 180 min. The average DAR and drug distribution was derived from interpretation of HPLC-HIC data and is shown in TABLE 4 herein.

### Example 4.7: Trastuzumab-MT-VC-PABC-Compound 106

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 106) with an incubation time of 180 min. The average DAR and drug distribution was derived from interpretation of HPLC-HIC data and is shown in TABLE 4 herein.

### Example 4.8: Trastuzumab-MT-VC-PABC-Compound 111

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 111) with an incubation time of 180 min. The average DAR and drug distribution was derived from interpretation of HPLC-HIC data and is shown in TABLE 4 herein.

### Example 4.9: Trastuzumab-MT-VC-PABC-Compound 144

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 144) with an incubation time of 180 min. The average DAR and drug distribution was derived from interpretation of HPLC-HIC data and is shown in TABLE 4 herein.

### Example 4.10: Trastuzumab-MT-VC-PABC-Compound 187

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 187) with an incubation time of 180 min. The average DAR and drug distribution was derived from interpretation of HPLC-HIC data and is shown in TABLE 4 herein.

### Example 4.11: Trastuzumab-MT-VC-PABC-Compound 188

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 188) with an incubation time of 180 min. The average DAR and drug distribution was derived from interpretation of HPLC-HIC data and is shown in TABLE 4 herein.

### Example 4.12: Trastuzumab-MT-VC-PABC-Compound 150

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 150) with an incubation time of 180 min. The average DAR and drug distribution was derived from interpretation of HPLC-HIC data and is shown in TABLE 4 herein.

### Example 4.13: Trastuzumab-MT-VC-PABC-Compound 145

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 145) with an incubation time of 180 min. The average DAR and drug distribution was derived from interpretation of HPLC-HIC data and is shown in TABLE 4 herein.

### Example 4.14: Trastuzumab-MT-VC-PABC-Compound 152

This conjugate was obtained following the General Procedure 9, using Trastuzumab as the antibody reduced with 2.2 equivalents of TCEP. The conjugation was performed with 20 equivalents of compound-linker construct (MT-VC-PABC-Compound 152) with an incubation time of 180 min. The average DAR and drug distribution was derived from interpretation of HPLC-HIC data and is shown in TABLE 4 herein.

### Example 4.15: Trastuzumab-MT-VC-PABC-Compound 154

This conjugate was obtained following the General Procedure 9, using Trastuzumab as the antibody reduced with 2.2 equivalents of TCEP. The conjugation was performed with 20 equivalents of compound-linker construct (MT-VC-PABC-Compound 154) with an incubation time of 180 min. The average DAR and drug distribution was derived from interpretation of HPLC-HIC data and is shown in TABLE 4 herein.

### Example 4.16: Trastuzumab-MT-VC-PABC-Compound 166

This conjugate was obtained following the General Procedure 9, using Trastuzumab as the antibody reduced with 2.2 equivalents of TCEP. The conjugation was performed with 20 equivalents of compound-linker construct (MT-VC-PABC-Compound 166) with an incubation time of 180 min. The average DAR and drug distribution was derived from interpretation of HPLC-HIC data and is shown in TABLE 4 herein.

### Example 4.17: Trastuzumab-MT-VC-PABC-Compound 155

This conjugate was obtained following the General Procedure 9, using Trastuzumab as the antibody reduced with 2.2 equivalents of TCEP. The conjugation was performed with 20 equivalents of compound-linker construct (MT-VC-PABC-Compound 155) with an incubation time of 180 min. The average DAR and drug distribution was derived from interpretation of HPLC-HIC data and is shown in TABLE 4 herein.

### Example 4.18: Trastuzumab-MT-VC-PABC-Compound 140

This conjugate was obtained following the General Procedure 9, using Trastuzumab as the antibody reduced with 2.2 equivalents of TCEP. The conjugation was performed with 20 equivalents of compound-linker construct (MT-VC-PABC-Compound 140) with an incubation time of 180 min. The average DAR and drug distribution was derived from interpretation of HPLC-HIC data and is shown in TABLE 4 herein.

### Example 4.19: Trastuzumab-MT-VC-PABC-Compound 139

This conjugate was obtained following the General Procedure 9, using Trastuzumab as the antibody reduced with 2.2 equivalents of TCEP. The conjugation was performed with 20 equivalents of compound-linker construct (MT-VC-PABC-Compound 139) with an incubation time of 180 min. The average DAR and drug distribution was derived from interpretation of HPLC-HIC data and is shown in TABLE 4 herein.

### Example 4.20: Trastuzumab-MT-VC-PABC-Compound 110

This conjugate was obtained following the General Procedure 9, using Trastuzumab as the antibody reduced with 2.2 equivalents of TCEP. The conjugation was performed with 20 equivalents of compound-linker construct (MT-VC-PABC-Compound 110) with an incubation time of 180 min. The average DAR and drug distribution was derived from interpretation of HPLC-HIC data and is shown in TABLE 4 herein.

### Example 4.21: Trastuzumab-MT-VC-PABC-Compound 172

This conjugate was obtained following the General Procedure 9, using Trastuzumab as the antibody reduced with 2.2 equivalents of TCEP. The conjugation was performed in the presence of 10 percent v/v propylene glycol with 20 equivalents of compound-linker construct (MT-VC-PABC-Compound 172) with an incubation time of 180 min. The average DAR and drug distribution was derived from interpretation of HPLC-HIC data and is shown in TABLE 4 herein.

### Example 4.22: Trastuzumab-MT-VK-PABC-Compound 161

This conjugate was obtained following the General Procedure 9, using Trastuzumab as the antibody reduced with 2.2 equivalents of TCEP. The conjugation was performed in the presence of 10 percent v/v propylene glycol with 20 equivalents of compound-linker construct (MT-VK-PABC-Compound 161) with an incubation time of 180 min. The average DAR and drug distribution was derived from interpretation of HPLC-HIC data and is shown in TABLE 4 herein.

### Example 4.23: Trastuzumab-MT-VK-PABC-Compound 182

This conjugate was obtained following the General Procedure 9, using Trastuzumab as the antibody reduced with 2.2 equivalents of TCEP. The conjugation was performed in the presence of 10 percent v/v propylene glycol with 20 equivalents of compound-linker construct (MT-VK-PABC-Compound 182) with an incubation time of 180 min. The average DAR and drug distribution was derived from interpretation of HPLC-HIC data and is shown in TABLE 4 herein.

### Example 4.24: Trastuzumab-MT-VK-PABC-Compound 159 (DAR 2)

This conjugate was obtained following the General Procedure 9, using Trastuzumab as the antibody reduced with 1.1 equivalents of TCEP. The conjugation was performed in the presence of 10 percent v/v propylene glycol with 20 equivalents of compound-linker construct (MT-VK-PABC-Compound 159) with an incubation time of 180 min. The average DAR and drug distribution was derived from interpretation of HPLC-HIC data and is shown in TABLE 4 herein.

### Example 4.25: Trastuzumab-MT-VK-PABC-Compound 167 (DAR 2)

This conjugate was obtained following the General Procedure 9, using Trastuzumab as the antibody reduced with 1.1 equivalents of TCEP. The conjugation was performed in the presence of 10 percent v/v propylene glycol with 20 equivalents of compound-linker construct (MT-VK-PABC-Compound 167) with an incubation time of 180 min. The average DAR and drug distribution was derived from interpretation of HPLC-HIC data and is shown in TABLE 4 herein.

### Example 4.26: Trastuzumab-MT-VC-PABC-Compound 180 (DAR 2)

This conjugate was obtained following the General Procedure 9, using Trastuzumab as the antibody reduced with 1.1 equivalents of TCEP. The conjugation was performed in the presence of 10 percent v/v propylene glycol with 20 equivalents of compound-linker construct (MT-VC-PABC-Compound 180) with an incubation time of 180 min. The average DAR and drug distribution was derived from interpretation of HPLC-HIC data and is shown in TABLE 4 herein.

### Example 4.27: Trastuzumab-MT-VK-PABC-Compound 169 (DAR 2)

This conjugate was obtained following the General Procedure 9, using Trastuzumab as the antibody reduced with 1.1 equivalents of TCEP. The conjugation was performed in the presence of 10 percent v/v propylene glycol with 20 equivalents of compound-linker construct (MT-VK-PABC-Compound 169) with an incubation time of 180 min. The average DAR and drug distribution was derived from interpretation of HPLC-HIC data and is shown in TABLE 4 herein.

### Example 4.28: Trastuzumab-MT-VC-PABC-Compound 141

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 141) with an incubation time of 120 min. The average DAR and drug distribution was derived from interpretation of HPLC-HIC data and is shown in TABLE 4 herein.

### Example 4.29: Trastuzumab-MT-VC-PABC-Compound 142

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 142) with an incubation time of 120 min. The average DAR and drug distribution was derived from interpretation of HPLC-HIC data and is shown in TABLE 4 herein.

### Example 4.30: Trastuzumab-MT-VC-PABC-Compound 143

This conjugate was obtained following the General Procedure 9, using Trastuzumab as the antibody reduced with 2.15 equivalents of TCEP. The conjugation was performed with 20 equivalents of compound-linker construct (MT-VC-PABC-Compound 143) with an incubation time of 120 min. The average DAR and drug distribution was derived from interpretation of HPLC-HIC data and is shown in TABLE 4 herein.

### Example 4.31: Trastuzumab-MT-VC-PABC-Compound 191

This conjugate was obtained following the General Procedure 9, using Trastuzumab as the antibody reduced with 2.2 equivalents of TCEP. The conjugation was performed with 15 equivalents of compound-linker construct (MT-VC-PABC-Compound 191) with an incubation time of 120 min. The average DAR and drug distribution was derived from interpretation of HPLC-HIC data and is shown in TABLE 4 herein.

### Example 4.32: Trastuzumab-MT-VC-PABC-Compound 132

This conjugate was obtained following the General Procedure 9, using Trastuzumab as the antibody reduced with 2.2 equivalents of TCEP. The conjugation was performed with 20 equivalents of compound-linker construct (MT-VC-PABC-Compound 132) with an incubation time of 180 min. The average DAR and drug distribution was derived from interpretation of HPLC-HIC data and is shown in TABLE 4 herein.

### Example 4.33: Trastuzumab-MT-VC-PABC-Compound 173 (DAR 2)

This conjugate was obtained following the General Procedure 9, using Trastuzumab as the antibody reduced with 1.1 equivalents of TCEP. The conjugation was performed in the presence of 10 percent v/v % propylene glycol with 20 equivalents of compound-linker construct (MT-VC-PABC-Compound 173) with an incubation time of 180 min. The average DAR and drug distribution was derived from interpretation of HPLC-HIC data and is shown in TABLE 4 herein.

### Example 4.34: Trastuzumab-MT-VC-PABC-Compound 100

This conjugate was obtained following the General Procedure 9, using Trastuzumab as the antibody reduced with 2.2 equivalents of TCEP. The conjugation was performed with 15 equivalents of compound-linker construct (MT-VC-PABC-Compound 100) with an incubation time of 130 min. The average DAR and drug distribution was derived from interpretation of HPLC-HIC data and is shown in TABLE 4 herein.

### Example 4.35: Trastuzumab-MT-VK-PABC-Compound 100

This conjugate was obtained following the General Procedure 9, using Trastuzumab as the antibody reduced with 2.15 equivalents of TCEP. The conjugation was performed with 15 equivalents of compound-linker construct (MT-VK-PABC-Compound 100) with an incubation time of 150 min. The average DAR and drug distribution was derived from interpretation of HPLC-HIC data and is shown in TABLE 4 herein.

### Example 4.36: Trastuzumab-MC-Compound 100

This conjugate was obtained following the General Procedure 9, using Trastuzumab as the antibody reduced with 2.2 equivalents of TCEP. The conjugation was performed with 19.4 equivalents of compound-linker construct (MC-Compound 100) with an incubation time of 180 min. The average DAR and drug distribution was derived from interpretation of HPLC-HIC data and is shown in TABLE 4 herein.

### Example 4.37: Trastuzumab-MT-Compound 100

This conjugate was obtained following the General Procedure 9, using Trastuzumab as the antibody reduced with 2.2 equivalents of TCEP. The conjugation was performed with 19.4 equivalents of compound-linker construct (MT-Compound 100) with an incubation time of 180 min. The average DAR and drug distribution was derived from interpretation of HPLC-HIC data and is shown in TABLE 4 herein.

TABLE 4
Determined DAR values for Produced and Tested ISACs

| Example # | Name | DAR |
|---|---|---|
| 4.1 | Tras-MT-VC-PABC-Compound 105 | 4.1 |
| 4.2 | Tras-ME2-Compound 100 | 3.6 |
| 4.3 | Tras-MT-VK-PABC-Compound 107 | 3.8 |
| 4.4 | Tras-MT-VK-PABC-Compound 108 | 3.9 |
| 4.5 | Tras-MT-VK-PABC-Compound 101 | 3.5 |
| 4.6 | Tras-MT-VC-PABC-Compound 112 | 3.9 |
| 4.7 | Tras-MT-VC-PABC-Compound 106 | 3.7 |
| 4.8 | Tras-MT-VC-PABC-Compound 111 | 3.6 |
| 4.9 | Tras-MT-VC-PABC-Compound 144 | 3.3 |
| 4.10 | Tras-MT-VC-PABC-Compound 187 | 3.5 |
| 4.11 | Tras-MT-VC-PABC-Compound 188 | 3.5 |
| 4.12 | Tras-MT-VC-PABC-Compound 150 | 3.5 |
| 4.13 | Tras-MT-VC-PABC-Compound 145 | 3.5 |
| 4.14 | Tras-MT-VC-PABC-Compound 152 | 4.0 |
| 4.15 | Tras-MT-VC-PABC-Compound 154 | 3.1 |
| 4.16 | Tras-MT-VC-PABC-Compound 166 | 4.0 |
| 4.17 | Tras-MT-VC-PABC-Compound 155 | 4.2 |
| 4.18 | Tras-MT-VC-PABC-Compound 140 | 3.9 |
| 4.19 | Tras-MT-VC-PABC-Compound 139 | 3.9 |
| 4.20 | Tras-MT-VC-PABC-Compound 110 | 3.9 |
| 4.21 | Tras-MT-VC-PABC-Compound 172 | 4.1 |
| 4.22 | Tras-MT-VK-PABC-Compound 161 | 4.3 |
| 4.23 | Tras-MT-VK-PABC-Compound 182 | 4.4 |
| 4.24 | Tras-MT-VK-PABC-Compound 159 | 1.9 |
| 4.25 | Tras-MT-VK-PABC-Compound 167 | 2.2 |
| 4.26 | Tras-MT-VC-PABC-Compound 180 | 2.0 |
| 4.27 | Tras-MT-VC-PABC-Compound 169 | 2.1 |
| 4.28 | Tras-MT-VC-PABC-Compound 141 | 4.1 |
| 4.29 | Tras-MT-VC-PABC-Compound 142 | 4.3 |
| 4.30 | Tras-MT-VC-PABC-Compound 143 | 4.2 |
| 4.31 | Tras-MT-VC-PABC-Compound 191 | 3.8 |
| 4.32 | Tras-MT-VC-PABC-Compound 132 | 4.0 |
| 4.33 | Tras-MT-VC-PABC-Compound 173 | 1.9 |
| 4.34 | Tras-MT-VC-PABC-Compound 100 | 4.0 |
| 4.35 | Tras-MT-VK-PABC-Compound 100 | 3.7 |
| 4.36 | Tras-MC-Compound 100 | 3.9 |
| 4.37 | Tras-MT-Compound 100 | 3.9 |

### Methods

### Determination of Drug-Antibody Ratio (DAR) by RP-UPLC-MS:

10 μL of conjugated ISAC samples at 1 mg/ml were deglycosylated using EndoS (1 μg) after a 1 h incubation at room temperature. 2 μL of 500 mM TCEP (in water) was added to 11 μl sample and incubated at 70° C. for 30 min for reduction and denaturation. Samples were then run on an LC-MS-QTOF using a 1 μl injection each, following the analytical method described below:
Column: PLRP-S 1000A 8 μm 50×2.1 mm
Column Temperature: 70° C.
Flow Rate: 0.3 mL/min
Mobile Phase C: 0.1% Formic Acid (FA) 0.025% Trifluoroacetic Acid (TFA) and 10% Isopropyl Alcohol (IPA) in water
Mobile Phase D: 0.1% FA and 10% IPA in acetonitrile (ACN)
Detection: Signal A (280 nm, 4.0 bandwidth), Signal B (220 nm, 4.0 bandwidth)
Gradient (post run time: 2 mins, percentages in v/v):

| Time | C (%) | D (%) |
|---|---|---|
| 0 | 80 | 20 |
| 20 | 60 | 40 |
| 22 | 10 | 90 |
| 22.5 | 1 | 99 |
| 24 | 1 | 99 |

MS data analysis was performed as described below. For cysteine conjugates, integrated TIC at two regions (Light Chain (LC) region between 4 to 8 mins, Heavy Chain (HC) region between 8 to 13 mins). Deconvolution Parameters:
Deconvolution Algorithm: Maximum Entropy
Mass Range: 20000-60000
Mass Step: 1.0
Used limited m/z range: 1000-7000
Subtract baseline: 7.0
Adduct: Proton
Isotope width: Automatic
Height Filter: Peak signal to noise >=30.0
Maximum number of peaks: Limited by height 100
Calculate average mass using top 90% of peak height
Average DAR was calculated by the peak height for each DAR species

### Determination of DAR by Hydrophobic Interaction Chromatography (HIC):

The average degree of conjugation of compound-linker construct to antibody (DAR) was assessed by hydrophobic interaction chromatography (HIC). These techniques are described in Antibody Drug Conjugates, Methods in Molecular Biology, 2013, vol. 1045, pp. 275-284. L. Ducry, Ed. Typically, the measured DAR values ranged from 3.6 to 4.2. Briefly, ISACs were subjected to HIC on a TSKgel® Butyl-NPR column (Tosoh Bioscience; 4.6 mm×3 mm i.d.; 2.5-micron particle size) connected to an Agilent 1100 series HPLC. Samples were injected (5 μL) at or above 2 mg/mL. A linear gradient elution was employed starting at 95% mobile phase A/5% mobile phase B, transitioning to 5% mobile phase A/95% mobile phase B over a period of 12 min (mobile phase A: 1.5 M ammonium sulfate+25 mM sodium phosphate, pH 6.95; mobile phase B: 25% isopropanol, 75% 25 mM sodium phosphate, pH 6.95). Injection of unmodified antibody provided a means of identifying the peak with DAR=0. Antibodies were detected on the basis of absorbance at 280 nm.

### General Procedure for Size Exclusion Chromatography (SEC) Analysis of ISACs:

Samples were run on an Agilent 1290 UHPLC system equipped with a quaternary pump and a DAD detector, using a 5 μl injection volume for each sample following the analytical method described below:
Column: Agilent AdvanceBio SEC, 300 Å, 2.7 μm, 7.8×150 mm
Column Temperature: 25° C.
Flow Rate: 1.0 mL/min
Mobile Phase: 150 mM NaₓPO₄ pH 6.95
Detection: Absorbance at 280 nm, 4.0 bandwidth
Isocratic run time: 7 min

### Example 5: In Vitro Activity of ISACS

This example demonstrates the ability of the tested ISACs produced as described in EXAMPLE 4 to induce the production of human IL6 (hIL6) from PBMCs and/or murine IL6 (mIL6) from mouse splenocytes, and/or to agonize TLR7 as determined by secreted embryonic alkaline phosphatase (SEAP) from RAW-Blue™ cells as part of a reporter gene assay.

### Methods

### Assess In Vitro Activity of Antibody-Drug Conjugates

Human PBMCs and mouse splenocytes were isolated as described above in EXAMPLE 3. On assay day, human PBMCs or mouse splenocytes were thawed in warm assay media (RPMI+10% FBS). Cells were spun down and resuspended in fresh assay buffer. 100,000 cells were added to each well of plates containing an ISAC. For ISAC preparation, a 5-fold 7-point titration from 100 nM in assay media was performed. For tumor cell preparation, NCI_N87 tumor cells were propagated in tissue culture plates until they reach 90% confluency and detached from tissue culture plates with TrypLE™ Express Enzyme (ThermoFisher). 10,000 tumor cells were added to wells containing antibody drug conjugate with human PBMCs or with mouse splenocytes. The co-culture was incubated with ISAC at 37° C.+5% CO₂ for 24 hours before supernatant was harvested. hIL6 and mIL6 were quantified from the human PBMC+NCI_N87 co-culture supernatant and mouse splenocyte+NCI_N87 co-culture supernatant, respectively, using homogeneous time resolved fluorescence (Cisbio). Dose response curves were plotted on PRISM (GraphPad Software, San Diego, CA) and EC₅₀ values were calculated as the concentration of the compound required to produce 50% maximal effect.

The in vitro activity of ISACs was also assessed in a reporter gene assay using mouse RAW-Blue™ cells (Invivogen, San Diego, CA). Briefly, RAW-Blue™ cells were maintained in growth media recommended by the vendor until assay day. On assay day, RAW-Blue™ cells were scraped from tissue culture flasks and resuspended in assay media (DMEM+10% FBS). 100,000 RAW-Blue™ cells were added to each well of plates containing an ISAC. For ISAC preparation, a 5-fold 7-point titration from 100 nM in assay media was performed. For tumor cell preparation, NCI_N87 tumor cells were propagated in tissue culture plates until they reach 90% confluency and detached from tissue culture plates with TrypLE™ Express Enzyme (ThermoFisher). 100,000 tumor cells were added to wells containing ISAC with RAW-Blue™ cells. The co-culture was incubated with ISAC at 37° C.+5% CO₂ for 48 hours before 10 μl supernatant was harvested. To the supernatant, 90 ul QUANTI-Blue™ Solution (Invivogen, San Diego, CA) was added and the amount of secreted embryonic alkaline phosphatase (SEAP) was determined by measuring absorbance (620 nm) using a Synergy™ H1 microplate reader. Dose response curves were plotted on PRISM (GraphPad Software, San Diego, CA) and EC₅₀ values were calculated as the concentration of the compound required to produce 50% maximal effect.

Trastuzumab coupled to the benchmark compound (e.g., without the cysteine-capped hemisuccinimide moiety, which can be a result of in vivo metabolism and constitute an active metabolite upon cleavage from the targeting moiety) was used as a positive control in this study. The following “activated” version of the benchmark was used for coupling to Trastuzumab:

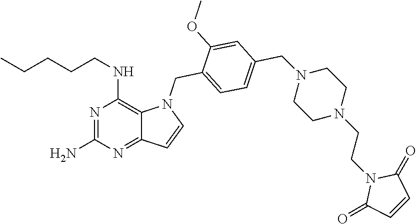 [SMILES: CCCCCNc1nc(N)nc2ccn(Cc3ccc(CN4CCN(CCN5C(=O)C=CC5=O)CC4)cc3OC)c12]

### Results

The EC₅₀ values that were determined for tested ISACs to induce the production of (i) hIL6 from PBMCs, (ii) mIL6 from mouse splenocytes, and (iii) secreted embryonic alkaline phosphatase (SEAP) from RAW-Blue™ cells is shown in TABLE 5 (Trastuzumab is abbreviated with “Tras”).

TABLE 5
In Vitro Activities of ISACs

| | hIL6 EC₅₀ | mIL6 EC₅₀ | RAW-Blue ™ |
|---|---|---|---|
| ISAC | (pM) | (pM) | EC₅₀ (pM) |
| Tras-Benchmark | 171.7 | 99.7 | 573.9 |
| Tras-MT-VC-PABC-Compound 100 | 160.4 | inactive | 249.1 |
| Tras-MT-VC-PABC-Compound 105 | 196.2 | 625.5 | 230.1 |
| Tras-ME2-Compound 100 | 34.6 | 25.7 | 250.6 |
| Tras-MT-VK-PABC-Compound 107 | inactive | inactive | inactive |
| Tras-MT-VK-PABC-Compound 100 | 30.5 | inactive | 129.1 |
| Tras-MT-VK-PABC-Compound 108 | inactive | inactive | Inactive |
| Tras-MT-VK-PABC-Compound 101 | 22.8 | 111 | 225.4 |
| Tras-MT-VC-PABC-Compound 112 | 44.2 | inactive | 295.5 |
| Tras-MT-VC-PABC-Compound 106 | 159.1 | 131.2 | 50 |
| Tras-MT-VC-PABC-Compound 111 | 171.6 | inactive | 65.9 |
| Tras-MT-VC-PABC-Compound 144 | 550.5 | inactive | 441.3 |
| Tras-MT-VC-PABC-Compound 187 | inactive | inactive | Inactive |
| Tras-MT-VC-PABC-Compound 188 | inactive | inactive | 471.9 |
| Tras-MT-VC-PABC-Compound 150 | 32 | inactive | 159.4 |
| Tras-MT-VC-PABC-Compound 145 | inactive | 36.4 | 161.7 |
| Tras-MT-VC-PABC-Compound 152 | 151.9 | inactive | 250.9 |
| Tras-MT-VC-PABC-Compound 154 | 50 | 174.2 | 1287 |
| Tras-MT-VC-PABC-Compound 166 | 95 | inactive | 7778 |
| Tras-MT-VC-PABC-Compound 155 | 139.7 | 77 | 2706 |
| Tras-MT-VC-PABC-Compound 132 | 180.3 | 131.1 | 359.5 |
| Tras-MT-VC-PABC-Compound 140 | 173.9 | 90.4 | 311.7 |
| Tras-MT-VC-PABC-Compound 139 | 156.8 | 154.8 | 629.6 |
| Tras-MT-VC-PABC-Compound 110 | 175.9 | 163 | 229.1 |
| Tras-MC-Compound 100 | 502 | 160.9 | 297.7 |
| Tras-MT-Compound 100 | 180.7 | 86.6 | 519.7 |
| Tras-MT-VC-PABC-Compound 172 | 176.4 | 470.9 | not tested |
| Tras-MT-VK-PABC-Compound 161 | 502.8 | 3866 | not tested |
| Tras-MT-VK-PABC-Compound 182 | 61.3 | 820.3 | not tested |
| Tras-MT-VC-PABC-Compound 100 (DAR2) | 147.2 | inactive | not tested |
| Tras-MT-VK-PABC-Compound 159 | 37.1 | 7655 | not tested |
| Tras-MT-VK-PABC-Compound 167 | 203.1 | 3931 | not tested |
| Tras-MT-VC-PABC-Compound 180 | 192.5 | 2618 | not tested |
| Tras-MT-VC-PABC-Compound 173 | 57.1 | 193.2 | not tested |
| Tras-MT-VC-PABC-Compound 169 | 94.3 | 2562 | not tested |

These data show that certain compounds that showed strong immunomodulatory activity as a free molecule (i.e., not in form of a conjugate), such as compounds 100, 101, 105, 106, 132, 139, 140, 154, 155 and 182, also demonstrated strong immunomodulatory activity when tested in an ISAC format in these assays. Some of the tested conjugates, e.g., Tras-ME2-Compound 100, Tras-MT-VK-PABC-Compound 101, Tras-MT-VC-PABC-Compound 106, outperformed the benchmark conjugate.

### Example 6: In Vivo Activity of ISACS

This example demonstrates the ability selected ISACs produced as described in EXAMPLE 4 to provide anti-tumor activity in an NCI-N87 HER2^{high} xenograft model of gastric cancer. Based on the results obtained and described in EXAMPLE 5, 12 different ISACs were selected and further tested in vivo.

### Methods and Study Design

Tumor cell suspensions (10⁷ cells in a 1:1 mix of PBS and matrigel) were implanted subcutaneously into balb/c nude mice. When mean tumor volume reached ˜160 mm³ the animals were randomly assigned to groups (n=6 per group) and treated as shown in TABLE 6 (Trastuzumab is abbreviated with “Tras”). Briefly, ISACs and antibody control were intravenously (iv) administered once on the study start date (day 0) using a dose of 2.5 mg/kg. Tumor volume and body weight were measured twice weekly with a study duration of 30 days.

TABLE 6
In Vivo Study Design and Tested ISACs

| Test Article | DAR | Dose (mg/kg) |
|---|---|---|
| Vehicle | / | 0 |
| Tras | / | 2.5 |
| Tras-benchmark compound | 3.9 | 2.5 |
| Tras-MT-VC-PABC-Compound 100 | 4.0 | 2.5 |
| Tras-ME2-Compound 100 | 3.6 | 2.5 |
| Tras-MT-VC-PABC-Compound 152 | 4.0 | 2.5 |
| Tras-MT-VC-PABC-Compound 154 | 3.1 | 2.5 |
| Tras-MT-VC-PABC-Compound 139 | 3.9 | 2.5 |
| Tras-MT-VC-PABC-Compound 132 | 4.0 | 2.5 |
| Tras-MT-VC-PABC-Compound 140 | 3.9 | 2.5 |
| Tras-MT-VC-PABC-Compound 155 | 3.8 | 2.5 |
| Tras-MT-VC-PABC-Compound 110 | 3.9 | 2.5 |
| Tras-MT-VK-PABC-Compound 101 | 3.5 | 2.5 |
| Tras-MT-VC-PABC-Compound 105 | 3.5 | 2.5 |
| Tras-MT-VC-PABC-Compound 106 | 3.7 | 2.5 |

### Results

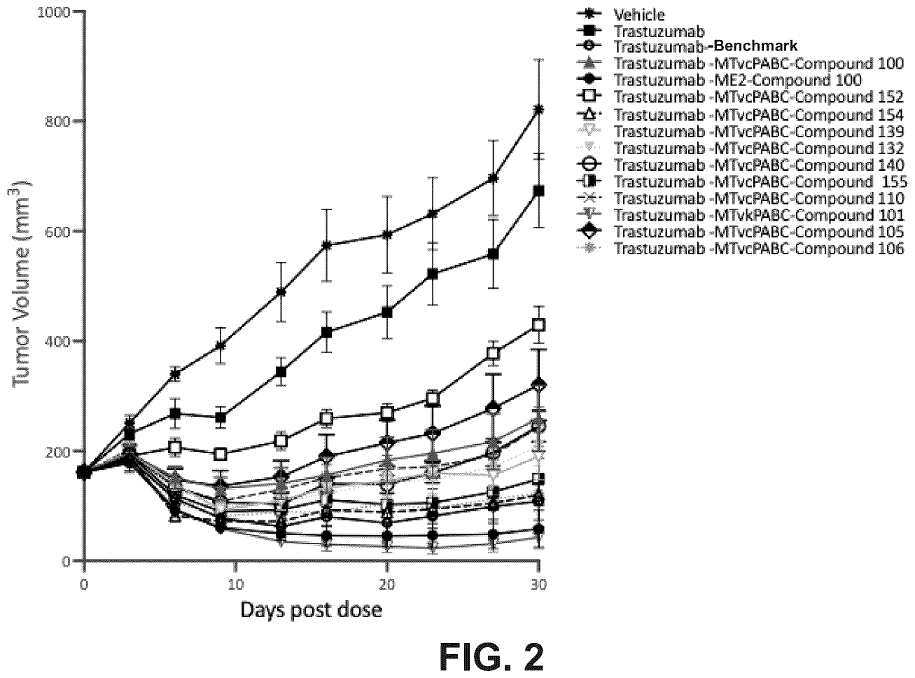

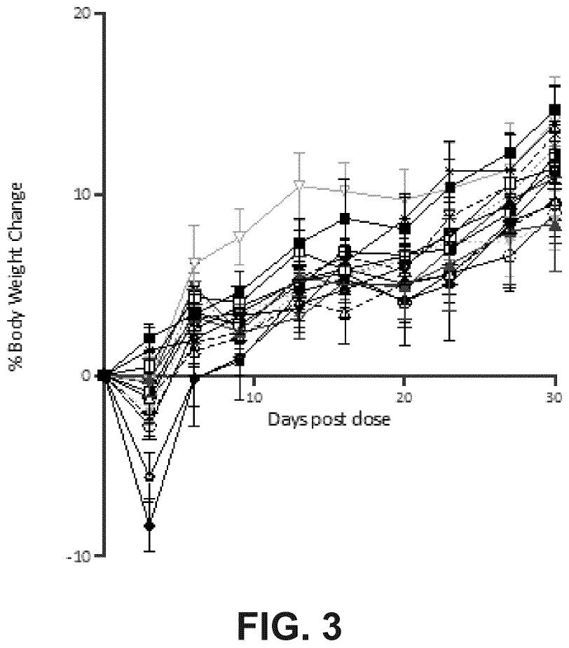

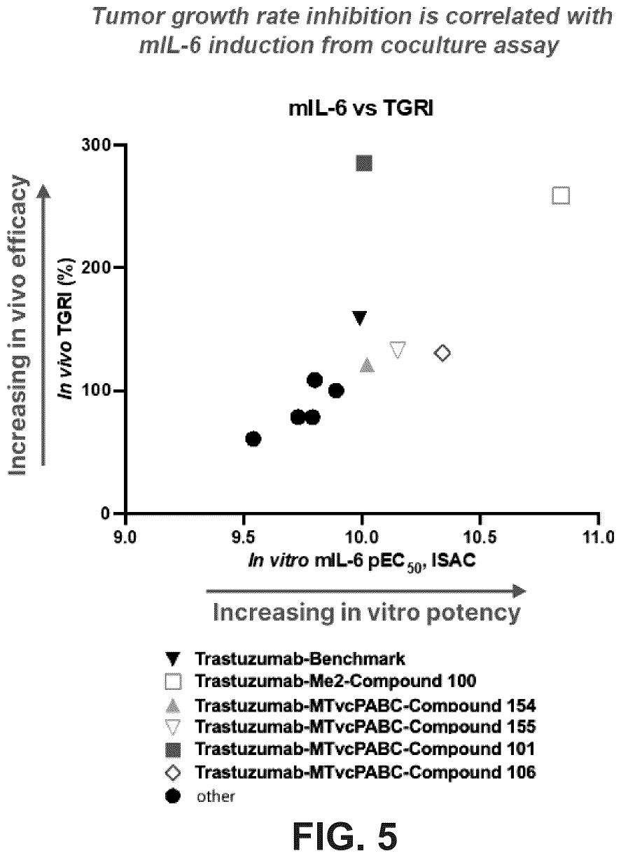

The results for in vivo anti-tumor activity and weight loss are shown in FIG. 2 and FIG. 3, respectively. All tested ISACs resulted in at least a reduction in the tumor growth rate compared to vehicle and unconjugated Trastuzumab controls, demonstrating at least some anti-tumor activity for all ISACs tested. Furthermore, FIG. 5 shows a correlation between tumor growth rate inhibition and murine IL-6 (mIL-6) induction for certain ISACs according to embodiments of the present disclosure.

Notably, the conjugates Tras-ME2-Compound 100 and Tras-MT-VK-PABC-Compound 101 each resulted in at least 50% (i.e., 3/6) complete responses, which significantly outperformed the benchmark ISAC (i.e., 0/6) complete responses, and lead to a significant reduction in tumor size of at least about 50%.

In addition, the benchmark control ISAC, Trastuzumab-benchmark compound, resulted in transient mean body weight loss of between 5-10% at 3 days post dose, which fully recovered by 7 days post dose. All other tested ISACs resulted in minor or no body weight loss indicating that they were well tolerated at the tested dose.

### Example 7: Tolerability of ISACS In Vivo

This example shows results of a tolerability study performed in vivo using selected ISACs described herein to more specifically determine the in vivo applicability of the compounds and conjugates disclosed herein, as well as to determine the in vivo tolerability of ISACs described herein relative to previously disclosed benchmark compounds.

Hence, tolerability of ISACs was assessed following a single iv injection of a solution containing the respective ISAC to balb/c mice (n=5) at the beginning of the study (study day 0) at doses outlined in TABLE 7. Body weights were recorded daily, where possible, until study termination at day 16 post dose for doses 3 mg/kg and 15 mg/kg, and day 11 post dose for doses of 45 mg/kg. Animals were sacrificed if body weight loss exceeded 20% from baseline. Vehicle and Trastuzumab at 45 mg/kg were administered as controls and were both well tolerated.

TABLE 7
In Vivo Tolerability Study Design and Tested ISACs

| Test Article | DAR | Dosage (mg/kg) |
|---|---|---|
| Vehicle | / | 0 |
| Trastuzumab | / | 45 |
| Trastuzumab-Benchmark | 3.9 | 3, 15 |
| Trastuzumab-ME2-Compound 100 | 3.6 | 3, 15, 45 |
| Trastuzumab-MT-VC-PABC-Compound 154 | 3.1 | 3, 15, 45 |
| Trastuzumab-MT-VC-PABC-Compound 155 | 3.8 | 3, 15 |
| Trastuzumab-MT-VK-PABC-Compound 101 | 3.5 | 3, 15, 45 |
| Trastuzumab-MT-VK-PABC-Compound 106 | 3.7 | 3, 15, 45 |

### Results

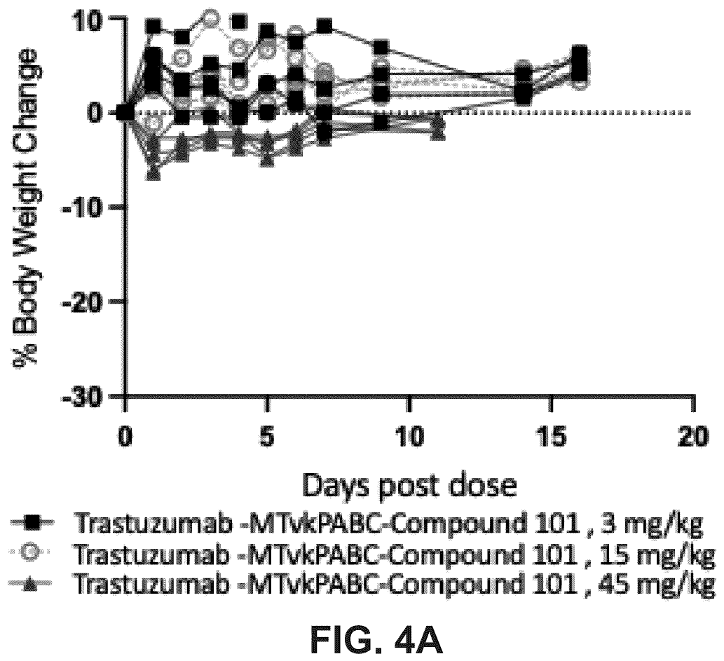

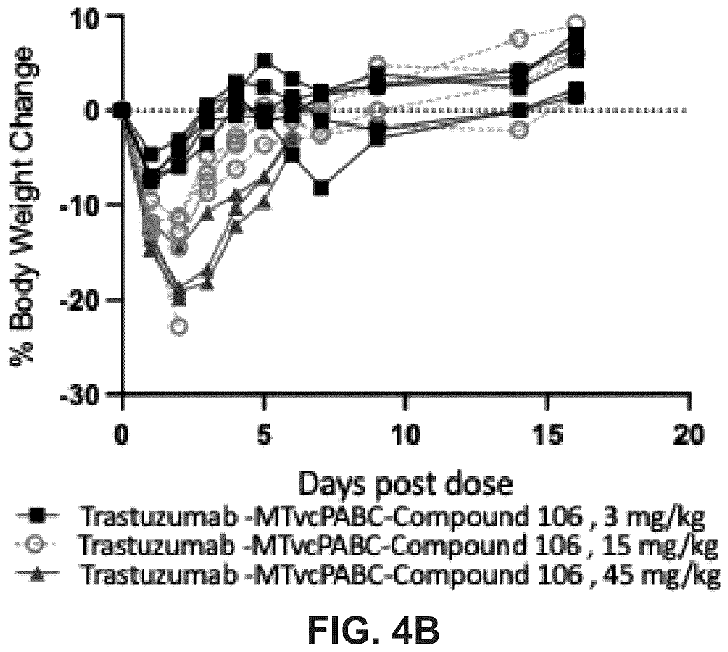

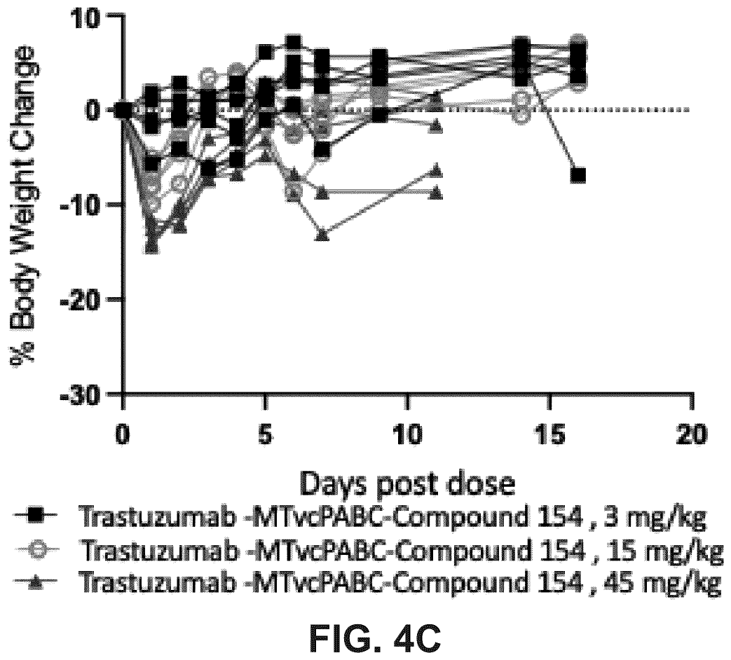

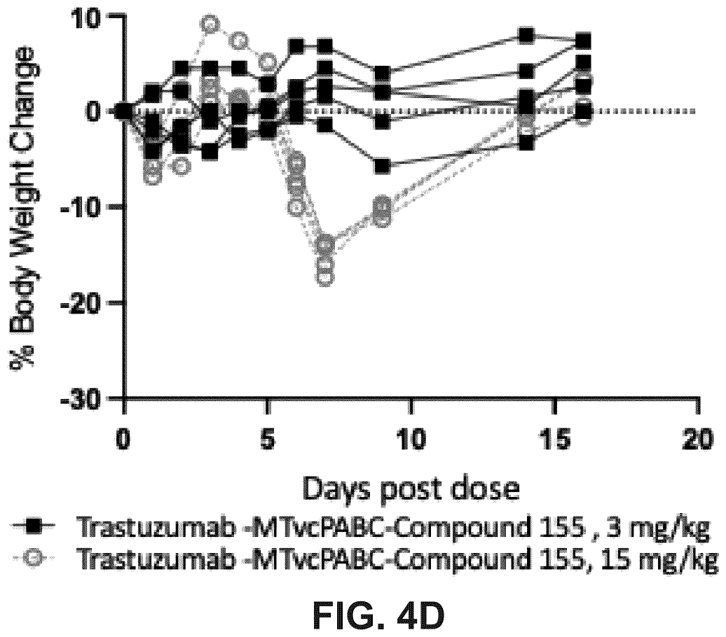

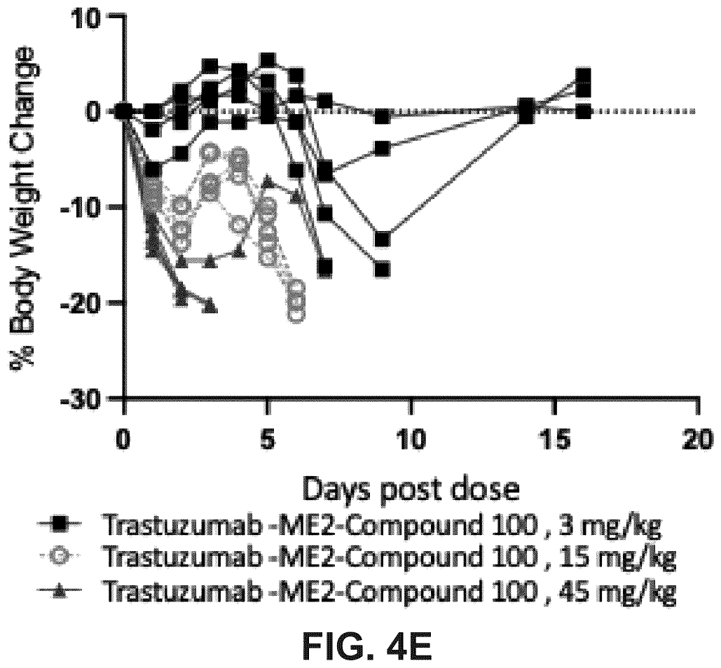

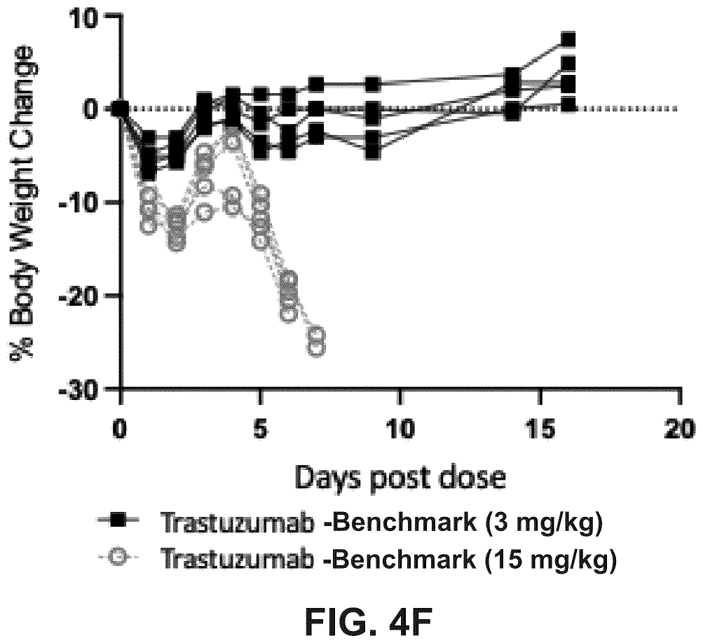

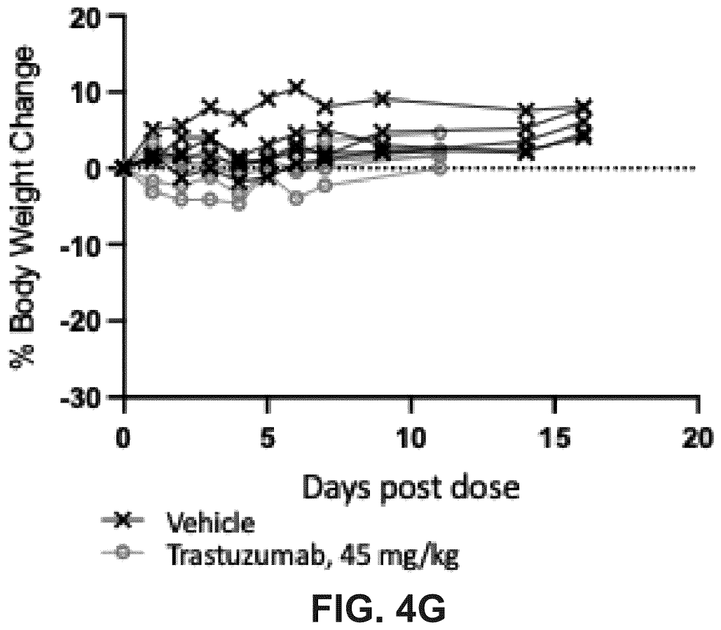

The changes in body weight (shown as %-change relative to baseline) over the course of this study and following administration of all tested ISACs at various dose levels are shown in FIGS. 4A-4G.

The data demonstrate that while the benchmark conjugate was well tolerated at 3 mg/kg, it was not tolerated at 15 mg/kg with all animals being terminated at day 7.

In contrast, Tras-MT-VK-PABC-Compound 101 was well tolerated at all dose levels tested, with only minor transient body weight loss (BWL) observed and survival of all mice at doses as high as 45 mg/kg, which was a significant improvement when compared to the benchmark conjugate.

The conjugate Tras-ME2-Compound 100, which comprised a non-cleavable linker, was not tolerated at any dose tested. At 3 mg/kg, 2/5 mice were terminated by Day 11. Remaining mice showed minor to strong BWL that recovered by 2 weeks post dose. At 15 mg/kg, animals showed 10-14% BWL at 2 days post dose that partially recovered before further BWL, but no survival was observed beyond study day 7. At 45 mg/kg, rapid BWL resulted in lethality, with 4/5 animals not surviving past day 3 of the study. It should be noted, however, that this conjugate was well tolerated at 2.5 mg/kg, which was a dose level that was effective in reducing tumor volume (see Example 6).

Doses of the ISAC Tras-MT-VC-PABC-Compound 154 of up to 45 mg/kg did not result in any loss of animals from the study. At doses of 3 mg/kg, minor BWL was observed at 3 days post dose that fully recovered. At doses of 15 mg/kg, rapid BWL loss of 5-10% was observed in all animals at day 2 that rapidly recovered by day 3. A secondary moderate reduction in BW was observed in most animals at day 6 that rapidly recovered. At 45 mg/kg, rapid BWL was observed at 1 day post dose of between 12-14% that partially recovered by˜day 5, before a secondary minor BWL at day 6-7 was observed, followed by recovery and survival of all animals on study.

The ISAC Tras-MT-VC-PABC-Compound 155 resulted in only minor BWL in most animals at doses of 3 mg/kg between day 1-3 post dose, followed by recovery. At doses of 15 mg/kg, rapid BWL of between 3-7% was observed at 1 day post dose, followed by rapid recovery and a secondary BWL at day 7 of between 14-17% with rapid recovery of 4/5 animals and loss of 1 animal.

Finally, the ISAC Tras-MT-VK-PABC-Compound 106 resulted in moderate BWL of between 5-8% at doses of 3 mg/kg at day 1 post dose, followed by recovery. At doses of 15 mg/kg, rapid BWL was observed at 1 day post dose of between 11-23%, resulting in loss of 1/5 animals from study, while 4/5 animals recovered. At 45 mg/kg, rapid BWL of between 14-20% resulted in 2/5 animals not surviving past day 2, with recovery of the remaining animals.

What is claimed is:

1. A compound of Formula (I): 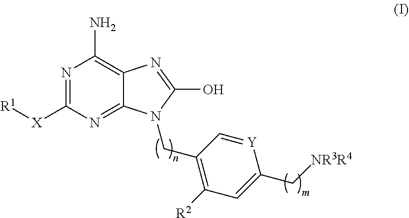
a tautomer, a protected version, and/or a pharmaceutically acceptable salt thereof, wherein:
X is O or NH;
Y is N or CH;
R¹ is optionally substituted C₂-C₆ alkyl or branched and optionally substituted C₃-C₈ hydroxyalkyl;
R² is H, halogen, optionally substituted C₁-C₆ alkyl, or optionally substituted C₁-C₆ alkoxy;
R³ and R⁴ are independently H, Q-R⁵, or R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or 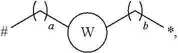
wherein * is the point of attachment to R⁵ and #is the point of attachment to N;
R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R⁶ and R⁷ are independently H, optionally substituted C₁-C₆ alkyl, optionally substituted C₁-C₆ alkoxycarbonyl, or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
n is an integer from 1 to 4;
m is an integer from 0 to 4; and
a and b are each independently integers from 0 to 4,
with the proviso that when R² is H, then:
(i) R¹ is branched and optionally substituted C₃-C₈ hydroxyalkyl;
(ii) X is NH and R¹ is optionally substituted C₅-C₆ alkyl; or
(iii) m is 0 or 1 and R³ and R⁴ together with the N form an unsubstituted piperazinyl ring or an optionally substituted C₆-C₁₀-heterospirocycloalkyl ring.
The compound of claim 1, wherein R² is halogen, optionally substituted C₁-C₆ alkyl, or optionally substituted C₁-C₆ alkoxy.

2. The compound of claim 1, wherein R² is halogen, optionally substituted C₁-C₆ alkyl, or optionally substituted C₁-C₆ alkoxy.

3. The compound of any one of claims 1-2, wherein R² is halogen or optionally substituted C₁-C₆ alkoxy.

4. The compound of any one of claims 1-3, wherein R² is halogen or unsubstituted C₁-C₆ alkoxy.

5. The compound of any one of claims 1-4, wherein R² is fluorine or methoxy.

6. The compound of any one of claims 1-5, wherein X is O.

7. The compound of any one of claims 1-5, wherein X is NH.

8. The compound of any one of claims 1-7, wherein R¹ is optionally substituted C₂-C₆ alkyl.

9. The compound of any one of claims 1-7, wherein R¹ is branched and optionally substituted C₃-C₈ hydroxyalkyl.

10. The compound of any one of claims 1-9, wherein R³ and R⁴ are independently H, or Q-R⁵.

11. The compound of any one of claims 1-10, wherein n is 1.

12. The compound of any one of claims 1-11, wherein m is an integer from 0 to 3 or from 1 to 3.

13. The compound of claim 1, wherein R² is H and R¹ is branched and optionally substituted C₃-C₈ hydroxyalkyl.

14. The compound of claim 1, wherein the compound is selected from any one of compounds 100-195 listed in TABLE 1.

15. The compound of claim 1, having the structure of Formula (II): 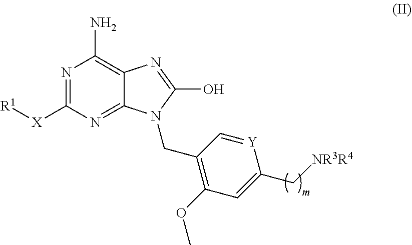
wherein:
X is O or NH;
Y is N or CH;
R¹ is optionally substituted C₂-C₆ alkyl or branched and optionally substituted C₃-C₈ hydroxyalkyl;
R³ and R⁴ are independently H, Q-R⁵, or R³ and R⁴ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or 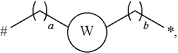
wherein * is the point of attachment to R⁵ and #is the point of attachment to N;
R⁵ is H, OH, NR⁶R⁷, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R⁶ and R⁷ are independently H, optionally substituted C₁-C₆ alkyl, optionally substituted C₁-C₆ alkoxycarbonyl, or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
m is an integer from 0 to 4; and
a and b are independently integers from 0 to 4.

16. The compound of claim 15, wherein the compound is selected from SUBTABLE 1A.

17. The compound of any one of claims 1-9, wherein the compound is selected from SUBTABLE 1B.

18. The compound of claim 15, wherein the compound is:

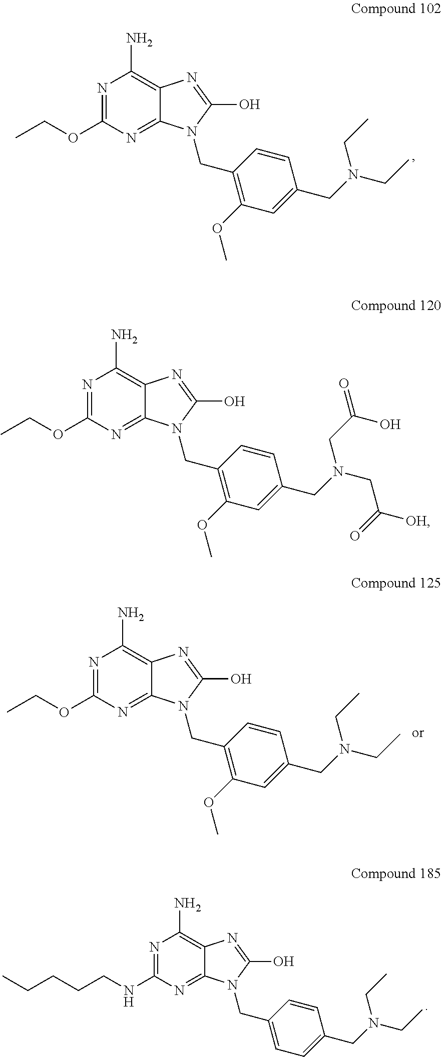

19. The compound of claim 1-10, wherein R³ and R⁴ are both H.

20. The compound of claim 19, wherein the compound is:

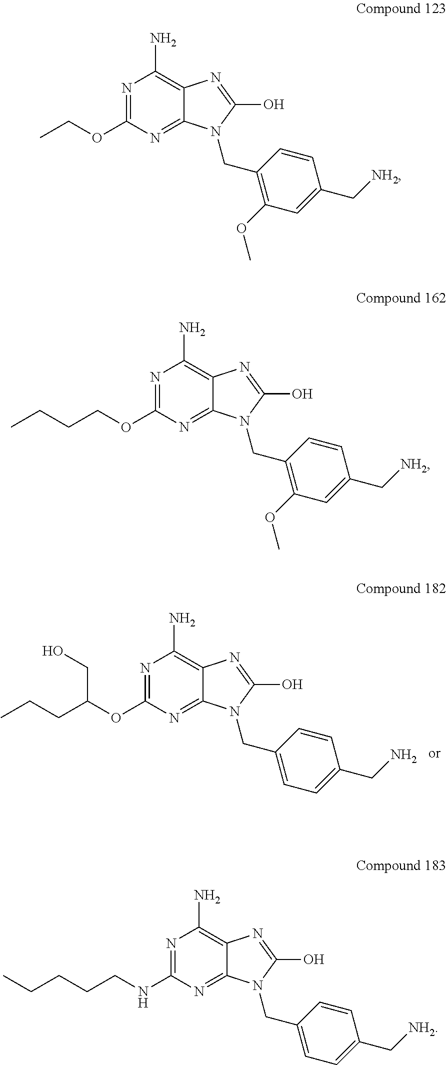 [SMILES: CCOc1nc(N)c2nc(O)n(Cc3ccc(CN)cc3OC)c2n1 ; CCCCOc1nc(N)c2nc(O)n(Cc3ccc(CN)cc3OC)c2n1 ; CCCC(CO)Oc1nc(N)c2nc(O)n(Cc3ccc(CN)cc3)c2n1 ; "or" ; CCCCCNc1nc(N)c2nc(O)n(Cc3ccc(CN)cc3)c2n1]

21. The compound of claim 1, wherein the compound is selected from SUBTABLE 1C.

22. The compound of claim 1 or claim 15, wherein the compound is:

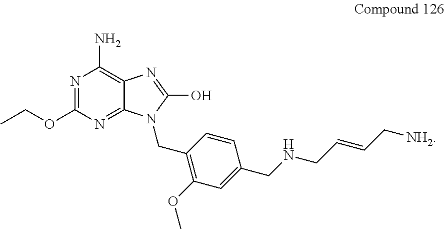 [SMILES: CCOc1nc(N)c2nc(O)n(Cc3ccc(CNC/C=C/CN)cc3OC)c2n1]

23. The compound of any one of claims 1-10, wherein the compound is selected from SUBTABLE 1D.

24. The compound of any one of claims 1-10, wherein R⁶ is H and R⁷ is H, optionally substituted C₁-C₆ alkyl or R⁶ and R⁷ together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring.

25. The compound of claim 24, wherein the compound is selected from SUBTABLE 1E.

26. The compound of any one of claims 1-9, wherein the compound is:

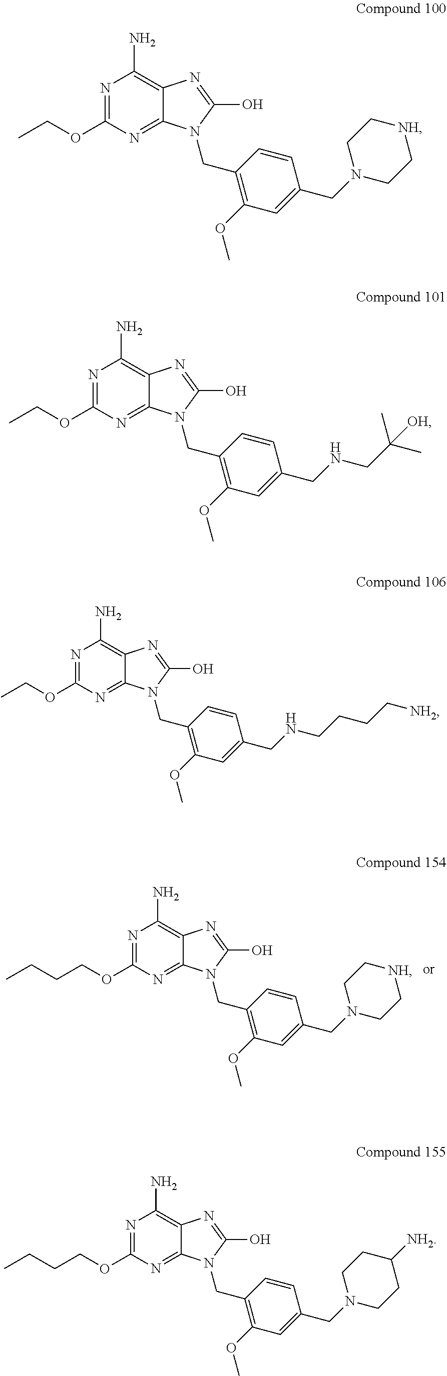

27. The compound of claim 1 or claim 15, wherein the compound is:

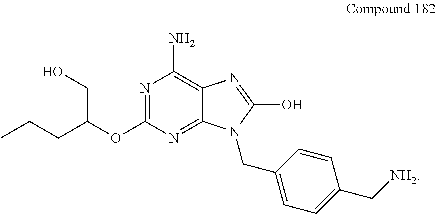 [SMILES: CCCC(CO)Oc1nc(N)c2nc(O)n(Cc3ccc(CN)cc3)c2n1]

28. A compound of Formula (III): 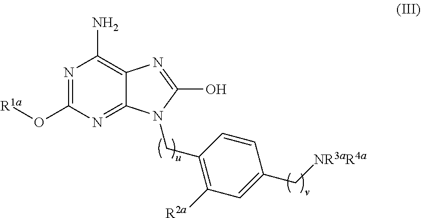
a tautomer, a protected version, and/or a pharmaceutically acceptable salt thereof, wherein:
R^{1a} is optionally substituted C₂-C₆ alkyl or branched and optionally substituted C₃-C₈ hydroxyalkyl;
R^{2a} is halogen, optionally substituted C₁-C₆ alkyl, or optionally substituted C₁-C₆ alkoxy;
R^{3a} and R^{4a} are independently H, Q-R^{5a}, or R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or 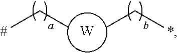
wherein * is the point of attachment to R⁵ and #is the point of attachment to N;
R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R^{6a} and R^{7a} are independently H, optionally substituted C₁-C₆ alkyl, optionally substituted C₁-C₆ alkoxycarbonyl, or R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₃-C₆-heterocycloalkyl, optionally substituted C₅-C₇-heterobicycloalkyl, optionally substituted C₆-C₁₀-heterospirocycloalkyl, optionally substituted aryl, or optionally substituted heteroaryl;
u is an integer from 1 to 3;
v is an integer from 0 to 4; and
a and b are independently integers from 0 to 4.

29. The compound of claim 28, having the structure of Formula (IV): 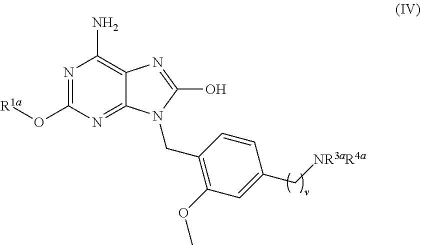
wherein:
R^{1a} is unsubstituted C₂-C₆ alkyl or branched and unsubstituted C₃-C₈ hydroxyalkyl;
R^{3a} and R^{4a} are independently H, Q-R^{5a}, or R^{3a} and R^{4a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
Q is a bond, optionally substituted C₁-C₆-alkyl, optionally substituted C₂-C₆-alkenyl or 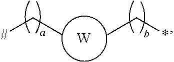
wherein * is the point of attachment to R⁵ and #is the point of attachment to N;
R^{5a} is H, OH, NR^{6a}R^{7a}, optionally substituted C₃-C₆-carboxyalkyl, optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
R^{6a} and R^{7a} are independently H, optionally substituted C₁-C₆ alkyl, optionally substituted C₁-C₆ alkoxycarbonyl, or R^{6a} and R^{7a} together with the N form an optionally substituted C₂-C₆-heterocycloalkyl ring, optionally substituted C₃-C₇-heterobicycloalkyl ring, optionally substituted C₆-C₁₀-heterospirocycloalkyl ring or an optionally substituted heteroaryl ring;
W is optionally substituted C₃-C₆-cycloalkyl, optionally substituted C₂-C₆-heterocycloalkyl, optionally substituted C₃-C₇-heterobicycloalkyl, optionally substituted C₆-C₁₀-heterospirocycloalkyl, optionally substituted aryl, or optionally substituted heteroaryl;
v is an integer from 0 to 4; and
a and b are independently integers from 0 to 4.

30. The compound of claim 28 or claim 29, wherein each alkyl, alkenyl, cycloalkyl, spirocycloalkyl, heterocycloalkyl, heterospirocycloalkyl, aryl and heteroaryl group is optionally substituted —NH₂, —CO₂H, —OH, carbonyl, halogen, C₁-C₄ alkyl, C₁-C₄ hydroxyalkyl, C₁-C₄ aminoalkyl, C₁-C₄ carboxyalkyl, (C₀-C₄ alkyl)-cycloalkyl, (C₀-C₄ alkyl)-heterocycloalkyl, (C₀-C₄ alkyl)-spirocycloalkyl, (C₀-C₄ alkyl)-heterospirocycloalkyl, (C₀-C₄ alkyl)-aryl, and (C₀-C₄ alkyl)-heteroaryl, and wherein each of the alkyl, hydroxyalkyl, C₁-C₄ aminoalkyl, C₁-C₄ carboxyalkyl, cycloalkyl, heterocycloalkyl, spirocycloalkyl, heterospirocycloalkyl, aryl or heteroaryl group can itself be substituted with one or more of: —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl, or unsubstituted C₁-C₄ carboxyalkyl.

31. The compound of claim 30, wherein each alkyl, alkenyl, cycloalkyl, spirocycloalkyl, heterocycloalkyl, heterospirocycloalkyl, aryl and heteroaryl group is optionally substituted with one or more substituents selected from the group consisting of —NH₂, —CO₂H, —OH, carbonyl, halogen, unsubstituted C₁-C₄ alkyl, unsubstituted C₁-C₄ hydroxyalkyl, unsubstituted C₁-C₄ aminoalkyl and unsubstituted C₁-C₄ carboxyalkyl.

32. The compound of claim 28 or claim 29, wherein the compound is selected from SUBTABLE 1A.

33. The compound of claim 28 or claim 29, wherein the compound is:

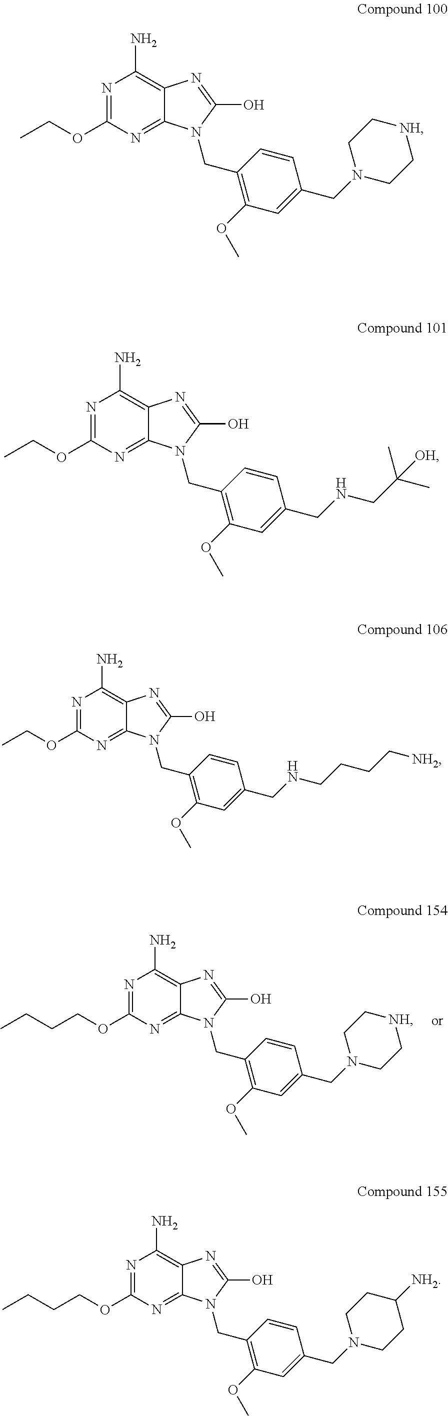

34. The compound of any one of claims 1-33, wherein the compound has an EC₅₀ value for agonizing human or murine TLR7 of <500 nM, <250 nM, or <100 nM as determined in a reporter gene assays, and/or an EC₅₀ value for inducing IL6 production in a human or murine immune cell population of <5 pM, <1 pM, <500 nM, or <100 nM, as determined in an immune cell assay.

35. A pharmaceutical composition comprising a compound of any one of claims 1-34, and a pharmaceutically acceptable carrier or diluent.

36. A compound of any one of claims 1-34 for use in therapy.

37. A compound of any one of claims 1-34 for use in the treatment of cancer.

38. Use of a compound of any one of claims 1-34 in the manufacture of a medicament for the treatment of cancer.

39. A compound-linker construct having Formula (A):
L-(C)ₚ (A)
wherein:
L is a linker;
C is a compound of any one of claims 1-34; and
p is an integer from 1 to 5.

40. The compound-linker construct of claim 39, wherein L is a cleavable linker.

41. The compound-linker construct of claim 39, wherein L is a non-cleavable linker.

42. The compound-linker construct of any one of claims 39-40, wherein L comprises a dipeptide, tripeptide, tetrapeptide, or a combination thereof.

43. The compound-linker construct of any one of claims 39-42, wherein L comprises a polyethylene glycol (PEG) moiety.

44. The compound-linker construct of any one of claims 39-40, wherein L is a protease cleavable linker.

45. The compound-linker construct of any one of claims 39-44, wherein C is the compound of claim 36.

46. The compound-linker construct of any one of claims 39-44, wherein C is the compound of claim 66.

47. The compound-linker construct of claim 39, wherein the compound-linker is selected from TABLE 2B.

48. The compound-linker construct of any one of claims 39-46, wherein p is 1, 2, or 3.

49. A conjugate having Formula (X):
T-[L-(C)ₚ]ᵣ (X)
wherein:
T is a targeting moiety;
L is a linker;
C is a compound of any one of claims 1-34;
p is an integer from 1 to 5; and
r has a value from about 1 to about 8.

50. The conjugate of claim 49, wherein L-(C), is the compound-linker construct of any one of claims 39-48.

51. The conjugate of any one of claims 49-50, wherein r is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0.

52. The conjugate of claim 49, wherein C is selected from SUBTABLE 1F.

53. The conjugate of any one of claims 49-52, wherein T binds to a tumor associated antigen (TAA).

54. The conjugate of any one of claims 49-53, wherein T is an antibody or antigen-binding antibody fragment.

55. The conjugate of claim 54, wherein the antibody is a bispecific or multispecific antibody.

56. The conjugate of any one of claims 49-55, wherein the conjugate has an EC₅₀ value for inducing production of a cytokine in a human or murine immune cell population of <1 nM, <500 pM, or <100 pM, as determined in a reporter gene assay.

57. A pharmaceutical composition comprising the conjugate of any one of claims 49-56, and a pharmaceutically acceptable carrier or diluent.

58. A conjugate of any one of claims 49-56 for use in therapy.

59. A conjugate of any one of claims 49-56 for use in the treatment of cancer.

60. Use of a conjugate of any one of claims 49-56 in the manufacture of a medicament for the treatment of cancer.
